(19) United States
(12) Patent Application Publication
(10) Pub. No.: US 2024/0263159 A1
(43) Pub. Date: Aug. 8, 2024

(54) GENE EDITING IN PRIMARY IMMUNE CELLS USING CELL PENETRATING CRISPR-CAS SYSTEM

(71) Applicant: THE TRUSTEES OF THE UNIVERSITY OF PENNSYLVANIA, Philadelphia, PA (US)

(72) Inventors: Shelley L. Berger, Wayne, PA (US); E. John Wherry, Havertown, PA (US); Junwei Shi, Philadelphia, PA (US); Zeyu Chen, Medford, MA (US); Zhen Zhang, Philadelphia, PA (US); Rahul M. Kohli, Penn Valley, PA (US); Jared B. Parker, Elkton, MD (US); Amy Elizabeth Baxter, Philadelphia, PA (US)

(21) Appl. No.: 18/566,410

(22) PCT Filed: Jun. 2, 2022
(86) PCT No.: PCT/US22/31983

§ 371 Date: Dec. 1, 2023

Related U.S. Application Data
(60) Provisional application No. 63/196,144, filed on Jun. 2, 2021.

(51) Int. Cl. C12N 9/22; C12N 15/10; C12N 15/11

(52) U.S. Cl. CPC C12N 9/22; C12N 15/102; C12N 15/111; C07K 2319/06; C07K 2319/09; C07K 2319/10; C12N 2310/20

(57) ABSTRACT

The present disclosure provides compositions and methods for in vitro and in vivo gene editing using a cell penetrating CRISPR-Cas system comprising a cell penetrating Cas and an endosomal escape peptide.

### CROSS-REFERENCE TO RELATED APPLICATION

The present application is entitled to priority under 35 U.S.C. § 119(e) to U.S. Provisional Patent Application No. 63/196,144, filed Jun. 2, 2021, which is incorporated by reference in its entirety herein.

### STATEMENT REGARDING FEDERALLY SPONSORED RESEARCH OR DEVELOPMENT

This invention was made with government support under AI117950, AI108565, and CA077831 awarded by the National Institutes of Health. The government has certain rights in the invention.

### BACKGROUND OF THE INVENTION

CRISPR-Cas systems provide a valuable tool for gene editing. However, most methods for editing primary T cells require electroporation, can cause off-target genomic effects, and can be costly. There is a need in the art for CRISPR-Cas systems that achieve high gene editing efficiency both in vitro and in vivo, and are less expensive and easily implemented into experimental workflows. The present invention addresses this need.

### SUMMARY OF THE INVENTION

As described herein, the present disclosure provides compositions and methods for Peptide-Assisted Genome Editing (PAGE). In one aspect, the disclosure provides a Peptide-Assisted Genome Editing (PAGE) system comprising a) a CRISPR associated (Cas) protein linked to a Cell Penetrating Peptide (CPP), and b) an endosomal escape peptide linked to a CPP.

In certain embodiments, the Cas is Cas9, or Cas12a, or a Cas derivative. In certain embodiments, the Cas derivative is a Cas protein linked to another protein or catalytic domain. In certain embodiments, the protein or catalytic domain is selected from the group consisting of an AID deaminase, an APOBEC deaminase, a TadA deaminase, a TET enzyme, a DNA methyltransferase, a transactivation domain, a reverse transcriptase, a histone acetyltransferase, a histone deacetylase, a sirtuin, a histone methyltransferase, a histone demethylase, a kinase, and a phosphatase.

In certain embodiments, the endosomal escape peptide comprises any of the amino acid sequences set forth in SEQ ID NOs: 1434-1523. In certain embodiments, the endosomal escape peptide comprises dTAT-HA2.

In certain embodiments, the Cas comprises a Nuclear Localization Signal (NLS) sequence. In certain embodiments, the NLS sequence comprises the amino acid sequence PAAKRVKLD (SEQ ID NO: 1). In certain embodiments, the NLS sequence further comprises a GGS linker.

In certain embodiments, the CPP comprises any of the amino acid sequences set forth in SEQ ID NOs: 10-1422. In certain embodiments, the CPP comprises a sequence derived from the trans-activating transcriptional activator (Tat) from HIV-1. In certain embodiments, the Tat sequence comprises the amino acid sequence GRKKRRQRRRPQ (SEQ ID NO: 2).

Another aspect of the disclosure provides an in vitro method of gene editing comprising introducing into a cell a PAGE system and at least one sgRNA or crRNA, wherein the PAGE system comprises a Cas protein linked to a CPP and an endosomal escape peptide linked to a CPP.

Another aspect of the disclosure provides an in vivo method of gene editing comprising introducing into a cell a PAGE system and at least one sgRNA or crRNA, wherein the PAGE system comprises a Cas protein linked to a CPP and an endosomal escape peptide linked to a CPP, and administering the cell to a subject.

In certain embodiments, the Cas is Cas9, or Cas12a, or a Cas derivative. In certain embodiments, the Cas derivative is a Cas protein linked to another protein or catalytic domain. In certain embodiments, the protein or catalytic domain is selected from the group consisting of an AID deaminase, an APOBEC deaminase, a TadA deaminase, a TET enzyme, a DNA methyltransferase, a transactivation domain, a reverse transcriptase, a histone acetyltransferase, a histone deacetylase, a sirtuin, a histone methyltransferase, a histone demethylase, a kinase, and a phosphatase.

In certain embodiments, the endosomal escape peptide comprises any of the amino acid sequences set forth in SEQ ID NOs: 1434-1523. In certain embodiments, the endosomal escape peptide comprises dTAT-HA2.

In certain embodiments, the Cas comprises a NLS sequence. In certain embodiments, the NLS sequence comprises the amino acid sequence PAAKRVKLD (SEQ ID NO: 1). In certain embodiments, the NLS sequence further comprises a GGS linker.

In certain embodiments, the CPP comprises any of the amino acid sequences set forth in SEQ ID NOs: 10-1422. In certain embodiments, the CPP comprises a sequence derived from the trans-activating transcriptional activator (Tat) from HIV-1. In certain embodiments, the Tat sequence comprises the amino acid sequence GRKKRRQRRRPQ (SEQ ID NO: 2).

In certain embodiments, the method does not require electroporation. In certain embodiments, the PAGE system is introduced into the cell in a medium that does not contain serum. In certain embodiments, the endosomal escape peptide is introduced into the cell at a concentration of about 25-75 μM. In certain embodiments, the Cas is introduced into the cell at a concentration of about 0.5-5 μM.

In certain embodiments, cell is an immune cell.

In certain embodiments, the cell is selected from the group consisting of a primary human CD8 T cell, a human iPSC, and a CAR T cell.

In certain embodiments, the sgRNA targets Ano9, Pdcd1, Thy1, Ptprc, PTPRC, or B2M.

In certain embodiments, the subject is in need of a treatment for a disease or disorder, and wherein when the edited cell is administered to the subject, the disease or disorder is treated in the subject. In certain embodiments, the disease or disorder is an infection. In certain embodiments, the disease or disorder is related to T cell exhaustion.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1A: Schematic of creation of a TAT-4×Myc NLS-Cas9 expression construct.

FIG. 1B: Purification of TAT-4×Myc NLS-Cas9. TAT-4×Myc NLS-Cas9 expression construct was transformed to Rosetta 2 (DE3) pLysS and induced by IPTG. Lane 1: bacterial lysate after IPTG induction; Lane 2: flowthrough of Strep-Tactin affinity purification; Lane 3: flowthrough after on column digestion by SUMO protease ULP1; Lane 4: TAT-4×Myc NLS-Cas9 after IEC (HiTrap SP HP); Lane 5: TAT-4×Myc NLS-Cas9 after SEC (Superdex 200 increase 10/300 GL).

FIG. 1C: TAT-4×Myc NLS-Cas9 in vitro cleavage assay. Cas9 RNPs were assembled by incubating purified spCas9 or TAT-4×Myc NLS-Cas9 with sgRNA targeting a 8.7 kb DNA fragment, and incubated with the DNA fragment for 1, 5, 10, and 15 min at 37° C. The reaction was stopped and the DNA products were separated on a 0.8% agarose gel. Uncut band (˜8.7 kb) and two cleaved bands (˜2.7 kb and 6 kb) are shown on the gel.

FIG. 2A: Schematic of EL4 mCherry reporter cell line for Cas9 editing efficiency and experiment workflow. EL4 cells were infected by a lentiviral reporter construct stably expressing mCherry and sgRNA targeting mCherry. When incubated with TAT-4×Myc NLS-Cas9 and dTAT-HA2, the cells lost mCherry fluorescence and resulted in an increase of frequency of mCherry⁻ cells after 4 days as measured by flow cytometry.

FIG. 2B: Higher concentration of endosome escape peptide (dTAT-HA2) and TAT-4×Myc NLS-Cas9 increased editing efficiency. EL4 reporter cells were incubated without or with 10 or 40 μM dTAT-HA2, and 0.5 or 4.0 μM TAT-4×Myc NLS-Cas9 for 1 hour. Completed medium was replaced after the incubation and flow analysis was performed 4 days post incubation.

FIG. 2C: Low FBS increased editing efficiency. EL4 reporter cells were incubated without or with 10 or 40 μM dTAT-HA2, and 0.5 or 4.0 μM TAT-4×Myc NLS-Cas9 for 1 hour in the presence of 10% FBS or no FBS. Flow analysis was performed 4 days post incubation.

FIG. 2D: EL4 reporter cells were incubated without or with 75 μM dTAT-HA2, and or 5.0 μM TAT-4×Myc NLSCas9 for 30 min in the presence of 10% FBS or no FBS. Flow analysis was performed 4 days post incubation.

FIG. 3A: Testing CD45.2 sgRNA efficiency in RN2-Cas9 cells. RN2-Cas9 cells stably expressing Cas9 were infected with retrovirus expressing mCherry and sgRNA targeting cell surface maker CD45.2 or a control sgRNA targeting Rosa26. CD45.2 expression level was measured by flow cytometry after 3 days of infection.

FIG. 3B: A schematic of the experiment showing in vitro editing of TAT-4×Myc-NLS Cas9 in mouse primary T cells.

FIG. 3C: An example of flow plots showing the percentage of sgRNA+Cas9+ for Rosa and CD45.2.

FIG. 3D: Target genes are knocked down during Days 1-5 in vitro culture as measured by flow cytometry.

FIG. 3E: Normalized MFI of GFP (TAT-4×Myc NLS-Cas9) of sorted sgRNA+ cells during in vitro culture. MFI of sgRNA+ cells is normalized to the MFI of sgRNA+ cells without TAT-4×Myc-NLS Cas9 treatment.

FIG. 4A: A schematic of workflow showing TAT-4×Myc NLS Cas9 in vivo editing in mouse primary T cells. Donor mice CD8 P14 cells were isolated and activated, and recipient mice were infected with LCMV-Clone 13 on Day −2. After 24 hours (Day −1), cells were infected with a retroviral vector (VEX+) expressing sgRNA targeting Ano9 or Pdcd1 (encoding PD-1) for 24 hours. Cells were then treated with TAT-4×Myc NLS-Cas9, dTAT-HA2, 0.25% trypsin, and DNase I, and sgRNA+Cas9+(VEX+GFP+) P14 cells were sorted. 5×10⁴ of sorted cells were adoptively transferred to LCMV-Clone 13 infected recipient mice. After 6 days, spleen and liver were harvested and analyzed by flow cytometry for PD-1 expression level and P14 cell expansion.

FIG. 4B: Flow plots of Cas9GFP+ transduction into the sgRNA+ P14 cells.

FIG. 4C: Histogram and statistical analysis of PD-1 knock down efficiency of P14 cells in the liver and spleen at D6 post cell transfer. Ano9_e3.2 sgRNA is used as control here.

FIG. 4D: Knocking down of PD-1 induces T cell expansion at Day 6 post cell transfer in the liver and spleen.

FIG. 5A: A schematic of workflow showing TAT-4×Myc NLS-Cas9 in vitro editing in human primary T cells. Human total T cells were isolated from normal donor PBMCs, and activated by CD3/CD28 Dynabeads, IL-2, and IL15 on Day 0. After 24 hours (Day 1), cells were infected by the lentiviral reporter construct as in FIG. 2A for 2 days. On Day 3-9, mCherry+ cells were selected by blasticidin, and subsequently treated by TAT-4×Myc NLS-Cas9 and dTAT-HA2. Frequency of mCherry⁻ cells was measured on Day 12-14 by flow cytometry.

FIG. 5B: Percentages of mCherry− human T cells were measured by flow cytometry on Day 3 and 5 post-treatment of cells with dTAT-HA2 and TAT-4×Myc NLS-Cas9. T cells were isolated from three normal donors.

FIG. 5C: Example histogram of mCherry− human T cells on Day 5 post-treatment of cells with 0.5 μM TAT-4×Myc NLS-Cas9 and 50 μM dTAT-HA2.

FIG. 6A: iPSCs were infected by the same lentiviral reporter construct as in FIG. 2A. When incubated with TAT-4×Myc NLS-Cas9 and dTAT-HA2, the cells lost mCherry fluorescence and resulted in an increase of frequency of mCherry⁻ cells after 4 days as measured by flow cytometry.

FIG. 6B: Example histogram of mCherry− iPSCs on Day 4 post treatment of cells with dTAT-HA2 and TAT-4×Myc NLS-Cas9.

FIG. 7: Schematic of Peptide-Assisted Genome Editing (PAGE) system constructs used in this study. TH: dTAT-HA2; T: TAT; H: HA2; Cas9-T6N^{CPP}: TAT-4×NLS^{MYC}-Cas9-2×NLS^{SV40}-sfGFP; Cas9-T8N^{CPP}: TAT-6×NLS^{MYC}-Cas9-2×NLS^{SV40}-sfGFP; Cas9-TH6N^{CPP}: TAT-HA2-4×NLS^{MYC}-Cas9-2×NLS^{SV40}-sfGFP; Cas9-R6N^{CPP}: R9-4×NLS^{MYC}-Cas9-2×NLS^{SV40}-sfGFP; Cas9-6N^{CPP}: 4×NLS^{MYC}-Cas9-2×NLS^{SV40}-sfGFP; Cas9-6S^{CPP}: 4×NLS^{SV40}-Cas9-2×NLS^{SV40}-sfGFP; opCas12a-T8N^{CPP}: TAT-6×NLS^{MYC}-opCas12a-2×NLS^{SV40}-sfGFP; Cas9-BE-T6N^{CPP}: TAT-4×NLS^{MYC}-evoA1-nCas9-2×NLS^{SV40}-sfGFP; RNP: ribonucleoprotein; sgRNA: single guide RNA (related to Cas9); crRNA: crispr RNA (related to opCas12a); P14: LCMV-P14 T Cell Receptor; Cas9-BE: Cas9-Base Editor; d2GFP: destabilized GFP fluorescence protein.

FIGS. 8A-8E: Optimization of peptide assisted cell-penetrating Cas9 system in EL4 reporter cells. FIG. 8A: A schematic of EL4 mCherry reporter cell line for Cas9-CPP editing efficiency. EL4, a murine T lymphoblast, was lentivirally transduced with a dual expression vector stably expressing a mCherry (mChe) fluorescence reporter and an sgRNA targeting the mCherry gene or an sgRNA targeting Ano9 gene as a negative control. EL4-mChe cells were incubated with various Cas9-CPP proteins together with various endosomal escaping or cell penetrating chemical compounds or peptides. Protein, chemical, and peptide were washed out after 30 min incubation. Gene editing efficiency was evaluated by loss of mChe fluorescence at day 4 post-treatment via flow cytometry. FIG. 8B: Quantification of the editing efficiency of Cas9-T6N^{CPP} with various endosomal escaping or cell penetrating chemical compounds and peptides in EL4-mChe reporter. EL4 mChe reporter cells were treated with 0.5 μM Cas9-T6N^{CPP} in the presence of the chemical compounds 200 mM chloroquine or 1 mg/ml polybrene, or 75 μM of the assisting peptides KALA, Transportan, Penetratin, Penetratin-Arg, dTAT-HA2E5, or TH. To measure editing efficiency, the percentage of cells with loss of mCherry was measured by flow cytometry at day 4 post-treatment. Among these chemical compounds and CPP peptides tested, TH (dTAT-HA2) incubation showed the highest percentage of mCherryOFF (>90%), suggesting that TH incubation led to robust gene editing. FIG. 8C: Western blotting of Cas9-T6N^{CPP}, Lamin-B1, and α-Tubulin levels in the nuclear fraction, cytosolic fraction, and whole-cell lysates prepared from EL4 cells treated with Cas9-T6N^{CPP} and TH. EL4 cells were treated with 5 μM of Cas9-T6N^{CPP} and 75 μM of TH at 37° C. for 30 min. Cells were washed with PBS and trypsinized to remove cell surface-bound Cas9-T6N^{CPP}. Nuclear and cytosolic fractions were separated and subject to immunoblotting analyses using antibodies against Cas9, nuclear marker Lamin B1, and cytosolic marker α-Tubulin. The data showed that the addition of TH increased the translocation of Cas9-T6N^{CPP} to cells, cytosolic fractions, and especially in the nuclear fraction, compared to the cells without TH treatment. FIG. 8D: Quantification of the editing efficiency of Cas9^{CPP} variants in EL4-mChe reporter with various TH peptide concentrations. EL4 mChe reporter cells were treated with 0.5 μM Cas9^{CPP} variants in the presence of 25-75 μM TH. To measure editing efficiency, the percentage of cells with loss of mCherry was measured by flow cytometry at day 4 post-treatment. FIG. 8E: Final workflow of Cas9-PAGE system for gene editing in EL4 mCherry reporter cell line. The combination of cell-penetrating Cas protein and the endosomal escaping peptide was termed Peptide-Assisted Genome Editing (PAGE).

FIGS. 9A-9E: Optimization of Cas9-PAGE system in EL4 reporter cells. FIGS. 9A-9B: Quantification of gene editing efficiency with titration of either TH or Cas9-T6N^{CPP}. The percentage of cells with loss of mCherry was measured by flow cytometry at day 2 post-treatment. FIG. 9A: EL4 mCherry reporter cells were incubated with 0.5 μM Cas9-T6N^{CPP} and various concentrations of TH from 5 to 100 μM. The concentration of TH was positively correlated with increased gene editing efficiency. FIG. 9B: EL4 mCherry reporter cells were treated with various concentrations of Cas9-T6N^{CPP} from 0.05 to 5 μM and 75 μM TH. Increasing Cas9-T6N^{CPP} concentration led to increased gene editing efficiency. FIG. 9C: Quantification of live cell recovery of EL4 cells treated with an increasing concentrations of TH. FIG. 9D: Quantification of GFP positive cell population as a function of increasing amounts of Cas9-T6N^{CPP}. The GFP positive cell percentage serves as a surrogate for cell-penetrating efficiency.

FIGS. 10A-10B: TH supports PAGE system in trans. FIG. 10A: Quantification of gene editing efficiency with truncation of TH. TH (dTAT-HA2), and neither T (dTAT) nor H (dHA2) peptides alone, enhanced Cas9-T6N^{CPP} editing efficiency in EL4 mCherry reporter cells. EL4 mCherry reporter cells were incubated with 0.5 μM Cas9-T6N^{CPP} in the presence of 75 μM T, H, or TH peptides. The percentage of cells with loss of mCherry was measured by flow cytometry at day 4 post-treatment. FIG. 10B: Quantification of gene editing efficiency with Cas9-T6N^{CPP} and Cas9-TH6N^{CPP}, where dTAT-HA2 (TH) is added in cis. The percentage of cells with loss of mCherry was measured by flow cytometry at day 4 post-treatment. TH facilitated the Cas9^{CPP} in trans, independent of whether the TH peptide was present in cis or not.

FIG. 11: PAGE system for gene editing in various cell types. Quantification of Cas9-PAGE system-mediated gene editing efficiency in various cell types. mCherry positive reporter was established in indicated cell types: the human myeloid cell line model MOLM-13, the human natural killer cell line NK-92, and human primary T cell, isolated from PBMCs of three healthy donors. mCherry reporter cells were incubated with indicated Cas9-T6N^{CPP} and TH for 30 min. The percentage of cells with loss of mCherry was measured by flow cytometry at day 4 post-treatment.

FIGS. 12A-12G: Cas9-PAGE with retroviral sgRNA mediated genome editing in murine primary CD8 T cells ex vivo. FIG. 12A: A schematic of the experimental workflow of evaluating the PAGE system in murine primary CD8 T cells ex vivo. Murine primary T cells were activated with anti-CD3, anti-CD28 and IL-2, followed by retroviral transduction with a sgRNA expression vector linked with mCherry fluorescent marker. The FACS-sorting enriched mCherry positive cells were incubated with Cas9-T6N^{CPP} and TH peptide, which were then washed out after 30 min incubation. Gene editing was evaluated at various time points by flow cytometry against indicated gene products or via direct sanger sequencing of the targeted genomic regions. FIGS. 12B-12E: TH promoted Cas9-T6N^{CPP} gene editing in murine primary CD8 T cells. Cells were transduced with either sgThy1_IG1 or sgNeg, followed by 30 min incubation with various concentrations of TH and 5 μM Cas9-T6N^{CPP}. Flow cytometry analysis was performed at the indicated days post-treatment. FIG. 12B: A time-course analysis of CD90 protein expression in CD8 T cells treated with an increased concentration of TH. FIG. 12C: Mean Fluorescent Intensity (MFI) quantification of the flow cytometry analysis at 4 days post-treatment in FIG. 12B (left panel). FIG. 12D: A representative flow cytometry plot of CD90 in cells transduced with either sgThy1_IG1 or sgNeg at 4 days post-treatment. FIG. 12E: Quantification of live cell recovery of CD8 T cells treated with an increased concentration of TH. FIG. 12F: Summary bar graph of gene editing efficiency of PAGE with additional sgRNAs targeting Thy1 and Ptprc genes in murine primary CD8 T cells at day 4 post-treatment. FIG. 12G: Tracking of Indels by DEcomposition (TIDE) mutagenesis assay of PAGE sgRNAs used in FIG. 12F. Dot plot depicted the TIDE assay score (indel %) for indicated sgRNA. Genomic DNA was isolated at day 6 post-treatment of PAGE, sanger sequenced, and followed by quantification via an online TIDE analysis tool. sgThy1_IG1, sgThy1_IG2, sgThy1_IG3, sgRNAs targeting the Immunoglobulin domain of Thy1 gene; sgPtprc_CAT1 and sgPtprc_TM1, sgRNAs targeting either the catalytic domain or transmembrane domain of Ptprc gene; sgNeg, an sgRNA targeting Ano9 gene was used as a negative control here.

FIGS. 13A-13D: Cell penetrating ribonucleoprotein (RNP) complex for PAGE genome editing in murine primary T cells ex vivo. FIG. 13A: A schematic of series Cas^{CPP} variants for RNP-PAGE experiments in murine primary T cells. Cas9-T6N^{CPP} (TAT-4×NLS^{MYC} NLS-Cas9-2×NLS^{SV40}-sfGFP); Cas9-T8N^{CPP} (TAT-6×NLS^{MYC} NLS-Cas9-2×NLS^{SV40}-sfGFP); opCas12a-T8N^{CPP} (TAT-6×NLS^{MYC} NLS-opCas12a-2×NLS^{SV40}-sfGFP). FIG. 13B: A schematic of the experiment showing ex vivo editing of Cas9/opCas12a-RNP-PAGE in mouse primary T cells. Murine primary CD8 T cells, either naive or activated for 2 days, were incubated with 5 μM RNP complex and various concentrations of TH for 30 min at 37° C. Cells were washed once and cultured for 5 days with or without sorting for GFP+ cells, and editing efficiency was measured by flow cytometry of target gene expression. FIG. 13C: Analysis of CD90 expression level in either naive CD8 or activated CD8 T cells treated with various Cas9/opCas12a-RNP-PAGE systems. Murine primary naive CD8 or activated CD8 T cells were treated with 5 μM Cas9-T6N^{CPP}, Cas9-T8N^{CPP}, or opCas12a-T8N^{CPP} RNP complex with guide RNA targeting CD90 IG domain together with 25 μM TH as described in FIG. 13B. CD90 expression was measured by flow cytometer at day 5 post-treatment. opCas12a-RNP-PAGE displayed superior gene editing efficiency over Cas9-RNP-PAGE in murine primary T cells. FIG. 13D: Optimization of TH concentration in primary mouse CD8 T cells for opCas12a-RNP-PAGE delivery. Murine primary CD8 T cells were activated for 2 days and treated with 5 μM opCas12a-T8N^{CPP} RNP targeting the CD90 IG domain in the presence of various concentrations of TH from 25 to 50 μM. CD90 expression was measured by flow cytometry at day 5 post-treatment.

FIG. 14A-14C: opCas12a-RNP-PAGE genome editing in human chimeric antigen receptor (CAR) T cells ex vivo. FIG. 14A: A schematic of the experiment showing ex vivo editing of opCas12a-RNP^{CPP} in CAR T cells. Human primary T cells from healthy donors were isolated and activated with anti-CD3, anti-CD28 and IL-2. Activated T cells were transduced with CAR19 lentivirus at day 1. On day 6, CAR19+ cells were FACS sorted prior to incubation with 5 μM opCas12a-T8N^{CPP} RNP and 25 μM TH for 30 min. Cells were cultured for an additional 10 days post-treatment and target gene expression was measured by flow cytometry. FIGS. 14B-14C: Human CAR T cells were treated with 5 μM opCas12a-T8N^{CPP} RNP targeting the catalytic domain of CD45 (encoded by PTPRC) or immunoglobulin domain of beta-2-microglobulin (encoded by B2M) in the presence of 25 μM TH. CD45 (FIG. 14B) or B2M (FIG. 14C) expression was measured by flow cytometry at day 6 post-treatment.

FIGS. 15A-15G: Highly efficient in vivo editing of clinically relevant genes by Cas9-PAGE system in murine primary T cells. FIG. 15A: Schematic of the experimental workflow to evaluate the PAGE system in murine primary CD8 T cells in vivo. Donor CD8 T cells from P14 transgenic (CD45.1+ or CD45.1/2+ congenic) mice were isolated and activated with anti-CD3, anti-CD28 and IL-2, followed by retroviral transduction with either experimental or negative control sgRNA expression vector linked with a fluorescent marker. sgRNA-transduced T cells were incubated with 5 μM Cas9-T6N^{CPP} and 25 μM TH peptide for 30 min prior to FACS-sorting to enrich the Cas9 positive and sgRNA positive (double positive) populations. Experimental and negative control sgRNA-transduced P14 T cells were mixed in a 1:1 ratio, followed by adoptive transfer to CD45.2+ congenic recipient mice that were infected with LCMV-clone13 virus. Gene editing and P14 T cell population were evaluated by flow cytometry over a time course of 30 days. FIG. 15B: Example flow cytometry plot and (FIG. 15C) quantification of CD90 surface expression following sgThy1_IG1 mediated editing at day 8 post-infection. FIG. 15D-15E: As FIG. 15B-15C except for PD-1 following sgPdcd1_IG44 mediated editing. FIG. 15F: Proportion of co-transferred P14 T cells transduced with indicated sgRNA in blood over time. FIG. 15G: P14 T cells transduced with indicated sgRNAs as a proportion of total CD8 T cells in blood over a time course of 30 days. n=5-10 per time point, data representative of two experiments.

FIGS. 16A-16C: Cas9-BE PAGE shows base editing in a K562 d2GFP reporter cell line. FIG. 16A: Schematic of Cas9-BE expression construct. FIG. 16B: Schematic of the experimental workflow of evaluating the base editing efficiency of Cas9-BE PAGE system in a K562 d2GFP reporter cell line. K562 cells were lentivirally transduced with a dual expression vector stably expressing d2GFP fluorescence reporter and a sgRNA targeting d2GFP fluorescence reporter gene or a sgRNA targeting Ano9 gene as a negative control. K562 d2GFP cells were incubated with Cas9-BE-T6N^{CPP} and TH peptide for 30 min, followed by washing out the protein and peptide. Base editing was evaluated by loss of d2GFP reporter fluorescence at day 5 post-treatment when the GFP-linked Cas9-BE protein degraded completely. FIG. 16C: Quantification of loss of d2GFP expression in K562 d2GFP reporter cell line as described in FIG. 16B.

### DETAILED DESCRIPTION

Herein, an optimized, highly efficient, inexpensive, and novel gene editing method was established in cells using a cell penetrating Cas protein linked to an endosomal escape peptide. This work has generated a novel composition of matter for this cell penetrating Cas tool. Compared to the published gene editing methods using cell penetrating CRISPR-Cas systems (Ramakrishna et al., (2014) Genome Res 24, 1020-1027; Staahl et al., (2017) Nat Biotechnol 35, 431-434), this method achieves high gene editing efficiency both in vitro and in vivo. Compared to the published gene editing method for mouse primary T cell using CRISPR-Cas system that requires electroporation of Ribonucleoprotein (RNP) complex (Kornete et al., (2018) J Immunol 200, 2489-2501; Nussing et al., (2020) J. Immunol), this method is less expensive and more easily implemented into experimental workflows. The present method does not require electroporation, but instead requires either (1) incubating the cell penetrating Cas protein and endosomal escape peptide with the cells infected with an sgRNA expressing construct or (2) incubating the cell penetrating Cas-sgRNA ribonucleoprotein (RNP) complex and endosomal escape peptide with the cells. Furthermore, since this method does not require transgenic mice expressing Cas protein to achieve gene editing, it saves the time and expense of generating a specific Cas transgenic mouse line. Importantly, since the cells lose the majority of cell penetrating Cas protein in two days after incubation, this reduces the Cas protein immunogenicity and/or decreases off-target genomic effects observed in other studies.

The method can be used by researchers to achieve gene editing in primary mouse and human T cells or other primary immune cells (including human immune cells) and enable CRISPR-CAS screening. The settings used in this method can also be applied to other Cas proteins in addition to Cas9, i.e. Cas12a, and Cas9-Base Editor.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook, and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockin” as used herein refers to an exogenous nucleic acid sequence that has been inserted into a target sequence (e.g., endogenous gene locus. In some embodiments, where the target sequence is a gene, a knockin is generated resulting in the exogenous nucleic acid sequence being in operable linkage with any upstream and/or downstream regulatory elements controlling expression of the target gene. In some embodiments, the knockin is generated resulting in the exogenous nucleic acid sequence not being in operable linkage with any upstream and/or downstream regulatory elements controlling expression of the target gene.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. In Vitro and In Vivo Methods of Gene Editing

Provided herein are methods of gene editing (in vitro, ex vivo, and in vivo) using a novel CRISPR-Cas system termed Peptide-Assisted Genome Editing (PAGE) system. The PAGE system comprises a cell penetrating Cas (e.g. a Cas (e.g. Cas 9 or Cas12a) linked to a cell penetrating peptide (CPP)), and an endosomal escape peptide (e.g. dTAT-HA2) linked to a CPP. Using this method, the Cas is introduced into a cell (e.g. a primary resting T cell) in a non-viral, non-electroporation dependent manner. A single-guide RNA (sgRNA) or CRISPR RNA (crRNA); or a plurality of sgRNAs or crRNAs can then be introduced into the cell (e.g. via a retroviral expression construct or RNP) to achieve in vitro, ex vivo, and in vivo editing of the cell (e.g primary CD8 T cell).

In one aspect, the disclosure provides an in vitro method of gene editing comprising introducing into a cell a PAGE system comprising a cell penetrating Cas and an endosomal escape peptide, and at least one sgRNA or crRNA. The cell penetrating Cas comprises a Cas (e.g. Cas9, Cas12a) linked to a CPP. The endosomal escape peptide is linked to a CPP.

In another aspect, the disclosure provides an ex vivo or in vivo method of gene editing comprising introducing into a cell a PAGE system comprising a cell penetrating Cas and an endosomal escape peptide, and at least one sgRNA or crRNA, and administering the cell to a subject. The cell penetrating Cas comprises a Cas (e.g. Cas9, Cas12a) linked to a CPP. The endosomal escape peptide is linked to a CPP.

In certain embodiments, the Cas is Cas9. Exemplary Cas9 nucleases that may be used in the present invention include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9). In certain embodiments, the Cas is Cas12a (Cpf1), including but not limited to Butyrivibrio sp (BsCas12a), Thiomicrospira sp). XS5 (TsCas12a, Moraxella bovoculi (MbCas12a), Prevotella bryantii (PbCas12a), Bacteroidetes oral (BoCas12a), Lachnospiraceae bacterium (LbCas12a), and Acidaminococcus sp (AsCas12a). In certain embodiments, the Cas is Cas12a. In certain embodiments, the Cas is selected from the group consisting of Cas12b, Cas12d, Cas12f, T7, Cas3, Cas8a, Cas8b, Cas10d, Cse1, Csy1, Csn2, Cas4, Cas10, Csm2, Cmr5, and Fok1.

In certain embodiments, the Cas is a Cas derivative. In certain embodiments, the Cas derivative is a Cas protein linked to another protein or catalytic domain. The Cas protein can be linked to another protein or catalytic domain by any means known in the art, such as, but not limited to chemical linkage, fusion, or post-translational modification. In certain embodiments, the protein or catalytic domain is selected from the group consisting of an AID deaminase, an APOBEC deaminase, a TadA deaminase, a TET enzyme, a DNA methyltransferase, a transactivation domain, a reverse transcriptase, a histone acetyltransferase, a histone deacetylase, a sirtuin, a histone methyltransferase, a histone demethylase, a kinase, and a phosphatase.

In certain embodiments, the Cas comprises a Nuclear Localization Signal (NLS) sequence. Any NLS known in the art or disclosed herein can be used. In certain embodiments, the Cas comprises a Myc NLS sequence. In certain embodiments, the Myc NLS sequence comprises or consists of the amino acid sequence PAAKRVKLD (SEQ ID NO: 1). In certain embodiments, the Cas comprises a 4×Myc or 6×Myc NLS sequence. In certain embodiments, the NLS (i.e. 4×Myc or 6×Myc) sequence further comprises a GGS linker.

In certain embodiments, the cell penetrating Cas comprises a nucleotide sequence encoding, or amino acid sequence comprising, a Cell Penetrating Peptide. Cell Penetrating Peptides (CPPs, also known as Protein Transduction Domains, PTDs), are carriers with small peptide domains (generally less than 40 amino acids) that can easily cross cell membranes. Multiple cell permeable peptides have been identified that facilitate cellular uptake of various molecular cargo, ranging from nanosize particles to small chemical molecules. Cell penetrating sequences can be used as extensions to peptide sequences thereby making them more permeable to cell membranes, or cell penetrating peptide can be attached to other cargo molecules to enhance their cellular uptake. Cell penetrating sequences can be either fused directly to the cargo molecules or chemically linked to cargo molecules. Examples of such cell penetrating peptides include, but are not limited to trans-activating transcriptional activator (Tat) from HIV-1, Oligo-Arg, KALA, Transportan, Penetratin, Penetratin-Arg, TAT-HA2, and dTAT-HA2E5. The PAGE system may comprise two different CPPs or two of the same CPPs. The CPP can be linked to the Cas or endosomal escape peptide by any means known in the art, such as, but not limited to chemical linkage, fusion, or post-translational modification. In certain embodiments, the CPP comprises a peptide listed in Table 2. In certain embodiments, the Cas is linked to a CPP listed in Table 2. In certain embodiments, the endosomal escape peptide is linked to a CPP listed in Table 2. In certain embodiments, the CPP comprises any of the amino acid sequences set forth in SEQ ID NOs: 10-1422. In certain embodiments, the Cas is linked to a CPP comprising any of the amino acid sequences set forth in SEQ ID NOs: 10-1422. In certain embodiments, the endosomal escape peptide is linked to a CPP comprising any of the amino acid sequences set forth in SEQ ID NOs: 10-1422.

In certain embodiments, the cell penetrating Cas comprises a sequence derived from the trans-activating transcriptional activator (Tat) from HIV-1. In certain embodiments, the Tat sequence comprises the amino acid sequence GRKKRRQRRRPQ (SEQ ID NO: 2).

In certain embodiments, the cell penetrating Cas is introduced into the cell at a concentration between 0.05 μM and 10 μM. In certain embodiments, the cell penetrating Cas is introduced into the cell at a concentration of about 0.5 μM.

In certain embodiments, the endosomal escape peptide comprises dTAT-HA2. Other endosomal escape peptides that could be used include, but are not limited to, EEDs, HA2-penetratin, GALA, INF-7, and the like. In certain embodiments, the endosomal escape peptide comprises any one of the peptides or sequences listed in Table 1. The endosomal escape peptide can include any and all chemical modifications to the peptide, or chemically-modified derivatives of the peptide, or special chemical-linkers within the peptide, or D form of amino acids, listed in Table 1. In certain embodiments, the endosomal escape peptide comprises any one of the sequences set forth in SEQ ID NOs: 1434-1523. In certain embodiments, the endosomal escape peptide comprises any one of the sequences set forth in SEQ ID NOs: 1434-1523 and a chemical modification and/or a chemical-linker. Examples of chemical modifications include but are not limited to: phosphate (PO3), trifluoromethyl-bicyclopent-[1.1.1]-1-ylglycine (CF3-Bpg), amino isobutyric acid (Aib), stearylation (Stearyl), 6-aminohexanoic acid (Ahx), L-2-naphthylalanine (Φ), and 3-amino-3-carboxypropyl (acp).

In certain embodiments, the endosomal escape peptide is introduced into the cell at a concentration between 10 μM to 100 μM. In certain embodiments, the endosomal escape peptide is introduced into the cell at a concentration of about 75 μM.

In certain embodiments, the cell is an immune cell. In certain embodiments, the cell is a murine primary CD8 T cell, human primary T cell, or human iPSC (induced pluripotent stem cell).

In certain embodiments, the method does not require electroporation. In certain embodiments, the PAGE system is introduced into the cell in a medium that does not contain Fetal Bovine Serum (FBS) or serum. In certain embodiments, the PAGE system is introduced into the cell in a medium contains FBS or serum.

The methods should be construed to target any gene/genomic region/nucleotide sequence in a cell (e.g. a eukaryotic/human cell). Thus, an sgRNA or crRNA, or plurality of sgRNAs or crRNAs can be designed to target any gene/genomic region/nucleotide sequence in a cell (e.g. a eukaryotic/human cell) for use with the methods herein. In certain embodiments, the sgRNA targets Ano9 or Pdcd1. In certain embodiments, the sgRNA targets human Ano9 or Pdcd1. In certain embodiments, the sgRNA comprises or consists of the nucleotide sequence GCCTGGCTCACAGTGTCAGA (SEQ ID NO: 8; Pdcd1 Ig_44). In certain embodiments, the sgRNA comprises or consists of the nucleotide sequence GGTATCATGAGTGCCCTAGT (SEQ ID NO: 9; Pdcd1 Tm_1). In certain embodiments, the sgRNA targets Ptprc or Thy1. In certain embodiments, the sgRNA comprises or consists of the nucleotide sequence CGTGTGCTCGGGTATCCCAA (SEQ ID NO: 1424; Thy1 IG1). In certain embodiments, the sgRNA comprises or consists of the nucleotide sequence CCGCCATGAGAATAACACCA (SEQ ID NO: 1425; Thy1 IG2). In certain embodiments, the sgRNA comprises or consists of the nucleotide sequence CCTTGGTGTTATTCTCATGG (SEQ ID NO: 1426; Thy1 IG3). In certain embodiments, the sgRNA comprises or consists of the nucleotide sequence TTGTCAAGCTAAGGCGACAG (SEQ ID NO: 1427; Ptprc CAT1). In certain embodiments, the sgRNA comprises or consists of the nucleotide sequence TCACAATAATCAGAAACACC (SEQ ID NO: 1428; Ptprc TM1). In certain embodiments, the crRNA targets PTPRC or B2M. In certain embodiments, the crRNA comprises or consists of the nucleotide sequence TTCAGTGGTCCCATTGTGGT (SEQ ID NO: 1429; PTPRC CAT1). In certain embodiments, the crRNA comprises or consists of the nucleotide sequence GTGGAATACAATCAGTTTGG (SEQ ID NO: 1430; PTPRC CAT2). In certain embodiments, the crRNA comprises or consists of the nucleotide sequence TTCTCGGCTTCCAGGCCTTC (SEQ ID NO: 1431; PTPRC CAT3). In certain embodiments, the crRNA comprises or consists of the nucleotide sequence CATTCTCTGCTGGATGACGT (SEQ ID NO: 1432; B2M IG1). In certain embodiments, the crRNA comprises or consists of the nucleotide sequence AATTCTCTCTCCATTCTTCA (SEQ ID NO: 1433; B2M IG2).

In certain embodiments, the methods disclosed herein are used to treat a subject for a disease or disorder. The method comprises introducing into a cell a PAGE system comprising a cell penetrating Cas and an endosomal escape peptide and at least one sgRNA or crRNA, then administering the cell to a subject. When the edited cell is administered to the subject, the disease or disorder is treated in the subject. In certain embodiments, the disease or disorder to be treated in the subject is an infection. In certain embodiments, the disease or disorder is related to T cell exhaustion.

In certain embodiments, the PAGE system comprises a CRISPR/Cas9 system. The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells) and primary cells. The CRISPR/Cas9 system can simultaneously target multiple genomic loci by co-expressing a single Cas9 protein with two or more gRNAs, making this system suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The REC I domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, a pAd5F35-CRISPR vector. In other embodiments, the Cas expression vector induces expression of Cas9 endonuclease. Other endonucleases may also be used, including but not limited to, Cas12a (Cpf1), T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). Other inducible promoters known by those of skill in the art can also be used. The inducing agent can be a selective condition (e.g., exposure to an agent, for example an antibiotic) that results in induction of the inducible promoter. This results in expression of the Cas expression vector.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” or “target sequence” is the DNA sequence in a genome of a cell that is complementary to the guide sequence of the gRNA.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of a CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas nuclease, a crRNA, and a tracrRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR associated (Cas) enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In some embodiments, the CRISPR/Cas is derived from a type II CRISPR/Cas system. In other embodiments, the CRISPR/Cas system is derived from a Cas9 nuclease. Exemplary Cas9 nucleases that may be used in the present invention include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9).

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

TABLE 1
Exemplary Endosomal Escape Peptide Sequences

| SEQ | |
|---|---|
| ID | Amino Acid |
| NO. | Sequence |
| 1434 | FLFPLITSFLSKVL |
| 1435 | FISAIASMLGKFL |
| 1436 | GWFDVVKHIASAV |
| 1437 | FFGSVLKLIPKIL |
| 1438 | GLFDIIKKIAESF |
| 1439 | HGVSGHGQHGVHG |
| 1440 | FLPLIGRVLSGIL |
| 1441 | GLFDIIKKIAESI |
| 1442 | GLLDIVKKVVGAFGSL |
| 1443 | GLFDIVKKVVGALGSL |
| 1444 | GLFDIVKKVVGAIGSL |
| 1445 | GLFDIVKKVVGTLAGL |
| 1446 | GLFDIVKKVVGAFGSL |
| 1447 | GLFDIAKKVIGVIGSL |
| 1448 | GLFDIVKKIAGHIAGSI |
| 1449 | GLFDIVKKIAGHIASSI |
| 1450 | GLFDIVKKIAGHIVSSI |
| 1451 | FVQWFSKFLGRIL |
| 1452 | GLFDVIKKVASVIGGL |
| 1453 | GLFDIIKKVASVVGGL |
| 1454 | GLFDIIKKVASVIGGL |
| 1455 | VWPLGLVICKALKIC |
| 1456 | NFLGTLVNLAKKIL |
| 1457 | FLPLIGKILGTIL |
| 1458 | FLPIIAKVLSGLL |
| 1459 | FLPIVGKLLSGLL |
| 1460 | FLSSIGKILGNLL |
| 1461 | FLSGIVGMLGKLF |
| 1462 | TPFKLSLHL |
| 1463 | GILDAIKAIAKAAG |
| 1464 | LFDIIKKIAESF |
| 1465 | LFDIIKKIAESGFLFDIIKKIAESF |
| 1466 | GLLNGLALRLGKRALKKIIKRLCR |
| 1467 | GHHHHHHHHHHHHH |
| 1468 | FKCRRWQWRM |
| 1469 | KTCENLADTY |
| 1470 | ALFDIIKKIAESF |
| 1471 | GAFDIIKKIAESF |
| 1472 | GLADIIKKIAESF |
| 1473 | GLFAIIKKIAESF |
| 1474 | GLFDAIKKIAESF |
| 1475 | GLFDIAKKIAESF |
| 1476 | GLFDIIAKIAESF |
| 1477 | GLFDIIKAIAESF |
| 1478 | GLFDIIKKAAESF |
| 1479 | GLFDIIKKIAASF |
| 1480 | GLFDIIKKIAEAF |
| 1481 | GLFDIIKKIAESA |
| 1482 | GLFDIIHKIAESF |
| 1483 | GLFDIIKHIAESF |
| 1484 | GLFDIIKKIAHSF |
| 1485 | GLFDIIRKIAESF |
| 1486 | GLFDIIKRIAESF |
| 1487 | GLFDIIKKIARSF |
| 1488 | GLFDIIKKIADSF |
| 1489 | GDIMGEWGNEIFGAIAGFLG |
| 1490 | GLFGAIAGFIENGWEGMIDG |
| 1491 | GLFEAIEGFIENGWEGMIDGWYG |
| 1492 | GLFEAIAEFIEGGWEGLIEGCAKKK |
| 1493 | GLFGAIAGFIENGQWGMIDG |
| 1494 | GLFEAIEGFIENGWEGMIDGWYGCGLFEAIEGFI |
| | ENGWEGMIDGWYGC |
| 1495 | GLLEALAELLE |
| 1496 | LAEALAEALEALAA |
| 1497 | GIGAVLKVLTTGLPALISWIKRKRQQ |
| 1498 | CIGAVLKVLTTGLPALISWIKRKRQQ |
| 1499 | RQIKIWFQNRRMKWKK |
| 1500 | MVKSKIGSWILVLFVAMWSDVGLCKKRPKP |
| 1501 | GALFLGWLGAAGSTMGAPKSKRKV |
| 1502 | LIRLWSHLIHIWFQNRRLKWKKK |
| 1503 | GLFEAIAEFIENGWEGLIEGWYG |
| 1504 | CKYGRRRQRRKKRGGDIMGEWGNEIFGAIAGFLG |
| 1505 | GLFEAIEGFIENGWEGMIWDYGSGSCG |
| 1506 | KETWWETWWTEWSQPKKKRKV |
| 1507 | LLIILRRRRIRKQAHAHSK |
| 1508 | DPKGDPKGVTVTVTVTVTGKGDPKPD |
| 1509 | CSIPPEVKFNKPFVYLI |
| 1510 | GWTLNSAGYLLGKINLKALAALAKKIL |
| 1511 | AGYLLGKINLKALAALAKKIL |
| 1512 | GALFLGFLGAAGSTMGA |
| 1513 | HGLASTLTRWAHYNALIRAF |
| 1514 | GLWRALWRLLRSLWRLLWRA |
| 1515 | WEAALAEALAEALAEHLAEALAEALEALAA |
| 1516 | GLFEAIEGFIENGWEGMIDGWYGC |
| 1517 | GLFGAIAGFIENGWEGMIDGWYG |
| 1518 | GLFGAIAGFIENGWEGMIDGRQIKIWFQNRRMKWKK |
| 1519 | GLFGAIAGFIENGWEGMIDGSSKKKK |
| 1520 | GLFEAIAGFIENGWEGMIDGGGYC |
| 1521 | GLFHAIAHFIHGGWHGLIHGWYG |
| 1522 | GLFEAIEGFIENGWEGLAEALAEALEALAA |
| 1523 | KWKLFKKIGAVLKVLTTGYGRKKRRQRRR |

### C. Sources of Cells

Any type of cell can be edited with the methods disclosed herein. In certain embodiments, the cell is an immune cell. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. Immune cells can be obtained from a number of sources, including blood, peripheral blood mononuclear cells, bone marrow, lymph node tissue, spleen tissue, umbilical cord, lymph, or lymphoid organs.

In certain embodiments, the immune cell is a T cell, e.g., a CD8+ T cell (e.g., a primary CD8+ T cell, a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In certain embodiments, the cell is a monocyte or granulocyte, e.g., myeloid cell, macrophage, neutrophil, dendritic cell, mast cell, eosinophil, and/or basophil. In certain embodiments, the cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a primary CD8+ T cell, a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of n CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In certain embodiments, the cell comprises a Chimeric Antigen Receptor (CAR). In certain embodiments, the cell is a CAR T cell. Exemplary CARs include, but are not limited to, those disclosed herein, those disclosed in U.S. Pat. Nos. 10,357,514B2, 10,221,245B2, 10,603,378B2, 8,916,381B1, 9,394,368B2, US20140050708A1, U.S. Pat. Nos. 9,598,489B2, 9,365,641B2, US20210079059A1, U.S. Pat. No. 9,783,591B2, WO2016028896A1, U.S. Pat. No. 9,446,105B2, WO2016014576A1, US20210284752A1, WO2016014565A2, WO2016014535A1, and U.S. Pat. No. 9,272,002B2, and any other CAR generally disclosed in the art. The disclosure should be construed to include any CAR known in the art.

In some embodiments, the methods include isolating immune cells from a subject, preparing, processing, culturing, and/or engineering them. In some embodiments, preparation of the cells includes one or more culture and/or preparation steps. The cells for engineering as described may be isolated from a sample, such as a biological sample, e.g., one obtained from or derived from a subject. In some embodiments, the subject from which the cell is isolated is one having a disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells, e.g., primary human CD8+ cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain embodiments, a source of immune cells is obtained from a subject for ex vivo manipulation. Sources of cells for ex vivo manipulation may also include, e.g., autologous or heterologous donor blood, cord blood, or bone marrow. For example the source of immune cells may be from a subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

In certain embodiments, a cell is modified with a method contemplated herein; e.g. by introducing into the cell a cell penetrating CRISPR-Cas9 or -Cas12a system comprising a cell penetrating Cas9 or Cas12a and an endosomal escape peptide, then the modified cell is administered to a subject. In certain embodiments, the subject is in need of a treatment for a disease or condition. With reference to the subject to be treated, the cells may be allogeneic and/or autologous. The cells typically are primary cells, such as those isolated directly from a subject and/or isolated from a subject and frozen.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acids. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker⁺) or express high levels (marker^{high}) of one or more particular markers, such as surface markers, or that are negative for (marker⁻) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CDI 1b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### D. Compositions

In one aspect, the disclosure provides a novel cell penetrating PAGE system capable of efficiently editing a cell (e.g. a primary CD8 T cell). The PAGE system comprises a cell penetrating Cas (e.g. a Cas (e.g. Cas9 or Cas12a) linked to a CPP) and an endosomal escape peptide linked to a CPP (e.g. dTAT-HA2).

In certain embodiments, the Cas is Cas9. Exemplary Cas9 nucleases that may be used in the present invention include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9). In certain embodiments, the Cas is Cas12a (Cpf1), including but not limited to, Butyrivibrio sp (BsCas12a), Thiomicrospira sp). XS5 (TsCas12a, Moraxella bovoculi (MbCas12a), Prevotella bryantii (PbCas12a), Bacteroidetes oral (BoCas12a), Lachnospiraceae bacterium (LbCas12a), and Acidaminococcus sp (AsCas12a). In certain embodiments, the Cas is selected from the group consist Cas12b, Cas12d, Cas12f, T7, Cas3, Cas8a, Cas8b, Cas10d, Cse1, Csy1, Csn2, Cas4, Cas10, Csm2, Cmr5, and Fok1.

In certain embodiments, the Cas protein (i.e. Cas9, Cas12a, Cas derivative) is either fused or chemically linked or post-translationally attached to DNA modifiers or catalytic domains thereof, including but not limited to, AID deaminase, an APOBEC deaminase, a TadA deaminase, a TET enzyme, a DNA methyltransferase, a transactivation domain, a reverse transcriptase, a histone acetyltransferase, a histone deacetylase, a sirtuin, a histone methyltransferase, a histone demethylase, a kinase, a phosphatase, and the like.

In certain embodiments, the endosomal escape peptide comprises dTAT-HA2. Other endosomal escape peptides that could be used include, but are not limited to, EEDs, HA2-penetratin, GALA, INF-7, and the like. In certain embodiments, the endosomal escape peptide is any one of the peptides listed in Table 1. In certain embodiments, the endosomal escape peptide comprises any one of the sequences set forth in SEQ ID NOs: 1434-1523. In certain embodiments, the endosomal escape peptide is linked to any of the CPPs listed in Table 2. In certain embodiments, the endosomal escape peptide is linked to a CPP comprising any of the amino acid sequences set forth in SEQ ID NOs: 10-1422.

In certain embodiments, the Cas comprises a nuclear localization sequence (NLS). The NLS can include any NLS known in the art or disclosed herein. In certain embodiments, the Cas comprises a 4× or 6×Myc NLS sequence. In certain embodiments, the Myc NLS sequence comprises the amino acid sequence PAAKRVKLD (SEQ ID NO: 1). In certain embodiments, the NLS (i.e. 4× or 6×Myc NLS) sequence further comprises a GGS linker.

In certain embodiments, the cell penetrating Cas comprises a nucleotide sequence encoding, or amino acid sequence comprising, a Cell Penetrating Peptide. Examples of CPPs include, but are not limited to trans-activating transcriptional activator (Tat) from HIV-1, Oligo-Arg, KALA, Transportan, Penetratin, Penetratin-Arg, TAT-HA2, and dTAT-HA2E5. Examples of CPPs are also listed in Table 2 herein. In certain embodiments, the CPP comprises any of the amino acid sequences set forth in SEQ ID NOs: 10-1422. In certain embodiments, the cell penetrating Cas comprises a sequence derived from the trans-activating transcriptional activator (Tat) from HIV-1. In certain embodiments, the Tat sequence comprises the amino acid sequence GRKKRRQRRRPQ (SEQ ID NO: 2). Other truncated or modified Tat peptides that could be used include, but are not limited to, Truncated Tat: YGRKKRRQRRR (SEQ ID NO: 3), CGRKKRRQRRR (SEQ ID NO: 4), GRKKRRQRRRPPQ (SEQ ID NO: 5), RKKRRQRRRPQ (SEQ ID NO: 6), and RKKRRQRRR (SEQ ID NO: 7), and Modified Tat: 2×Tat, 3×Tat, 4×Tat, n×Tat, and the like.

The PAGE system may comprise two different CPPs or two of the same CPPs. The CPP can be linked to the Cas or endosomal escape peptide by any means known in the art, such as, but not limited to chemical linkage, fusion, or post-translational modification.

Also provided are kits comprising the composition and/or for practicing the methods of the invention, as described herein. For example, in some embodiments, kits for practicing the invention methods include a composition comprising a cell penetrating PAGE system comprising a cell penetrating Cas and an endosomal escape peptide.

Furthermore, additional reagents that are required or desired in the protocol to be practiced with the kit components may be present, which additional reagents include, but are not limited to: sgRNAs, nuclease-free water, carriers, and reagents (e.g., nucleotides, buffers, cations, etc.), and the like. The kit components may be present in separate containers, or one or more of the components may be present in the same container, where the containers may be storage containers and/or containers that are employed during the assay for which the kit is designed.

In addition to the above components, the kit may further include instructions for practicing the methods described herein. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of instructions may include a computer readable medium, e.g., CD, etc., on which the information has been recorded. Yet another form of instructions may include a website address which may be used via the internet to access the information at a removed site. Any convenient means may be present in the kits.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Purification of TAT-4×Myc NLS-Cas9

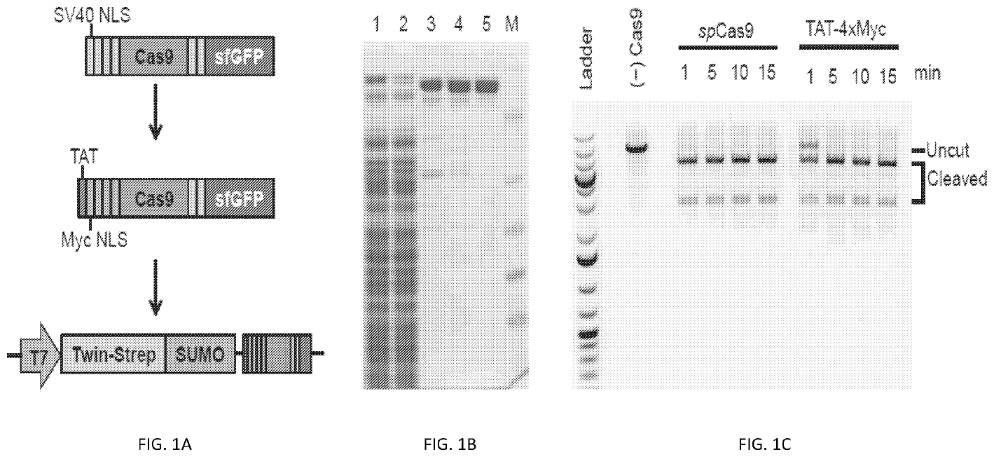

TAT-4×Myc NLS-Cas9 expression construct was created by replacing the 4×SV40 NLS (PKKKRKV (SEQ ID NO: 1423)) at the N-terminus of Cas9 (Staahl et al., (2017) Nat Biotechnol 35, 431-434) with 4×Myc NLS (PAAKRVKLD (SEQ ID NO: 1)) with linker -G-G-S- between Myc NLS. The sequence of the TAT cell penetrating peptide (GRKKRRQRRRPQ (SEQ ID NO: 2)), derived from the trans-activating transcriptional activator (Tat) from HIV-1 (Frankel and Pabo, 1988; Green and Loewenstein, 1988), was added to the N-terminus, and TAT-4×Myc NLS-Cas9 was cloned into a bacterial recombinant protein expression vector (Gootenberg et al., 2017) with a Twin-Strep and SUMO tag (FIG. 1A). Next, TAT-4×Myc NLS-Cas9 protein was purified by Strep-Tactin affinity chromatography followed by on-column SUMO protease digestion, ion exchange chromatography (IEC), and size exclusion chromatography (SEC) (FIG. 1B) (Gootenberg et al., (2017) Science 356, 438-442). The purified TAT-4×Myc NLS-Cas9 protein efficiently cleaved DNA in an in vitro DNA cleavage assay (FIG. 1C).

### Example 2: TAT-4×Myc NLS-Cas9 Genome Editing in EL4 Cells

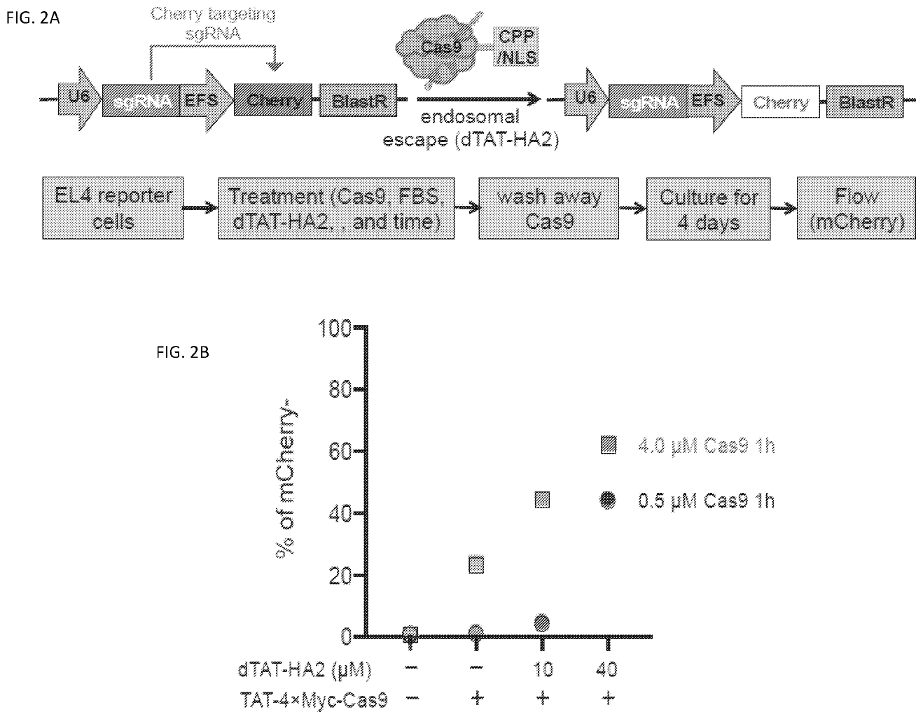

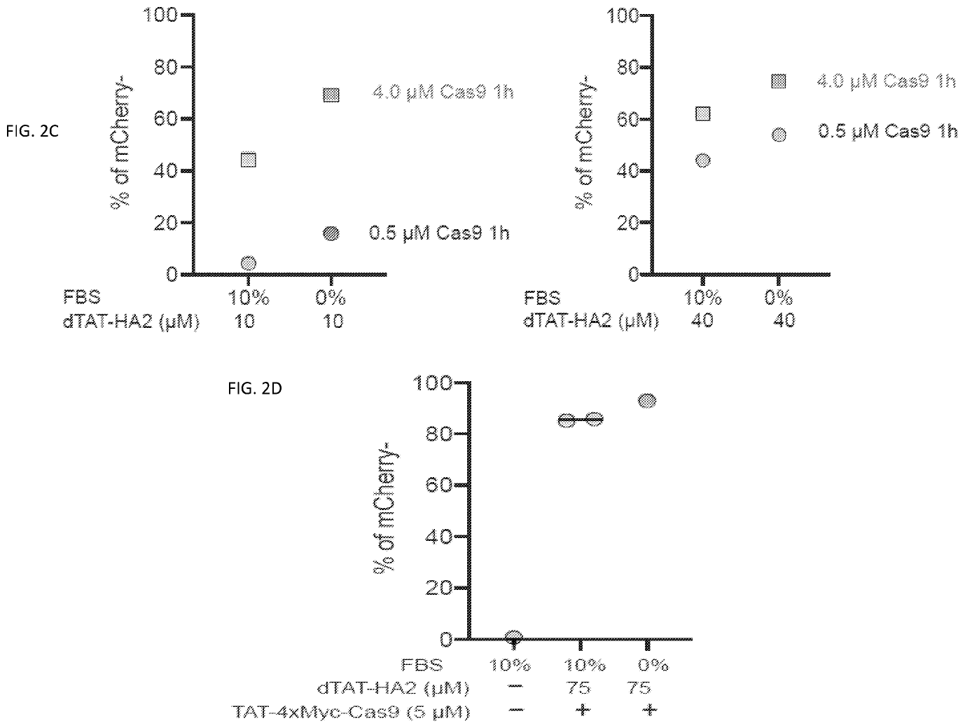

To determine whether TAT-4×Myc NLS-Cas9 has the ability to edit a genome, the EL4 thymoma cell line was used. EL4 cells were infected with a lentiviral reporter construct stably expressing mCherry and sgRNA targeting mCherry. If TAT-4×Myc NLS-Cas9 penetrates the cell membrane and edits mCherry in EL4 reporter cells, the frequency of mCherry cells increases due to loss of mCherry fluorescence, as measured by flow cytometry (FIG. 2A). Here, when the cells were incubated with 0.5 μM TAT-4×Myc NLS-Cas9 alone for 1 hour, an increased frequency of mCherry⁻ cells compared to untreated cells was not observed (FIG. 2B). However, when endosomal escape peptide dTAT-HA2 (up to 40 μM) was added during incubation, the percentage of mCherry cells increased to 44.1% (FIG. 2B). When the cells were incubated with 4.0 μM TAT-4×Myc NLS-Cas9, an even more robust increase of mCherry⁻ cells was observed, with 62% at 40 μM dTAT-HA2 (FIG. 2B).

It was also tested whether the percentage of FBS during incubation has an effect on TAT-4×Myc NLS-Cas9 editing efficiency. When the percentage of FBS decreased from 10% to 0, and the cells were co-treated with 10 μM dTAT-HA2 and 0.5 μM TAT-4×Myc NLSCas9, the percentage of mCherry⁻ cells increased from 4.26% to 15.9% (FIG. 2C, left panel). When the TAT-4×Myc NLS-Cas9 concentration was increased to 4.0 μM, the percentage of mCherry⁻ cells increased from 44.3% to 69.1% (FIG. 2C, left panel). dTAT-HA2 concentration was further increased to 40 μM, and the percentage of mCherry⁻ cells was also increased compared to 10 μM dTAT-HA2 treatment (FIG. 2C, right panel). To achieve the best editing efficiency, the TAT-4×Myc NLS-Cas9 concentration was increased to 5 μM, dTAT-HA2 increased to 75 μM, the incubation time decreased to 30 min, and the percentage of mCherry⁻ cells reached to 92.9% when no FBS was added during incubation (FIG. 2D).

### Example 3: In Vitro Editing by TAT-4×Myc NLS-Cas9 in Mouse Primary T Cells

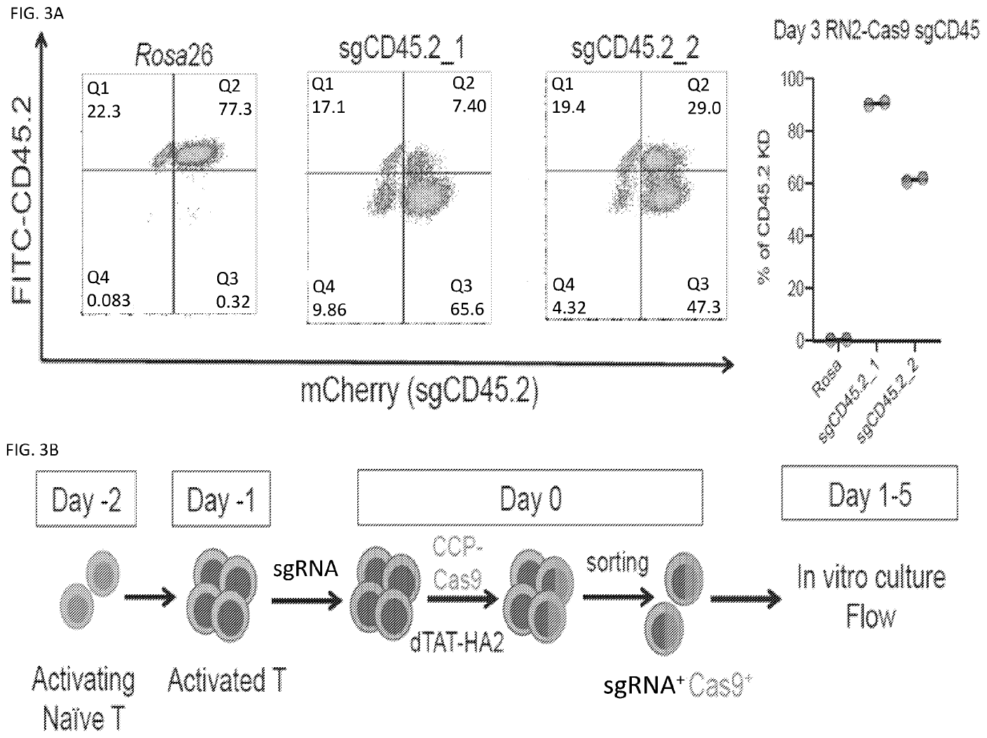

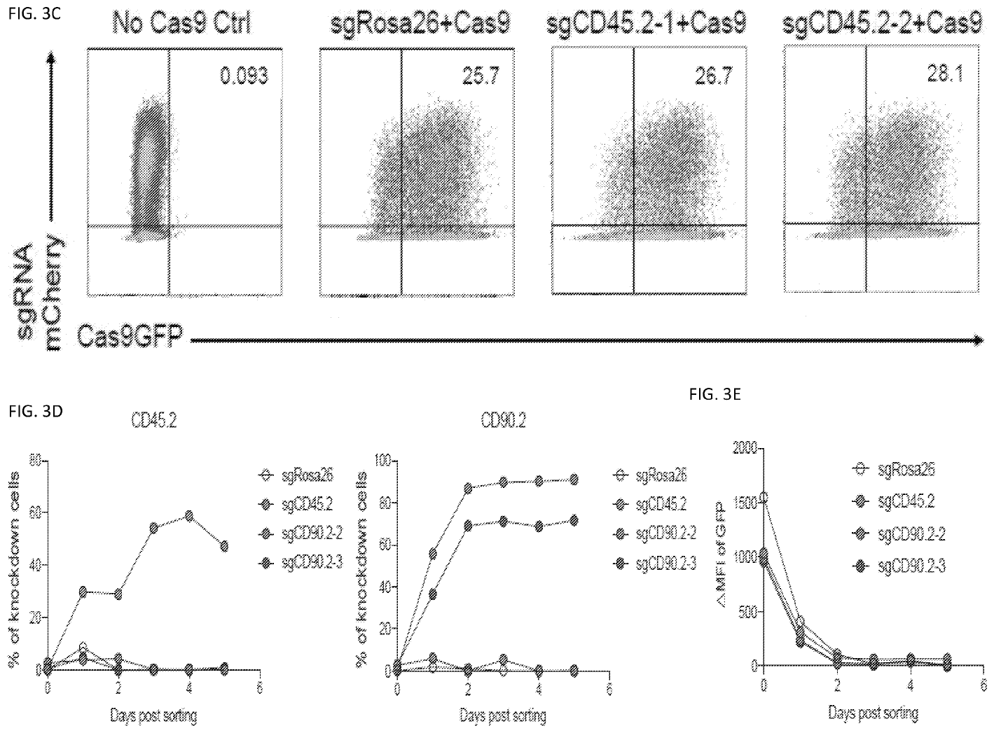

Before testing TAT-4×Myc NLS-Cas9 in vitro editing in mouse primary T cells, the editing efficiency of two sgRNAs targeting cell surface marker CD45.2 were designed and tested in RN2-Cas9 cells, which stably express Cas9. RN2-Cas9 cells were infected with retrovirus expressing sgRNA and mCherry, and CD45.2 expression level was measured by flow cytometry after 3 days of infection. Both sgRNAs targeting CD45.2 efficiently knocked down CD45.2 compared to sgRNAs targeting Rosa26 (FIG. 3A). It was next tested whether TAT-4×Myc Cas9 can edit the genome in mouse primary CD8 T cells. A schematic of the experiment is shown in FIG. 3B. Briefly, on Day −2, primary CD8 T cells were isolated from 3-month-old mouse spleen and activated by CD3, CD8, and IL-2 for 24 hours. On Day −1, the activated cells were infected with retrovirus expressing sgRNA and mCherry for 24 hours. Cells were then treated with 5 μM TAT-4×Myc NLS-Cas9 (with GFP tag) and 75 μM dTAT-HA2, and incubated in RPMI 1640 supplied with 1% FBS and 50 μM 2-mercaptoethanol in a 37° C. incubator for 40 min. Cells were washed twice with PBS, trypsinized for 10 min at 37° C. to remove cell surface bound TAT-4×Myc NLSCas9, treated with DNase I (400 U/ml) for 3 min at room temperature, neutralized, and washed once with completed RPMI 1640 medium. Immediately after washing, 100,000 sgRNA⁺Cas9⁺ (mCherry⁺GFP⁺) cells were sorted at 37° C. (FIG. 3C) for in vitro culture. CD45.2 expression level was measured by flow cytometry from Day 1 to Day 5. For the cells infected with CD45.2 sgRNA_1, the percentage of CD45.2 knockdown cells increased after Day 1 and reached the maximum ˜60% at Day 4. No increase was seen in the cells infected with Rosa26 sgRNA or CD90.2 sgRNA (FIG. 3D, left panel). For cells infected with CD90.2 sgRNA_2 or sgRNA_3, the percentage of CD90.2 knockdown cells dramatically increased after Day 1 and reached the maximum at Day 3 (˜70% for sgRNA_3 and ˜90% for sgRNA_2). No increase of CD90.2 knockdown in the cells infected with Rosa26 sgRNA or CD45.2 sgRNA was seen (FIG. 3D, right panel). The stability of TAT-4×Myc NLS-Cas9 was evaluated in the cells by measuring normalized Mean Fluorescence Intensity (MFI) of GFP. The MFI of GFP decreased ˜75% at Day 1 and more than 90% at Day 2 (FIG. 3E), which indicated that TAT-4×Myc NLS-Cas9 will be less immunogenic than the constitutively expressed Cas in other systems (Ajina et al., (2019) Oncoimmunology 8, e1577127; Chew et al., (2016) Nat Methods 13, 868-874; Wang et al., (2015) Hum Gene Ther 26, 432-442).

### Example 4: In Vivo Editing by TAT-4×Myc NLS-Cas9 in Mouse Primary T Cells

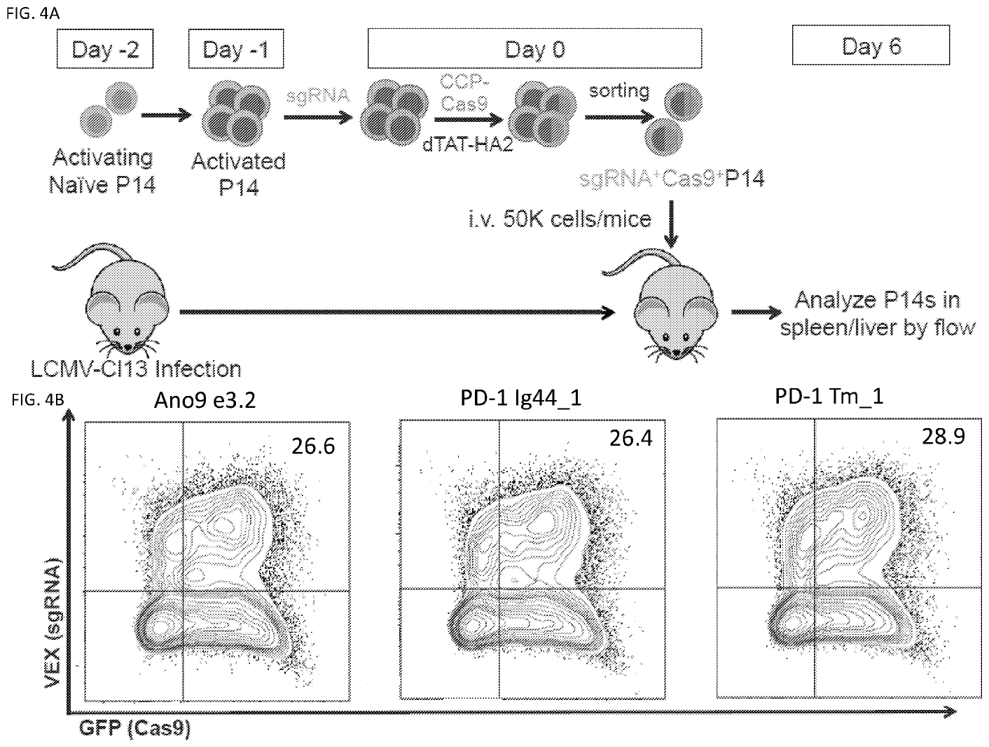

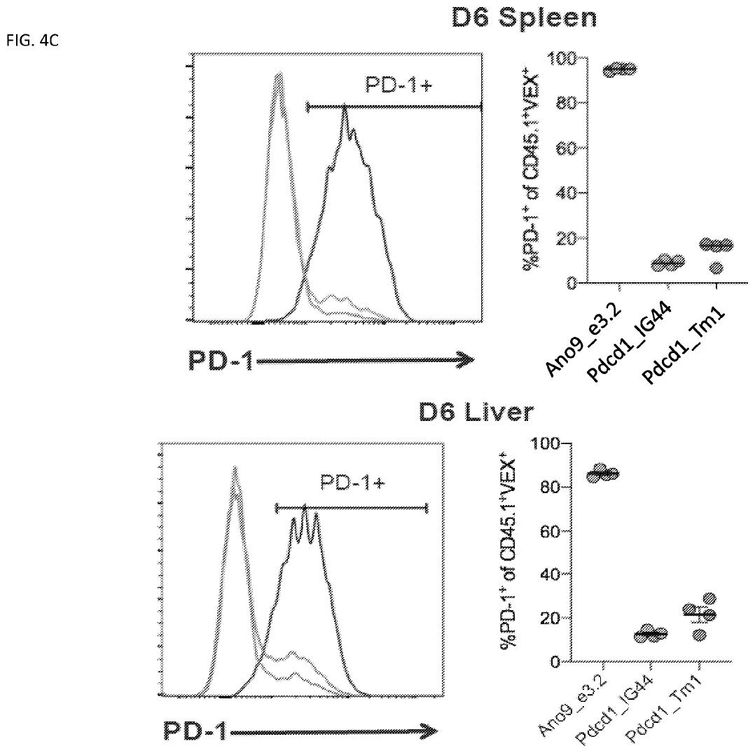

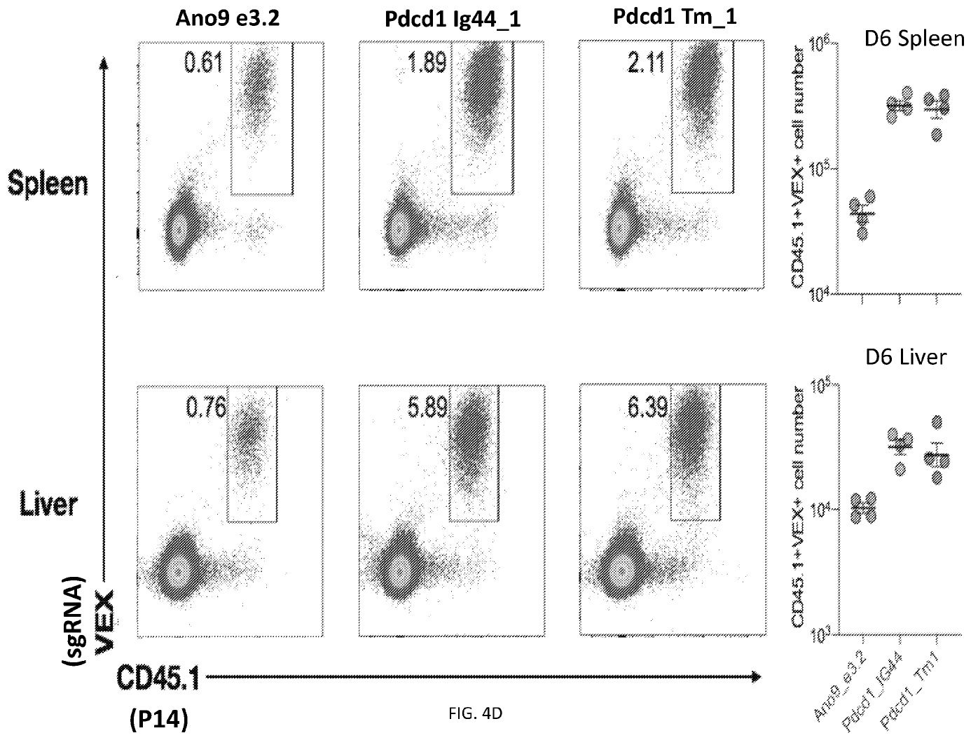

A schematic workflow for testing the in vivo editing efficiency of TAT-4×Myc-NLS Cas9 is shown in FIG. 4A. Donor mice CD8 P14 cells, which express a T cell receptor (TCR) specific for the LCMV GP33-41 epitope, were isolated and activated. On the same day (Day −2) recipient mice were infected with LCMV-Clone 13 to induce a chronic infection and T cell exhaustion. After 24 hours (Day −1), cells were infected with a retroviral vector (with a VEX reporter) expressing sgRNA targeting Ano9 or Pdcd1 (encoding PD-1) for 24 hours. Cells were treated with TAT-4×Myc NLS-Cas9, dTAT-HA2, 0.25% trypsin, and DNase I as described herein, and sgRNA⁺Cas9⁺(VEX⁺GFP⁺) P14 cells were sorted (FIG. 4B). Fifty thousand sorted cells were adoptively transferred to LCMV-Clone 13 infected recipient mice through tail vein injection. After 6 days, spleen and liver were harvested and analyzed by flow cytometry for PD-1 expression and P14 cell expansion. A dramatic decrease of PD-1 expression was observed; down to ˜20% on the cells infected with both sgRNA targeting Pdcd1, compared to cells infected with sgRNA targeting Ano9 in both spleen and liver (FIG. 4C). Importantly, the percentage of the sgRNA⁺P14 T cell population and the total number of sgRNA⁺P14 T cells increased in both sgRNA targeting Pdcd1, compared to sgRNA targeting Ano9 in both spleen and liver (FIG. 4D). This is consistent with enhanced antigen specific CD8 T cell expansion during early chronic infection as a result of genetic depletion of PD-1.

### Example 5: In Vitro Editing by TAT-4×Myc NLS-Cas9 in Human Primary T Cells

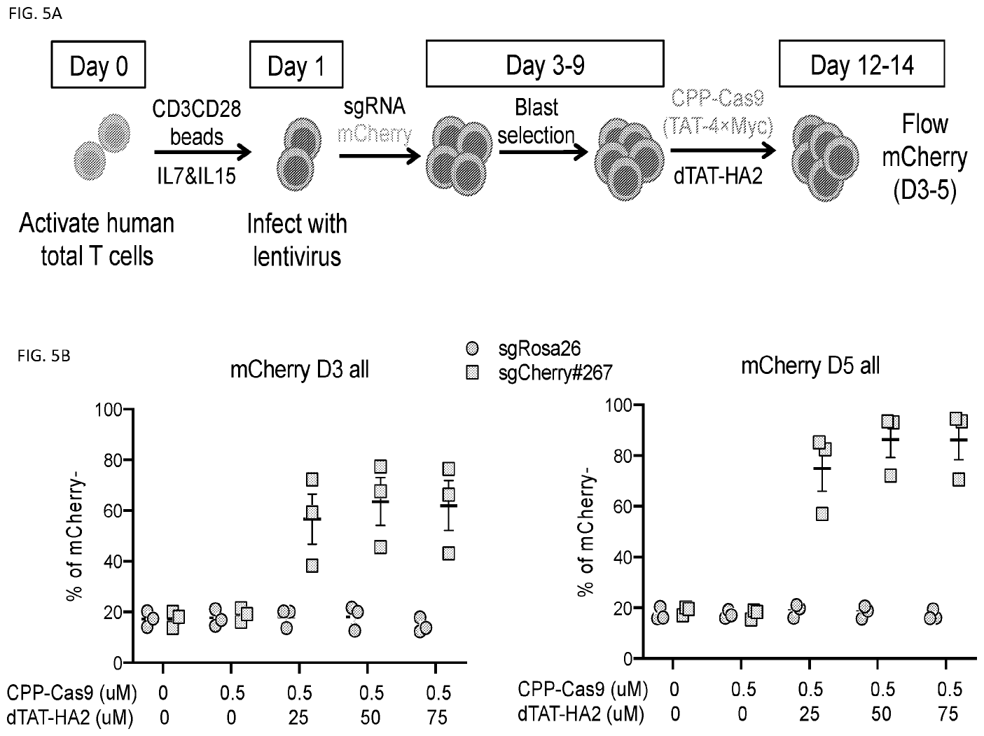

A schematic workflow for testing the in vitro editing efficiency of TAT-4×Myc NLS-Cas9 in human primary T cells is shown in FIG. 5A. Human total T cells were isolated from normal donor PBMCs by human T cell isolation kit, and activated by CD3/CD28 Dynabeads, IL-7, and IL-15 on Day 0 in OpTmizer T cell expansion medium supplied with 5% human serum and 1× Glutamax I. After 24 hours (Day 1), cells were infected with the lentiviral reporter construct as in FIG. 2A for 2 days. On Days 3-9, mCherry+ cells were selected by blasticidin, and subsequently treated by 0.5 μM TAT-4×Myc NLS-Cas9 and 25-75 μM dTAT-HA2 for 30 minutes at 37° C. in completed T cell expansion medium. Cells were washed once with PBS and cultured for an extra 5 days. The frequency of mCherry⁻ cells was measured on Days 12-14 (mCherry D3-5) by flow cytometry. The frequency of mCherry⁻ cells increased from about 20% to about 35˜70% (0.5 μM TAT-4×Myc NLS-Cas9 and 25 μM dTAT-HA2) on mCherry D3 in T cells isolated from three normal donors, and increasing the concentration of dTAT-HA2 from 25 μM to 50 μM or 75 μM increased the frequency of mCherry⁻ cells to 45˜75% (FIG. 5B, left panel). On mCherry D5, the frequency of mCherry⁻ cells increased to about 70-90% (0.5 μM TAT-4×Myc NLS-Cas9 and 25 μM dTAT-HA2) (FIG. 5B, right panel). However, the frequency of mCherry cells did not increase in T cells infected with sgRosa26 (FIG. 5B). An example histogram of mCherry- human T cells on day 5 post treatment of cells with 0.5 μM TAT-4×Myc NLS-Cas9 and 50 μM dTAT-HA2 is shown in FIG. 5C.

### Example 6: In Vitro Editing by TAT-4×Myc NLS-Cas9 in iPSCs

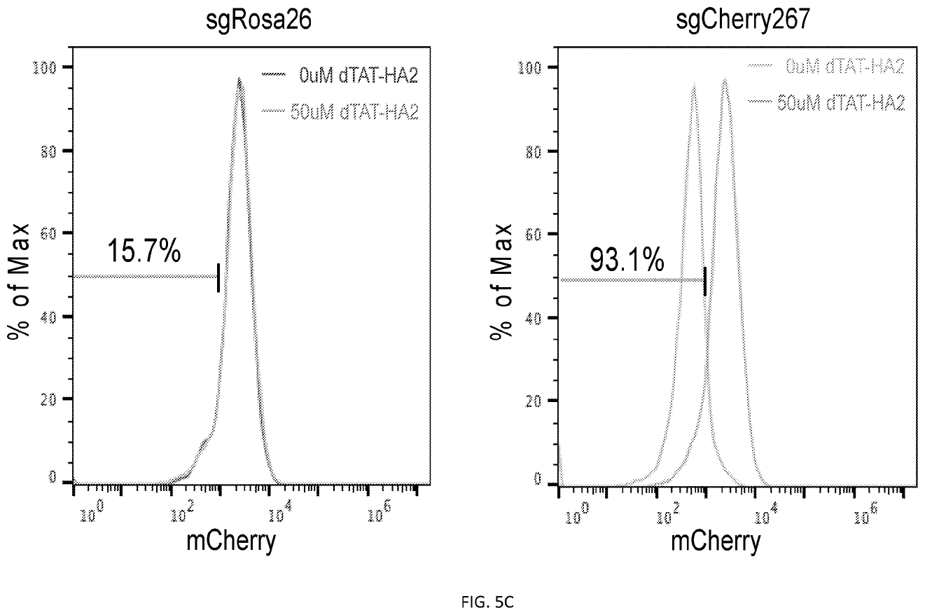

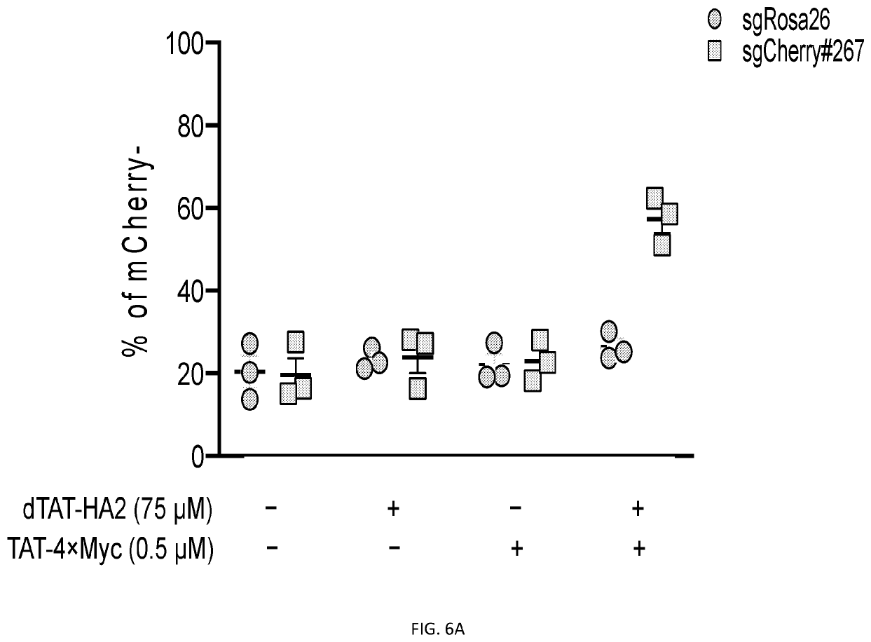

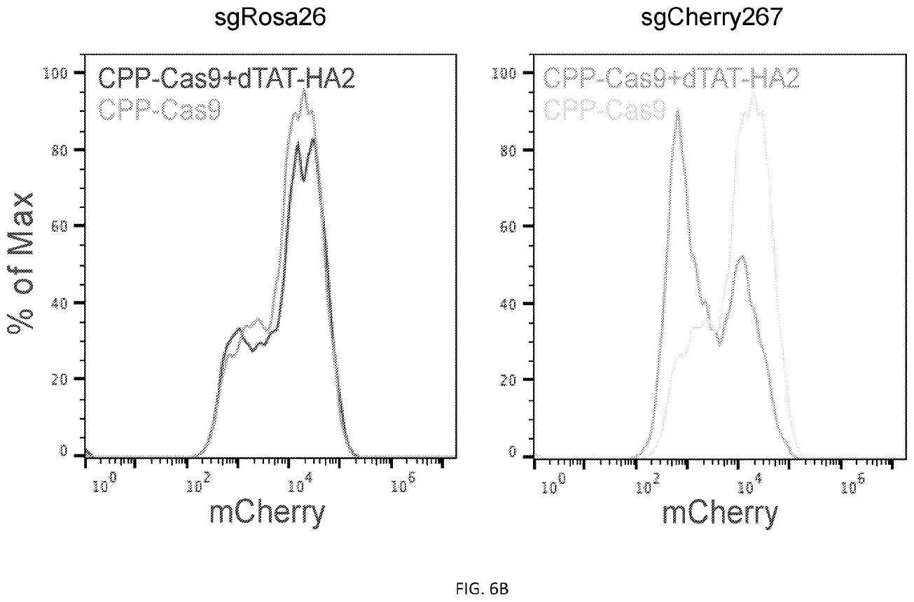

iPSCs were infected by the same lentiviral reporter construct and treated as in FIG. 2A. When incubated with 0.5 μM TAT-4×Myc NLS-Cas9 and 75 μM dTAT-HA2, the frequency of mCherry⁻ cells increased from about 20% to 60% at day 4 post-treatment. However, the frequency of mCherry⁻ cells did not increase in iPSCs infected with sgRosa26 (FIG. 6A). An example histogram of mCherry− iPSCs on day 4 post treatment of cells with 0.5 μM TAT-4×Myc NLS-Cas9 and 75 μM dTAT-HA2 is shown in FIG. 6B.

This disclosure provides a new method for in vitro and in vivo CRISPR editing of mouse and human CD8 T cells, human primary T cells, and human iPSCs. The efficiency of this editing can reach up to 90% for in vitro CD90.2 editing and in vivo PD-1 editing, which is much higher than other published methods using cell penetrating Cas9 for genome editing (Staahl et al., (2017) Nat Biotechnol 35, 431-434). In addition, this method can achieve genome editing in a timely and economic manner, since it does not require electroporation or Cas9 transgenic mice required by previously described methods for mouse CD8 T cell genome editing. Therefore, this disclosure describes a simple, efficient, and economic way to edit CD8 T cell genomes both in vitro and in vivo.

### Example 7: Peptide-Assisted Genome Editing (PAGE)

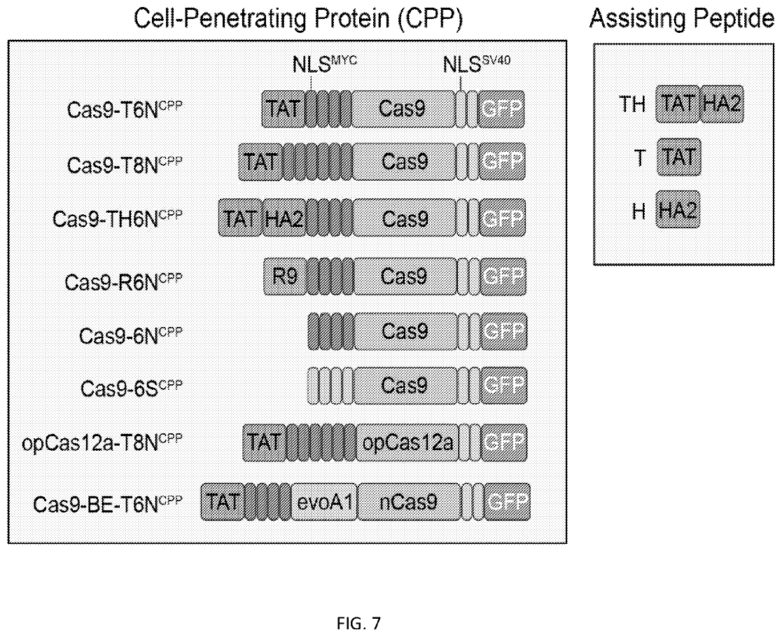

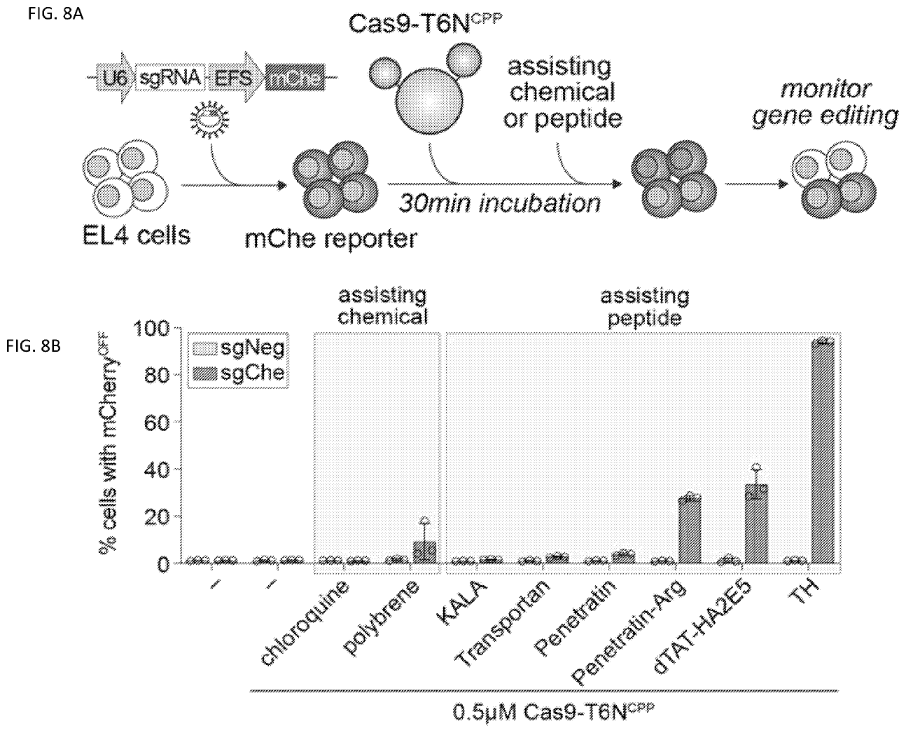

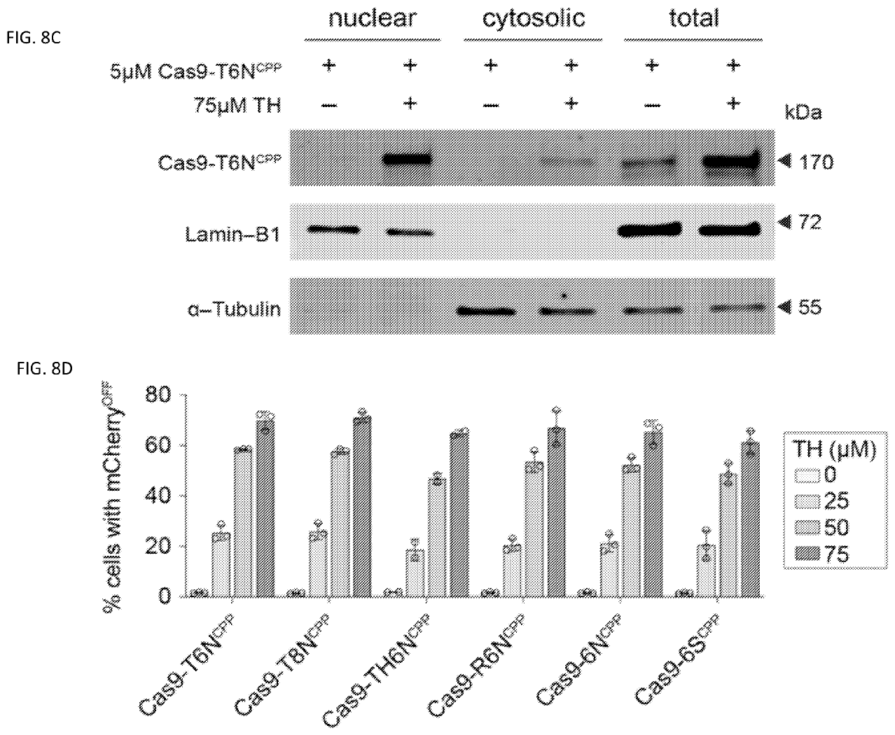

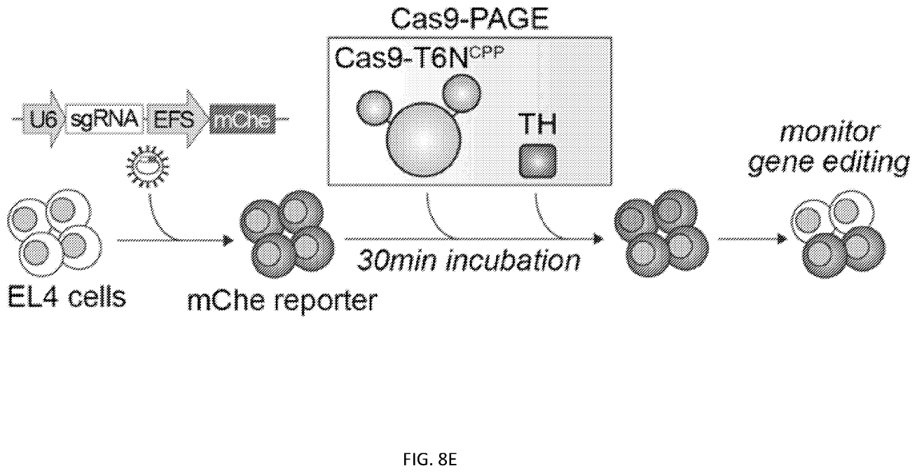

PAGE system constructs were generated comprising cell penetrating CRISPR-associated (Cas) proteins (Cas9, Cas12) and assisting/endosomal escape peptide(s) (TAT, HA2) (FIG. 7). A peptide assisted cell-penetrating Cas9 system was optimized in EL4 reporter cells (FIGS. 8A-8E). EL4, a murine T lymphoblast, was lentivirally transduced with a dual expression vector stably expressing a mCherry (mChe) fluorescence reporter and an sgRNA targeting the mCherry gene or an sgRNA targeting Ano9 gene as a negative control (FIG. 8A). EL4-mChe cells were incubated with various Cas9-CPP proteins together with various endosomal escaping or cell penetrating chemical compounds or peptides. Proteins, chemicals, and peptides were washed out after 30 minutes incubation. Gene editing efficiency was evaluated by loss of mChe fluorescence at day 4 post-treatment via flow cytometry (FIG. 8A).

The editing efficiency of Cas9-T6N^{CPP} (TAT-4×NLS^{MYC}-Cas9-2×NLS^{SV40}-sfGFP) was quantified with various endosomal escaping or cell penetrating chemical compounds and peptides in EL4-mChe reporter cells (FIG. 8B). EL4 mChe reporter cells were treated with 0.5 μM Cas9-T6N^{CPP} in the presence of the chemical compounds 200 mM chloroquine or 1 mg/ml polybrene, or 75 μM of the assisting peptides KALA, Transportan, Penetratin, Penetratin-Arg, dTAT-HA2E5, or TH (dTAT-HA2). To measure editing efficiency, the percentage of cells with loss of mCherry was measured by flow cytometry at day 4 post-treatment. Among these chemical compounds and CPP peptides tested, TH (dTAT-HA2) incubation showed the highest percentage of mCherryOFF (>90%), suggesting that TH incubation led to robust gene editing (FIG. 8B).

EL4 cells were treated with 5 μM of Cas9-T6N^{CPP} and 75 μM of TH at 37° C. for 30 minutes, then cells were washed with PBS and trypsinized to remove cell surface-bound Cas9-T6N^{CPP}. Nuclear and cytosolic fractions were separated and subject to immunoblotting analyses using antibodies against Cas9, nuclear marker Lamin B1, and cytosolic marker a-Tubulin. Western blots of Cas9-T6N^{CPP}, Lamin-B1, and α-Tubulin levels in nuclear fraction, cytosolic fraction, and whole-cell lysates prepared from EL4 cells treated with Cas9-T6N^{CPP} and TH are shown in FIG. 8C. The data showed that the addition of TH increased the translocation of Cas9-T6N^{CPP} to cells, cytosolic fractions, and especially in the nuclear fraction, compared to the cells without TH treatment (FIG. 8C). The editing efficiency of 0.5 μM Cas9-CPP variants was quantified in EL4-mChe reporter cells with various TH peptide concentration (FIG. 8D). The combination of cell-penetrating Cas protein and the endosomal escaping peptide was termed Peptide-Assisted Genome Editing (PAGE) (FIG. 8E).

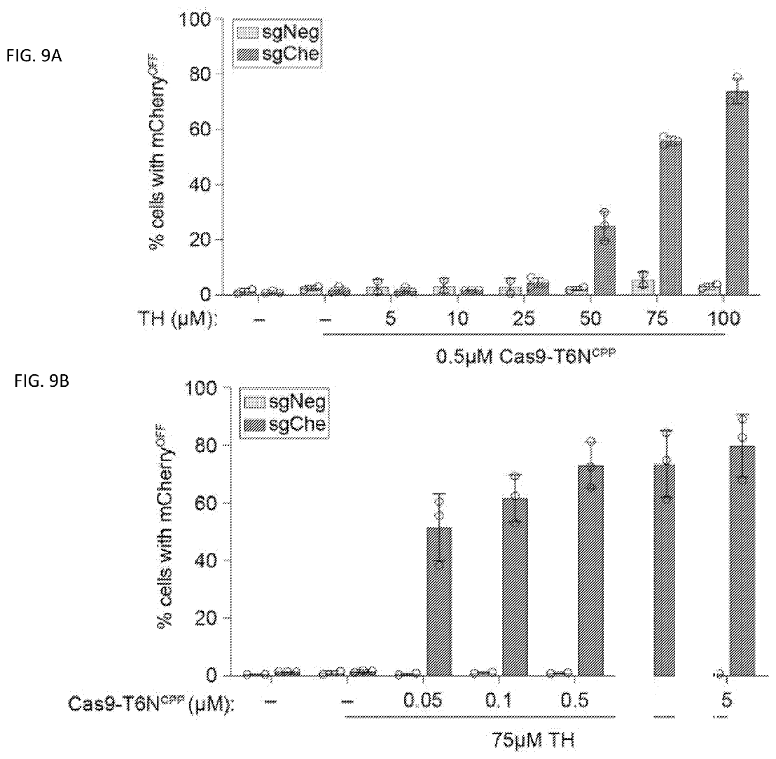

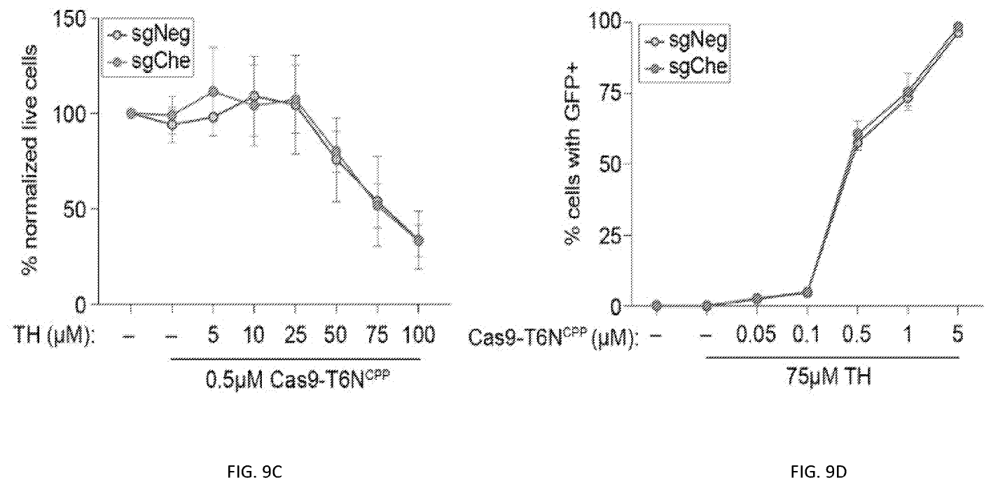

The Cas9-PAGE system was optimized in EL4 reporter cells (FIGS. 9A-9E). Gene editing efficiency was quantified with titration of either TH or Cas9-T6N^{CPP} (FIGS. 9A-9B). The percentage of cells with loss of mCherry was measured by flow cytometry at day 2 post-treatment. EL4 mCherry reporter cells were incubated with 0.5 μM Cas9-T6N^{CPP} and various concentrations of TH from 5 to 100 μM. The concentration of TH was positively correlated with increased gene editing efficiency (FIG. 9A). EL4 mCherry reporter cells were treated with n various concentrations of Cas9-T6N^{CPP} from 0.05 to 5 μM and 75 μM TH. Increasing Cas9-T6N^{CPP} concentration led to increased gene editing efficiency (FIG. 9B). Quantification of live cell recovery of EL4 cells treated with an increasing concentrations of TH is shown in FIG. 9C. Quantification of GFP positive cell population as a function of increasing amounts of Cas9-T6N^{CPP} (FIG. 9D). The GFP positive cell percentage serves as a surrogate for cell-penetrating efficiency.

TH (dTAT-HA2) supports the PAGE system in trans. Gene editing efficiency was quantified after truncation of TH. EL4 mCherry reporter cells were incubated with 0.5 μM Cas9-T6N^{CPP} in the presence of 75 μM T, H, or TH peptides. The percentage of cells with loss of mCherry was measured by flow cytometry at day 4 post-treatment. TH (dTAT-HA2), but neither T (dTAT) nor H (dHA2) peptides alone, enhanced Cas9-T6N^{CPP} editing efficiency in EL4 mCherry reporter cells (FIG. 10A). Gene editing efficiency was quantified with Cas9-T6N^{CPP} and Cas9-TH6N^{CPP}, where dTAT-HA2 (TH) was added in cis. The percentage of cells with loss of mCherry was measured by flow cytometry at day 4 post-treatment. TH facilitated the Cas9-^{CPP} in trans, independent of whether the TH peptide was present in cis or not (FIG. 10B).

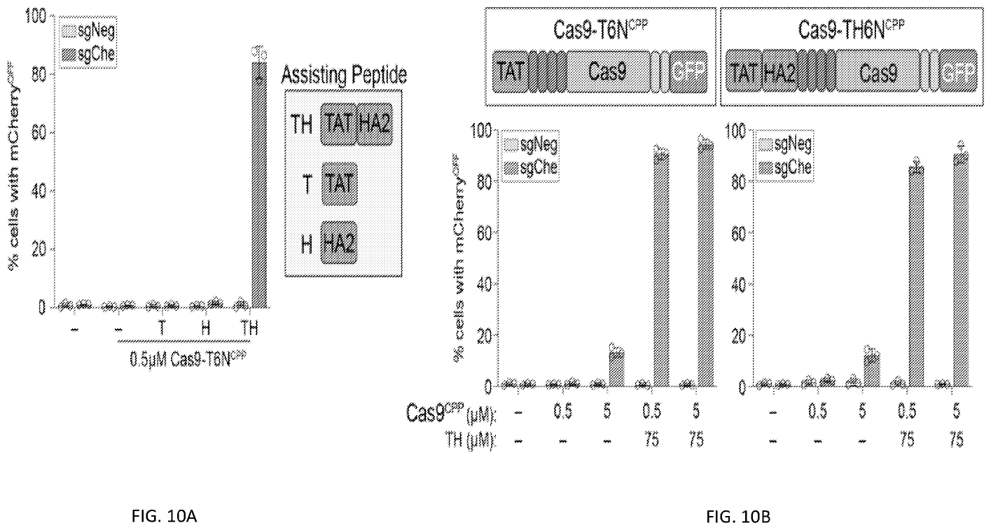

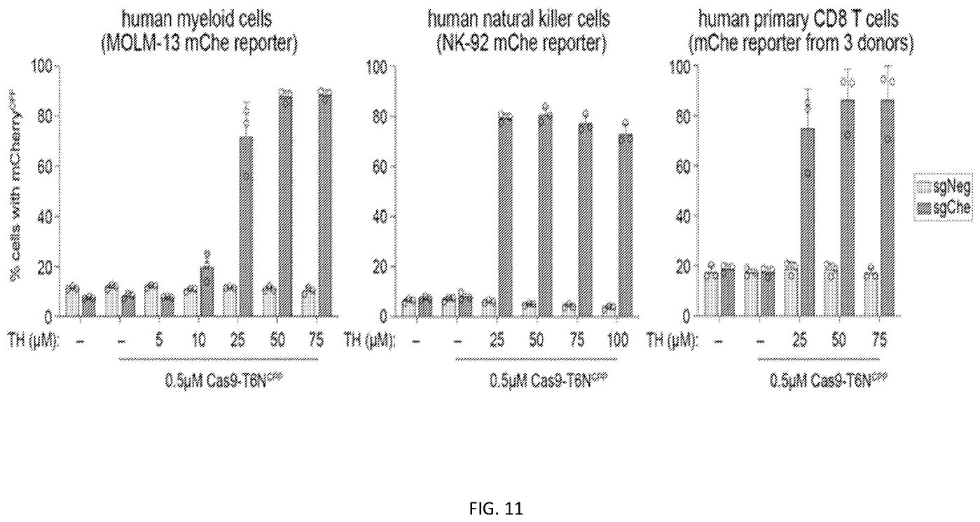

Cas9-PAGE system-mediated gene editing efficiency was quantified in various cell types (FIG. 11). The mCherry positive reporter was established in the following cell types: the human myeloid cell line model MOLM-13, the human natural killer cell line NK-92, and human primary T cells, isolated from PBMCs of three healthy donors. mCherry reporter cells were incubated with Cas9-T6N^{CPP} and TH for 30 minutes and the percentage of cells with loss of mCherry was measured by flow cytometry at day 4 post-treatment. Data demonstrated that the PAGE system can be utilized for gene editing in various cell types (FIG. 11).

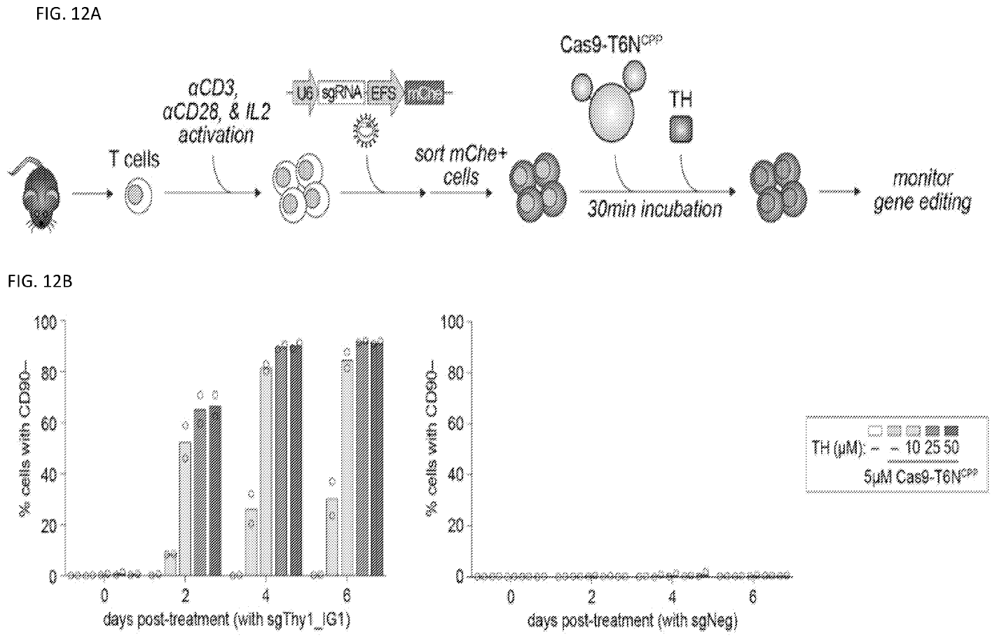

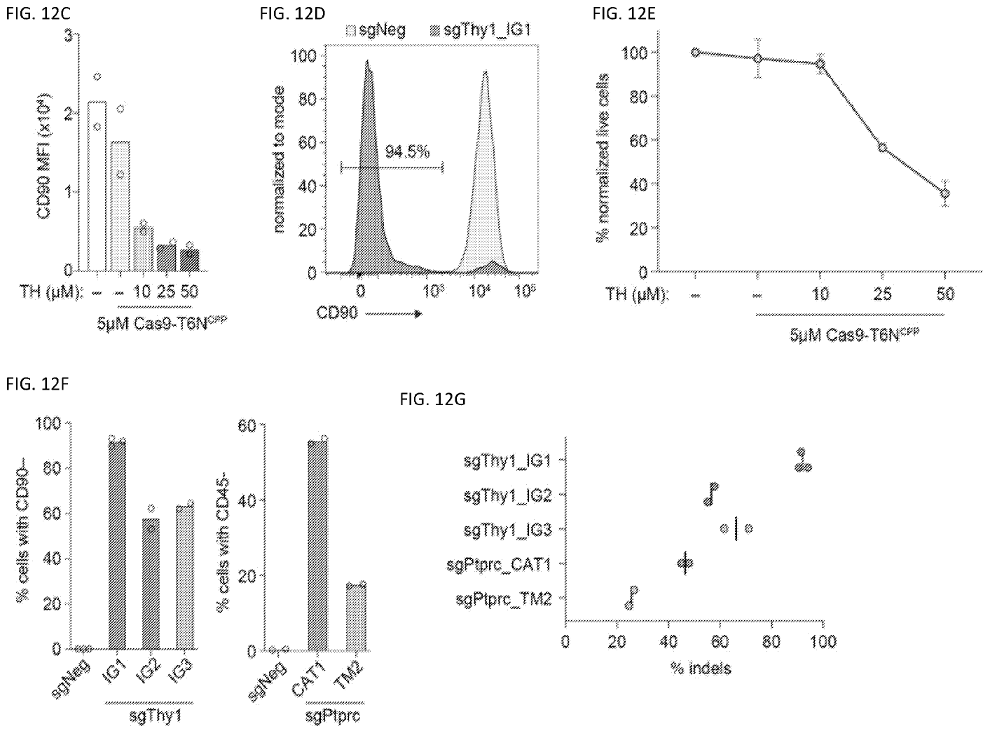

The PAGE system was evaluated in murine primary CD8 T cells ex vivo. Murine primary T cells were activated with anti-CD3, anti-CD28 and IL-2, followed by retroviral transduction with a sgRNA expression vector linked with mCherry fluorescent marker. The FACS-sorted enriched mCherry positive cells were incubated with Cas9-T6N^{CPP} and TH peptide, which were then washed out after 30 minutes incubation. Gene editing was evaluated at various time points by flow cytometry against indicated gene products or via direct sanger sequencing of the targeted genomic regions (FIG. 12A). Primary CD8 T cells were transduced with either sgThy1_IG1 or sgNeg, followed by 30 minutes incubation with various concentrations of TH and 5 μM Cas9-T6N^{CPP}. Flow cytometry analysis was performed at days 2, 4, and 6 post-treatment. A time-course analysis of CD90 protein expression in CD8 T cells treated with an increased concentration of TH is shown in FIG. 12B. Mean Fluorescent Intensity (MFI) quantification of the flow cytometry analysis at 4 days post-treatment is shown in FIG. 12C. A representative flow cytometry plot of CD90 in cells transduced with either sgThy1_IG1 or sgNeg at 4 days post-treatment is shown in FIG. 12D. Quantification of live cell recovery of CD8 T cells treated with an increased concentration of TH is shown in FIG. 12E. Additional sgRNAs were tested, which targeted the Thy1 gene (sgThy1_IG1, sgThy1_IG2, sgThy1_IG3: targeting the Immunoglobulin domain of Thy1) and the Ptprc gene (sgPtprc_CAT1 and sgPtprc_TM1: targeting either the catalytic domain or transmembrane domain of Ptprc); and a sgRNA targeting Ano9 gene was used as a negative control. A summary bar graph of gene editing efficiency of PAGE with additional sgRNAs targeting Thy1 and Ptprc genes in murine primary CD8 T cells at day 4 post-treatment is shown in FIG. 12F. Tracking of Indels by DEcomposition (TIDE) mutagenesis assays were performed using PAGE sgRNAs from FIG. 12F. Results are depicted in a dot plot showing the TIDE assay score (indel %) for each sgRNA (FIG. 12G). Genomic DNA was isolated at day 6 post-treatment of PAGE, sanger sequenced, and followed by quantification via an online TIDE analysis tool. Results demonstrated that Cas9-PAGE with retroviral sgRNA mediated genome editing in murine primary CD8 T cells ex vivo (FIGS. 12A-12G).

A cell penetrating ribonucleoprotein (RNP) complex for PAGE genome editing was tested in murine primary T cells ex vivo (FIGS. 13A-13D). A series of Cas^{CPP} variants for RNP-PAGE experiments in murine primary T cells were generated including: Cas9-T6N^{CPP} (TAT-4×NLS^{MYC} NLS-Cas9-2×NLS^{SV40}-sfGFP), Cas9-T8N^{CPP} (TAT-6×NLS^{MYC} NLS-Cas9-2×NLS^{SV40}-sfGFP), and opCas12a-T8N^{CPP} (TAT-6×NLS^{MYC} NLS-opCas12a-2×NLS^{SV40}-sfGFP) (FIG. 13A). Ex vivo editing of Cas9/opCas12a-RNP-PAGE was performed in mouse primary T cells (FIG. 13B). Murine primary CD8 T cells, either naive or activated for 2 days, were incubated with 5 μM RNP complex and various concentrations of TH for 30 minutes at 37° C. Cells were washed once and cultured for 5 days with or without sorting for GFP+ cells, and editing efficiency was measured by flow cytometry of target gene expression (FIG. 13B). CD90 expression levels were measuring in either naive CD8 or activated CD8 T cells treated with various Cas9/opCas12a-RNP-PAGE systems (FIG. 13C). Murine primary naive CD8 or activated CD8 T cells were treated with 5 μM Cas9-T6N^{CPP}, Cas9-T8N^{CPP}, or opCas12a-T8N^{CPP} RNP complex with guide RNA targeting CD90 IG domain together with 25 μM TH as described in FIG. 13B. CD90 expression was measured by flow cytometery at day 5 post-treatment. Results showed opCas12a-RNP-PAGE displayed superior gene editing efficiency over Cas9-RNP-PAGE in murine primary T cells (FIG. 13C). TH concentration was optimized in primary mouse CD8 T cells for opCas12a-RNP-PAGE delivery (FIG. 13D). Murine primary CD8 T cells were activated for 2 days and treated with 5 μM opCas12a-T8N^{CPP} RNP targeting the CD90 IG domain in the presence of various concentrations of TH from 25 to 50 μM. CD90 expression was measured by flow cytometry at day 5 post-treatment.

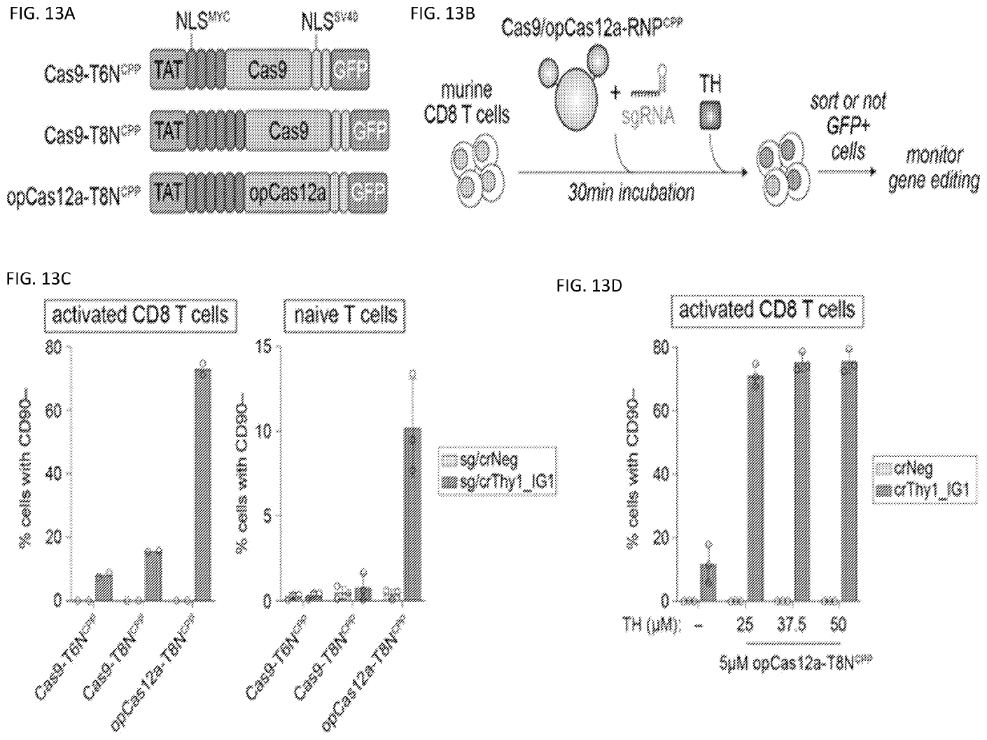

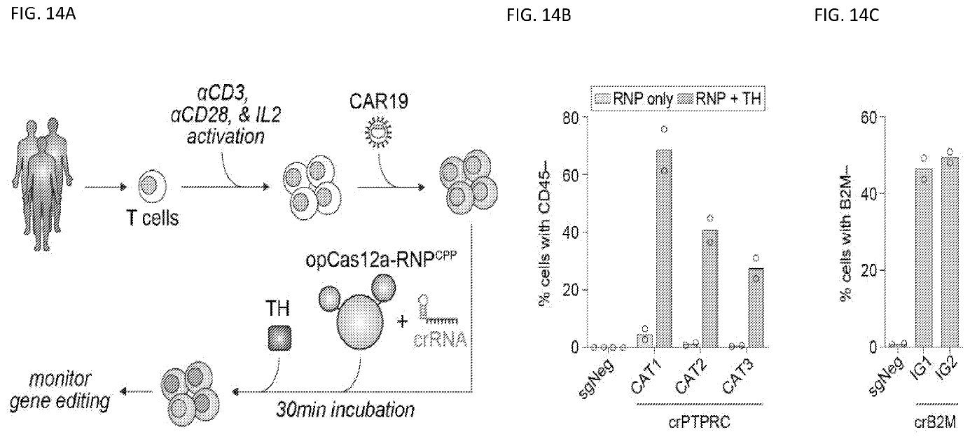

opCas12a-RNP-PAGE genome editing was demonstrated in human chimeric antigen receptor (CAR) T cells ex vivo (FIG. 14A-14C). A schematic of an experiment showing ex vivo editing of opCas12a-RNP^{CPP} in CAR T cells is shown in FIG. 14A. Human primary T cells from healthy donors were isolated and activated with anti-CD3, anti-CD28 and IL-2. Activated T cells were transduced with CAR19 lentivirus at day 1. On day 6, CAR19+ cells were FACS sorted prior to incubation with 5 μM opCas12a-T8N^{CPP} RNP and 25 μM TH for 30 minutes. Cells were cultured for an additional 10 days post-treatment and target gene expression was measured by flow cytometry. Human CAR T cells were treated with 5 μM opCas12a-T8N^{CPP} RNP targeting the catalytic domain of CD45 (encoded by PTPRC) or immunoglobulin domain of beta-2-microglobulin (encoded by B2M) in the presence of 25 μM TH (FIGS. 14B-14C). CD45 (FIG. 14B) or B2M (FIG. 14C) expression was measured by flow cytometry at day 6 post-treatment.

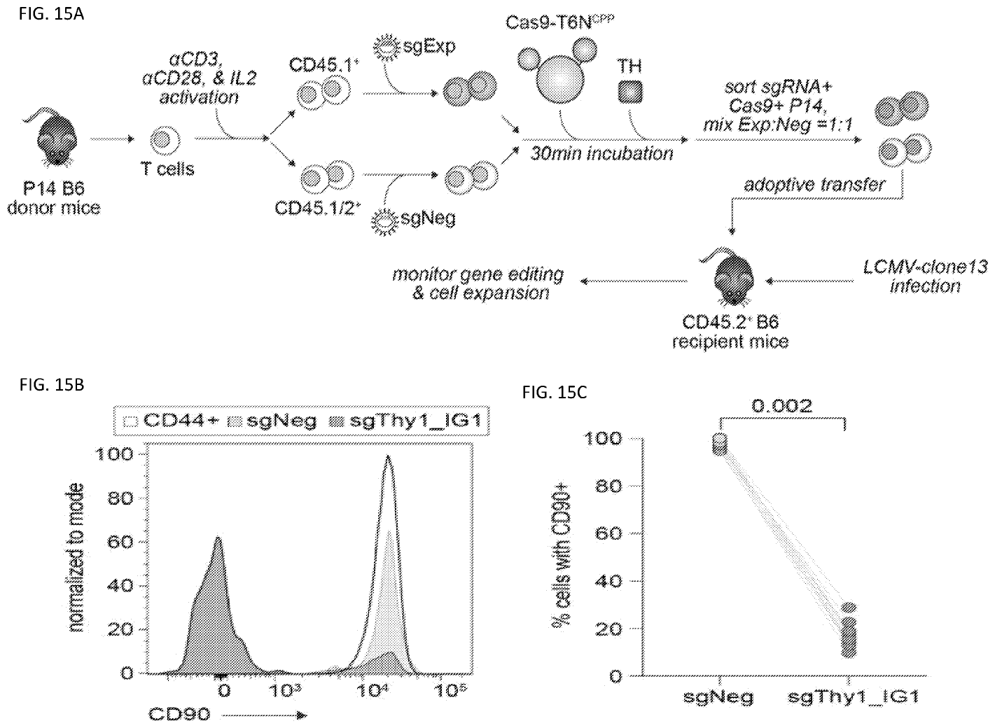

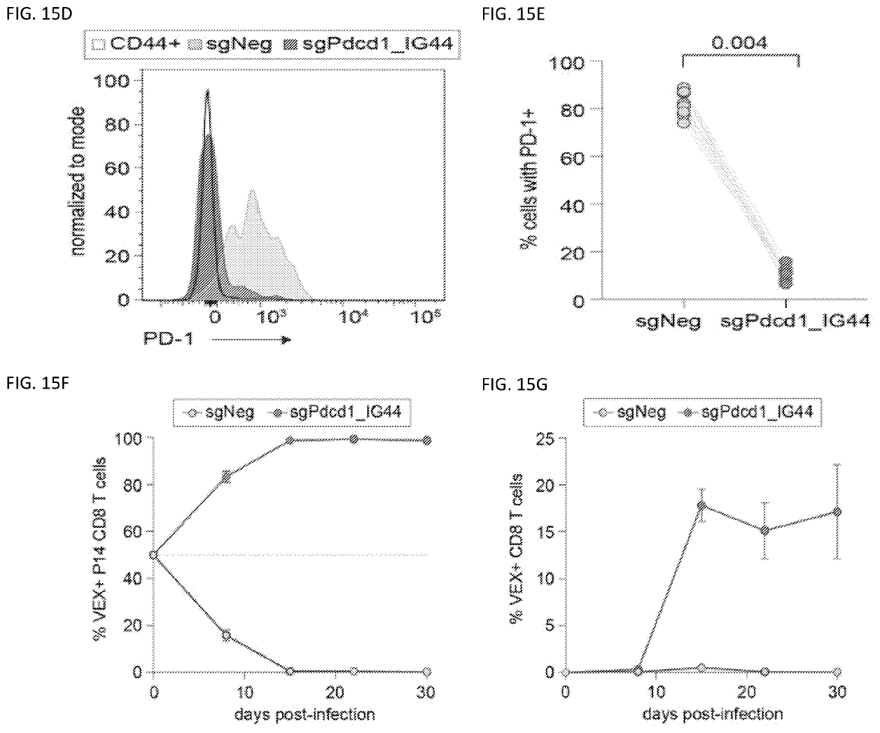

Highly efficient in vivo editing of clinically relevant genes by the Cas9-PAGE system was demonstrated in murine primary T cells (FIGS. 15A-15G). A schematic of the experimental workflow evaluating the PAGE system in murine primary CD8 T cells in vivo is shown in FIG. 15A. Donor CD8 T cells from P14 transgenic (CD45.1+ or CD45.1/2+ congenic) mice were isolated and activated with anti-CD3, anti-CD28 and IL-2, followed by retroviral transduction with either an experimental or negative control sgRNA expression vector linked with a fluorescent marker. sgRNA-transduced T cells were incubated with 5 μM Cas9-T6N^{CPP} and 25 μM TH peptide for 30 minutes prior to FACS-sorting to enrich the Cas9 positive and sgRNA positive (double positive) populations. Experimental and negative control sgRNA-transduced P14 T cells were mixed in a 1:1 ratio, followed by adoptive transfer to CD45.2+ congenic recipient mice that were infected with LCMV-clone13 virus. Gene editing and P14 T cell populations were evaluated by flow cytometry over a time course of 30 days. CD90 surface expression decreased following sgThy1_IG1 mediated editing at day 8 post-infection (FIGS. 15B-15C) as did PD-1 following sgPdcd1_IG44 mediated editing (FIG. 15D-15E). Proportion of co-transferred P14 T cells transduced with indicated sgRNA in blood over time is shown in FIG. 15F. P14 T cells transduced with indicated sgRNAs as a proportion of total CD8 T cells in blood over a time course of 30 days are depicted in FIG. 15G.

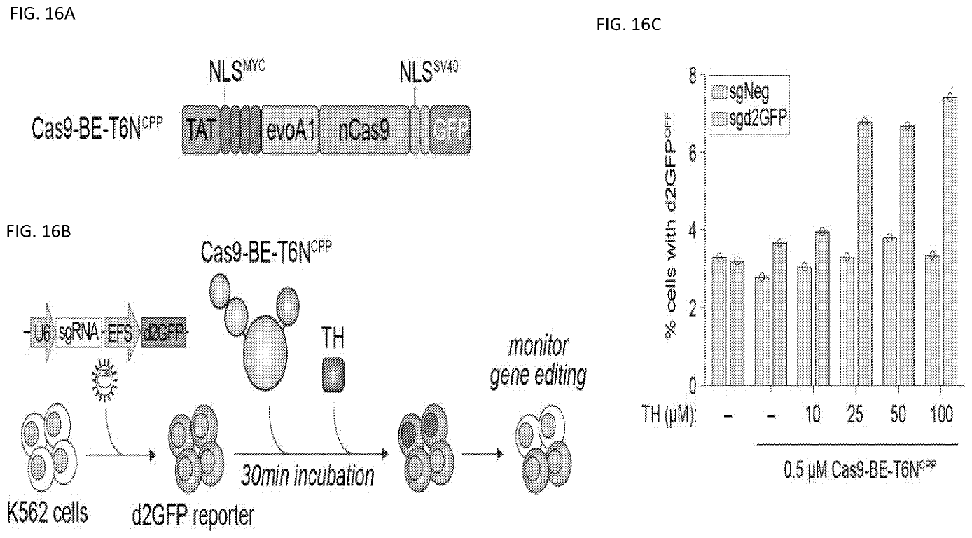

Cas9-BE PAGE base editing was demonstrated in a K562 d2GFP reporter cell line (FIGS. 16A-16C). A Cas9-BE expression construct was generated (FIG. 16A) and the base editing efficiency of the Cas9-BE PAGE system was evaluated in a K562 d2GFP reporter cell line (FIG. 16B). K562 cells were lentivirally transduced with a dual expression vector stably expressing the d2GFP fluorescence reporter and a sgRNA targeting d2GFP fluorescence reporter gene or a sgRNA targeting the Ano9 gene as a negative control. K562 d2GFP cells were incubated with Cas9-BE-T6N^{CPP} and TH peptide for 30 minutes, then the protein and peptide were washed out. Base editing was evaluated by loss of d2GFP reporter fluorescence at day 5 post-treatment when the GFP-linked Cas9-BE protein degraded completely (FIG. 16C).

### Enumerated Embodiments

The following enumerated embodiments are provided, the numbering of which is not to be construed as designating levels of importance.

Embodiment 1 provides a Peptide-Assisted Genome Editing (PAGE) system comprising a) a CRISPR associated (Cas) protein linked to a Cell Penetrating Peptide (CPP), and b) an endosomal escape peptide linked to a CPP.

Embodiment 2 provides the PAGE system of embodiment 1, wherein the Cas is Cas9, or Cas12a, or a Cas derivative.

Embodiment 3 provides the PAGE system of embodiment 2, wherein the Cas derivative is a Cas protein linked to another protein or catalytic domain.

Embodiment 4 provides the PAGE system of embodiment 3, wherein the protein or catalytic domain is selected from the group consisting of an AID deaminase, an APOBEC deaminase, a TadA deaminase, a TET enzyme, a DNA methyltransferase, a transactivation domain, a reverse transcriptase, a histone acetyltransferase, a histone deacetylase, a sirtuin, a histone methyltransferase, a histone demethylase, a kinase, and a phosphatase.

Embodiment 5 provides the PAGE system of any of the preceding embodiments, wherein the endosomal escape peptide comprises any of the amino acid sequences set forth in SEQ ID NOs: 1434-1523.

Embodiment 6 provides the PAGE system of any of the preceding embodiments, wherein the endosomal escape peptide comprises dTAT-HA2.

Embodiment 7 provides the PAGE system of any of the preceding embodiments, wherein the Cas comprises a Nuclear Localization Signal (NLS) sequence.

Embodiment 8 provides the PAGE system of embodiment 7, wherein the NLS sequence comprises the amino acid sequence PAAKRVKLD (SEQ ID NO: 1).

Embodiment 9 provides the PAGE system of embodiment 7 or 8, wherein the NLS sequence further comprises a GGS linker.

Embodiment 10 provides the PAGE system of any of the preceding embodiments, wherein the CPP comprises any of the amino acid sequences set forth in SEQ ID NOs: 10-1422.

Embodiment 11 provides the PAGE system of any of the preceding embodiments, wherein the CPP comprises a sequence derived from the trans-activating transcriptional activator (Tat) from HIV-1.

Embodiment 12 provides the PAGE system of embodiment, 11, wherein the Tat sequence comprises the amino acid sequence GRKKRRQRRRPQ (SEQ ID NO: 2).

Embodiment 13 provides an in vitro method of gene editing comprising introducing into a cell a PAGE system and at least one sgRNA or crRNA, wherein the PAGE system comprises a Cas protein linked to a CPP and an endosomal escape peptide linked to a CPP.

Embodiment 14 provides an in vivo method of gene editing comprising introducing into a cell a PAGE system and at least one sgRNA or crRNA, wherein the PAGE system comprises a Cas protein linked to a CPP and an endosomal escape peptide linked to a CPP, and administering the cell to a subject.

Embodiment 15 provides the method of embodiment 13 or 14, wherein the Cas is Cas9, or Cas12a, or a Cas derivative.

Embodiment 16 provides the method of embodiment 15, wherein the Cas derivative is a Cas protein linked to another protein or catalytic domain.

Embodiment 17 provides the method of embodiment 16, wherein the protein or catalytic domain is selected from the group consisting of an AID deaminase, an APOBEC deaminase, a TadA deaminase, a TET enzyme, a DNA methyltransferase, a transactivation domain, a reverse transcriptase, a histone acetyltransferase, a histone deacetylase, a sirtuin, a histone methyltransferase, a histone demethylase, a kinase, and a phosphatase.

Embodiment 18 provides the method of any of embodiments 13-17, wherein the endosomal escape peptide comprises any of the amino acid sequences set forth in SEQ ID NOs: 1434-1523.

Embodiment 19 provides the method of any of embodiments 13-18, wherein the endosomal escape peptide comprises dTAT-HA2.

Embodiment 20 provides the method of any of embodiments 13-19, wherein the Cas comprises a NLS sequence.

Embodiment 21 provides the method of embodiment 20, wherein the NLS sequence comprises the amino acid sequence PAAKRVKLD (SEQ ID NO: 1).

Embodiment 22 provides the method of embodiments 20 or 21, wherein the NLS sequence further comprises a GGS linker.

Embodiment 23 provides the method of any of embodiments 13-22, wherein the CPP comprises any of the amino acid sequences set forth in SEQ ID NOs: 10-1422.

Embodiment 24 provides the method of any of embodiments 13-23, wherein the CPP comprises a sequence derived from the trans-activating transcriptional activator (Tat) from HIV-1.

Embodiment 25 provides the method of embodiment 24, wherein the Tat sequence comprises the amino acid sequence GRKKRRQRRRPQ (SEQ ID NO: 2).

Embodiment 26 provides the method of any of embodiments 13-25, wherein the method does not require electroporation.

Embodiment 27 provides the method of any of embodiments 13-26, wherein the PAGE system is introduced into the cell in a medium that does not contain serum.

Embodiment 28 provides the method of any of embodiments 13-27, wherein the endosomal escape peptide is introduced into the cell at a concentration of about 25-75 μM.

Embodiment 29 provides the method of any of embodiments 13-28, wherein the Cas is introduced into the cell at a concentration of about 0.5-5 μM.

Embodiment 30 provides the method of any of embodiments 13-29, wherein the cell is an immune cell.

Embodiment 31 provides the method of any of embodiments 13-30, wherein the cell is selected from the group consisting of a primary human CD8 T cell, a human iPSC, and a CAR T cell.

Embodiment 32 provides the method of any of embodiments 13-31, wherein the sgRNA targets Ano9, Pdcd1, Thy1, Ptprc, PTPRC, or B2M.

Embodiment 33 provides the method of any of embodiments 13-32, wherein the subject is in need of a treatment for a disease or disorder, and wherein when the edited cell is administered to the subject, the disease or disorder is treated in the subject.

Embodiment 34 provides the method of embodiment 33, wherein the disease or disorder is an infection.

Embodiment 35 provides the method of embodiment 34, wherein the disease or disorder is related to T cell exhaustion.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

TABLE 2
Cell Penetrating Peptides (CPP):
Exemplary Sequences

| SEQ | |
|---|---|
| ID | |
| NO: | Amino Acid Sequence |
| 10 | ALYLLGKINLKALAALAKKIL |
| 11 | ANIIPLLPIC |
| 12 | AAAWFW |
| 13 | AAVACRICMRNFSTRQARRNHRRRHRR |
| 14 | AAVALLPAVLLALLAK |
| 15 | AAVALLPAVLLALLAKKNNLKDCGLF |
| 16 | AAVALLPAVLLALLAKKNNLKECGLY |
| 17 | AAVALLPAVLLALLAP |
| 18 | AAVALLPAVLLALLAPEILLPNNYNAYESYKYPGMFIALSK |
| 19 | AAVALLPAVLLALLAPRKKRRQRRRPPQ |
| 20 | AAVALLPAVLLALLAPRKKRRQRRRPPQC |
| 21 | AAVALLPAVLLALLAPRRRRRR |
| 22 | AAVALLPAVLLALLAPSGASGLDKRDYV |
| 23 | AAVALLPAVLLALLAPVQRKRQKLMP |
| 24 | AAVALLPAVLLALLAVTDQLGEDFFAVDLEAFLQEFGLLPEKE |
| 25 | WELVVLYGRKKRRQRRR |
| 26 | ACGRGRGRCGRGRGRCG |
| 27 | ACGRGRGRCRGRGRGCG |
| 28 | ACHGRRWGCGRHRGRCG |
| 29 | ACRDRFRNCPADEALCG |
| 30 | ACRDRFRNCPADERLCG |
| 31 | ACRDRFRRCPADERLCG |
| 32 | ACRDRFRRCPADRRLCG |
| 33 | ACRGRGRGCGRGRGRCG |
| 34 | ACRGRGRGCGSGSGSCG |
| 35 | ACRGRGRGCGSGSRSCG |
| 36 | ACRGRGRGCRGRGRGCG |
| 37 | ACRGRGRRCGSGRRSCG |
| 38 | ACRGRGRRCGSGSRSCG |
| 39 | ACRGRRRGCGRRRGRCG |
| 40 | ACRGSGRGCGRGSGRCG |
| 41 | ACRRSRRGCGRRSRRCG |
| 42 | ACSDRFRNCPADEALCG |
| 43 | ACSDRFRNCPADEALCGRRRRRRRR |
| 44 | ACSGRGRGCGRGRGSCG |
| 45 | ACSGRGRGCGSGSGSCG |
| 46 | ACSGRGSGCGSGRGSCG |
| 47 | ACSGSGSGCGSGSGSCG |
| 48 | ACSGSGSGCGSGSGSCGRRRRRRRR |
| 49 | ACSHSGHGCGHGSHSCGRRRRRRRR |
| 50 | ACSHSGWGCGHGSWSCGRRRRRRRR |
| 51 | ACSSSPSKHCG |
| 52 | ACSSSPSKHCGGGGRRRRRRRRR |
| 53 | ADVFDRGGPYLQRGVADLVPTATLLDTYSP |
| 54 | AEAEAEAEAKAKAKAK |
| 55 | AEAEAEAEAKAKAKAKAGGGHRRRRRRR |
| 56 | AEKVDPVKLNLTLSAAAEALTGLGDK |
| 57 | AGYLGKINLKALAALAKKIL |
| 58 | AGYLLGKINLKALAALAKKIL |
| 59 | AGYLLGKTNLKALAALAKKIL |
| 60 | AGYLLGKTNLKALAALAKKIL |
| 61 | AGYLLGKINLKALAALAKKIL |
| 62 | AGYLLGHINLHHLAHLHHIL |
| 63 | AGYLLGHINLHHLAHLHHILC |
| 64 | AGYLLGKINLKALAALAKKIL |
| 65 | AGYLLGKINLKALAALAKKIL |
| 66 | AGYLLGKINLKALAALAKKIL |
| 67 | AGYLLGKINLKALAALAKKILGGC |
| 68 | AGYLLGKINLKALAALAKKILTYADFIASGRTGRRNAI |
| 69 | AGYLLGKINLKKLAKLLLIL |
| 70 | AGYLLGKLKALAALAKKIL |
| 71 | AGYLLGKLLKKLAAAALKKLL |
| 72 | AGYLLGKTNLKALAALAKKIL |
| 73 | AGYLLGKTNLKALAALAKKIL |
| 74 | AGYLLGKINLKALAALAKKIL |
| 75 | AHALCLTERQIKIWFQNRRMKWKKEN |
| 76 | AHALCPPERQIKIWFQNRRMKWKKEN |
| 77 | RRRRRRRRR |
| 78 | NIIAPLLPIC |
| 79 | NIILLIC |
| 80 | NIILLPIC |
| 81 | NIIPLLAPIC |
| 82 | NIIPLLIC |
| 83 | NIIPLLPIC |
| 84 | AIIYRDLIS |
| 85 | AIPNNQLGFPFK |
| 86 | AKKAKAAKKAKAAKKAKAAKKAKAAKKAKA |
| 87 | AKKKAAKAAKKKAAKAAKKKAAKA |
| 88 | AKKKAAKAAKKKAAKAAKKKAAKAAKKKAAKA |
| 89 | AKKRRQRRR |
| 90 | AKKRRQRRRAKKRRQRRR |
| 91 | AKVKDEPQRRSARLSAKPAPPKPEPKPKKAPAKK |
| 92 | ALALALALALALALALKIKKIKKIKKIKKLAKLAKKIK |
| 93 | ALFDIIKKIAESF |
| 94 | ALIILRRRIRKQAHAHSK |
| 95 | ALWKTLLKKVLKA |
| 96 | ALWKTLLKKVLKAPKKKRKV |
| 97 | ALWMRWYSPTTRRYG |
| 98 | ALWMTLLKKVLKAAAKAALNAVLVGANA |
| 99 | APWHLSSQYSRT |
| 100 | AQIKIWFQNRRMKWKK |
| 101 | ARCSDRFRNCPADEALCGR |
| 102 | ARCSGSGSGCGSGSGSCGR |
| 103 | ARRARAARRARAARRARAARRARAARRARA |
| 104 | ARRCSDRFRNCPADEALCGRR |
| 105 | ARRCSGSGSGCGSGSGSCGRR |
| 106 | ARRRAARAARRRAARAARRRAARA |
| 107 | ARRRAARAARRRAARAARRRAARAARRRAARA |
| 108 | ARRRCSDRFRNCPADEALCGRRR |
| 109 | ARRRCSGSGSGCGSGSGSCGRRR |
| 110 | ARRRRCSDRFRNCPADEALCGRRRR |
| 111 | ARRRRCSGSGSGCGSGSGSCGRRRR |
| 112 | ARTINAQQAELDSALLAAAGFGNTTADVFDRG |
| 113 | ASMWERVKSIIKSSLAAASNI |
| 114 | AVPAENALNNPF |
| 115 | AVPAKKRZKSV |
| 116 | AYALCLTERQIKIWFANRRMKWKKEN |
| 117 | AYGRKKRRQRRR |
| 118 | AYLLGKINLKALAALAKKIL |
| 119 | AYRIKPTFRRLKWKYKGKFW |
| 120 | ERRRKKRRRE |
| 121 | ERRRKKRRRE |
| 122 | ERRRKKRRRE |
| 123 | ERRRKKRRRE |
| 124 | KRRRRE |
| 125 | KRRRRRRE |
| 126 | KRRRRRRRE |
| 127 | KRRRRRRRRE |
| 128 | KRRRRRRRRRE |
| 129 | GLRKRLRKFRNKIKEK |
| 130 | GLRKRLRKFRNKIKEK |
| 131 | RKRRRRRRE |
| 132 | RRKRRRRRE |
| 133 | RRRKRRRRE |
| 134 | RRRRKRRRE |
| 135 | RRRRRRR |
| 136 | GGGGRRFFRRFRR |
| 137 | GGGGRRFFRRWRR |
| 138 | GGGGRRFWRRFRR |
| 139 | GGGGRRFWRRWRR |
| 140 | GGGGRRWFRRFRR |
| 141 | GGGGRRWFRRWRR |
| 142 | GGGGRRWWRRFRR |
| 143 | GGGGRRWWRRWRR |
| 144 | BRRRRRR |
| 145 | BRRRRRRR |
| 146 | BRRRRRRRR |
| 147 | BRRXRRXRRX |
| 148 | BXRXRXXRXRXXRXRX |
| 149 | CALNNYGRKKRRQRRR |
| 150 | CARSKNKDC |
| 151 | CASGQQGLLKLC |
| 152 | CAYGGQQGGQGGG |
| 153 | CAYGRKKRRQRRR |
| 154 | CCTGRKKRRQRRR |
| 155 | CELAGIGILTVKKKKKQKKK |
| 156 | CELAGIGILTVRKKRRQRRR |
| 157 | CGAYDLRRRERQSRLRRRERQSR |
| 158 | CGGGARKKAAKAARKKAAKAARKKAAKAARKKAAKA |
| 159 | CGGGGYGRKKRRQRRR |
| 160 | CGGGRRRRRRRRRLLLL |
| 161 | CGGGYGRKKRRQRRR |
| 162 | CGGKDCERRFSRSDQLKRHQRRHTGVKPFQ |
| 163 | CGGMVTVLFRRLRIRRASGPPRVRV |
| 164 | CGNKRTR |
| 165 | CGNKRTRGC |
| 166 | CGNVVRQGCGYGRKKRRQRRRGTALDWSWLQTE |
| 167 | CGRKKRRQRRRPPQ |
| 168 | CGRKKRRQRRRPPQ |
| 169 | CGRKKRAARQRAARAARPPQ |
| 170 | CGRKKRAARQRRRPPQ |
| 171 | CGRKKRLLRQRLLRLLRPPQ |
| 172 | CGRKKRLLRQRRRPPQ |
| 173 | CGRKKRRQRRRPPQ |
| 174 | CGRKKRRQRAARRPPQ |
| 175 | CGRKKRRQRLLRRPPQ |
| 176 | CGRKKRRQRRRPPQ |
| 177 | CGRKKRRQRRAARPPQ |
| 178 | CGRKKRRQRRLLRPPQ |
| 179 | CGRKKRRQRRRPPQ |
| 180 | CGRKKRRQRRWWRPPQ |
| 181 | CGRKKRRQRWWRRPPQ |
| 182 | CGRKKRWWRQRRRPPQ |
| 183 | CGRKKRWWRQRWWRWWRPPQ |
| 184 | CGYGRKKRRQRRRGC |
| 185 | CHAIYPRH |
| 186 | CHHHHHRRRRRRRRRHHHHHC |
| 187 | CHHRRRRHHC |
| 188 | CIGAVLKVLTTGLPALISWIKRKRQQ |
| 189 | CIISRDLISH |
| 190 | CKDEPQRRSARLSAKPAPPKPEPKPKKAPAKK |
| 191 | ckkkkkkkkk |
| 192 | CKYGRKKRRQRRR |
| 193 | CLLIILRRRIRKQAHAHSKNHQQQNPHQPPM |
| 194 | CLLYWFRRRHRHHRRRHRRC |
| 195 | CNGRC |
| 196 | CREKAKKLFKKILKKL |
| 197 | CRFRFKCCKK |
| 198 | CRFRWKCCKK |
| 199 | CRGDC |
| 200 | CRGDK |
| 201 | CRGDKGDPC |
| 202 | CRGDKGPDC |
| 203 | CRKARYRGRKRQR |
| 204 | crkkrrqrrr |
| 205 | CRNGRGPDC |
| 206 | CRQIKIWFPNRRMKWKKC |
| 207 | CRQIKIWFQNRRMKWKK |
| 208 | CRQIKIWFQNRRMKWKKKLAKLAKKLAKLAK |
| 209 | CRRLRHLRHHYRRRWHRFRC |
| 210 | CRRRRRRRR |
| 211 | crrrrrrrrr |
| 212 | CRWRFKCCKK |
| 213 | CRWRWKCCKK |
| 214 | CRWRWKCG |
| 215 | CRWRWKCGCKK |
| 216 | CRWRWKCSKK |
| 217 | CRWRWKCXKK |
| 218 | CRWRWKSSKK |
| 219 | CRWRWKXCKK |
| 220 | CRWRWKXXKK |
| 221 | CSIPPEVKFNKPFVYLI |
| 222 | CSIPPEVKFNPFVYLI |
| 223 | CSKSSDYQC |
| 224 | CSSLDEPGRGGFSSESKV |
| 225 | CTSTTAKRKKRKLK |
| 226 | CTWLKY |
| 227 | CTWLKYH |
| 228 | CVKRGLKLRHVRPRVTRDV |
| 229 | CVQWSLLRGYQPC |
| 230 | CVSRRRRRRGGRRRR |
| 231 | CWKKK |
| 232 | CWKKKKKKKK |
| 233 | CWKKKKKKKKKKKKK |
| 234 | CWKKKKKKKKKKKKKKKKKK |
| 235 | FRRRRQ |
| 236 | CYGRKKRRQRRR |
| 237 | DAATARGRGRSAASRPTERPRAPARSASRPRRPVD |
| 238 | DAATATRGRSAASRPTQRPRAPARSASRPRRPVE |
| 239 | DCRWRWKCCKK |
| 240 | DCRWRWKCXKK |
| 241 | DCRWRWKXCKK |
| 242 | DCRWRWKXXKK |
| 243 | DFNKFHTFPQTAIGVGAP |
| 244 | DITYRFRGPDWL |
| 245 | DPATNPGPHFPR |
| 246 | DPKGDPKGVTVTVTVTVTGKGDPKPD |
| 247 | DPVDTPNPTRRKPGK |
| 248 | DRDDRDDRDDRDDRDDR |
| 249 | DRDRDRDRDR |
| 250 | DRRRRGSRPSGAERRRR |
| 251 | DRRRRGSRPSGAERRRRRAAAA |
| 252 | DSLKSYWYLQKFSWR |
| 253 | DTWAGVEAIIRILQQLLFIHFR |
| 254 | EARPALLTSRLRFIPK |
| 255 | ECYPKKGQDP |
| 256 | EEE |
| 257 | EEEAA |
| 258 | EEEAAGRKRKKRT |
| 259 | EEEAAKKK |
| 260 | EEEEEEEEEEPLGLAGVSRRRRRRGGRRRR |
| 261 | EEEEEEEEPLGLAGRRRRRRRRN |
| 262 | EKGKKIFIMK |
| 263 | EKRPRTAFSSEQLARLKREFNENRYLTTERRRQQLSSELGLN |
| | EAQIKIWFQNKRAK |
| 264 | ELALELALEALEAALELA |
| 265 | ELPVM |
| 266 | ELVVLGKLYGRKKRRQRRR |
| 267 | EPDNWSLDFPRR |
| 268 | ERERERERERERER |
| 269 | ERKKRRRE |
| 270 | ESGGGGSPGRRRRRRRRRRR |
| 271 | EXREXRILFQYEXREXR |
| 272 | FAPWDTASFMLG |
| 273 | FDPFFWKYSPRD |
| 274 | FFFAAGRKRKKRT |
| 275 | FFFFFFGRRRRRRRRGC |
| 276 | FFFFGRRRRRRRRGC |
| 277 | FFGRRRRRRRGC |
| 278 | FFGSVLKLIPKIL |
| 279 | FFKKLALHALHLLALLWLHLAHLALKK |
| 280 | FFLIGRRRRRRRRGC |
| 281 | fflipkgrrrrrrrr |
| 282 | FFLIPKGRRRRRRRRGC |
| 283 | FFLIPKGRRRRRRRRR |
| 284 | FHFHFRFR |
| 285 | FIIFRIAASHKK |
| 286 | FIRIGC |
| 287 | FISAIASMLGKFL |
| 288 | FITKALGISYGRKKRR |
| 289 | FITKALGISYGRKKRRQRRRPPQ |
| 290 | FKCRRWQWRM |
| 291 | FKKFRKF |
| 292 | FKKLALHALHLLALLWLHLAHLALKK |
| 293 | FKQqQqQqQqQq |
| 294 | FLFPLITSFLSKVL |
| 295 | FLGKKFKKYFLQLLK |
| 296 | FLIFIRVICIVIAKLKANLMCKT |
| 297 | FLKLLKKFLKLFKKLLKLF |
| 298 | FLPIIAKVLSGLL |
| 299 | FLPIVGKLLSGLL |
| 300 | FLPLIGKILGTIL |
| 301 | FLPLIGRVLSGIL |
| 302 | FLSGIVGMLGKLF |
| 303 | FLSSIGKILGNLL |
| 304 | FQFNFQFNGGGHRRRRRRR |
| 305 | FQNRRMKWKK |
| 306 | FQPYDHPAEVSY |
| 307 | FQWQRNMRKVRGPPVS |
| 308 | FrFKFrFK |
| 309 | FRVPLRIRPCVVAPRLVMVRHTFGRIARWVAGPLETR |
| 310 | FTFHFTFHF |
| 311 | FTYKNFFWLPEL |
| 312 | FVQWFSKFLGRIL |
| 313 | FVTRGCPRRLVARLIRVMVPRR |
| 314 | FXrFXrFXr |
| 315 | FXrFXrFXrFXr |
| 316 | FXrFXrFXrFXrFXr |
| 317 | FXrFXrFXrFXrFXrFXr |
| 318 | GACTKSIPPICFPD |
| 319 | GAFDIIKKIAESF |
| 320 | GALFLAFLAAALSLMGLWSQPKKKRKV |
| 321 | GALFLAFLAAALSLMGLWSQPKKKRRV |
| 322 | GALFLGFLGAAGSTMGAWSQPKKKRKV |
| 323 | GALFLGFLGAAGSTMGAWSQPKSKRKV |
| 324 | GALFLGWLGAAGSTMGAPKKKRKV |
| 325 | GALFLGWLGAAGSTMGAPKSKRKVGGC |
| 326 | GAYDLRRRERQSRLRRRERQSR |
| 327 | GCGGGYGRKKRRQRRR |
| 328 | GDLPHLKLC |
| 329 | GDVYADAAPDLFDFLDSSVTTARTINA |
| 330 | GEQIAQLIAGYIDIILKKKKSK |
| 331 | GGAYVTRSSAVRLRSSVPGVRLLQ |
| 332 | GGGARKKAAKAARKKAAKAARKKAAKAARKKAAKA |
| 333 | GGGGRRRRRRRRRLLLL |
| 334 | GGGRRRRRRYGRKKRRQRR |
| 335 | GGRRARRRRRR |
| 336 | GGVCPAILKKCRRDSDCPGACICRGNGYCGSGSD |
| 337 | GGVCPKILAACRRDSDCPGACICRGNGYCGSGSD |
| 338 | GGVCPKILAKCRRDSDCPGACICRGNGYCGSGSD |
| 339 | GGVCPKILKACRRDSDCPGACICRGNGYCGSGSD |
| 340 | GGVCPKILKKCRRDSDCPGACICRGNGWCGSGSD |
| 341 | GGVCPKILKKCRRDSDCPGACICRGNGYCGSGSD |
| 342 | GGVCPKILRRCRRDSDCPGACICRGNGWCGSGSD |
| 343 | GGVCPKILRRCRRDSDCPGACICRGNGYCGSGSD |
| 344 | GGVCPKILRRCRRDSDCPGACICRGNGYCGSGSR |
| 345 | GGVCPRILRRCRRDSDCPGACICRGNGYCGSGSK |
| 346 | GHHHHHHHHHHHHH |
| 347 | GIGKFLHSAKKFGKAFVGEIMNSGGKKWKMRRNQFWVKVQRG |
| 348 | GIGKFLHSAKKWGKAFVGQIMNC |
| 349 | GILDAIKAIAKAAG |
| 350 | GKHRHERGHHRDRRER |
| 351 | GKINLKALAALAKKIL |
| 352 | GKKALKLAAKLLKKC |
| 353 | GKKKKKKKKK |
| 354 | GKKKKRKREKL |
| 355 | GKKKRKLSNRESAKRSR |
| 356 | GKKTNLFSALIKKKKTA |
| 357 | GKRARNTEAARRSRARKL |
| 358 | GKRKKKGKGLGKKRDPCLRKYK |
| 359 | GKRKKKGKLGKKRDP |
| 360 | GKRKKKGKLGKKRPRSR |
| 361 | GKRRRRATAKYRSAH |
| 362 | GKRVAKRKLIEQNRERRR |
| 363 | GKYVSLTTPKNPTKRRITPKDV |
| 364 | GLADIIKKIAESF |
| 365 | GLFAIIKKIAESF |
| 366 | GLFDAIKKIAESF |
| 367 | GLFDIAKKIAESF |
| 368 | GLFDIAKKVIGVIGSL |
| 369 | GLFDIIAKIAESF |
| 370 | GLFDIIHKIAESF |
| 371 | GLFDIIKAIAESF |
| 372 | GLFDIIKHIAESF |
| 373 | GLFDIIKKAAESF |
| 374 | GLFDIIKKIAASF |
| 375 | GLFDIIKKIADSF |
| 376 | GLFDIIKKIAEAF |
| 377 | GLFDIIKKIAESA |
| 378 | GLFDIIKKIAESF |
| 379 | GLFDIIKKIAESI |
| 380 | GLFDIIKKIAHSF |
| 381 | GLFDIIKKIARSF |
| 382 | GLFDIIKKVASVIGGL |
| 383 | GLFDIIKKVASVVGGL |
| 384 | GLFDIIKRIAESF |
| 385 | GLFDIIRKIAESF |
| 386 | GLFDIVKKIAGHIAGSI |
| 387 | GLFDIVKKIAGHIASSI |
| 388 | GLFDIVKKIAGHIVSSI |
| 389 | GLFDIVKKVVGAFGSL |
| 390 | GLFDIVKKVVGAIGSL |
| 391 | GLFDIVKKVVGALGSL |
| 392 | GLFDIVKKVVGTLAGL |
| 393 | GLFDVIKKVASVIGGL |
| 394 | GLFEAIEGFIENGWEGMIDGWYGGGGrrrrrrrrrK |
| 395 | GLFEALLELLESLWELLLEA |
| 396 | GLFKALLKLLKSLWKLLLKA |
| 397 | GLFKALLKLLKSLWKLLLKAGGC |
| 398 | GLFRALLRLLRSLWRLLLRA |
| 399 | GLGDKFGESIVNANTVLDDLNSRMPQSRHDIQQL |
| 400 | GLGSLLKKAGKKLKQPKSKRKV |
| 401 | GLKKLAELAHKLLKLG |
| 402 | GLKKLAELAHKLLKLGC |
| 403 | GLKKLAELFHKLLKLG |
| 404 | GLKKLAELFHKLLKLGC |
| 405 | GLKKLARLAHKLLKLGC |
| 406 | GLKKLARLFHKLLKLGC |
| 407 | GLLDIVKKVVGAFGSL |
| 408 | GLLEALAELLEGLRKRLRKFRNKIKEK |
| 409 | GLLNGLALRLGKRALKKIIKRLCR |
| 410 | GLPRRRRRRRRR |
| 411 | GLPVCGETCVGGTCNTPGCKCSWPVCTRN |
| 412 | GLPVCGETCVGGTCNTPGCTCSWPKCTRN |
| 413 | GLRKRLRKFRNKIKEK |
| 414 | GLWRALWRALRSLWKLKRKV |
| 415 | GLWRALWRALWRSLWKKKRKV |
| 416 | GLWRALWRALWRSLWKLKRKV |
| 417 | GLWRALWRALWRSLWKLKWKV |
| 418 | GLWRALWRALWRSLWKSKRKV |
| 419 | GLWRALWRGLRSLWKKKRKV |
| 420 | GLWRALWRGLRSLWKLKRKV |
| 421 | GLWRALWRLLRSLWRLLWKA |
| 422 | GLWRALWRLLRSLWRLLWRA |
| 423 | GLWRALWRLLRSLWRLLWSQPKKKRKV |
| 424 | GLWWKAWWKAWWKSLWWRKRKRKA |
| 425 | GLWWRLWWRLRSWFRLWFRA |
| 426 | GNYAHRVGAGAPVWL |
| 427 | GPFHFYQFLFPPV |
| 428 | GRCTKSIPPICFPA |
| 429 | GRCTKSIPPICFPD |
| 430 | GRCTKSIPPICWPD |
| 431 | GRCTKSIPPICWPK |
| 432 | GRCTRSIPPKCWPD |
| 433 | GRGDGPRRKKKKGPRRKKKKGPRR |
| 434 | GRGDSPRR |
| 435 | GRGDSPRRKKKKSPRRKKKKSPRR |
| 436 | GRGDSPRRSPRR |
| 437 | GRKGKHKRKKLP |
| 438 | GRKKRRERRRPPERKCX |
| 439 | GRKKRRQARAPPQC |
| 440 | GRKKRRQPPQC |
| 441 | GRKKRRQRARPPQC |
| 442 | GRKKRRQRPPQC |
| 443 | GRKKRRQRRPPQC |
| 444 | GRKKRRQRRR |
| 445 | GRKKRRQRRRC |
| 446 | GRKKRRQRRRCG |
| 447 | GRKKRRQRRRG |
| 448 | GRKKRRQRRRMVSAL |
| 449 | GRKKRRQRRRP |
| 450 | GRKKRRQRRRPP |
| 451 | GRKKRRQRRRPPQ |
| 452 | GRKKRRQRRRPPQC |
| 453 | GRKKRRQRRRPPQGRKKRRQRRRPPQGRKKRRQRRRPPQ |
| 454 | GRKKRRQRRRPPQK |
| 455 | GRKKRRQRRRPPQRKC |
| 456 | GRKKRRQRRRPPQTYADFIASGRTGRRNAI |
| 457 | GRKKRRQRRRPPQY |
| 458 | GRKKRRQRRRPQ |
| 459 | GRKKRRQRRRPWQ |
| 460 | GRKLKKKKNEKEDKRPRT |
| 461 | GRKRKKRT |
| 462 | GRPRESGKKRKRKRLKP |
| 463 | GRQLRIAGKRLEGRSK |
| 464 | GRQLRIAGKRLRGRSK |
| 465 | GRQLRIAGRRLRGRSR |
| 466 | GRQLRIAGRRLRRRSR |
| 467 | GRQLRRAGRRLRGRSR |
| 468 | GRQLRRAGRRLRRRSR |
| 469 | GRRERNKMAAAKCRNRRR |
| 470 | GRRHHCRSKAKRSRHH |
| 471 | GRRRRATAKYRTAH |
| 472 | GRRRRKRLSHRT |
| 473 | GRRRRRERNK |
| 474 | GRRRRRRRRR |
| 475 | GRRRRRRRRRPPQ |
| 476 | GRXTKSIPPIXFPD |
| 477 | GSGKKGGKKHCQKY |
| 478 | GSGKKGGKKICQKY |
| 479 | GSPWGLQHHPPRT |
| 480 | GSRHPSLIIPRQ |
| 481 | GSRVQIRCRFRNSTR |
| 482 | GSVSRRRRRRGGRRRR |
| 483 | GTKMIFVGIKKKEERADLIAYLKKA |
| 484 | GWFDVVKHIASAV |
| 485 | GWTLNPAGYLLGKINLKALAALAKKIL |
| 486 | GWTLNPPGYLLGKINLKALAALAKKIL |
| 487 | GWTLNSAGYLLGKFLPLILRKIVTAL |
| 488 | GWTLNSAGYLLGKINLKALAALAKKIL |
| 489 | GWTLNSAGYLLGKINLKALAALAKKLL |
| 490 | GWTLNSAGYLLGKINLKAPAALAKKIL |
| 491 | GWTLNSAGYLLGKLKALAALAKKIL |
| 492 | GWTLNSAGYLLGPHAVGNHRSFSDKNGLTS |
| 493 | GWTLNSKINLKALAALAKKIL |
| 494 | GYGNCRHFKQKPRRD |
| 495 | GYGRKKRRGRRRTHRLPRRRRRR |
| 496 | GYGRKKRRQRRRG |
| 497 | GYGYGYGYGYGYGYGYKKRKKRKKRKKRKQQKQQKRRK |
| 498 | HALAHKLKHLLHRLRHLLHRHLRHALAH |
| 499 | HARIKPTFRRLKWKYKGKFW |
| 500 | HATKSQNINF |
| 501 | HEHEHEHEHE |
| 502 | HEHEHEHEHEHEHEHEEFGGGGGYGRGRGRGRGRGRG |
| 503 | HEHEHEHEHEHEHEHEEFGGGGGYGRRRRRRGGGGGG |
| 504 | HEHEHEHEHEHEHEHEHEHEEFGGGGGYGRGRGRGRGRGRG |
| 505 | HEHEHEHEHEHEHEHEHEHEEFGGGGGYGRRRRRRGGGGGG |
| 506 | HEHEHEHEHEHEHEHEHEHEGGGGGKLALKLALKALKAALKL |
| | A |
| 507 | HEHEHEHEHEHEHEHEHEHEHEHEEFGGGGGYGRGRGRGRGR |
| | GRG |
| 508 | HEHEHEHEHEHEHEHEHEHEHEHEEFGGGGGYGRKKRRQRRR |
| 509 | HEHEHEHEHEHEHEHEHEHEHEHEEFGGGGGYGRRRRRRGGG |
| | GGG |
| 510 | HFAAWGGWSLVH |
| 511 | HGVSGHGQHGVHG |
| 512 | HGWZIHGLLHRA |
| 513 | HHHHHHESGGGGSPGRRRRRRRRRRR |
| 514 | HHHHHHHHHHHHHHHHHHHHRRRRRRRRRRRRRRR |
| 515 | HHHHHHHHHHHHHHHHRRRRRRRRRRRRRRR |
| 516 | HHHHHHHHHHHHRRRRRRRRRRRRRRR |
| 517 | HHHHHHHHRRRRRRRR |
| 518 | HHHHHHHHRRRRRRRRRRRRRRR |
| 519 | HHHHHHRRRRRRRRR |
| 520 | HHHHHHTKRRITPKDVIDVRSVTTEINT |
| 521 | HHHRRRRRRRR |
| 522 | HHHRRRRRRRRRHHH |
| 523 | HILPWKWPWWPWRR |
| 524 | HIQLSPFSQSWR |
| 525 | HPGSPFPPEHRP |
| 526 | HQHKPPPLTNNW |
| 527 | HRHIRRQSLIML |
| 528 | HRLRHALAHLLHKLKHLLHALAHRLRH |
| 529 | HSDAVFTDNYTALRKQMAVKKYLNSILNYGRKKRRQRRR |
| 530 | HSDGIFTDSYSRYRKQMAVKKYLAAVLGKRYKQRVKNK |
| 531 | HXRHXRILFQYHXRHXR |
| 532 | HYRIKPTARRLKWKYKGKFW |
| 533 | HYRIKPTFRRLAWKYKGKFW |
| 534 | HYRIKPTFRRLKWKYKGKFA |
| 535 | AGYLLGKINLKALAALAKKIL |
| 536 | AGYLLGKINLKALAALAKKIL |
| 537 | IAWVKAFIRKLRKGPLG |
| 538 | icir |
| 539 | IGCRH |
| 540 | IIIR |
| 541 | IIYRDLISH |
| 542 | IKIKIKIKIKIKIKIKKLAKLAKLAKLAKLAKLAKKIK |
| 543 | IKIWFQNRRMKWKK |
| 544 | INLKALAALAKKIL |
| 545 | INLKKLAKLKKIL |
| 546 | IPALK |
| 547 | IPLVVPLC |
| 548 | IPLVVPLRRRRRRRRC |
| 549 | IPMIK |
| 550 | IPMLK |
| 551 | IPSRWKDQFWKRWHY |
| 552 | IRQRRRR |
| 553 | ISFELLDYYED |
| 554 | ISFELLDYYESGS |
| 555 | ISFEWLQAYEDE |
| 556 | ISFDELLDYYGESGS |
| 557 | IWFQNRRMKWKK |
| 558 | IWRYSLASQQ |
| 559 | IYLATALAKWALKQGFGGRRRRRRR |
| 560 | IYLATALAKWALKQGGRRRRRRR |
| 561 | IYRDLISH |
| 562 | AGYLLGKINLKALAALAKKIL |
| 563 | KFQWQRNMRKVRGPPVSIKR |
| 564 | KAFAKLAARLYRKALARQLGVAA |
| 565 | KALAALLKKLAKLLAALK |
| 566 | KALAALLKKWAKLLAALK |
| 567 | KALAKALAKLWKALAKAA |
| 568 | KALKKLLAKWLAAAKALL |
| 569 | KALKLKLALALLAKLKLA |
| 570 | KCCKWRWRCK |
| 571 | KCFMWQEMLNKAGVPKLRCARK |
| 572 | KCFQWQRNMRKVR |
| 573 | KCFQWQRNMRKVRGPPVSC |
| 574 | KCFQWQRNMRKVRGPPVSCIKR |
| 575 | KCFQWQRNMRKVRGPPVSSIKR |
| 576 | KCGCRWRWKCGCKK |
| 577 | KCPSRRPKR |
| 578 | KCRWRWKCCKK |
| 579 | KDCERRFSRSDQLKRHQRRHTGVKPFQK |
| 580 | KDCRWRWKCCKK |
| 581 | KETWFETWFTEWSQPKKKRKV |
| 582 | KETWWETWWTEWSQPGRKKRRQRRRPPQ |
| 583 | KETWWETWWTEWSQPKKKRKV |
| 584 | KETWWETWWTEWSQPKKKRKVC |
| 585 | KFFKFFKFFK |
| 586 | KFHTFPQTAIGVGAP |
| 587 | KFLNRFWHWLQLKPGQPMY |
| 588 | KGKKIFIMK |
| 589 | KGRKKRRQRRRPPQ |
| 590 | KGRTPIKFGKADCDRPPKHSQNGMGK |
| 591 | KGSKKAVTKAQKKDGKKRKRSRKESYSVYVYKVLKQ |
| 592 | KHHWHHVRLPPPVRLPPPGNHHHHHH |
| 593 | KHKALHALHLLALLWLHLAHLAKHK |
| 594 | KHKHKHKHKHKHKHKHKHKKLFKKILKYL |
| 595 | KHKLLHLLHLLALLWLHLLHLLKHK |
| 596 | KIAAKSIAKIWKSILKIA |
| 597 | KIAKLKAKIQKLKQKIAKLK |
| 598 | KITLKLAIKAWKLALKAA |
| 599 | KIWFQNRRMKWKK |
| 600 | KKAAQIRSQVMTHLRVI |
| 601 | KKALLAHALHLLALLALHLAHALKKA |
| 602 | KKALLALALHHLAHLALHLALALKKA |
| 603 | KKDGKKRKRSRKESYSVYVYKVLKQ |
| 604 | KKICTRKPRFMSAWAQ |
| 605 | KKKEERADLIAYLKKA |
| 606 | KKKKKKGGFLGFWRGENGRKTRSAYERMCILKGK |
| 607 | KKKKKKKK |
| 608 | KKKKKKKKK |
| 609 | KKKKKKKKKKKKKKKKKKK |
| 610 | KKKKKKNKKLQQRGD |
| 611 | KKLALHALHLLALLWLHLAHLALKK |
| 612 | KKLFKKILKKL |
| 613 | KKPGKKTTTKPTKK |
| 614 | KKPGKKTTTKPTKKPTIKTTKK |
| 615 | KKPTIKTTKK |
| 616 | KKRRQRRR |
| 617 | KKTTTKPTKK |
| 618 | KKWALLALALHHLAHLALHLALALKKAHHHHHH |
| 619 | KKWKMRILFQYRXRRXR |
| 620 | kkwkmrrGaGrrrrrrrrr |
| 621 | KKWKMRRNQFWIKIQR |
| 622 | KLAAALLKKWKKLAAALL |
| 623 | KLAKLAKKLAKLAK |
| 624 | KLAKLAKKLAKLAKGGRRRRRRR |
| 625 | KLAKLAKKLAKLAKGRKKRRQRRRP |
| 626 | KLAKLAKKLAKLAKNYRWRCKNQN |
| 627 | KLALKAAAKAWKAAAKAA |
| 628 | KLALKAALKAWKAAAKLA |
| 629 | KLALKALKAALKLA |
| 630 | KLALKLALKALKAA |
| 631 | KLALKLALKALKAALK |
| 632 | KLALKLALKALKAALKLA |
| 633 | KLALKLALKALKAALKLAGC |
| 634 | KLALKLALKALQAALQLA |
| 635 | KLALKLALKAWKAALKLA |
| 636 | KLALKLALKWAKLALKAA |
| 637 | KLALQLALQALQAALQLA |
| 638 | KLFMALVAFLRFLTIPPTAGILKRWGTI |
| 639 | KLGLKLGLKGLKGGLKLG |
| 640 | KLGVM |
| 641 | KLIKGRTPIKFGK |
| 642 | KLIKGRTPIKFGKADCDRPPKHSGK |
| 643 | KLIKGRTPIKFGKADCDRPPKHSQNGK |
| 644 | KLIKGRTPIKFGKADCDRPPKHSQNGM |
| 645 | KLIKGRTPIKFGKADCDRPPKHSQNGMGK |
| 646 | KLIKGRTPIKFGKARCRRPPKHSGK |
| 647 | KLLAKAAKKWLLLALKAA |
| 648 | KLLAKAALKWLLKALKAA |
| 649 | KLLKLLLKLWKKLLKLLK |
| 650 | KLLKLLLKLWKKLLKLLKGGGRRRRRRR |
| 651 | KLPCRSNTFLNIFRRKKPG |
| 652 | KLPVM |
| 653 | KLPVT |
| 654 | KLTRAQRRAAARKNKRNTRGC |
| 655 | KLALKLALKALKAALKLA |
| 656 | KLALKLALKALKAALKLAGC |
| 657 | KLWMRWWSPTTRRYG |
| 658 | KLWMRWYSATTRRYG |
| 659 | KLWMRWYSPTTRRYG |
| 660 | KLWMRWYSPWTRRYG |
| 661 | KLWSAWPSLWSSLWKP |
| 662 | KMDCRPRPKCCKK |
| 663 | KMDCRPRPKCXKK |
| 664 | KMDCRPRPKXCKK |
| 665 | KMDCRWRPKCCKK |
| 666 | KMDCRWRWKCCKK |
| 667 | KMDCRWRWKCKK |
| 668 | KMDCRWRWKCSKK |
| 669 | KMDCRWRWKKK |
| 670 | KMDCRWRWKSCKK |
| 671 | KMDCRWRWKSSKK |
| 672 | KMDRWRWKKK |
| 673 | KMDSRWRWKCCKK |
| 674 | KMDSRWRWKCSKK |
| 675 | KMDSRWRWKSCKK |
| 676 | KMDSRWRWKSSKK |
| 677 | KMDXRPRPKCCKK |
| 678 | KMDXRPRPKCXKK |
| 679 | KMDXRPRPKXCKK |
| 680 | KMDXRWRWKCCKK |
| 681 | KMDXRWRWKCXKK |
| 682 | KMDXRWRWKXCKK |
| 683 | KMDXRWRWKXXKK |
| 684 | KMIFVGIKKK |
| 685 | KMIFVGIKKKEERA |
| 686 | KMTRAQRRAAARRNRWTARGC |
| 687 | KNAWKHSSCHHRHQI |
| 688 | KPRSKNPPKKPK |
| 689 | KRARNTEAARRSRARKLQRMKQGC |
| 690 | KRIHPRLTRSIR |
| 691 | KRIIQRILSRNS |
| 692 | KRIPNKKPGKK |
| 693 | KRIPNKKPGKKT |
| 694 | KRIPNKKPGKKTTTKPTKK |
| 695 | KRIPNKKPGKKTTTKPTKKPTIK |
| 696 | KRIPNKKPGKKTTTKPTKKPTIKTTKK |
| 697 | KRIPNKKPGKKTTTKPTKKPTIKTTKKDLK |
| 698 | KRIPNKKPGKKTTTKPTKKPTIKTTKKDLKPQTTKPK |
| 699 | KRIPNKKPKK |
| 700 | KRKRWHW |
| 701 | KRPAAIKKAGQAKKKK |
| 702 | KRPTMRFRYTWNPMK |
| 703 | KRRIRRERNKMAAAKSRNRRRELTDTGC |
| 704 | KRRQRRR |
| 705 | KRVSRNKSEKKRR |
| 706 | KRWRWKCCKK |
| 707 | KSHAHAQKRIRRRLIILL |
| 708 | KSICKTIPSNKPKKK |
| 709 | KSTGKANKITITNDKGRLSK |
| 710 | KTCENLADTY |
| 711 | KTIEAHPPYYAS |
| 712 | KTIPSNKPKKK |
| 713 | KTVLLRKLLKLLVRKI |
| 714 | KWCFAVCYAGICYAACAGK |
| 715 | KWCFRVCYRGICYRRCRGK |
| 716 | KWFETWFTEWPKKRK |
| 717 | KWFETWFTEWPKKRKGGC |
| 718 | KWFKIQMQIRRWKNKR |
| 719 | KWFRVYRGIYRRRGK |
| 720 | KWSFRVSYRGISYRRSRGK |
| 721 | KXRKXRILFQYKXRKXR |
| 722 | LAELLAELLAELGGGGRRRRRRRRR |
| 723 | LAIILRRRIRKQAHAHSK |
| 724 | LALALALALALALAKLAKLAKLAKLAKIKKIKKKIK |
| 725 | LALALALALALALALAKIKKIKKIKKIKKLAKLAKKIK |
| 726 | LALALALALALALALAKKLKKLKKLKKLKKLKKLKYAK |
| 727 | LALALALALALALALAKLAKLAKLAKLAKLAKKIK |
| 728 | LAQLLAQLLAQLGGGGRRRRRRRRR |
| 729 | RRRRrrrrr |
| 730 | rrrrrRRRR |
| 731 | rrrrrrr |
| 732 | RRRRRRRRR |
| 733 | lcl |
| 734 | LCLE |
| 735 | LCLH |
| 736 | LCLK |
| 737 | LCLN |
| 738 | LCLQ |
| 739 | LCLR |
| 740 | LCLRP |
| 741 | LCLRPVG |
| 742 | LDITPFLSLTLP |
| 743 | LDTYSPELFCTIRNFYDADRPDRGAAA |
| 744 | LFDIIKKIAESF |
| 745 | LFDIIKKIAESGFLFDIIKKIAESF |
| 746 | LGISYGRKKRRQRRRPPQ |
| 747 | LGLLLRHLRHHSNLLANI |
| 748 | LGTYTQDFNKFHTFPQTAIGVGAP |
| 749 | LHHLLHHLLHLLHHLLHHLHHL |
| 750 | LIIFAIAASHKK |
| 751 | LIIFAILISHKK |
| 752 | LIIFRIAASHKK |
| 753 | LIIFRILISH |
| 754 | LIIFRILISHHH |
| 755 | LIIFRILISHK |
| 756 | LIIFRILISHKK |
| 757 | LIIFRILISHR |
| 758 | LIIFRILISHRR |
| 759 | LIKKLKALKKLNI |
| 760 | LIKKALAALAKLNI |
| 761 | LILIGRRRRRRRRGC |
| 762 | LILILILILILILILIKRKKRKKRKKRKKRAKRAKHSK |
| 763 | LIRLWSHLIHIWFQNRRLKWKKK |
| 764 | LIRLWSHLIHIWFQNRRLKWKKKC |
| 765 | LIRLWSHLIHIWFQNRRLKWKKKGGC |
| 766 | LKKLAELAHKLLKLG |
| 767 | LKKLCKLLKKLCKLAG |
| 768 | LKKLLKLLKKLLKLAG |
| 769 | LKlLKkLlkKLLkLL |
| 770 | LKRWGTIKKSKAINVLRGFRKEIGRMLNILNRRRR |
| 771 | LKTLATALTKLAKTLTTL |
| 772 | LKTLTETLKELTKTLTEL |
| 773 | LLAILRRRIRKQAHAHSK |
| 774 | LLETLLKPFQCRICMRNFSTRQARRNHRRRHRR |
| 775 | LLGDFFRKSKEKIGKEFKRIVQRIKDFLRNLVPRTESC |
| 776 | LLGKINLKALAALAKKIL |
| 777 | LLHILRRSIRKQAHAIRK |
| 778 | LLHILRRSIRRQAHAIRR |
| 779 | LLIALRRRIRKQAHAHSK |
| 780 | LLIIARRRIRKQAHAHSK |
| 781 | LLIILARRIRKQAHAHSK |
| 782 | LLIILRARIRKQAHAHSK |
| 783 | LLIILRRAIRKQAHAHSK |
| 784 | LLIILRRRARKQAHAHSK |
| 785 | LLIILRRRIARKQAHAHSK |
| 786 | LLIILRRRIRAQAHAHSK |
| 787 | LLIILRRRIRKAAHAHSK |
| 788 | LLIILRRRIRKQAAAHSK |
| 789 | LLIILRRRIRKQAHAASK |
| 790 | LLIILRRRIRKQAHAHAK |
| 791 | LLIILRRRIRKQAHAHSA |
| 792 | LLIILRRRIRKQAHAHSK |
| 793 | LLIILRRRIRKQAHAHSKNHQQQNPHQPPM |
| 794 | LLIILRRRIRRRARARSR |
| 795 | LLKKRKVVRLIKFLLK |
| 796 | LLKLLKKLLKLLKKLLKLL |
| 797 | LLKTTALLKTTALLKTTA |
| 798 | LLKTTELLKTTELLKTTE |
| 799 | LLLLR |
| 800 | LLLR |
| 801 | LLLRR |
| 802 | LLKKLAAAALKKLL |
| 803 | LLR |
| 804 | LLRARWRRRRSRRFR |
| 805 | LLRHLRRHIRRARRHIRR |
| 806 | LLRILRRSIRRARRAIRR |
| 807 | LLYWFRRRHRHHRRRHRR |
| 808 | LNSAGYLLGKALAALAKKIL |
| 809 | LNSAGYLLGKINLKALAALAKKIL |
| 810 | LNSAGYLLGKLKALAALAK |
| 811 | LNSAGYLLGKLKALAALAKIL |
| 812 | LNVPPSWFLSQR |
| 813 | LPHPVLHMGPLR |
| 814 | LRHHLRHLLRHLRHLLRHLRHHLRHLLRH |
| 815 | LRHLLRHLLRHLRHL |
| 816 | LRHLLRHLLRHLRHLLRHLRHLLRHLLRH |
| 817 | LRRERQSRLRRERQSR |
| 818 | LSTAADMQGVVTDGMASGLDKDYLKPDD |
| 819 | LTMPSDLQPVLW |
| 820 | LTRNYEAWVPTP |
| 821 | LVVLGKLYGRKKRRQRRR |
| 822 | MAARL |
| 823 | MAARLCCQ |
| 824 | MAARLCCQLDPARDV |
| 825 | MAARLCCQLDPARDVLCLRP |
| 826 | MAIYRDLIS |
| 827 | MAMPGEPRRANVMAHKLEPASLQLRNSCA |
| 828 | MANLGCWMLVLFVATWSDLGLCKKRPKP |
| 829 | MANLGYWLLALFVTMWTDVGLCKKRPKP |
| 830 | MAPQRDTVGGRTTPPSWGPAKAQLRNSCA |
| 831 | MDAQTRRRERRAEKQAQWKAANGC |
| 832 | MDCRWRWKCCKK |
| 833 | MDCRWRWKCXKK |
| 834 | MDCRWRWKXCKK |
| 835 | MDCRWRWKXXKK |
| 836 | RKKRRRESWVHLPPPVHLPPPGGHHHHHH |
| 837 | MGLGLHLLVLAAALQGAKKKRKV |
| 838 | MGLGLHLLVLAAALQGAWSQPKKKRKV |
| 839 | MGVADLIKKFESISKEEGGGGKGGrRrRrRRR |
| 840 | MGVADLIKKFESISKEEGGGGKGGrRrRrRRR |
| 841 | MGVADLIKKFESISKEEGGGGKGGrRrRrRRR |
| 842 | MHKRPTTPSRKM |
| 843 | MIAYRDLIS |
| 844 | MIIARDLIS |
| 845 | MIIFAIAASHKK |
| 846 | MIIFKIAASHKK |
| 847 | MIIFRAAASHKK |
| 848 | MIIFRALISHKK |
| 849 | MIIFRDLISH |
| 850 | MIIFRIAASHKK |
| 851 | MIIFRIAATHKK |
| 852 | MIIFRIAAYHKK |
| 853 | MIIFRILISHKK |
| 854 | MIIRRDLISE |
| 855 | MIISRDLISH |
| 856 | MIIYADLIS |
| 857 | MIIYARRAEE |
| 858 | MIIYRAEISH |
| 859 | MIIYRALIS |
| 860 | MIIYRALISHKK |
| 861 | MIIYRD |
| 862 | MIIYRDAIS |
| 863 | MIIYRDKKSH |
| 864 | MIIYRDL |
| 865 | MIIYRDLAS |
| 866 | MIIYRDLI |
| 867 | MIIYRDLIA |
| 868 | MIIYRDLIS |
| 869 | MIIYRDLISH |
| 870 | MIIYRDLISKK |
| 871 | MIIYRIAASHKK |
| 872 | MLLLTRRRST |
| 873 | HEHEHEHEHE |
| 874 | RGRGRGRGRG |
| 875 | MRRIRPRPPRLPRPRPRPLPFPRPGGCYPG |
| 876 | MTPSSLSTLPWP |
| 877 | MVKSKIGSWILVLFVAMWSDVGLCKKRPKP |
| 878 | MVRRFLVTLRIRRACGPPRVRV |
| 879 | MVRRFLVTLRIRRACGPPRVRVFVVHIPRLTGEWAAP |
| 880 | MVTVLFKRLRIRRACGPPRVKV |
| 881 | MVTVLFRRLRIRRACGPPRVRV |
| 882 | RRRRRRRRRRR |
| 883 | NAKTRRHERRRKLAIERGC |
| 884 | GGGGGGGG |
| 885 | GGGGGGGGGGGG |
| 886 | GGGGGGGGGGGG |
| 887 | NFLGTLVNLAKKIL |
| 888 | NHQQQNPHQPPM |
| 889 | NHQQQNPHQPPMLLIILRRRIRKQAHAHSK |
| 890 | NIENSTLATPLS |
| 891 | NKPILVFY |
| 892 | NKRILIRIMTRP |
| 893 | GGGGGGGGGGGGG |
| 894 | GGGGGGGGGGGG |
| 895 | GGGGGGGGGGGG |
| 896 | GGGGGGGGGGGG |
| 897 | GGGGGGGGG |
| 898 | GGGGGGGGGGGG |
| 899 | GGGGGGGGGGGG |
| 900 | NNNAAGRKRKKRT |
| 901 | NRARRNRRRVR |
| 902 | NRHFRFFFNFTNR |
| 903 | NRRMKWKK |
| 904 | NSGTMQSASRAT |
| 905 | NTCTWLKYH |
| 906 | NTCTWLKYHS |
| 907 | NTGTWLKYHS |
| 908 | NYQRRCKNQN |
| 909 | NYQWRCKNQN |
| 910 | NYRRRCKNON |
| 911 | NYRWRCK |
| 912 | NYRWRCKN |
| 913 | NYRWRCKNQ |
| 914 | NYRWRCKNQN |
| 915 | NYTTYKSHFQDR |
| 916 | PARAARRAARR |
| 917 | PFVYLI |
| 918 | PIRRRKKLRRLK |
| 919 | PKKKRKV |
| 920 | PKKKRKVAGYLLGKINLKALAALAKKILPQMQQNVFQYPGAG |
| | MVPQGEANF |
| 921 | PKKKRKVRRRRRRRPQMQQNVFQYPGAGMVPQGEANF |
| 922 | PKKKRKVRRRRRRRYSQTSHKLVQLLTTAEQQ |
| 923 | PKKKRKVALWKTLLKKVLKA |
| 924 | PKKKRKVWKLLQQFFGLM |
| 925 | PLSSIFSRIGDP |
| 926 | PMLKE |
| 927 | PNTRVRPDVSF |
| 928 | PPHNRIQRRLNM |
| 929 | PPKKSAQCLRYKKPE |
| 930 | PPRLPRPRPRPLPFPRPG |
| 931 | PPRLRKRRQLNM |
| 932 | PQNRLQIRRHSK |
| 933 | PRPLPFPRPG |
| 934 | PRPPRLPRPRPRPLPFPRPG |
| 935 | PRPRPLPFPRPG |
| 936 | PRPRPRPLPFPRPG |
| 937 | PSKRLLHNNLRR |
| 938 | PSSSSSSRIGDP |
| 939 | QAASRVENYMHR |
| 940 | QIISRDLISH |
| 941 | QIKIWFQNRRMKWKK |
| 942 | QLALQLALQALQAALQLA |
| 943 | QLPVM |
| 944 | QNRRMKWKK |
| 945 | QPIIITSPYLPS |
| 946 | QQHLLIAINGYPRYN |
| 947 | QRIRKSKISRTL |
| 948 | QSPTDFTFPNPL |
| 949 | QTRRRERRAEKQAQW |
| 950 | QWQRNMRKVR |
| 951 | QWQRNMRKVRGPPVSCIKR |
| 952 | RAGLQFPVGRVHRLLRK |
| 953 | RAIKIWFQNRRMKWKK |
| 954 | RAKRRQRRR |
| 955 | RARARARARARARARARARARARARARARARA |
| 956 | RAWMRWYSPTTRRYG |
| 957 | RFTFHFRFEFTFHFE |
| 958 | RFTFHFRFEFTFHFEGGGRRRRRRR |
| 959 | RGDADDARRRRRRRR |
| 960 | RGDRRRRRRRR |
| 961 | RGDRRRRRRRR |
| 962 | RGDfK |
| 963 | RGDGPRRRPRKRRGR |
| 964 | RGDRGDRRDLRLDRGDLRC |
| 965 | RGDRLDRRDLRLDRRDLRC |
| 966 | RGERGERRELRLERGELRC |
| 967 | RGERLERRELRLERRELRC |
| 968 | RGGRLAYLRRRWAVLGR |
| 969 | RGGRLSYSRRRFSTSTGR |
| 970 | RGGRLSYSRRRFSTSTGRA |
| 971 | RGPRRQPRRHRRPRR |
| 972 | RGRGRGRGRG |
| 973 | RGSRRAVTRAQRRDGRRRRRSRRESYSVYVYRVLRQ |
| 974 | RHHLRHLRRHL |
| 975 | RHHLRHLRRHLRHLLRHLRHHL |
| 976 | RHHLRHLRRHLRHLLRHLRHHLRHLRRHLRHLL |
| 977 | RHHRRHHRRHRRHHRRHHRHHR |
| 978 | RHIKIWFQNRRMKWKK |
| 979 | RHNFRFFFNFRTNR |
| 980 | rHNHrFNFrFFFNFrFNTrTN |
| 981 | rHrHrrHrHrrHrHr |
| 982 | RHVYHVLLSQ |
| 983 | RIFIGC |
| 984 | RIFIHFRIGC |
| 985 | RIFIRIGC |
| 986 | RIKAERKRMRNRIAASKSRKRKLERIARGC |
| 987 | RILQQLLFIHF |
| 988 | RILQQLLFIHFRIGC |
| 989 | RILQQLLFIHFRIGCRH |
| 990 | RILQQLLFIHFRIGCRHSRI |
| 991 | RIMRILRILKLAR |
| 992 | RIRMIQNLIKKT |
| 993 | RKARRQRRR |
| 994 | RKKAAA |
| 995 | RKKARQRRR |
| 996 | RKKNPNCRRH |
| 997 | RKKRAQRRR |
| 998 | RKKRKKKRXRHXRHXRHXR |
| 999 | RKKRRARRR |
| 1000 | RKKRRQARR |
| 1001 | RKKRRQR |
| 1002 | RKKRRQRAR |
| 1003 | RKKRRQRR |
| 1004 | RKKRRQRRA |
| 1005 | RKKRRQRRR |
| 1006 | RKKRRQRRRGC |
| 1007 | RKKRRQRRRGGG |
| 1008 | RKKRRQRRRGGGKLLKLLLKLLLKLLK |
| 1009 | RKKRRQRRRHRRKKR |
| 1010 | RKKRRQRRRPPQCAAVALLPAVLLALLAP |
| 1011 | RKKRRQRRRRKKRRQRRR |
| 1012 | RKKRRRESRKKRRRES |
| 1013 | RKKRRRESRKKRRRESC |
| 1014 | RKKRRRESRRARRSPRHL |
| 1015 | RKKRRRESWVHLPPPVHLPPPGGHHHHHH |
| 1016 | RKKWFW |
| 1017 | RKLTTIFPLNWKYRKALSLG |
| 1018 | RLALRLALRALRAALRLA |
| 1019 | RLAMRWYSPTTRRYG |
| 1020 | RLFMRFYSPTTRRYG |
| 1021 | RLHHRLHRRLHRLHR |
| 1022 | RLHHRLHRRLHRLHRRLHRLHHRLHRRLH |
| 1023 | RLHLRLHLRHLRHHLRLH |
| 1024 | RLHRRLHRRLHRLHR |
| 1025 | RLHRRLHRRLHRLHRRLHRLHRRLHRRLH |
| 1026 | RLIMRIYAPTTRRYG |
| 1027 | RLIMRIYSPTTRRYG |
| 1028 | RLLMRLYSPTTRRYG |
| 1029 | RLLRLLLRLWRRLLRLLR |
| 1030 | RLLRLLRLL |
| 1031 | RLLRLLRLX |
| 1032 | RLLRLLRRLLRLLRRLLRC |
| 1033 | RLLRLXRLX |
| 1034 | RLPRPRPRPLPFPRPG |
| 1035 | RLRLRLRLRLRLRLRLKLLKLLKLLKLLKKKKKKKGYK |
| 1036 | RLRLRLRLRLRLRLRLKNNKNNKNNKNNKKKKKKKGYK |
| 1037 | RLRLRLRLRLRLRLRLKRLKRLKRLKRLKKKKKKKGYK |
| 1038 | RLSGMNEVLSFRWL |
| 1039 | RLVMRVYSPTTRRYG |
| 1040 | RLWARWYSPTTRRYG |
| 1041 | RLWMAWYSPTTRRYG |
| 1042 | RLWMRAYSPTTRRYG |
| 1043 | RLWMRWASPTTRRYG |
| 1044 | RLWMRWYAPTTRRYG |
| 1045 | RLWMRWYSPATRRYG |
| 1046 | RLWMRWYSPRTRAYG |
| 1047 | RLWMRWYSPTARRYG |
| 1048 | RLWMRWYSPTTARYG |
| 1049 | RLWMRWYSPTTRAYG |
| 1050 | RLWMRWYSPTTRRAG |
| 1051 | RLWMRWYSPTTRRYA |
| 1052 | RLWMRWYSPTTRRYG |
| 1053 | RLWMRWYSPWTRRWG |
| 1054 | RLWMRWYSPWTRRYG |
| 1055 | RLWRALPRVLRRLLRP |
| 1056 | RLXRLXRLX |
| 1057 | RLXRLXRXX |
| 1058 | RLXRXRXX |
| 1059 | RLYMRYYSPTTRRYG |
| 1060 | RMKWKK |
| 1061 | RMKWKKILFQYRXRRXR |
| 1062 | RNRSRHRR |
| 1063 | RPARPAR |
| 1064 | RQAKIWFQNRRMKWKK |
| 1065 | RQARRNRRRALWKTLLKKVLKA |
| 1066 | RQARRNRRRC |
| 1067 | RQGAARVTSWLGRQLRIAGKRLEGRSK |
| 1068 | RQIAIWFQNRRMKWKK |
| 1069 | RQIKAWFQNRRMKWKK |
| 1070 | RQIKIAFQNRRMKWKK |
| 1071 | RQIKIFFQNRRMKFKK |
| 1072 | RQIKIFFQNRRMKWKK |
| 1073 | RQIKIQFQNRRKWKK |
| 1074 | RQIKIW |
| 1075 | RQIKIWAQNRRMKWKK |
| 1076 | RQIKIWFANRRMKWKK |
| 1077 | RQIKIWFPNRRMKWKK |
| 1078 | RQIKIWFQ |
| 1079 | RQIKIWFQARRMKWKK |
| 1080 | RQIKIWFQN |
| 1081 | RQIKIWFQNARMKWKK |
| 1082 | RQIKIWFQNMRRKWKK |
| 1083 | RQIKIWFQNR |
| 1084 | RQIKIWFQNRAMKWKK |
| 1085 | RQIKIWFQNRR |
| 1086 | RQIKIWFQNRRAKWKK |
| 1087 | RQIKIWFQNRRM |
| 1088 | RQIKIWFQNRRMAWKK |
| 1089 | RQIKIWFQNRRMK |
| 1090 | RQIKIWFQNRRMKAKK |
| 1091 | RQIKIWFQNRRMKW |
| 1092 | RQIKIWFQNRRMKWAK |
| 1093 | RQIKIWFQNRRMKWK |
| 1094 | RQIKIWFQNRRMKWKA |
| 1095 | RQIKIWFQNRRMKWKK |
| 1096 | RQIKIWFQNRRMKWKKC |
| 1097 | RQIKIWFQNRRMKWKKDIMGEWGNEIFGAIAGFLG |
| 1098 | RQIKIWFQNRRMKWKKGC |
| 1099 | RQIKIWFQNRRMKWKKGG |
| 1100 | RQIKIWFQNRRMKWKKK |
| 1101 | RQIKIWFQNRRMKWKKRQIKIWFQNRRMKWK |
| 1102 | RQIKIWFQNRRMKWKKTYADFIASGRTGRRNAI |
| 1103 | RQIRIWFQNRRMRWRR |
| 1104 | RQIRIWFQNRRMRWRRC |
| 1105 | RQLRIAGRRLRGRSR |
| 1106 | RQPKIWFPNRRKPWKK |
| 1107 | RQRSRRRPLNIR |
| 1108 | RRARRPRRLRPAPGR |
| 1109 | RRGC |
| 1110 | RRGRRG |
| 1111 | RRHHCRSKAKRSR |
| 1112 | RRHLRRHLRHLRRHLRRHLRHL |
| 1113 | RRIPNRRPRR |
| 1114 | RRIRPRP |
| 1115 | RRIRPRPPRLPRPRP |
| 1116 | RRIRPRPPRLPRPRPRP |
| 1117 | RRIRPRPPRLPRPRPRPLPFPRPG |
| 1118 | RRKLSQQKEKK |
| 1119 | RRLLRRLRR |
| 1120 | RRLRHLRHHYRRRWHRFR |
| 1121 | RRLSYSRRRF |
| 1122 | RRMKWKK |
| 1123 | RRQRRTSKLMKR |
| 1124 | RRR |
| 1125 | RRRERRAEK |
| 1126 | rRrGrKkRr |
| 1127 | RRRQKRIVVRRRLIR |
| 1128 | RRRQRRKKR |
| 1129 | RRRQRRKKRGYCKCKYGRKKRRQRRR |
| 1130 | RRRQRRKRGGDIMGEWGNEIFGAIAGFLG |
| 1131 | RRRR |
| 1132 | RRRRNRTRRNRRRVRGC |
| 1133 | RRRRR |
| 1134 | RRRRRHHH |
| 1135 | RRRRRR |
| 1136 | RRRRRRHHH |
| 1137 | RRRRRRR |
| 1138 | RRRRRRRGGIYLATALAKWALKQ |
| 1139 | RRRRRRRGGIYLATALAKWALKQGF |
| 1140 | RRRRRRRGGKLAKLAKKLAKLAK |
| 1141 | RRRRRRRHHH |
| 1142 | RRRRRRRQIKILFQNRRMKWKKGGC |
| 1143 | RRRRRRRR |
| 1144 | RRRRRRRRRGDfK |
| 1145 | RRRRRRRRRGD |
| 1146 | RRRRRRRRC |
| 1147 | RRRRRRRRGC |
| 1148 | RRRRRRRRHHH |
| 1149 | RRRRRRRRK |
| 1150 | RRRRRRRRR |
| 1151 | RRRRRRRRRC |
| 1152 | rrrrrrrrrcqcrrkn |
| 1153 | RRRRRRRRRGGLAASGWKHHHHHH |
| 1154 | RRRRRRRRRGPGVTWTPQAWFQWV |
| 1155 | RRRRRRRRRHHH |
| 1156 | rrrrrrrrrk |
| 1157 | RRRRRRRRRR |
| 1158 | RRRRRRRRRRR |
| 1159 | RRRRRRRRRRRR |
| 1160 | RRRRRRRRRRRRGC |
| 1161 | RRRRRRRRRRRRRRR |
| 1162 | RRRRRRRRRRRRRRRR |
| 1163 | RRRRRRRRRRRRRRRRGC |
| 1164 | RRRRRRRRRRRTYADFIASGRTGRRNAI |
| 1165 | RRRRRRRW |
| 1166 | RRRRWWWW |
| 1167 | RRRRWWWWRRRR |
| 1168 | RRVTSWLGRQLRIAGKRLEGRSK |
| 1169 | RRVWRRYRRQRWCRR |
| 1170 | RRWRRWNRFNRRRCR |
| 1171 | RRWRRWWRRWWRRWRR |
| 1172 | RRWWRRWRR |
| 1173 | rsrgrlrrgairlqrg |
| 1174 | RSVTTEINTLFQTLTSIAEKVDP |
| 1175 | RTLVNEYKNTLKFSK |
| 1176 | RTRRNRRRVR |
| 1177 | RVIRVWFQNKRCKDKK |
| 1178 | RVIRWFQNKRCKDKK |
| 1179 | RVIRWFQNKRSKDKK |
| 1180 | RVREWWYTITLKQES |
| 1181 | RVRILARFLRTRV |
| 1182 | RVRSWLGRQLRIAGKRLEGRSK |
| 1183 | RVRVFVVHIPRLT |
| 1184 | RVTSWLGRQLRIAGKRLEGRSK |
| 1185 | RWRCKNQN |
| 1186 | RWRRWRRWRRWR |
| 1187 | RWRRWWRRW |
| 1188 | RWRWKCCKK |
| 1189 | RWRWKXCKK |
| 1190 | RWRWKXXKK |
| 1191 | RWRWRWRW |
| 1192 | RXRRBRRXRRBRXB |
| 1193 | RXRRBRRXRYQFLIRXRBRXRB |
| 1194 | RXRRXRAAAAARXRRXR |
| 1195 | RXRRXRFLQIYRXRRXR |
| 1196 | RXRRXRIEFQYRXRRXR |
| 1197 | RXRRXRIKFQYRXRRXR |
| 1198 | RXRRXRILFQYKKWKMR |
| 1199 | RXRRXRILFQYRMKWKK |
| 1200 | RXRRXRILFQYRXRRXR |
| 1201 | RXRRXRIPFQYRXRRXR |
| 1202 | RXRRXRIWFQYRXRRXR |
| 1203 | RXRRXRRXRRXR |
| 1204 | RXRRXRRXRRXRXB |
| 1205 | RXRRXRYQFLIRXRRXR |
| 1206 | RXRXRXRXRXRXRXRXB |
| 1207 | RXXRXRXX |
| 1208 | SAETVESCLAKSH |
| 1209 | SARHHCRSKAKRSRHH |
| 1210 | SATGAPWKMWVR |
| 1211 | SFHQFARATLAS |
| 1212 | SGRGKQGGKARAKAKTRSSRAGLQFPVGRVHRLLRKG |
| 1213 | SGRGKQGGKARAKAKTRSSRAGLQFPVGRVHRLLRKGC |
| 1214 | SHAFTWPTYLQL |
| 1215 | SHNWLPLWPLRP |
| 1216 | SKKKKTKV |
| 1217 | SKRTRQTYTRYQTLELEKEFHFNRYITRRRRIDIANALSLSE |
| | RQIKIWFQNRRMKSKKDR |
| 1218 | SLGWMLPFSPPF |
| 1219 | SMLKRNHSTSNR |
| 1220 | SNPWDSLLSVST |
| 1221 | SPMQKTMNLPPM |
| 1222 | SQMTRQARRLYBGC |
| 1223 | SRAHHCRSKAKRSRHH |
| 1224 | SRRAHCRSKAKRSRHH |
| 1225 | SRRARRSPRESGKKRKRKR |
| 1226 | SRRARRSPRHLGSG |
| 1227 | SRRHACRSKAKRSRHH |
| 1228 | SRRHHARSKAKRSRHH |
| 1229 | SRRHHCRAKAKRSRHH |
| 1230 | SRRHHCRSAAKRSRHH |
| 1231 | SRRHHCRSKAARSRHH |
| 1232 | SRRHHCRSKAKASRHH |
| 1233 | SRRHHCRSKAKRARHH |
| 1234 | SRRHHCRSKAKRSAHH |
| 1235 | SRRKRQRSNMRI |
| 1236 | SRRRRRRRRR |
| 1237 | SRWRWKCCKK |
| 1238 | SRWRWKCSKK |
| 1239 | SRWRWKSCKK |
| 1240 | SRWRWKSSKK |
| 1241 | SSSIFPPWLSFF |
| 1242 | SWAQHLSLPPVL |
| 1243 | SWLPYPWHVPSS |
| 1244 | SWWTPWHVHSES |
| 1245 | SXRSXRILFQYSXRSXR |
| 1246 | SYIQRTPSTTLP |
| 1247 | TAKTRYKARRAELIAERRGC |
| 1248 | TAMRAVDKLLLHLKKLFREGQFNRNFESIIICRDRT |
| 1249 | TARRITPKDVIDVRSVTTEINT |
| 1250 | TCTWLKYH |
| 1251 | TCTWLKYHS |
| 1252 | TFPQTAIGVGAP |
| 1253 | TKAARITPKDVIDVRSVTTEINT |
| 1254 | TKRRITPDDVIDVRSVTTEINT |
| 1255 | TKRRITPKDVIDV |
| 1256 | TKRRITPKDVIDVESVTTEINT |
| 1257 | TKRRITPKDVIDVRSVTTEINT |
| 1258 | TKRRITPKDVIDVRSVTTKINT |
| 1259 | TKRRITPKKVIDVRSVTTEINT |
| 1260 | TLPSPLALLTVH |
| 1261 | TPFKLSLHL |
| 1262 | TPKTMTQTYDFS |
| 1263 | TPWWRLWTKWHHKRRDLPRKPEGC |
| 1264 | TRQARRNRRRRWRERQR |
| 1265 | TRQARRNRRRRWRERQRGC |
| 1266 | TRRQRTRRARRNRGC |
| 1267 | TRRSKRRSHRKF |
| 1268 | TRSSRAGLQWPVGRVHRLLRKGGC |
| 1269 | TSHTDAPPARSP |
| 1270 | TSPLNIHNGQKL |
| 1271 | TVDNPASTTNKDKLFAVRK |
| 1272 | TWLKYH |
| 1273 | vcvr |
| 1274 | VELPPPVELPPPVELPPP |
| 1275 | VGAlAvVvWlWlWlWAGSGPKKKRKVC |
| 1276 | VHLPPP |
| 1277 | VHLPPPVHLPPP |
| 1278 | VHLPPPVHLPPPVHLPPP |
| 1279 | VIRVHFRLPVRTV |
| 1280 | VKLPPP |
| 1281 | VKLPPPVKLPPP |
| 1282 | VKLPPPVKLPPPVKLPPP |
| 1283 | VKRFKKFFRKLKKKV |
| 1284 | VKRFKKFFRKLKKLV |
| 1285 | VKRFKKFFRKLKKSV |
| 1286 | VKRGLKLRHVRPRVTRMDV |
| 1287 | VKRKKKPALWKTLLKKVLKA |
| 1288 | vlclr |
| 1289 | VLGQSGYLMPMR |
| 1290 | VNADIKATTVFGGKYVSLTTP |
| 1291 | VPALK |
| 1292 | VPALR |
| 1293 | VPMIK |
| 1294 | VPMLK |
| 1295 | VPTLE |
| 1296 | VPTLK |
| 1297 | VPTLQ |
| 1298 | VQAILRRNWNQYKIQ |
| 1299 | VQLRRRWC |
| 1300 | VQRKRQKLMP |
| 1301 | VRLPPP |
| 1302 | VRLPPPVRLPPP |
| 1303 | VRLPPPVRLPPPVRLPPP |
| 1304 | VRRFLVTLRIRRA |
| 1305 | VSALK |
| 1306 | VSGKK |
| 1307 | VSKQPYYMWNGN |
| 1308 | VSLKK |
| 1309 | VSRRRRRRGGRRRR |
| 1310 | VSRRRRRRGGRRRRK |
| 1311 | VTPHHVLVDEYTGEWVDSQFK |
| 1312 | VVLGKLYGRKKRRQRRR |
| 1313 | VVVR |
| 1314 | VWPLGLVICKALKIC |
| 1315 | WEYGRKKRRQRRR |
| 1316 | WEAALAEALAEALAEHLAEALAEALEALAA |
| 1317 | WEAKLAKALAKALAKHLAKALAKALKACEA |
| 1318 | WEARLARALARALARHLARALARA |
| 1319 | WEARLARALARALARHLARALARALRACEA |
| 1320 | WEAVVAYGRKKRRQRRR |
| 1321 | WEAVVLYGRKKRRQRRR |
| 1322 | WELYGRKKRRQRRR |
| 1323 | WELVYGRKKRRQRRR |
| 1324 | WELVVYGRKKRRQRRR |
| 1325 | WELVVAYGRKKRRQRRR |
| 1326 | WELVVLYGRKKRRQRRR |
| 1327 | WELVVLGYGRKKRRQRRR |
| 1328 | WELVVLGKYGRKKRRQRRR |
| 1329 | WELVVLGKLYGRKKRRQRRR |
| 1330 | WFQNRRMKWKK |
| 1331 | WIIFKIAASHKK |
| 1332 | WIIFRAAASHKK |
| 1333 | WIIFRALISHKK |
| 1334 | WIIFRIAASHKK |
| 1335 | WIIFRIAATHKK |
| 1336 | WIIFRIAAYHKK |
| 1337 | WKARRQCFRVLHHWN |
| 1338 | WKCRRQAFRVLHHWN |
| 1339 | WKCRRQCFRVLHHWN |
| 1340 | WKQSHKKGGKKGSG |
| 1341 | WLKLLKKWLKLWKKLLKLW |
| 1342 | WLKLWKKWLKLW |
| 1343 | WLKYLLKKWLKLWKKLLKLW |
| 1344 | WLKLLKKWLKLWKKLLKLW |
| 1345 | WLKLLRKWLRLWKRLLKLW |
| 1346 | WLRLLKRWLKLWRKLLRLW |
| 1347 | WLRRIKAWLRRIKALNRQLGVAA |
| 1348 | WRFKAAVALLPAVLLALLAP |
| 1349 | WRFKKSKRKV |
| 1350 | WRFKWRFK |
| 1351 | WRFKWRFKWRFK |
| 1352 | WRRRRRRRR |
| 1353 | WRWKKKKA |
| 1354 | WRWRWRWRWRWRWR |
| 1355 | WWRRRRRRRR |
| 1356 | WWWRRRRRRRR |
| 1357 | WWWWRRRRRRRR |
| 1358 | YWLKLLKKWLKLWKKLLKLW |
| 1359 | YARAAARQARA |
| 1360 | YARAAARQARAKALARQLGVAA |
| 1361 | YARAARRAARR |
| 1362 | YAREARRAARR |
| 1363 | YARKARRAARR |
| 1364 | YARVRRRGPRR |
| 1365 | YEREARRAARR |
| 1366 | YGDCLPHLKLCKENKDCCSKKCKRRGTNIEKRCR |
| 1367 | YGRAARRAARR |
| 1368 | YGRGGRRGRRR |
| 1369 | YGRKKKRRQRRR |
| 1370 | YGRKKRPQRRR |
| 1371 | YGRKKRRQRRR |
| 1372 | YGRKKRRQRRRDYQQD |
| 1373 | YGRKKRRQRRRENAEYLR |
| 1374 | YGRKKRRQRRRNYQQN |
| 1375 | YGRKKRRQRRR |
| 1376 | YGRKKRRQRRRQNAQYLR |
| 1377 | YGRKKRRQRRRC |
| 1378 | YGRKKRRQRRRAYFNGCSSPTAPLSPMSP |
| 1379 | YGRKKRRQRRRC |
| 1380 | YGRKKRRQRRRDPYHATSGALSPAKDCGSQKYAYFNGCSSPT |
| | LSPMSP |
| 1381 | YGRKKRRQRRRGC |
| 1382 | YGRKKRRQRRRGCYGRKKRRQRRRG |
| 1383 | YGRKKRRQRRRGLFGAIAGFIENGWEGMIDGWYG |
| 1384 | YGRKKRRQRRRGTALDWSWLQTE |
| 1385 | YGRKKRRQRRRPPQG |
| 1386 | YGRKKRRQRRRQRRRPTAPLSPMSP |
| 1387 | YGRKKRRQRRRYGRKKRRQRRR |
| 1388 | YGRKKRRQRRRYGRKKRRQRRRYGRKKRRQRRR |
| 1389 | YGRKKRRQRRTALDASALQTE |
| 1390 | YGRKKRRQRRTALDWSWLQTE |
| 1391 | YGRRARRAARR |
| 1392 | YGRRARRRARR |
| 1393 | YGRRARRRRRR |
| 1394 | YGRRRRRRRRR |
| 1395 | YIVLRRRRKRVNTKRS |
| 1396 | YKALRISRKLAK |
| 1397 | YKQCHKKGGKKGSG |
| 1398 | YKQCHKKGGHCFPKEKICLPPSSDFGKMDCRWRWKCCKKGSG |
| 1399 | YKQCHKKGGKKGSG |
| 1400 | YKQCHKKGGXKKGSG |
| 1401 | YKQSHKKGGKKGSG |
| 1402 | YKRAARRAARR |
| 1403 | YKRKARRAARR |
| 1404 | YNNFAYSVFL |
| 1405 | YPRAARRAARR |
| 1406 | YPYDANHTRSPT |
| 1407 | YQKQAKIMCS |
| 1408 | YRDRFAFQPH |
| 1409 | YRFK |
| 1410 | YRFKYRFKYRLFK |
| 1411 | YRQSHRRGGRRGSG |
| 1412 | YRRAARRAARA |
| 1413 | YRRRRRRRRRR |
| 1414 | YRWRCKNQ |
| 1415 | YRWRCKNQN |
| 1416 | YSHIATLPFTPT |
| 1417 | YSSYSAPVSSSLSVRRSYSSSSGS |
| 1418 | YTAIAWVKAFIRKLRK |
| 1419 | YTFGLKTSFNVQ |
| 1420 | YTFGLKTSFNVQYTFGLKTSFNVQ |
| 1421 | YTQDFNKFHTFPQTAIGVGAP |
| 1422 | YYYAAGRKRKKRT |

What is claimed is:

1. A Peptide-Assisted Genome Editing (PAGE) system comprising a) a CRISPR associated (Cas) protein linked to a Cell Penetrating Peptide (CPP), and b) an endosomal escape peptide linked to a CPP.

2. The PAGE system of claim 1, wherein the Cas is Cas9, or Cas12a, or a Cas derivative.

3. The PAGE system of claim 2, wherein the Cas derivative is a Cas protein linked to another protein or catalytic domain.

4. The PAGE system of claim 3, wherein the protein or catalytic domain is selected from the group consisting of an AID deaminase, an APOBEC deaminase, a TadA deaminase, a TET enzyme, a DNA methyltransferase, a transactivation domain, a reverse transcriptase, a histone acetyltransferase, a histone deacetylase, a sirtuin, a histone methyltransferase, a histone demethylase, a kinase, and a phosphatase.

5. The PAGE system of claim 1, wherein the endosomal escape peptide comprises any of the amino acid sequences set forth in SEQ ID NOs: 1434-1523.

6. The PAGE system of claim 1, wherein the endosomal escape peptide comprises dTAT-HA2.

7. The PAGE system of claim 1, wherein the Cas comprises a Nuclear Localization Signal (NLS) sequence.

8. The PAGE system of claim 7, wherein the NLS sequence comprises the amino acid sequence PAAKRVKLD (SEQ ID NO: 1).

9. The PAGE system of claim 7, wherein the NLS sequence further comprises a GGS linker.

10. The PAGE system of claim 1, wherein the CPP comprises any of the amino acid sequences set forth in SEQ ID NOs: 10-1422.

11. The PAGE system of claim 1, wherein the CPP comprises a sequence derived from the trans-activating transcriptional activator (Tat) from HIV-1.

12. The PAGE system of claim 11, wherein the Tat sequence comprises the amino acid sequence GRKKRRQRRRPQ (SEQ ID NO: 2).

13. An in vitro method of gene editing comprising introducing into a cell a PAGE system and at least one sgRNA or crRNA, wherein the PAGE system comprises a Cas protein linked to a CPP and an endosomal escape peptide linked to a CPP.

14. An in vivo method of gene editing comprising
introducing into a cell a PAGE system and at least one sgRNA or crRNA, wherein the PAGE system comprises a Cas protein linked to a CPP and an endosomal escape peptide linked to a CPP, and
administering the cell to a subject.

15. The method of claim 13, wherein the Cas is Cas9, or Cas12a, or a Cas derivative.

16. The method of claim 15, wherein the Cas derivative is a Cas protein linked to another protein or catalytic domain.

17. The method of claim 16, wherein the protein or catalytic domain is selected from the group consisting of an AID deaminase, an APOBEC deaminase, a TadA deaminase, a TET enzyme, a DNA methyltransferase, a transactivation domain, a reverse transcriptase, a histone acetyltransferase, a histone deacetylase, a sirtuin, a histone methyltransferase, a histone demethylase, a kinase, and a phosphatase.

18. The method of claim 13, wherein the endosomal escape peptide comprises any of the amino acid sequences set forth in SEQ ID NOs: 1434-1523.

19. The method of claim 13, wherein the endosomal escape peptide comprises dTAT-HA2.

20. The method of claim 13, wherein the Cas comprises a NLS sequence.

21. The method of claim 20, wherein the NLS sequence comprises the amino acid sequence PAAKRVKLD (SEQ ID NO: 1).

22. The method of claim 20, wherein the NLS sequence further comprises a GGS linker.

23. The method of claim 13, wherein the CPP comprises any of the amino acid sequences set forth in SEQ ID NOs: 10-1422.

24. The method of claim 13, wherein the CPP comprises a sequence derived from the trans-activating transcriptional activator (Tat) from HIV-1.

25. The method of claim 24, wherein the Tat sequence comprises the amino acid sequence GRKKRRQRRRPQ (SEQ ID NO: 2).

26. The method of claim 13, wherein the method does not require electroporation.

27. The method of claim 13, wherein the PAGE system is introduced into the cell in a medium that does not contain serum.

28. The method of claim 13, wherein the endosomal escape peptide is introduced into the cell at a concentration of about 25-75 μM.

29. The method of claim 13, wherein the Cas is introduced into the cell at a concentration of about 0.5-5 μM.

30. The method of claim 13, wherein the cell is an immune cell.

31. The method of claim 13, wherein the cell is selected from the group consisting of a primary human CD8 T cell, a human iPSC, and a CAR T cell.

32. The method of claim 13, wherein the sgRNA targets Ano9, Pdcd1, Thy1, Ptprc, PTPRC, or B2M.

33. The method of claim 13, wherein the subject is in need of a treatment for a disease or disorder, and wherein when the edited cell is administered to the subject, the disease or disorder is treated in the subject.

34. The method of claim 33, wherein the disease or disorder is an infection.

35. The method of claim 34, wherein the disease or disorder is related to T cell exhaustion.


SEQUENCE LISTING (1523 sequences)

SEQ ID NO: 1 - protein, 9 aa, Artificial Sequence
  Myc NLS
PAAKRVKLD

SEQ ID NO: 2 - protein, 12 aa, Artificial Sequence
  Tat
GRKKRRQRRRPQ

SEQ ID NO: 3 - protein, 11 aa, Artificial Sequence
  Tat
YGRKKRRQRRR

SEQ ID NO: 4 - protein, 11 aa, Artificial Sequence
  Tat
CGRKKRRQRRR

SEQ ID NO: 5 - protein, 13 aa, Artificial Sequence
  Tat
GRKKRRQRRRPPQ

SEQ ID NO: 6 - protein, 11 aa, Artificial Sequence
  Tat
RKKRRQRRRPQ

SEQ ID NO: 7 - protein, 9 aa, Artificial Sequence
  Tat
RKKRRQRRR

SEQ ID NO: 8 - DNA, 20 bp, Artificial Sequence
  sgRNA
gcctggctcacagtgtcaga

SEQ ID NO: 9 - DNA, 20 bp, Artificial Sequence
  sgRNA
ggtatcatgagtgccctagt

SEQ ID NO: 10 - protein, 21 aa, Artificial Sequence
  CPP
ALYLLGKINLKALAALAKKIL

SEQ ID NO: 11 - protein, 10 aa, Artificial Sequence
  CPP
ANIIPLLPIC

SEQ ID NO: 12 - protein, 6 aa, Artificial Sequence
  CPP
AAAWFW

SEQ ID NO: 13 - protein, 27 aa, Artificial Sequence
  CPP
AAVACRICMRNFSTRQARRNHRRRHRR

SEQ ID NO: 14 - protein, 16 aa, Artificial Sequence
  CPP
AAVALLPAVLLALLAK

SEQ ID NO: 15 - protein, 26 aa, Artificial Sequence
  CPP
AAVALLPAVLLALLAKKNNLKDCGLF

SEQ ID NO: 16 - protein, 26 aa, Artificial Sequence
  CPP
AAVALLPAVLLALLAKKNNLKECGLY

SEQ ID NO: 17 - protein, 16 aa, Artificial Sequence
  CPP
AAVALLPAVLLALLAP

SEQ ID NO: 18 - protein, 41 aa, Artificial Sequence
  CPP
AAVALLPAVLLALLAPEILLPNNYNAYESYKYPGMFIALSK

SEQ ID NO: 19 - protein, 28 aa, Artificial Sequence
  CPP
AAVALLPAVLLALLAPRKKRRQRRRPPQ

SEQ ID NO: 20 - protein, 29 aa, Artificial Sequence
  CPP
AAVALLPAVLLALLAPRKKRRQRRRPPQC

SEQ ID NO: 21 - protein, 22 aa, Artificial Sequence
  CPP
AAVALLPAVLLALLAPRRRRRR

SEQ ID NO: 22 - protein, 28 aa, Artificial Sequence
  CPP
AAVALLPAVLLALLAPSGASGLDKRDYV

SEQ ID NO: 23 - protein, 26 aa, Artificial Sequence
  CPP
AAVALLPAVLLALLAPVQRKRQKLMP

SEQ ID NO: 24 - protein, 43 aa, Artificial Sequence
  CPP
AAVALLPAVLLALLAVTDQLGEDFFAVDLEAFLQEFGLLPEKE

SEQ ID NO: 25 - protein, 17 aa, Artificial Sequence
  CPP
WELVVLYGRKKRRQRRR

SEQ ID NO: 26 - protein, 17 aa, Artificial Sequence
  CPP
ACGRGRGRCGRGRGRCG

SEQ ID NO: 27 - protein, 17 aa, Artificial Sequence
  CPP
ACGRGRGRCRGRGRGCG

SEQ ID NO: 28 - protein, 17 aa, Artificial Sequence
  CPP
ACHGRRWGCGRHRGRCG

SEQ ID NO: 29 - protein, 17 aa, Artificial Sequence
  CPP
ACRDRFRNCPADEALCG

SEQ ID NO: 30 - protein, 17 aa, Artificial Sequence
  CPP
ACRDRFRNCPADERLCG

SEQ ID NO: 31 - protein, 17 aa, Artificial Sequence
  CPP
ACRDRFRRCPADERLCG

SEQ ID NO: 32 - protein, 17 aa, Artificial Sequence
  CPP
ACRDRFRRCPADRRLCG

SEQ ID NO: 33 - protein, 17 aa, Artificial Sequence
  CPP
ACRGRGRGCGRGRGRCG

SEQ ID NO: 34 - protein, 17 aa, Artificial Sequence
  CPP
ACRGRGRGCGSGSGSCG

SEQ ID NO: 35 - protein, 17 aa, Artificial Sequence
  CPP
ACRGRGRGCGSGSRSCG

SEQ ID NO: 36 - protein, 17 aa, Artificial Sequence
  CPP
ACRGRGRGCRGRGRGCG

SEQ ID NO: 37 - protein, 17 aa, Artificial Sequence
  CPP
ACRGRGRRCGSGRRSCG

SEQ ID NO: 38 - protein, 17 aa, Artificial Sequence
  CPP
ACRGRGRRCGSGSRSCG

SEQ ID NO: 39 - protein, 17 aa, Artificial Sequence
  CPP
ACRGRRRGCGRRRGRCG

SEQ ID NO: 40 - protein, 17 aa, Artificial Sequence
  CPP
ACRGSGRGCGRGSGRCG

SEQ ID NO: 41 - protein, 17 aa, Artificial Sequence
  CPP
ACRRSRRGCGRRSRRCG

SEQ ID NO: 42 - protein, 17 aa, Artificial Sequence
  CPP
ACSDRFRNCPADEALCG

SEQ ID NO: 43 - protein, 25 aa, Artificial Sequence
  CPP
ACSDRFRNCPADEALCGRRRRRRRR

SEQ ID NO: 44 - protein, 17 aa, Artificial Sequence
  CPP
ACSGRGRGCGRGRGSCG

SEQ ID NO: 45 - protein, 17 aa, Artificial Sequence
  CPP
ACSGRGRGCGSGSGSCG

SEQ ID NO: 46 - protein, 17 aa, Artificial Sequence
  CPP
ACSGRGSGCGSGRGSCG

SEQ ID NO: 47 - protein, 17 aa, Artificial Sequence
  CPP
ACSGSGSGCGSGSGSCG

SEQ ID NO: 48 - protein, 25 aa, Artificial Sequence
  CPP
ACSGSGSGCGSGSGSCGRRRRRRRR

SEQ ID NO: 49 - protein, 25 aa, Artificial Sequence
  CPP
ACSHSGHGCGHGSHSCGRRRRRRRR

SEQ ID NO: 50 - protein, 25 aa, Artificial Sequence
  CPP
ACSHSGWGCGHGSWSCGRRRRRRRR

SEQ ID NO: 51 - protein, 11 aa, Artificial Sequence
  CPP
ACSSSPSKHCG

SEQ ID NO: 52 - protein, 23 aa, Artificial Sequence
  CPP
ACSSSPSKHCGGGGRRRRRRRRR

SEQ ID NO: 53 - protein, 30 aa, Artificial Sequence
  CPP
ADVFDRGGPYLQRGVADLVPTATLLDTYSP

SEQ ID NO: 54 - protein, 16 aa, Artificial Sequence
  CPP
AEAEAEAEAKAKAKAK

SEQ ID NO: 55 - protein, 28 aa, Artificial Sequence
  CPP
AEAEAEAEAKAKAKAKAGGGHRRRRRRR

SEQ ID NO: 56 - protein, 26 aa, Artificial Sequence
  CPP
AEKVDPVKLNLTLSAAAEALTGLGDK

SEQ ID NO: 57 - protein, 20 aa, Artificial Sequence
  CPP
AGYLGKINLKALAALAKKIL

SEQ ID NO: 58 - protein, 21 aa, Artificial Sequence
  CPP
AGYLLGKINLKALAALAKKIL

SEQ ID NO: 59 - protein, 21 aa, Artificial Sequence
  CPP
AGYLLGKTNLKALAALAKKIL

SEQ ID NO: 60 - protein, 21 aa, Artificial Sequence
  CPP
AGYLLGKTNLKALAALAKKIL

SEQ ID NO: 61 - protein, 21 aa, Artificial Sequence
  CPP
AGYLLGKINLKALAALAKKIL

SEQ ID NO: 62 - protein, 20 aa, Artificial Sequence
  CPP
AGYLLGHINLHHLAHLHHIL

SEQ ID NO: 63 - protein, 21 aa, Artificial Sequence
  CPP
AGYLLGHINLHHLAHLHHILC

SEQ ID NO: 64 - protein, 21 aa, Artificial Sequence
  CPP
AGYLLGKINLKALAALAKKIL

SEQ ID NO: 65 - protein, 21 aa, Artificial Sequence
  CPP
AGYLLGKINLKALAALAKKIL

SEQ ID NO: 66 - protein, 21 aa, Artificial Sequence
  CPP
AGYLLGKINLKALAALAKKIL

SEQ ID NO: 67 - protein, 24 aa, Artificial Sequence
  CPP
AGYLLGKINLKALAALAKKILGGC

SEQ ID NO: 68 - protein, 38 aa, Artificial Sequence
  CPP
AGYLLGKINLKALAALAKKILTYADFIASGRTGRRNAI

SEQ ID NO: 69 - protein, 20 aa, Artificial Sequence
  CPP
AGYLLGKINLKKLAKLLLIL

SEQ ID NO: 70 - protein, 19 aa, Artificial Sequence
  CPP
AGYLLGKLKALAALAKKIL

SEQ ID NO: 71 - protein, 21 aa, Artificial Sequence
  CPP
AGYLLGKLLKKLAAAALKKLL

SEQ ID NO: 72 - protein, 21 aa, Artificial Sequence
  CPP
AGYLLGKTNLKALAALAKKIL

SEQ ID NO: 73 - protein, 21 aa, Artificial Sequence
  CPP
AGYLLGKTNLKALAALAKKIL

SEQ ID NO: 74 - protein, 21 aa, Artificial Sequence
  CPP
AGYLLGKINLKALAALAKKIL

SEQ ID NO: 75 - protein, 26 aa, Artificial Sequence
  CPP
AHALCLTERQIKIWFQNRRMKWKKEN

SEQ ID NO: 76 - protein, 26 aa, Artificial Sequence
  CPP
AHALCPPERQIKIWFQNRRMKWKKEN

SEQ ID NO: 77 - protein, 9 aa, Artificial Sequence
  CPP
RRRRRRRRR

SEQ ID NO: 78 - protein, 10 aa, Artificial Sequence
  CPP
NIIAPLLPIC

SEQ ID NO: 79 - protein, 7 aa, Artificial Sequence
  CPP
NIILLIC

SEQ ID NO: 80 - protein, 8 aa, Artificial Sequence
  CPP
NIILLPIC

SEQ ID NO: 81 - protein, 10 aa, Artificial Sequence
  CPP
NIIPLLAPIC

SEQ ID NO: 82 - protein, 8 aa, Artificial Sequence
  CPP
NIIPLLIC

SEQ ID NO: 83 - protein, 9 aa, Artificial Sequence
  CPP
NIIPLLPIC

SEQ ID NO: 84 - protein, 9 aa, Artificial Sequence
  CPP
AIIYRDLIS

SEQ ID NO: 85 - protein, 12 aa, Artificial Sequence
  CPP
AIPNNQLGFPFK

SEQ ID NO: 86 - protein, 30 aa, Artificial Sequence
  CPP
AKKAKAAKKAKAAKKAKAAKKAKAAKKAKA

SEQ ID NO: 87 - protein, 24 aa, Artificial Sequence
  CPP
AKKKAAKAAKKKAAKAAKKKAAKA

SEQ ID NO: 88 - protein, 32 aa, Artificial Sequence
  CPP
AKKKAAKAAKKKAAKAAKKKAAKAAKKKAAKA

SEQ ID NO: 89 - protein, 9 aa, Artificial Sequence
  CPP
AKKRRQRRR

SEQ ID NO: 90 - protein, 18 aa, Artificial Sequence
  CPP
AKKRRQRRRAKKRRQRRR

SEQ ID NO: 91 - protein, 34 aa, Artificial Sequence
  CPP
AKVKDEPQRRSARLSAKPAPPKPEPKPKKAPAKK

SEQ ID NO: 92 - protein, 38 aa, Artificial Sequence
  CPP
ALALALALALALALALKIKKIKKIKKIKKLAKLAKKIK

SEQ ID NO: 93 - protein, 13 aa, Artificial Sequence
  CPP
ALFDIIKKIAESF

SEQ ID NO: 94 - protein, 18 aa, Artificial Sequence
  CPP
ALIILRRRIRKQAHAHSK

SEQ ID NO: 95 - protein, 13 aa, Artificial Sequence
  CPP
ALWKTLLKKVLKA

SEQ ID NO: 96 - protein, 20 aa, Artificial Sequence
  CPP
ALWKTLLKKVLKAPKKKRKV

SEQ ID NO: 97 - protein, 15 aa, Artificial Sequence
  CPP
ALWMRWYSPTTRRYG

SEQ ID NO: 98 - protein, 28 aa, Artificial Sequence
  CPP
ALWMTLLKKVLKAAAKAALNAVLVGANA

SEQ ID NO: 99 - protein, 12 aa, Artificial Sequence
  CPP
APWHLSSQYSRT

SEQ ID NO: 100 - protein, 16 aa, Artificial Sequence
  CPP
AQIKIWFQNRRMKWKK

SEQ ID NO: 101 - protein, 19 aa, Artificial Sequence
  CPP
ARCSDRFRNCPADEALCGR

SEQ ID NO: 102 - protein, 19 aa, Artificial Sequence
  CPP
ARCSGSGSGCGSGSGSCGR

SEQ ID NO: 103 - protein, 30 aa, Artificial Sequence
  CPP
ARRARAARRARAARRARAARRARAARRARA

SEQ ID NO: 104 - protein, 21 aa, Artificial Sequence
  CPP
ARRCSDRFRNCPADEALCGRR

SEQ ID NO: 105 - protein, 21 aa, Artificial Sequence
  CPP
ARRCSGSGSGCGSGSGSCGRR

SEQ ID NO: 106 - protein, 24 aa, Artificial Sequence
  CPP
ARRRAARAARRRAARAARRRAARA

SEQ ID NO: 107 - protein, 32 aa, Artificial Sequence
  CPP
ARRRAARAARRRAARAARRRAARAARRRAARA

SEQ ID NO: 108 - protein, 23 aa, Artificial Sequence
  CPP
ARRRCSDRFRNCPADEALCGRRR

SEQ ID NO: 109 - protein, 23 aa, Artificial Sequence
  CPP
ARRRCSGSGSGCGSGSGSCGRRR

SEQ ID NO: 110 - protein, 25 aa, Artificial Sequence
  CPP
ARRRRCSDRFRNCPADEALCGRRRR

SEQ ID NO: 111 - protein, 25 aa, Artificial Sequence
  CPP
ARRRRCSGSGSGCGSGSGSCGRRRR

SEQ ID NO: 112 - protein, 32 aa, Artificial Sequence
  CPP
ARTINAQQAELDSALLAAAGFGNTTADVFDRG

SEQ ID NO: 113 - protein, 21 aa, Artificial Sequence
  CPP
ASMWERVKSIIKSSLAAASNI

SEQ ID NO: 114 - protein, 12 aa, Artificial Sequence
  CPP
AVPAENALNNPF

SEQ ID NO: 115 - protein, 11 aa, Artificial Sequence
  CPP
AVPAKKRZKSV

SEQ ID NO: 116 - protein, 26 aa, Artificial Sequence
  CPP
AYALCLTERQIKIWFANRRMKWKKEN

SEQ ID NO: 117 - protein, 12 aa, Artificial Sequence
  CPP
AYGRKKRRQRRR

SEQ ID NO: 118 - protein, 20 aa, Artificial Sequence
  CPP
AYLLGKINLKALAALAKKIL

SEQ ID NO: 119 - protein, 20 aa, Artificial Sequence
  CPP
AYRIKPTFRRLKWKYKGKFW

SEQ ID NO: 120 - protein, 10 aa, Artificial Sequence
  CPP
ERRRKKRRRE

SEQ ID NO: 121 - protein, 10 aa, Artificial Sequence
  CPP
ERRRKKRRRE

SEQ ID NO: 122 - protein, 10 aa, Artificial Sequence
  CPP
ERRRKKRRRE

SEQ ID NO: 123 - protein, 10 aa, Artificial Sequence
  CPP
ERRRKKRRRE

SEQ ID NO: 124 - protein, 6 aa, Artificial Sequence
  CPP
KRRRRE

SEQ ID NO: 125 - protein, 8 aa, Artificial Sequence
  CPP
KRRRRRRE

SEQ ID NO: 126 - protein, 9 aa, Artificial Sequence
  CPP
KRRRRRRRE

SEQ ID NO: 127 - protein, 10 aa, Artificial Sequence
  CPP
KRRRRRRRRE

SEQ ID NO: 128 - protein, 11 aa, Artificial Sequence
  CPP
KRRRRRRRRRE

SEQ ID NO: 129 - protein, 16 aa, Artificial Sequence
  CPP
GLRKRLRKFRNKIKEK

SEQ ID NO: 130 - protein, 16 aa, Artificial Sequence
  CPP
GLRKRLRKFRNKIKEK

SEQ ID NO: 131 - protein, 9 aa, Artificial Sequence
  CPP
RKRRRRRRE

SEQ ID NO: 132 - protein, 9 aa, Artificial Sequence
  CPP
RRKRRRRRE

SEQ ID NO: 133 - protein, 9 aa, Artificial Sequence
  CPP
RRRKRRRRE

SEQ ID NO: 134 - protein, 9 aa, Artificial Sequence
  CPP
RRRRKRRRE

SEQ ID NO: 135 - protein, 7 aa, Artificial Sequence
  CPP
RRRRRRR

SEQ ID NO: 136 - protein, 13 aa, Artificial Sequence
  CPP
GGGGRRFFRRFRR

SEQ ID NO: 137 - protein, 13 aa, Artificial Sequence
  CPP
GGGGRRFFRRWRR

SEQ ID NO: 138 - protein, 13 aa, Artificial Sequence
  CPP
GGGGRRFWRRFRR

SEQ ID NO: 139 - protein, 13 aa, Artificial Sequence
  CPP
GGGGRRFWRRWRR

SEQ ID NO: 140 - protein, 13 aa, Artificial Sequence
  CPP
GGGGRRWFRRFRR

SEQ ID NO: 141 - protein, 13 aa, Artificial Sequence
  CPP
GGGGRRWFRRWRR

SEQ ID NO: 142 - protein, 13 aa, Artificial Sequence
  CPP
GGGGRRWWRRFRR

SEQ ID NO: 143 - protein, 13 aa, Artificial Sequence
  CPP
GGGGRRWWRRWRR

SEQ ID NO: 144 - protein, 7 aa, Artificial Sequence
  CPP
BRRRRRR

SEQ ID NO: 145 - protein, 8 aa, Artificial Sequence
  CPP
BRRRRRRR

SEQ ID NO: 146 - protein, 9 aa, Artificial Sequence
  CPP
BRRRRRRRR

SEQ ID NO: 147 - protein, 10 aa, Artificial Sequence
  CPP
  misc_feature (4)..(4): Xaa can be any naturally occurring amino acid
  misc_feature (7)..(7): Xaa can be any naturally occurring amino acid
  misc_feature (10)..(10): Xaa can be any naturally occurring amino acid
BRRXRRXRRX

SEQ ID NO: 148 - protein, 16 aa, Artificial Sequence
  CPP
  misc_feature (2)..(2): Xaa can be any naturally occurring amino acid
  misc_feature (4)..(4): Xaa can be any naturally occurring amino acid
  misc_feature (6)..(7): Xaa can be any naturally occurring amino acid
  misc_feature (9)..(9): Xaa can be any naturally occurring amino acid
  misc_feature (11)..(12): Xaa can be any naturally occurring amino acid
  misc_feature (14)..(14): Xaa can be any naturally occurring amino acid
  misc_feature (16)..(16): Xaa can be any naturally occurring amino acid
BXRXRXXRXRXXRXRX

SEQ ID NO: 149 - protein, 16 aa, Artificial Sequence
  CPP
CALNNYGRKKRRQRRR

SEQ ID NO: 150 - protein, 9 aa, Artificial Sequence
  CPP
CARSKNKDC

SEQ ID NO: 151 - protein, 12 aa, Artificial Sequence
  CPP
CASGQQGLLKLC

SEQ ID NO: 152 - protein, 13 aa, Artificial Sequence
  CPP
CAYGGQQGGQGGG

SEQ ID NO: 153 - protein, 13 aa, Artificial Sequence
  CPP
CAYGRKKRRQRRR

SEQ ID NO: 154 - protein, 13 aa, Artificial Sequence
  CPP
CCTGRKKRRQRRR

SEQ ID NO: 155 - protein, 20 aa, Artificial Sequence
  CPP
CELAGIGILTVKKKKKQKKK

SEQ ID NO: 156 - protein, 20 aa, Artificial Sequence
  CPP
CELAGIGILTVRKKRRQRRR

SEQ ID NO: 157 - protein, 23 aa, Artificial Sequence
  CPP
CGAYDLRRRERQSRLRRRERQSR

SEQ ID NO: 158 - protein, 36 aa, Artificial Sequence
  CPP
CGGGARKKAAKAARKKAAKAARKKAAKAARKKAAKA

SEQ ID NO: 159 - protein, 16 aa, Artificial Sequence
  CPP
CGGGGYGRKKRRQRRR

SEQ ID NO: 160 - protein, 17 aa, Artificial Sequence
  CPP
CGGGRRRRRRRRRLLLL

SEQ ID NO: 161 - protein, 15 aa, Artificial Sequence
  CPP
CGGGYGRKKRRQRRR

SEQ ID NO: 162 - protein, 30 aa, Artificial Sequence
  CPP
CGGKDCERRFSRSDQLKRHQRRHTGVKPFQ

SEQ ID NO: 163 - protein, 25 aa, Artificial Sequence
  CPP
CGGMVTVLFRRLRIRRASGPPRVRV

SEQ ID NO: 164 - protein, 7 aa, Artificial Sequence
  CPP
CGNKRTR

SEQ ID NO: 165 - protein, 9 aa, Artificial Sequence
  CPP
CGNKRTRGC

SEQ ID NO: 166 - protein, 33 aa, Artificial Sequence
  CPP
CGNVVRQGCGYGRKKRRQRRRGTALDWSWLQTE

SEQ ID NO: 167 - protein, 14 aa, Artificial Sequence
  CPP
CGRKKRRQRRRPPQ

SEQ ID NO: 168 - protein, 14 aa, Artificial Sequence
  CPP
CGRKKRRQRRRPPQ

SEQ ID NO: 169 - protein, 20 aa, Artificial Sequence
  CPP
CGRKKRAARQRAARAARPPQ

SEQ ID NO: 170 - protein, 16 aa, Artificial Sequence
  CPP
CGRKKRAARQRRRPPQ

SEQ ID NO: 171 - protein, 20 aa, Artificial Sequence
  CPP
CGRKKRLLRQRLLRLLRPPQ

SEQ ID NO: 172 - protein, 16 aa, Artificial Sequence
  CPP
CGRKKRLLRQRRRPPQ

SEQ ID NO: 173 - protein, 14 aa, Artificial Sequence
  CPP
CGRKKRRQRRRPPQ

SEQ ID NO: 174 - protein, 16 aa, Artificial Sequence
  CPP
CGRKKRRQRAARRPPQ

SEQ ID NO: 175 - protein, 16 aa, Artificial Sequence
  CPP
CGRKKRRQRLLRRPPQ

SEQ ID NO: 176 - protein, 14 aa, Artificial Sequence
  CPP
CGRKKRRQRRRPPQ

SEQ ID NO: 177 - protein, 16 aa, Artificial Sequence
  CPP
CGRKKRRQRRAARPPQ

SEQ ID NO: 178 - protein, 16 aa, Artificial Sequence
  CPP
CGRKKRRQRRLLRPPQ

SEQ ID NO: 179 - protein, 14 aa, Artificial Sequence
  CPP
CGRKKRRQRRRPPQ

SEQ ID NO: 180 - protein, 16 aa, Artificial Sequence
  CPP
CGRKKRRQRRWWRPPQ

SEQ ID NO: 181 - protein, 16 aa, Artificial Sequence
  CPP
CGRKKRRQRWWRRPPQ

SEQ ID NO: 182 - protein, 16 aa, Artificial Sequence
  CPP
CGRKKRWWRQRRRPPQ

SEQ ID NO: 183 - protein, 20 aa, Artificial Sequence
  CPP
CGRKKRWWRQRWWRWWRPPQ

SEQ ID NO: 184 - protein, 15 aa, Artificial Sequence
  CPP
CGYGRKKRRQRRRGC

SEQ ID NO: 185 - protein, 8 aa, Artificial Sequence
  CPP
CHAIYPRH

SEQ ID NO: 186 - protein, 21 aa, Artificial Sequence
  CPP
CHHHHHRRRRRRRRRHHHHHC

SEQ ID NO: 187 - protein, 10 aa, Artificial Sequence
  CPP
CHHRRRRHHC

SEQ ID NO: 188 - protein, 26 aa, Artificial Sequence
  CPP
CIGAVLKVLTTGLPALISWIKRKRQQ

SEQ ID NO: 189 - protein, 10 aa, Artificial Sequence
  CPP
CIISRDLISH

SEQ ID NO: 190 - protein, 32 aa, Artificial Sequence
  CPP
CKDEPQRRSARLSAKPAPPKPEPKPKKAPAKK

SEQ ID NO: 191 - protein, 10 aa, Artificial Sequence
  CPP
CKKKKKKKKK

SEQ ID NO: 192 - protein, 13 aa, Artificial Sequence
  CPP
CKYGRKKRRQRRR

SEQ ID NO: 193 - protein, 31 aa, Artificial Sequence
  CPP
CLLIILRRRIRKQAHAHSKNHQQQNPHQPPM

SEQ ID NO: 194 - protein, 20 aa, Artificial Sequence
  CPP
CLLYWFRRRHRHHRRRHRRC

SEQ ID NO: 195 - protein, 5 aa, Artificial Sequence
  CPP
CNGRC

SEQ ID NO: 196 - protein, 16 aa, Artificial Sequence
  CPP
CREKAKKLFKKILKKL

SEQ ID NO: 197 - protein, 10 aa, Artificial Sequence
  CPP
CRFRFKCCKK

SEQ ID NO: 198 - protein, 10 aa, Artificial Sequence
  CPP
CRFRWKCCKK

SEQ ID NO: 199 - protein, 5 aa, Artificial Sequence
  CPP
CRGDC

SEQ ID NO: 200 - protein, 5 aa, Artificial Sequence
  CPP
CRGDK

SEQ ID NO: 201 - protein, 9 aa, Artificial Sequence
  CPP
CRGDKGDPC

SEQ ID NO: 202 - protein, 9 aa, Artificial Sequence
  CPP
CRGDKGPDC

SEQ ID NO: 203 - protein, 13 aa, Artificial Sequence
  CPP
CRKARYRGRKRQR

SEQ ID NO: 204 - protein, 10 aa, Artificial Sequence
  CPP
CRKKRRQRRR

SEQ ID NO: 205 - protein, 9 aa, Artificial Sequence
  CPP
CRNGRGPDC

SEQ ID NO: 206 - protein, 18 aa, Artificial Sequence
  CPP
CRQIKIWFPNRRMKWKKC

SEQ ID NO: 207 - protein, 17 aa, Artificial Sequence
  CPP
CRQIKIWFQNRRMKWKK

SEQ ID NO: 208 - protein, 31 aa, Artificial Sequence
  CPP
CRQIKIWFQNRRMKWKKKLAKLAKKLAKLAK

SEQ ID NO: 209 - protein, 20 aa, Artificial Sequence
  CPP
CRRLRHLRHHYRRRWHRFRC

SEQ ID NO: 210 - protein, 9 aa, Artificial Sequence
  CPP
CRRRRRRRR

SEQ ID NO: 211 - protein, 10 aa, Artificial Sequence
  CPP
CRRRRRRRRR

SEQ ID NO: 212 - protein, 10 aa, Artificial Sequence
  CPP
CRWRFKCCKK

SEQ ID NO: 213 - protein, 10 aa, Artificial Sequence
  CPP
CRWRWKCCKK

SEQ ID NO: 214 - protein, 8 aa, Artificial Sequence
  CPP
CRWRWKCG

SEQ ID NO: 215 - protein, 11 aa, Artificial Sequence
  CPP
CRWRWKCGCKK

SEQ ID NO: 216 - protein, 10 aa, Artificial Sequence
  CPP
CRWRWKCSKK

SEQ ID NO: 217 - protein, 10 aa, Artificial Sequence
  CPP
  misc_feature (8)..(8): Xaa can be any naturally occurring amino acid
CRWRWKCXKK

SEQ ID NO: 218 - protein, 10 aa, Artificial Sequence
  CPP
CRWRWKSSKK

SEQ ID NO: 219 - protein, 10 aa, Artificial Sequence
  CPP
  misc_feature (7)..(7): Xaa can be any naturally occurring amino acid
CRWRWKXCKK

SEQ ID NO: 220 - protein, 10 aa, Artificial Sequence
  CPP
  misc_feature (7)..(8): Xaa can be any naturally occurring amino acid
CRWRWKXXKK

SEQ ID NO: 221 - protein, 17 aa, Artificial Sequence
  CPP
CSIPPEVKFNKPFVYLI

SEQ ID NO: 222 - protein, 16 aa, Artificial Sequence
  CPP
CSIPPEVKFNPFVYLI

SEQ ID NO: 223 - protein, 9 aa, Artificial Sequence
  CPP
CSKSSDYQC

SEQ ID NO: 224 - protein, 18 aa, Artificial Sequence
  CPP
CSSLDEPGRGGFSSESKV

SEQ ID NO: 225 - protein, 14 aa, Artificial Sequence
  CPP
CTSTTAKRKKRKLK

SEQ ID NO: 226 - protein, 6 aa, Artificial Sequence
  CPP
CTWLKY

SEQ ID NO: 227 - protein, 7 aa, Artificial Sequence
  CPP
CTWLKYH

SEQ ID NO: 228 - protein, 19 aa, Artificial Sequence
  CPP
CVKRGLKLRHVRPRVTRDV

SEQ ID NO: 229 - protein, 13 aa, Artificial Sequence
  CPP
CVQWSLLRGYQPC

SEQ ID NO: 230 - protein, 15 aa, Artificial Sequence
  CPP
CVSRRRRRRGGRRRR

SEQ ID NO: 231 - protein, 5 aa, Artificial Sequence
  CPP
CWKKK

SEQ ID NO: 232 - protein, 10 aa, Artificial Sequence
  CPP
CWKKKKKKKK

SEQ ID NO: 233 - protein, 15 aa, Artificial Sequence
  CPP
CWKKKKKKKKKKKKK

SEQ ID NO: 234 - protein, 20 aa, Artificial Sequence
  CPP
CWKKKKKKKKKKKKKKKKKK

SEQ ID NO: 235 - protein, 6 aa, Artificial Sequence
  CPP
FRRRRQ

SEQ ID NO: 236 - protein, 12 aa, Artificial Sequence
  CPP
CYGRKKRRQRRR

SEQ ID NO: 237 - protein, 35 aa, Artificial Sequence
  CPP
DAATARGRGRSAASRPTERPRAPARSASRPRRPVD

SEQ ID NO: 238 - protein, 34 aa, Artificial Sequence
  CPP
DAATATRGRSAASRPTQRPRAPARSASRPRRPVE

SEQ ID NO: 239 - protein, 11 aa, Artificial Sequence
  CPP
DCRWRWKCCKK

SEQ ID NO: 240 - protein, 11 aa, Artificial Sequence
  CPP
  misc_feature (9)..(9): Xaa can be any naturally occurring amino acid
DCRWRWKCXKK

SEQ ID NO: 241 - protein, 11 aa, Artificial Sequence
  CPP
  misc_feature (8)..(8): Xaa can be any naturally occurring amino acid
DCRWRWKXCKK

SEQ ID NO: 242 - protein, 11 aa, Artificial Sequence
  CPP
  misc_feature (8)..(9): Xaa can be any naturally occurring amino acid
DCRWRWKXXKK

SEQ ID NO: 243 - protein, 18 aa, Artificial Sequence
  CPP
DFNKFHTFPQTAIGVGAP

SEQ ID NO: 244 - protein, 12 aa, Artificial Sequence
  CPP
DITYRFRGPDWL

SEQ ID NO: 245 - protein, 12 aa, Artificial Sequence
  CPP
DPATNPGPHFPR

SEQ ID NO: 246 - protein, 26 aa, Artificial Sequence
  CPP
DPKGDPKGVTVTVTVTVTGKGDPKPD

SEQ ID NO: 247 - protein, 15 aa, Artificial Sequence
  CPP
DPVDTPNPTRRKPGK

SEQ ID NO: 248 - protein, 17 aa, Artificial Sequence
  CPP
DRDDRDDRDDRDDRDDR

SEQ ID NO: 249 - protein, 10 aa, Artificial Sequence
  CPP
DRDRDRDRDR

SEQ ID NO: 250 - protein, 17 aa, Artificial Sequence
  CPP
DRRRRGSRPSGAERRRR

SEQ ID NO: 251 - protein, 22 aa, Artificial Sequence
  CPP
DRRRRGSRPSGAERRRRRAAAA

SEQ ID NO: 252 - protein, 15 aa, Artificial Sequence
  CPP
DSLKSYWYLQKFSWR

SEQ ID NO: 253 - protein, 22 aa, Artificial Sequence
  CPP
DTWAGVEAIIRILQQLLFIHFR

SEQ ID NO: 254 - protein, 16 aa, Artificial Sequence
  CPP
EARPALLTSRLRFIPK

SEQ ID NO: 255 - protein, 10 aa, Artificial Sequence
  CPP
ECYPKKGQDP

SEQ ID NO: 256 - protein, 3 aa, Artificial Sequence
  CPP
EEE

SEQ ID NO: 257 - protein, 5 aa, Artificial Sequence
  CPP
EEEAA

SEQ ID NO: 258 - protein, 13 aa, Artificial Sequence
  CPP
EEEAAGRKRKKRT

SEQ ID NO: 259 - protein, 8 aa, Artificial Sequence
  CPP
EEEAAKKK

SEQ ID NO: 260 - protein, 30 aa, Artificial Sequence
  CPP
EEEEEEEEEEPLGLAGVSRRRRRRGGRRRR

SEQ ID NO: 261 - protein, 23 aa, Artificial Sequence
  CPP
EEEEEEEEPLGLAGRRRRRRRRN

SEQ ID NO: 262 - protein, 10 aa, Artificial Sequence
  CPP
EKGKKIFIMK

SEQ ID NO: 263 - protein, 61 aa, Artificial Sequence
  CPP
EKRPRTAFSSEQLARLKREFNENRYLTTERRRQQLSSELGLNEAQIKIWFQNKRAKIKKST

SEQ ID NO: 264 - protein, 18 aa, Artificial Sequence
  CPP
ELALELALEALEAALELA

SEQ ID NO: 265 - protein, 5 aa, Artificial Sequence
  CPP
ELPVM

SEQ ID NO: 266 - protein, 19 aa, Artificial Sequence
  CPP
ELVVLGKLYGRKKRRQRRR

SEQ ID NO: 267 - protein, 12 aa, Artificial Sequence
  CPP
EPDNWSLDFPRR

SEQ ID NO: 268 - protein, 14 aa, Artificial Sequence
  CPP
ERERERERERERER

SEQ ID NO: 269 - protein, 8 aa, Artificial Sequence
  CPP
ERKKRRRE

SEQ ID NO: 270 - protein, 20 aa, Artificial Sequence
  CPP
ESGGGGSPGRRRRRRRRRRR

SEQ ID NO: 271 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
EXREXRILFQYEXREXR

SEQ ID NO: 272 - protein, 12 aa, Artificial Sequence
  CPP
FAPWDTASFMLG

SEQ ID NO: 273 - protein, 12 aa, Artificial Sequence
  CPP
FDPFFWKYSPRD

SEQ ID NO: 274 - protein, 13 aa, Artificial Sequence
  CPP
FFFAAGRKRKKRT

SEQ ID NO: 275 - protein, 17 aa, Artificial Sequence
  CPP
FFFFFFGRRRRRRRRGC

SEQ ID NO: 276 - protein, 15 aa, Artificial Sequence
  CPP
FFFFGRRRRRRRRGC

SEQ ID NO: 277 - protein, 12 aa, Artificial Sequence
  CPP
FFGRRRRRRRGC

SEQ ID NO: 278 - protein, 13 aa, Artificial Sequence
  CPP
FFGSVLKLIPKIL

SEQ ID NO: 279 - protein, 27 aa, Artificial Sequence
  CPP
FFKKLALHALHLLALLWLHLAHLALKK

SEQ ID NO: 280 - protein, 15 aa, Artificial Sequence
  CPP
FFLIGRRRRRRRRGC

SEQ ID NO: 281 - protein, 15 aa, Artificial Sequence
  CPP
FFLIPKGRRRRRRRR

SEQ ID NO: 282 - protein, 17 aa, Artificial Sequence
  CPP
FFLIPKGRRRRRRRRGC

SEQ ID NO: 283 - protein, 16 aa, Artificial Sequence
  CPP
FFLIPKGRRRRRRRRR

SEQ ID NO: 284 - protein, 8 aa, Artificial Sequence
  CPP
FHFHFRFR

SEQ ID NO: 285 - protein, 12 aa, Artificial Sequence
  CPP
FIIFRIAASHKK

SEQ ID NO: 286 - protein, 6 aa, Artificial Sequence
  CPP
FIRIGC

SEQ ID NO: 287 - protein, 13 aa, Artificial Sequence
  CPP
FISAIASMLGKFL

SEQ ID NO: 288 - protein, 16 aa, Artificial Sequence
  CPP
FITKALGISYGRKKRR

SEQ ID NO: 289 - protein, 23 aa, Artificial Sequence
  CPP
FITKALGISYGRKKRRQRRRPPQ

SEQ ID NO: 290 - protein, 10 aa, Artificial Sequence
  CPP
FKCRRWQWRM

SEQ ID NO: 291 - protein, 7 aa, Artificial Sequence
  CPP
FKKFRKF

SEQ ID NO: 292 - protein, 26 aa, Artificial Sequence
  CPP
FKKLALHALHLLALLWLHLAHLALKK

SEQ ID NO: 293 - protein, 12 aa, Artificial Sequence
  CPP
FKQQQQQQQQQQ

SEQ ID NO: 294 - protein, 14 aa, Artificial Sequence
  CPP
FLFPLITSFLSKVL

SEQ ID NO: 295 - protein, 15 aa, Artificial Sequence
  CPP
FLGKKFKKYFLQLLK

SEQ ID NO: 296 - protein, 23 aa, Artificial Sequence
  CPP
FLIFIRVICIVIAKLKANLMCKT

SEQ ID NO: 297 - protein, 19 aa, Artificial Sequence
  CPP
FLKLLKKFLKLFKKLLKLF

SEQ ID NO: 298 - protein, 13 aa, Artificial Sequence
  CPP
FLPIIAKVLSGLL

SEQ ID NO: 299 - protein, 13 aa, Artificial Sequence
  CPP
FLPIVGKLLSGLL

SEQ ID NO: 300 - protein, 13 aa, Artificial Sequence
  CPP
FLPLIGKILGTIL

SEQ ID NO: 301 - protein, 13 aa, Artificial Sequence
  CPP
FLPLIGRVLSGIL

SEQ ID NO: 302 - protein, 13 aa, Artificial Sequence
  CPP
FLSGIVGMLGKLF

SEQ ID NO: 303 - protein, 13 aa, Artificial Sequence
  CPP
FLSSIGKILGNLL

SEQ ID NO: 304 - protein, 19 aa, Artificial Sequence
  CPP
FQFNFQFNGGGHRRRRRRR

SEQ ID NO: 305 - protein, 10 aa, Artificial Sequence
  CPP
FQNRRMKWKK

SEQ ID NO: 306 - protein, 12 aa, Artificial Sequence
  CPP
FQPYDHPAEVSY

SEQ ID NO: 307 - protein, 16 aa, Artificial Sequence
  CPP
FQWQRNMRKVRGPPVS

SEQ ID NO: 308 - protein, 8 aa, Artificial Sequence
  CPP
FRFKFRFK

SEQ ID NO: 309 - protein, 37 aa, Artificial Sequence
  CPP
FRVPLRIRPCVVAPRLVMVRHTFGRIARWVAGPLETR

SEQ ID NO: 310 - protein, 9 aa, Artificial Sequence
  CPP
FTFHFTFHF

SEQ ID NO: 311 - protein, 12 aa, Artificial Sequence
  CPP
FTYKNFFWLPEL

SEQ ID NO: 312 - protein, 13 aa, Artificial Sequence
  CPP
FVQWFSKFLGRIL

SEQ ID NO: 313 - protein, 22 aa, Artificial Sequence
  CPP
FVTRGCPRRLVARLIRVMVPRR

SEQ ID NO: 314 - protein, 9 aa, Artificial Sequence
  CPP
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
FXRFXRFXR

SEQ ID NO: 315 - protein, 12 aa, Artificial Sequence
  CPP
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
  misc_feature (11)..(11): Xaa can be any naturally occurring amino acid
FXRFXRFXRFXR

SEQ ID NO: 316 - protein, 15 aa, Artificial Sequence
  CPP
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
  misc_feature (11)..(11): Xaa can be any naturally occurring amino acid
  misc_feature (14)..(14): Xaa can be any naturally occurring amino acid
FXRFXRFXRFXRFXR

SEQ ID NO: 317 - protein, 18 aa, Artificial Sequence
  CPP
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
  misc_feature (11)..(11): Xaa can be any naturally occurring amino acid
  misc_feature (14)..(14): Xaa can be any naturally occurring amino acid
  misc_feature (17)..(17): Xaa can be any naturally occurring amino acid
FXRFXRFXRFXRFXRFXR

SEQ ID NO: 318 - protein, 14 aa, Artificial Sequence
  CPP
GACTKSIPPICFPD

SEQ ID NO: 319 - protein, 13 aa, Artificial Sequence
  CPP
GAFDIIKKIAESF

SEQ ID NO: 320 - protein, 27 aa, Artificial Sequence
  CPP
GALFLAFLAAALSLMGLWSQPKKKRKV

SEQ ID NO: 321 - protein, 27 aa, Artificial Sequence
  CPP
GALFLAFLAAALSLMGLWSQPKKKRRV

SEQ ID NO: 322 - protein, 27 aa, Artificial Sequence
  CPP
GALFLGFLGAAGSTMGAWSQPKKKRKV

SEQ ID NO: 323 - protein, 27 aa, Artificial Sequence
  CPP
GALFLGFLGAAGSTMGAWSQPKSKRKV

SEQ ID NO: 324 - protein, 24 aa, Artificial Sequence
  CPP
GALFLGWLGAAGSTMGAPKKKRKV

SEQ ID NO: 325 - protein, 27 aa, Artificial Sequence
  CPP
GALFLGWLGAAGSTMGAPKSKRKVGGC

SEQ ID NO: 326 - protein, 22 aa, Artificial Sequence
  CPP
GAYDLRRRERQSRLRRRERQSR

SEQ ID NO: 327 - protein, 16 aa, Artificial Sequence
  CPP
GCGGGYGRKKRRQRRR

SEQ ID NO: 328 - protein, 9 aa, Artificial Sequence
  CPP
GDLPHLKLC

SEQ ID NO: 329 - protein, 27 aa, Artificial Sequence
  CPP
GDVYADAAPDLFDFLDSSVTTARTINA

SEQ ID NO: 330 - protein, 22 aa, Artificial Sequence
  CPP
GEQIAQLIAGYIDIILKKKKSK

SEQ ID NO: 331 - protein, 24 aa, Artificial Sequence
  CPP
GGAYVTRSSAVRLRSSVPGVRLLQ

SEQ ID NO: 332 - protein, 35 aa, Artificial Sequence
  CPP
GGGARKKAAKAARKKAAKAARKKAAKAARKKAAKA

SEQ ID NO: 333 - protein, 17 aa, Artificial Sequence
  CPP
GGGGRRRRRRRRRLLLL

SEQ ID NO: 334 - protein, 19 aa, Artificial Sequence
  CPP
GGGRRRRRRYGRKKRRQRR

SEQ ID NO: 335 - protein, 11 aa, Artificial Sequence
  CPP
GGRRARRRRRR

SEQ ID NO: 336 - protein, 34 aa, Artificial Sequence
  CPP
GGVCPAILKKCRRDSDCPGACICRGNGYCGSGSD

SEQ ID NO: 337 - protein, 34 aa, Artificial Sequence
  CPP
GGVCPKILAACRRDSDCPGACICRGNGYCGSGSD

SEQ ID NO: 338 - protein, 34 aa, Artificial Sequence
  CPP
GGVCPKILAKCRRDSDCPGACICRGNGYCGSGSD

SEQ ID NO: 339 - protein, 34 aa, Artificial Sequence
  CPP
GGVCPKILKACRRDSDCPGACICRGNGYCGSGSD

SEQ ID NO: 340 - protein, 34 aa, Artificial Sequence
  CPP
GGVCPKILKKCRRDSDCPGACICRGNGWCGSGSD

SEQ ID NO: 341 - protein, 34 aa, Artificial Sequence
  CPP
GGVCPKILKKCRRDSDCPGACICRGNGYCGSGSD

SEQ ID NO: 342 - protein, 34 aa, Artificial Sequence
  CPP
GGVCPKILRRCRRDSDCPGACICRGNGWCGSGSD

SEQ ID NO: 343 - protein, 34 aa, Artificial Sequence
  CPP
GGVCPKILRRCRRDSDCPGACICRGNGYCGSGSD

SEQ ID NO: 344 - protein, 34 aa, Artificial Sequence
  CPP
GGVCPKILRRCRRDSDCPGACICRGNGYCGSGSR

SEQ ID NO: 345 - protein, 34 aa, Artificial Sequence
  CPP
GGVCPRILRRCRRDSDCPGACICRGNGYCGSGSK

SEQ ID NO: 346 - protein, 14 aa, Artificial Sequence
  CPP
GHHHHHHHHHHHHH

SEQ ID NO: 347 - protein, 42 aa, Artificial Sequence
  CPP
GIGKFLHSAKKFGKAFVGEIMNSGGKKWKMRRNQFWVKVQRG

SEQ ID NO: 348 - protein, 23 aa, Artificial Sequence
  CPP
GIGKFLHSAKKWGKAFVGQIMNC

SEQ ID NO: 349 - protein, 14 aa, Artificial Sequence
  CPP
GILDAIKAIAKAAG

SEQ ID NO: 350 - protein, 16 aa, Artificial Sequence
  CPP
GKHRHERGHHRDRRER

SEQ ID NO: 351 - protein, 16 aa, Artificial Sequence
  CPP
GKINLKALAALAKKIL

SEQ ID NO: 352 - protein, 15 aa, Artificial Sequence
  CPP
GKKALKLAAKLLKKC

SEQ ID NO: 353 - protein, 10 aa, Artificial Sequence
  CPP
GKKKKKKKKK

SEQ ID NO: 354 - protein, 11 aa, Artificial Sequence
  CPP
GKKKKRKREKL

SEQ ID NO: 355 - protein, 17 aa, Artificial Sequence
  CPP
GKKKRKLSNRESAKRSR

SEQ ID NO: 356 - protein, 17 aa, Artificial Sequence
  CPP
GKKTNLFSALIKKKKTA

SEQ ID NO: 357 - protein, 18 aa, Artificial Sequence
  CPP
GKRARNTEAARRSRARKL

SEQ ID NO: 358 - protein, 22 aa, Artificial Sequence
  CPP
GKRKKKGKGLGKKRDPCLRKYK

SEQ ID NO: 359 - protein, 15 aa, Artificial Sequence
  CPP
GKRKKKGKLGKKRDP

SEQ ID NO: 360 - protein, 17 aa, Artificial Sequence
  CPP
GKRKKKGKLGKKRPRSR

SEQ ID NO: 361 - protein, 15 aa, Artificial Sequence
  CPP
GKRRRRATAKYRSAH

SEQ ID NO: 362 - protein, 18 aa, Artificial Sequence
  CPP
GKRVAKRKLIEQNRERRR

SEQ ID NO: 363 - protein, 22 aa, Artificial Sequence
  CPP
GKYVSLTTPKNPTKRRITPKDV

SEQ ID NO: 364 - protein, 13 aa, Artificial Sequence
  CPP
GLADIIKKIAESF

SEQ ID NO: 365 - protein, 13 aa, Artificial Sequence
  CPP
GLFAIIKKIAESF

SEQ ID NO: 366 - protein, 13 aa, Artificial Sequence
  CPP
GLFDAIKKIAESF

SEQ ID NO: 367 - protein, 13 aa, Artificial Sequence
  CPP
GLFDIAKKIAESF

SEQ ID NO: 368 - protein, 16 aa, Artificial Sequence
  CPP
GLFDIAKKVIGVIGSL

SEQ ID NO: 369 - protein, 13 aa, Artificial Sequence
  CPP
GLFDIIAKIAESF

SEQ ID NO: 370 - protein, 13 aa, Artificial Sequence
  CPP
GLFDIIHKIAESF

SEQ ID NO: 371 - protein, 13 aa, Artificial Sequence
  CPP
GLFDIIKAIAESF

SEQ ID NO: 372 - protein, 13 aa, Artificial Sequence
  CPP
GLFDIIKHIAESF

SEQ ID NO: 373 - protein, 13 aa, Artificial Sequence
  CPP
GLFDIIKKAAESF

SEQ ID NO: 374 - protein, 13 aa, Artificial Sequence
  CPP
GLFDIIKKIAASF

SEQ ID NO: 375 - protein, 13 aa, Artificial Sequence
  CPP
GLFDIIKKIADSF

SEQ ID NO: 376 - protein, 13 aa, Artificial Sequence
  CPP
GLFDIIKKIAEAF

SEQ ID NO: 377 - protein, 13 aa, Artificial Sequence
  CPP
GLFDIIKKIAESA

SEQ ID NO: 378 - protein, 13 aa, Artificial Sequence
  CPP
GLFDIIKKIAESF

SEQ ID NO: 379 - protein, 13 aa, Artificial Sequence
  CPP
GLFDIIKKIAESI

SEQ ID NO: 380 - protein, 13 aa, Artificial Sequence
  CPP
GLFDIIKKIAHSF

SEQ ID NO: 381 - protein, 13 aa, Artificial Sequence
  CPP
GLFDIIKKIARSF

SEQ ID NO: 382 - protein, 16 aa, Artificial Sequence
  CPP
GLFDIIKKVASVIGGL

SEQ ID NO: 383 - protein, 16 aa, Artificial Sequence
  CPP
GLFDIIKKVASVVGGL

SEQ ID NO: 384 - protein, 13 aa, Artificial Sequence
  CPP
GLFDIIKRIAESF

SEQ ID NO: 385 - protein, 13 aa, Artificial Sequence
  CPP
GLFDIIRKIAESF

SEQ ID NO: 386 - protein, 17 aa, Artificial Sequence
  CPP
GLFDIVKKIAGHIAGSI

SEQ ID NO: 387 - protein, 17 aa, Artificial Sequence
  CPP
GLFDIVKKIAGHIASSI

SEQ ID NO: 388 - protein, 17 aa, Artificial Sequence
  CPP
GLFDIVKKIAGHIVSSI

SEQ ID NO: 389 - protein, 16 aa, Artificial Sequence
  CPP
GLFDIVKKVVGAFGSL

SEQ ID NO: 390 - protein, 16 aa, Artificial Sequence
  CPP
GLFDIVKKVVGAIGSL

SEQ ID NO: 391 - protein, 16 aa, Artificial Sequence
  CPP
GLFDIVKKVVGALGSL

SEQ ID NO: 392 - protein, 16 aa, Artificial Sequence
  CPP
GLFDIVKKVVGTLAGL

SEQ ID NO: 393 - protein, 16 aa, Artificial Sequence
  CPP
GLFDVIKKVASVIGGL

SEQ ID NO: 394 - protein, 36 aa, Artificial Sequence
  CPP
GLFEAIEGFIENGWEGMIDGWYGGGGRRRRRRRRRK

SEQ ID NO: 395 - protein, 20 aa, Artificial Sequence
  CPP
GLFEALLELLESLWELLLEA

SEQ ID NO: 396 - protein, 20 aa, Artificial Sequence
  CPP
GLFKALLKLLKSLWKLLLKA

SEQ ID NO: 397 - protein, 23 aa, Artificial Sequence
  CPP
GLFKALLKLLKSLWKLLLKAGGC

SEQ ID NO: 398 - protein, 20 aa, Artificial Sequence
  CPP
GLFRALLRLLRSLWRLLLRA

SEQ ID NO: 399 - protein, 34 aa, Artificial Sequence
  CPP
GLGDKFGESIVNANTVLDDLNSRMPQSRHDIQQL

SEQ ID NO: 400 - protein, 22 aa, Artificial Sequence
  CPP
GLGSLLKKAGKKLKQPKSKRKV

SEQ ID NO: 401 - protein, 16 aa, Artificial Sequence
  CPP
GLKKLAELAHKLLKLG

SEQ ID NO: 402 - protein, 17 aa, Artificial Sequence
  CPP
GLKKLAELAHKLLKLGC

SEQ ID NO: 403 - protein, 16 aa, Artificial Sequence
  CPP
GLKKLAELFHKLLKLG

SEQ ID NO: 404 - protein, 17 aa, Artificial Sequence
  CPP
GLKKLAELFHKLLKLGC

SEQ ID NO: 405 - protein, 17 aa, Artificial Sequence
  CPP
GLKKLARLAHKLLKLGC

SEQ ID NO: 406 - protein, 17 aa, Artificial Sequence
  CPP
GLKKLARLFHKLLKLGC

SEQ ID NO: 407 - protein, 16 aa, Artificial Sequence
  CPP
GLLDIVKKVVGAFGSL

SEQ ID NO: 408 - protein, 27 aa, Artificial Sequence
  CPP
GLLEALAELLEGLRKRLRKFRNKIKEK

SEQ ID NO: 409 - protein, 24 aa, Artificial Sequence
  CPP
GLLNGLALRLGKRALKKIIKRLCR

SEQ ID NO: 410 - protein, 12 aa, Artificial Sequence
  CPP
GLPRRRRRRRRR

SEQ ID NO: 411 - protein, 29 aa, Artificial Sequence
  CPP
GLPVCGETCVGGTCNTPGCKCSWPVCTRN

SEQ ID NO: 412 - protein, 29 aa, Artificial Sequence
  CPP
GLPVCGETCVGGTCNTPGCTCSWPKCTRN

SEQ ID NO: 413 - protein, 16 aa, Artificial Sequence
  CPP
GLRKRLRKFRNKIKEK

SEQ ID NO: 414 - protein, 20 aa, Artificial Sequence
  CPP
GLWRALWRALRSLWKLKRKV

SEQ ID NO: 415 - protein, 21 aa, Artificial Sequence
  CPP
GLWRALWRALWRSLWKKKRKV

SEQ ID NO: 416 - protein, 21 aa, Artificial Sequence
  CPP
GLWRALWRALWRSLWKLKRKV

SEQ ID NO: 417 - protein, 21 aa, Artificial Sequence
  CPP
GLWRALWRALWRSLWKLKWKV

SEQ ID NO: 418 - protein, 21 aa, Artificial Sequence
  CPP
GLWRALWRALWRSLWKSKRKV

SEQ ID NO: 419 - protein, 20 aa, Artificial Sequence
  CPP
GLWRALWRGLRSLWKKKRKV

SEQ ID NO: 420 - protein, 20 aa, Artificial Sequence
  CPP
GLWRALWRGLRSLWKLKRKV

SEQ ID NO: 421 - protein, 20 aa, Artificial Sequence
  CPP
GLWRALWRLLRSLWRLLWKA

SEQ ID NO: 422 - protein, 20 aa, Artificial Sequence
  CPP
GLWRALWRLLRSLWRLLWRA

SEQ ID NO: 423 - protein, 27 aa, Artificial Sequence
  CPP
GLWRALWRLLRSLWRLLWSQPKKKRKV

SEQ ID NO: 424 - protein, 24 aa, Artificial Sequence
  CPP
GLWWKAWWKAWWKSLWWRKRKRKA

SEQ ID NO: 425 - protein, 20 aa, Artificial Sequence
  CPP
GLWWRLWWRLRSWFRLWFRA

SEQ ID NO: 426 - protein, 15 aa, Artificial Sequence
  CPP
GNYAHRVGAGAPVWL

SEQ ID NO: 427 - protein, 13 aa, Artificial Sequence
  CPP
GPFHFYQFLFPPV

SEQ ID NO: 428 - protein, 14 aa, Artificial Sequence
  CPP
GRCTKSIPPICFPA

SEQ ID NO: 429 - protein, 14 aa, Artificial Sequence
  CPP
GRCTKSIPPICFPD

SEQ ID NO: 430 - protein, 14 aa, Artificial Sequence
  CPP
GRCTKSIPPICWPD

SEQ ID NO: 431 - protein, 14 aa, Artificial Sequence
  CPP
GRCTKSIPPICWPK

SEQ ID NO: 432 - protein, 14 aa, Artificial Sequence
  CPP
GRCTRSIPPKCWPD

SEQ ID NO: 433 - protein, 24 aa, Artificial Sequence
  CPP
GRGDGPRRKKKKGPRRKKKKGPRR

SEQ ID NO: 434 - protein, 8 aa, Artificial Sequence
  CPP
GRGDSPRR

SEQ ID NO: 435 - protein, 24 aa, Artificial Sequence
  CPP
GRGDSPRRKKKKSPRRKKKKSPRR

SEQ ID NO: 436 - protein, 12 aa, Artificial Sequence
  CPP
GRGDSPRRSPRR

SEQ ID NO: 437 - protein, 12 aa, Artificial Sequence
  CPP
GRKGKHKRKKLP

SEQ ID NO: 438 - protein, 17 aa, Artificial Sequence
  CPP
  misc_feature (17)..(17): Xaa can be any naturally occurring amino acid
GRKKRRERRRPPERKCX

SEQ ID NO: 439 - protein, 14 aa, Artificial Sequence
  CPP
GRKKRRQARAPPQC

SEQ ID NO: 440 - protein, 11 aa, Artificial Sequence
  CPP
GRKKRRQPPQC

SEQ ID NO: 441 - protein, 14 aa, Artificial Sequence
  CPP
GRKKRRQRARPPQC

SEQ ID NO: 442 - protein, 12 aa, Artificial Sequence
  CPP
GRKKRRQRPPQC

SEQ ID NO: 443 - protein, 13 aa, Artificial Sequence
  CPP
GRKKRRQRRPPQC

SEQ ID NO: 444 - protein, 10 aa, Artificial Sequence
  CPP
GRKKRRQRRR

SEQ ID NO: 445 - protein, 11 aa, Artificial Sequence
  CPP
GRKKRRQRRRC

SEQ ID NO: 446 - protein, 12 aa, Artificial Sequence
  CPP
GRKKRRQRRRCG

SEQ ID NO: 447 - protein, 11 aa, Artificial Sequence
  CPP
GRKKRRQRRRG

SEQ ID NO: 448 - protein, 15 aa, Artificial Sequence
  CPP
GRKKRRQRRRMVSAL

SEQ ID NO: 449 - protein, 11 aa, Artificial Sequence
  CPP
GRKKRRQRRRP

SEQ ID NO: 450 - protein, 12 aa, Artificial Sequence
  CPP
GRKKRRQRRRPP

SEQ ID NO: 451 - protein, 13 aa, Artificial Sequence
  CPP
GRKKRRQRRRPPQ

SEQ ID NO: 452 - protein, 14 aa, Artificial Sequence
  CPP
GRKKRRQRRRPPQC

SEQ ID NO: 453 - protein, 39 aa, Artificial Sequence
  CPP
GRKKRRQRRRPPQGRKKRRQRRRPPQGRKKRRQRRRPPQ

SEQ ID NO: 454 - protein, 14 aa, Artificial Sequence
  CPP
GRKKRRQRRRPPQK

SEQ ID NO: 455 - protein, 16 aa, Artificial Sequence
  CPP
GRKKRRQRRRPPQRKC

SEQ ID NO: 456 - protein, 30 aa, Artificial Sequence
  CPP
GRKKRRQRRRPPQTYADFIASGRTGRRNAI

SEQ ID NO: 457 - protein, 14 aa, Artificial Sequence
  CPP
GRKKRRQRRRPPQY

SEQ ID NO: 458 - protein, 12 aa, Artificial Sequence
  CPP
GRKKRRQRRRPQ

SEQ ID NO: 459 - protein, 13 aa, Artificial Sequence
  CPP
GRKKRRQRRRPWQ

SEQ ID NO: 460 - protein, 18 aa, Artificial Sequence
  CPP
GRKLKKKKNEKEDKRPRT

SEQ ID NO: 461 - protein, 8 aa, Artificial Sequence
  CPP
GRKRKKRT

SEQ ID NO: 462 - protein, 17 aa, Artificial Sequence
  CPP
GRPRESGKKRKRKRLKP

SEQ ID NO: 463 - protein, 16 aa, Artificial Sequence
  CPP
GRQLRIAGKRLEGRSK

SEQ ID NO: 464 - protein, 16 aa, Artificial Sequence
  CPP
GRQLRIAGKRLRGRSK

SEQ ID NO: 465 - protein, 16 aa, Artificial Sequence
  CPP
GRQLRIAGRRLRGRSR

SEQ ID NO: 466 - protein, 16 aa, Artificial Sequence
  CPP
GRQLRIAGRRLRRRSR

SEQ ID NO: 467 - protein, 16 aa, Artificial Sequence
  CPP
GRQLRRAGRRLRGRSR

SEQ ID NO: 468 - protein, 16 aa, Artificial Sequence
  CPP
GRQLRRAGRRLRRRSR

SEQ ID NO: 469 - protein, 18 aa, Artificial Sequence
  CPP
GRRERNKMAAAKCRNRRR

SEQ ID NO: 470 - protein, 16 aa, Artificial Sequence
  CPP
GRRHHCRSKAKRSRHH

SEQ ID NO: 471 - protein, 14 aa, Artificial Sequence
  CPP
GRRRRATAKYRTAH

SEQ ID NO: 472 - protein, 12 aa, Artificial Sequence
  CPP
GRRRRKRLSHRT

SEQ ID NO: 473 - protein, 10 aa, Artificial Sequence
  CPP
GRRRRRERNK

SEQ ID NO: 474 - protein, 10 aa, Artificial Sequence
  CPP
GRRRRRRRRR

SEQ ID NO: 475 - protein, 13 aa, Artificial Sequence
  CPP
GRRRRRRRRRPPQ

SEQ ID NO: 476 - protein, 14 aa, Artificial Sequence
  CPP
  misc_feature (3)..(3): Xaa can be any naturally occurring amino acid
  misc_feature (11)..(11): Xaa can be any naturally occurring amino acid
GRXTKSIPPIXFPD

SEQ ID NO: 477 - protein, 14 aa, Artificial Sequence
  CPP
GSGKKGGKKHCQKY

SEQ ID NO: 478 - protein, 14 aa, Artificial Sequence
  CPP
GSGKKGGKKICQKY

SEQ ID NO: 479 - protein, 13 aa, Artificial Sequence
  CPP
GSPWGLQHHPPRT

SEQ ID NO: 480 - protein, 12 aa, Artificial Sequence
  CPP
GSRHPSLIIPRQ

SEQ ID NO: 481 - protein, 15 aa, Artificial Sequence
  CPP
GSRVQIRCRFRNSTR

SEQ ID NO: 482 - protein, 16 aa, Artificial Sequence
  CPP
GSVSRRRRRRGGRRRR

SEQ ID NO: 483 - protein, 25 aa, Artificial Sequence
  CPP
GTKMIFVGIKKKEERADLIAYLKKA

SEQ ID NO: 484 - protein, 13 aa, Artificial Sequence
  CPP
GWFDVVKHIASAV

SEQ ID NO: 485 - protein, 27 aa, Artificial Sequence
  CPP
GWTLNPAGYLLGKINLKALAALAKKIL

SEQ ID NO: 486 - protein, 27 aa, Artificial Sequence
  CPP
GWTLNPPGYLLGKINLKALAALAKKIL

SEQ ID NO: 487 - protein, 26 aa, Artificial Sequence
  CPP
GWTLNSAGYLLGKFLPLILRKIVTAL

SEQ ID NO: 488 - protein, 27 aa, Artificial Sequence
  CPP
GWTLNSAGYLLGKINLKALAALAKKIL

SEQ ID NO: 489 - protein, 27 aa, Artificial Sequence
  CPP
GWTLNSAGYLLGKINLKALAALAKKLL

SEQ ID NO: 490 - protein, 27 aa, Artificial Sequence
  CPP
GWTLNSAGYLLGKINLKAPAALAKKIL

SEQ ID NO: 491 - protein, 25 aa, Artificial Sequence
  CPP
GWTLNSAGYLLGKLKALAALAKKIL

SEQ ID NO: 492 - protein, 30 aa, Artificial Sequence
  CPP
GWTLNSAGYLLGPHAVGNHRSFSDKNGLTS

SEQ ID NO: 493 - protein, 21 aa, Artificial Sequence
  CPP
GWTLNSKINLKALAALAKKIL

SEQ ID NO: 494 - protein, 15 aa, Artificial Sequence
  CPP
GYGNCRHFKQKPRRD

SEQ ID NO: 495 - protein, 23 aa, Artificial Sequence
  CPP
GYGRKKRRGRRRTHRLPRRRRRR

SEQ ID NO: 496 - protein, 13 aa, Artificial Sequence
  CPP
GYGRKKRRQRRRG

SEQ ID NO: 497 - protein, 38 aa, Artificial Sequence
  CPP
GYGYGYGYGYGYGYGYKKRKKRKKRKKRKQQKQQKRRK

SEQ ID NO: 498 - protein, 28 aa, Artificial Sequence
  CPP
HALAHKLKHLLHRLRHLLHRHLRHALAH

SEQ ID NO: 499 - protein, 20 aa, Artificial Sequence
  CPP
HARIKPTFRRLKWKYKGKFW

SEQ ID NO: 500 - protein, 10 aa, Artificial Sequence
  CPP
HATKSQNINF

SEQ ID NO: 501 - protein, 10 aa, Artificial Sequence
  CPP
HEHEHEHEHE

SEQ ID NO: 502 - protein, 37 aa, Artificial Sequence
  CPP
HEHEHEHEHEHEHEHEEFGGGGGYGRGRGRGRGRGRG

SEQ ID NO: 503 - protein, 37 aa, Artificial Sequence
  CPP
HEHEHEHEHEHEHEHEEFGGGGGYGRRRRRRGGGGGG

SEQ ID NO: 504 - protein, 41 aa, Artificial Sequence
  CPP
HEHEHEHEHEHEHEHEHEHEEFGGGGGYGRGRGRGRGRGRG

SEQ ID NO: 505 - protein, 41 aa, Artificial Sequence
  CPP
HEHEHEHEHEHEHEHEHEHEEFGGGGGYGRRRRRRGGGGGG

SEQ ID NO: 506 - protein, 43 aa, Artificial Sequence
  CPP
HEHEHEHEHEHEHEHEHEHEGGGGGKLALKLALKALKAALKLA

SEQ ID NO: 507 - protein, 45 aa, Artificial Sequence
  CPP
HEHEHEHEHEHEHEHEHEHEHEHEEFGGGGGYGRGRGRGRGRGRG

SEQ ID NO: 508 - protein, 42 aa, Artificial Sequence
  CPP
HEHEHEHEHEHEHEHEHEHEHEHEEFGGGGGYGRKKRRQRRR

SEQ ID NO: 509 - protein, 45 aa, Artificial Sequence
  CPP
HEHEHEHEHEHEHEHEHEHEHEHEEFGGGGGYGRRRRRRGGGGGG

SEQ ID NO: 510 - protein, 12 aa, Artificial Sequence
  CPP
HFAAWGGWSLVH

SEQ ID NO: 511 - protein, 13 aa, Artificial Sequence
  CPP
HGVSGHGQHGVHG

SEQ ID NO: 512 - protein, 12 aa, Artificial Sequence
  CPP
HGWZIHGLLHRA

SEQ ID NO: 513 - protein, 26 aa, Artificial Sequence
  CPP
HHHHHHESGGGGSPGRRRRRRRRRRR

SEQ ID NO: 514 - protein, 35 aa, Artificial Sequence
  CPP
HHHHHHHHHHHHHHHHHHHHRRRRRRRRRRRRRRR

SEQ ID NO: 515 - protein, 31 aa, Artificial Sequence
  CPP
HHHHHHHHHHHHHHHHRRRRRRRRRRRRRRR

SEQ ID NO: 516 - protein, 27 aa, Artificial Sequence
  CPP
HHHHHHHHHHHHRRRRRRRRRRRRRRR

SEQ ID NO: 517 - protein, 16 aa, Artificial Sequence
  CPP
HHHHHHHHRRRRRRRR

SEQ ID NO: 518 - protein, 23 aa, Artificial Sequence
  CPP
HHHHHHHHRRRRRRRRRRRRRRR

SEQ ID NO: 519 - protein, 15 aa, Artificial Sequence
  CPP
HHHHHHRRRRRRRRR

SEQ ID NO: 520 - protein, 28 aa, Artificial Sequence
  CPP
HHHHHHTKRRITPKDVIDVRSVTTEINT

SEQ ID NO: 521 - protein, 11 aa, Artificial Sequence
  CPP
HHHRRRRRRRR

SEQ ID NO: 522 - protein, 15 aa, Artificial Sequence
  CPP
HHHRRRRRRRRRHHH

SEQ ID NO: 523 - protein, 14 aa, Artificial Sequence
  CPP
HILPWKWPWWPWRR

SEQ ID NO: 524 - protein, 12 aa, Artificial Sequence
  CPP
HIQLSPFSQSWR

SEQ ID NO: 525 - protein, 12 aa, Artificial Sequence
  CPP
HPGSPFPPEHRP

SEQ ID NO: 526 - protein, 12 aa, Artificial Sequence
  CPP
HQHKPPPLTNNW

SEQ ID NO: 527 - protein, 12 aa, Artificial Sequence
  CPP
HRHIRRQSLIML

SEQ ID NO: 528 - protein, 27 aa, Artificial Sequence
  CPP
HRLRHALAHLLHKLKHLLHALAHRLRH

SEQ ID NO: 529 - protein, 39 aa, Artificial Sequence
  CPP
HSDAVFTDNYTALRKQMAVKKYLNSILNYGRKKRRQRRR

SEQ ID NO: 530 - protein, 38 aa, Artificial Sequence
  CPP
HSDGIFTDSYSRYRKQMAVKKYLAAVLGKRYKQRVKNK

SEQ ID NO: 531 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
HXRHXRILFQYHXRHXR

SEQ ID NO: 532 - protein, 20 aa, Artificial Sequence
  CPP
HYRIKPTARRLKWKYKGKFW

SEQ ID NO: 533 - protein, 20 aa, Artificial Sequence
  CPP
HYRIKPTFRRLAWKYKGKFW

SEQ ID NO: 534 - protein, 20 aa, Artificial Sequence
  CPP
HYRIKPTFRRLKWKYKGKFA

SEQ ID NO: 535 - protein, 21 aa, Artificial Sequence
  CPP
AGYLLGKINLKALAALAKKIL

SEQ ID NO: 536 - protein, 21 aa, Artificial Sequence
  CPP
AGYLLGKINLKALAALAKKIL

SEQ ID NO: 537 - protein, 17 aa, Artificial Sequence
  CPP
IAWVKAFIRKLRKGPLG

SEQ ID NO: 538 - protein, 4 aa, Artificial Sequence
  CPP
ICIR

SEQ ID NO: 539 - protein, 5 aa, Artificial Sequence
  CPP
IGCRH

SEQ ID NO: 540 - protein, 4 aa, Artificial Sequence
  CPP
IIIR

SEQ ID NO: 541 - protein, 9 aa, Artificial Sequence
  CPP
IIYRDLISH

SEQ ID NO: 542 - protein, 38 aa, Artificial Sequence
  CPP
IKIKIKIKIKIKIKIKKLAKLAKLAKLAKLAKLAKKIK

SEQ ID NO: 543 - protein, 14 aa, Artificial Sequence
  CPP
IKIWFQNRRMKWKK

SEQ ID NO: 544 - protein, 14 aa, Artificial Sequence
  CPP
INLKALAALAKKIL

SEQ ID NO: 545 - protein, 13 aa, Artificial Sequence
  CPP
INLKKLAKLKKIL

SEQ ID NO: 546 - protein, 5 aa, Artificial Sequence
  CPP
IPALK

SEQ ID NO: 547 - protein, 8 aa, Artificial Sequence
  CPP
IPLVVPLC

SEQ ID NO: 548 - protein, 16 aa, Artificial Sequence
  CPP
IPLVVPLRRRRRRRRC

SEQ ID NO: 549 - protein, 5 aa, Artificial Sequence
  CPP
IPMIK

SEQ ID NO: 550 - protein, 5 aa, Artificial Sequence
  CPP
IPMLK

SEQ ID NO: 551 - protein, 15 aa, Artificial Sequence
  CPP
IPSRWKDQFWKRWHY

SEQ ID NO: 552 - protein, 7 aa, Artificial Sequence
  CPP
IRQRRRR

SEQ ID NO: 553 - protein, 11 aa, Artificial Sequence
  CPP
ISFELLDYYED

SEQ ID NO: 554 - protein, 13 aa, Artificial Sequence
  CPP
ISFELLDYYESGS

SEQ ID NO: 555 - protein, 12 aa, Artificial Sequence
  CPP
ISFEWLQAYEDE

SEQ ID NO: 556 - protein, 15 aa, Artificial Sequence
  CPP
ISFDELLDYYGESGS

SEQ ID NO: 557 - protein, 12 aa, Artificial Sequence
  CPP
IWFQNRRMKWKK

SEQ ID NO: 558 - protein, 10 aa, Artificial Sequence
  CPP
IWRYSLASQQ

SEQ ID NO: 559 - protein, 25 aa, Artificial Sequence
  CPP
IYLATALAKWALKQGFGGRRRRRRR

SEQ ID NO: 560 - protein, 23 aa, Artificial Sequence
  CPP
IYLATALAKWALKQGGRRRRRRR

SEQ ID NO: 561 - protein, 8 aa, Artificial Sequence
  CPP
IYRDLISH

SEQ ID NO: 562 - protein, 21 aa, Artificial Sequence
  CPP
AGYLLGKINLKALAALAKKIL

SEQ ID NO: 563 - protein, 20 aa, Artificial Sequence
  CPP
KFQWQRNMRKVRGPPVSIKR

SEQ ID NO: 564 - protein, 23 aa, Artificial Sequence
  CPP
KAFAKLAARLYRKALARQLGVAA

SEQ ID NO: 565 - protein, 18 aa, Artificial Sequence
  CPP
KALAALLKKLAKLLAALK

SEQ ID NO: 566 - protein, 18 aa, Artificial Sequence
  CPP
KALAALLKKWAKLLAALK

SEQ ID NO: 567 - protein, 18 aa, Artificial Sequence
  CPP
KALAKALAKLWKALAKAA

SEQ ID NO: 568 - protein, 18 aa, Artificial Sequence
  CPP
KALKKLLAKWLAAAKALL

SEQ ID NO: 569 - protein, 18 aa, Artificial Sequence
  CPP
KALKLKLALALLAKLKLA

SEQ ID NO: 570 - protein, 10 aa, Artificial Sequence
  CPP
KCCKWRWRCK

SEQ ID NO: 571 - protein, 22 aa, Artificial Sequence
  CPP
KCFMWQEMLNKAGVPKLRCARK

SEQ ID NO: 572 - protein, 13 aa, Artificial Sequence
  CPP
KCFQWQRNMRKVR

SEQ ID NO: 573 - protein, 19 aa, Artificial Sequence
  CPP
KCFQWQRNMRKVRGPPVSC

SEQ ID NO: 574 - protein, 22 aa, Artificial Sequence
  CPP
KCFQWQRNMRKVRGPPVSCIKR

SEQ ID NO: 575 - protein, 22 aa, Artificial Sequence
  CPP
KCFQWQRNMRKVRGPPVSSIKR

SEQ ID NO: 576 - protein, 14 aa, Artificial Sequence
  CPP
KCGCRWRWKCGCKK

SEQ ID NO: 577 - protein, 9 aa, Artificial Sequence
  CPP
KCPSRRPKR

SEQ ID NO: 578 - protein, 11 aa, Artificial Sequence
  CPP
KCRWRWKCCKK

SEQ ID NO: 579 - protein, 28 aa, Artificial Sequence
  CPP
KDCERRFSRSDQLKRHQRRHTGVKPFQK

SEQ ID NO: 580 - protein, 12 aa, Artificial Sequence
  CPP
KDCRWRWKCCKK

SEQ ID NO: 581 - protein, 21 aa, Artificial Sequence
  CPP
KETWFETWFTEWSQPKKKRKV

SEQ ID NO: 582 - protein, 28 aa, Artificial Sequence
  CPP
KETWWETWWTEWSQPGRKKRRQRRRPPQ

SEQ ID NO: 583 - protein, 21 aa, Artificial Sequence
  CPP
KETWWETWWTEWSQPKKKRKV

SEQ ID NO: 584 - protein, 22 aa, Artificial Sequence
  CPP
KETWWETWWTEWSQPKKKRKVC

SEQ ID NO: 585 - protein, 10 aa, Artificial Sequence
  CPP
KFFKFFKFFK

SEQ ID NO: 586 - protein, 15 aa, Artificial Sequence
  CPP
KFHTFPQTAIGVGAP

SEQ ID NO: 587 - protein, 19 aa, Artificial Sequence
  CPP
KFLNRFWHWLQLKPGQPMY

SEQ ID NO: 588 - protein, 9 aa, Artificial Sequence
  CPP
KGKKIFIMK

SEQ ID NO: 589 - protein, 14 aa, Artificial Sequence
  CPP
KGRKKRRQRRRPPQ

SEQ ID NO: 590 - protein, 26 aa, Artificial Sequence
  CPP
KGRTPIKFGKADCDRPPKHSQNGMGK

SEQ ID NO: 591 - protein, 36 aa, Artificial Sequence
  CPP
KGSKKAVTKAQKKDGKKRKRSRKESYSVYVYKVLKQ

SEQ ID NO: 592 - protein, 26 aa, Artificial Sequence
  CPP
KHHWHHVRLPPPVRLPPPGNHHHHHH

SEQ ID NO: 593 - protein, 25 aa, Artificial Sequence
  CPP
KHKALHALHLLALLWLHLAHLAKHK

SEQ ID NO: 594 - protein, 29 aa, Artificial Sequence
  CPP
KHKHKHKHKHKHKHKHKHKKLFKKILKYL

SEQ ID NO: 595 - protein, 25 aa, Artificial Sequence
  CPP
KHKLLHLLHLLALLWLHLLHLLKHK

SEQ ID NO: 596 - protein, 18 aa, Artificial Sequence
  CPP
KIAAKSIAKIWKSILKIA

SEQ ID NO: 597 - protein, 20 aa, Artificial Sequence
  CPP
KIAKLKAKIQKLKQKIAKLK

SEQ ID NO: 598 - protein, 18 aa, Artificial Sequence
  CPP
KITLKLAIKAWKLALKAA

SEQ ID NO: 599 - protein, 13 aa, Artificial Sequence
  CPP
KIWFQNRRMKWKK

SEQ ID NO: 600 - protein, 17 aa, Artificial Sequence
  CPP
KKAAQIRSQVMTHLRVI

SEQ ID NO: 601 - protein, 26 aa, Artificial Sequence
  CPP
KKALLAHALHLLALLALHLAHALKKA

SEQ ID NO: 602 - protein, 26 aa, Artificial Sequence
  CPP
KKALLALALHHLAHLALHLALALKKA

SEQ ID NO: 603 - protein, 25 aa, Artificial Sequence
  CPP
KKDGKKRKRSRKESYSVYVYKVLKQ

SEQ ID NO: 604 - protein, 16 aa, Artificial Sequence
  CPP
KKICTRKPRFMSAWAQ

SEQ ID NO: 605 - protein, 16 aa, Artificial Sequence
  CPP
KKKEERADLIAYLKKA

SEQ ID NO: 606 - protein, 34 aa, Artificial Sequence
  CPP
KKKKKKGGFLGFWRGENGRKTRSAYERMCILKGK

SEQ ID NO: 607 - protein, 8 aa, Artificial Sequence
  CPP
KKKKKKKK

SEQ ID NO: 608 - protein, 9 aa, Artificial Sequence
  CPP
KKKKKKKKK

SEQ ID NO: 609 - protein, 19 aa, Artificial Sequence
  CPP
KKKKKKKKKKKKKKKKKKK

SEQ ID NO: 610 - protein, 15 aa, Artificial Sequence
  CPP
KKKKKKNKKLQQRGD

SEQ ID NO: 611 - protein, 25 aa, Artificial Sequence
  CPP
KKLALHALHLLALLWLHLAHLALKK

SEQ ID NO: 612 - protein, 11 aa, Artificial Sequence
  CPP
KKLFKKILKKL

SEQ ID NO: 613 - protein, 14 aa, Artificial Sequence
  CPP
KKPGKKTTTKPTKK

SEQ ID NO: 614 - protein, 22 aa, Artificial Sequence
  CPP
KKPGKKTTTKPTKKPTIKTTKK

SEQ ID NO: 615 - protein, 10 aa, Artificial Sequence
  CPP
KKPTIKTTKK

SEQ ID NO: 616 - protein, 8 aa, Artificial Sequence
  CPP
KKRRQRRR

SEQ ID NO: 617 - protein, 10 aa, Artificial Sequence
  CPP
KKTTTKPTKK

SEQ ID NO: 618 - protein, 33 aa, Artificial Sequence
  CPP
KKWALLALALHHLAHLALHLALALKKAHHHHHH

SEQ ID NO: 619 - protein, 17 aa, Artificial Sequence
  CPP
  misc_feature (13)..(13): Xaa can be any naturally occurring amino acid
  misc_feature (16)..(16): Xaa can be any naturally occurring amino acid
KKWKMRILFQYRXRRXR

SEQ ID NO: 620 - protein, 19 aa, Artificial Sequence
  CPP
KKWKMRRGAGRRRRRRRRR

SEQ ID NO: 621 - protein, 16 aa, Artificial Sequence
  CPP
KKWKMRRNQFWIKIQR

SEQ ID NO: 622 - protein, 18 aa, Artificial Sequence
  CPP
KLAAALLKKWKKLAAALL

SEQ ID NO: 623 - protein, 14 aa, Artificial Sequence
  CPP
KLAKLAKKLAKLAK

SEQ ID NO: 624 - protein, 23 aa, Artificial Sequence
  CPP
KLAKLAKKLAKLAKGGRRRRRRR

SEQ ID NO: 625 - protein, 25 aa, Artificial Sequence
  CPP
KLAKLAKKLAKLAKGRKKRRQRRRP

SEQ ID NO: 626 - protein, 24 aa, Artificial Sequence
  CPP
KLAKLAKKLAKLAKNYRWRCKNQN

SEQ ID NO: 627 - protein, 18 aa, Artificial Sequence
  CPP
KLALKAAAKAWKAAAKAA

SEQ ID NO: 628 - protein, 18 aa, Artificial Sequence
  CPP
KLALKAALKAWKAAAKLA

SEQ ID NO: 629 - protein, 14 aa, Artificial Sequence
  CPP
KLALKALKAALKLA

SEQ ID NO: 630 - protein, 14 aa, Artificial Sequence
  CPP
KLALKLALKALKAA

SEQ ID NO: 631 - protein, 16 aa, Artificial Sequence
  CPP
KLALKLALKALKAALK

SEQ ID NO: 632 - protein, 18 aa, Artificial Sequence
  CPP
KLALKLALKALKAALKLA

SEQ ID NO: 633 - protein, 20 aa, Artificial Sequence
  CPP
KLALKLALKALKAALKLAGC

SEQ ID NO: 634 - protein, 18 aa, Artificial Sequence
  CPP
KLALKLALKALQAALQLA

SEQ ID NO: 635 - protein, 18 aa, Artificial Sequence
  CPP
KLALKLALKAWKAALKLA

SEQ ID NO: 636 - protein, 18 aa, Artificial Sequence
  CPP
KLALKLALKWAKLALKAA

SEQ ID NO: 637 - protein, 18 aa, Artificial Sequence
  CPP
KLALQLALQALQAALQLA

SEQ ID NO: 638 - protein, 28 aa, Artificial Sequence
  CPP
KLFMALVAFLRFLTIPPTAGILKRWGTI

SEQ ID NO: 639 - protein, 18 aa, Artificial Sequence
  CPP
KLGLKLGLKGLKGGLKLG

SEQ ID NO: 640 - protein, 5 aa, Artificial Sequence
  CPP
KLGVM

SEQ ID NO: 641 - protein, 13 aa, Artificial Sequence
  CPP
KLIKGRTPIKFGK

SEQ ID NO: 642 - protein, 25 aa, Artificial Sequence
  CPP
KLIKGRTPIKFGKADCDRPPKHSGK

SEQ ID NO: 643 - protein, 27 aa, Artificial Sequence
  CPP
KLIKGRTPIKFGKADCDRPPKHSQNGK

SEQ ID NO: 644 - protein, 27 aa, Artificial Sequence
  CPP
KLIKGRTPIKFGKADCDRPPKHSQNGM

SEQ ID NO: 645 - protein, 29 aa, Artificial Sequence
  CPP
KLIKGRTPIKFGKADCDRPPKHSQNGMGK

SEQ ID NO: 646 - protein, 25 aa, Artificial Sequence
  CPP
KLIKGRTPIKFGKARCRRPPKHSGK

SEQ ID NO: 647 - protein, 18 aa, Artificial Sequence
  CPP
KLLAKAAKKWLLLALKAA

SEQ ID NO: 648 - protein, 18 aa, Artificial Sequence
  CPP
KLLAKAALKWLLKALKAA

SEQ ID NO: 649 - protein, 18 aa, Artificial Sequence
  CPP
KLLKLLLKLWKKLLKLLK

SEQ ID NO: 650 - protein, 28 aa, Artificial Sequence
  CPP
KLLKLLLKLWKKLLKLLKGGGRRRRRRR

SEQ ID NO: 651 - protein, 19 aa, Artificial Sequence
  CPP
KLPCRSNTFLNIFRRKKPG

SEQ ID NO: 652 - protein, 5 aa, Artificial Sequence
  CPP
KLPVM

SEQ ID NO: 653 - protein, 5 aa, Artificial Sequence
  CPP
KLPVT

SEQ ID NO: 654 - protein, 21 aa, Artificial Sequence
  CPP
KLTRAQRRAAARKNKRNTRGC

SEQ ID NO: 655 - protein, 18 aa, Artificial Sequence
  CPP
KLALKLALKALKAALKLA

SEQ ID NO: 656 - protein, 20 aa, Artificial Sequence
  CPP
KLALKLALKALKAALKLAGC

SEQ ID NO: 657 - protein, 15 aa, Artificial Sequence
  CPP
KLWMRWWSPTTRRYG

SEQ ID NO: 658 - protein, 15 aa, Artificial Sequence
  CPP
KLWMRWYSATTRRYG

SEQ ID NO: 659 - protein, 15 aa, Artificial Sequence
  CPP
KLWMRWYSPTTRRYG

SEQ ID NO: 660 - protein, 15 aa, Artificial Sequence
  CPP
KLWMRWYSPWTRRYG

SEQ ID NO: 661 - protein, 16 aa, Artificial Sequence
  CPP
KLWSAWPSLWSSLWKP

SEQ ID NO: 662 - protein, 13 aa, Artificial Sequence
  CPP
KMDCRPRPKCCKK

SEQ ID NO: 663 - protein, 13 aa, Artificial Sequence
  CPP
  misc_feature (11)..(11): Xaa can be any naturally occurring amino acid
KMDCRPRPKCXKK

SEQ ID NO: 664 - protein, 13 aa, Artificial Sequence
  CPP
  misc_feature (10)..(10): Xaa can be any naturally occurring amino acid
KMDCRPRPKXCKK

SEQ ID NO: 665 - protein, 13 aa, Artificial Sequence
  CPP
KMDCRWRPKCCKK

SEQ ID NO: 666 - protein, 13 aa, Artificial Sequence
  CPP
KMDCRWRWKCCKK

SEQ ID NO: 667 - protein, 12 aa, Artificial Sequence
  CPP
KMDCRWRWKCKK

SEQ ID NO: 668 - protein, 13 aa, Artificial Sequence
  CPP
KMDCRWRWKCSKK

SEQ ID NO: 669 - protein, 11 aa, Artificial Sequence
  CPP
KMDCRWRWKKK

SEQ ID NO: 670 - protein, 13 aa, Artificial Sequence
  CPP
KMDCRWRWKSCKK

SEQ ID NO: 671 - protein, 13 aa, Artificial Sequence
  CPP
KMDCRWRWKSSKK

SEQ ID NO: 672 - protein, 10 aa, Artificial Sequence
  CPP
KMDRWRWKKK

SEQ ID NO: 673 - protein, 13 aa, Artificial Sequence
  CPP
KMDSRWRWKCCKK

SEQ ID NO: 674 - protein, 13 aa, Artificial Sequence
  CPP
KMDSRWRWKCSKK

SEQ ID NO: 675 - protein, 13 aa, Artificial Sequence
  CPP
KMDSRWRWKSCKK

SEQ ID NO: 676 - protein, 13 aa, Artificial Sequence
  CPP
KMDSRWRWKSSKK

SEQ ID NO: 677 - protein, 13 aa, Artificial Sequence
  CPP
  misc_feature (4)..(4): Xaa can be any naturally occurring amino acid
KMDXRPRPKCCKK

SEQ ID NO: 678 - protein, 13 aa, Artificial Sequence
  CPP
  misc_feature (4)..(4): Xaa can be any naturally occurring amino acid
  misc_feature (11)..(11): Xaa can be any naturally occurring amino acid
KMDXRPRPKCXKK

SEQ ID NO: 679 - protein, 13 aa, Artificial Sequence
  CPP
  misc_feature (4)..(4): Xaa can be any naturally occurring amino acid
  misc_feature (10)..(10): Xaa can be any naturally occurring amino acid
KMDXRPRPKXCKK

SEQ ID NO: 680 - protein, 13 aa, Artificial Sequence
  CPP
  misc_feature (4)..(4): Xaa can be any naturally occurring amino acid
KMDXRWRWKCCKK

SEQ ID NO: 681 - protein, 13 aa, Artificial Sequence
  CPP
  misc_feature (4)..(4): Xaa can be any naturally occurring amino acid
  misc_feature (11)..(11): Xaa can be any naturally occurring amino acid
KMDXRWRWKCXKK

SEQ ID NO: 682 - protein, 13 aa, Artificial Sequence
  CPP
  misc_feature (4)..(4): Xaa can be any naturally occurring amino acid
  misc_feature (10)..(10): Xaa can be any naturally occurring amino acid
KMDXRWRWKXCKK

SEQ ID NO: 683 - protein, 13 aa, Artificial Sequence
  CPP
  misc_feature (4)..(4): Xaa can be any naturally occurring amino acid
  misc_feature (10)..(11): Xaa can be any naturally occurring amino acid
KMDXRWRWKXXKK

SEQ ID NO: 684 - protein, 10 aa, Artificial Sequence
  CPP
KMIFVGIKKK

SEQ ID NO: 685 - protein, 14 aa, Artificial Sequence
  CPP
KMIFVGIKKKEERA

SEQ ID NO: 686 - protein, 21 aa, Artificial Sequence
  CPP
KMTRAQRRAAARRNRWTARGC

SEQ ID NO: 687 - protein, 15 aa, Artificial Sequence
  CPP
KNAWKHSSCHHRHQI

SEQ ID NO: 688 - protein, 12 aa, Artificial Sequence
  CPP
KPRSKNPPKKPK

SEQ ID NO: 689 - protein, 24 aa, Artificial Sequence
  CPP
KRARNTEAARRSRARKLQRMKQGC

SEQ ID NO: 690 - protein, 12 aa, Artificial Sequence
  CPP
KRIHPRLTRSIR

SEQ ID NO: 691 - protein, 12 aa, Artificial Sequence
  CPP
KRIIQRILSRNS

SEQ ID NO: 692 - protein, 11 aa, Artificial Sequence
  CPP
KRIPNKKPGKK

SEQ ID NO: 693 - protein, 12 aa, Artificial Sequence
  CPP
KRIPNKKPGKKT

SEQ ID NO: 694 - protein, 19 aa, Artificial Sequence
  CPP
KRIPNKKPGKKTTTKPTKK

SEQ ID NO: 695 - protein, 23 aa, Artificial Sequence
  CPP
KRIPNKKPGKKTTTKPTKKPTIK

SEQ ID NO: 696 - protein, 27 aa, Artificial Sequence
  CPP
KRIPNKKPGKKTTTKPTKKPTIKTTKK

SEQ ID NO: 697 - protein, 30 aa, Artificial Sequence
  CPP
KRIPNKKPGKKTTTKPTKKPTIKTTKKDLK

SEQ ID NO: 698 - protein, 37 aa, Artificial Sequence
  CPP
KRIPNKKPGKKTTTKPTKKPTIKTTKKDLKPQTTKPK

SEQ ID NO: 699 - protein, 10 aa, Artificial Sequence
  CPP
KRIPNKKPKK

SEQ ID NO: 700 - protein, 7 aa, Artificial Sequence
  CPP
KRKRWHW

SEQ ID NO: 701 - protein, 16 aa, Artificial Sequence
  CPP
KRPAAIKKAGQAKKKK

SEQ ID NO: 702 - protein, 15 aa, Artificial Sequence
  CPP
KRPTMRFRYTWNPMK

SEQ ID NO: 703 - protein, 28 aa, Artificial Sequence
  CPP
KRRIRRERNKMAAAKSRNRRRELTDTGC

SEQ ID NO: 704 - protein, 7 aa, Artificial Sequence
  CPP
KRRQRRR

SEQ ID NO: 705 - protein, 13 aa, Artificial Sequence
  CPP
KRVSRNKSEKKRR

SEQ ID NO: 706 - protein, 10 aa, Artificial Sequence
  CPP
KRWRWKCCKK

SEQ ID NO: 707 - protein, 18 aa, Artificial Sequence
  CPP
KSHAHAQKRIRRRLIILL

SEQ ID NO: 708 - protein, 15 aa, Artificial Sequence
  CPP
KSICKTIPSNKPKKK

SEQ ID NO: 709 - protein, 20 aa, Artificial Sequence
  CPP
KSTGKANKITITNDKGRLSK

SEQ ID NO: 710 - protein, 10 aa, Artificial Sequence
  CPP
KTCENLADTY

SEQ ID NO: 711 - protein, 12 aa, Artificial Sequence
  CPP
KTIEAHPPYYAS

SEQ ID NO: 712 - protein, 11 aa, Artificial Sequence
  CPP
KTIPSNKPKKK

SEQ ID NO: 713 - protein, 16 aa, Artificial Sequence
  CPP
KTVLLRKLLKLLVRKI

SEQ ID NO: 714 - protein, 19 aa, Artificial Sequence
  CPP
KWCFAVCYAGICYAACAGK

SEQ ID NO: 715 - protein, 19 aa, Artificial Sequence
  CPP
KWCFRVCYRGICYRRCRGK

SEQ ID NO: 716 - protein, 15 aa, Artificial Sequence
  CPP
KWFETWFTEWPKKRK

SEQ ID NO: 717 - protein, 18 aa, Artificial Sequence
  CPP
KWFETWFTEWPKKRKGGC

SEQ ID NO: 718 - protein, 16 aa, Artificial Sequence
  CPP
KWFKIQMQIRRWKNKR

SEQ ID NO: 719 - protein, 15 aa, Artificial Sequence
  CPP
KWFRVYRGIYRRRGK

SEQ ID NO: 720 - protein, 19 aa, Artificial Sequence
  CPP
KWSFRVSYRGISYRRSRGK

SEQ ID NO: 721 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
KXRKXRILFQYKXRKXR

SEQ ID NO: 722 - protein, 25 aa, Artificial Sequence
  CPP
LAELLAELLAELGGGGRRRRRRRRR

SEQ ID NO: 723 - protein, 18 aa, Artificial Sequence
  CPP
LAIILRRRIRKQAHAHSK

SEQ ID NO: 724 - protein, 36 aa, Artificial Sequence
  CPP
LALALALALALALAKLAKLAKLAKLAKIKKIKKKIK

SEQ ID NO: 725 - protein, 38 aa, Artificial Sequence
  CPP
LALALALALALALALAKIKKIKKIKKIKKLAKLAKKIK

SEQ ID NO: 726 - protein, 38 aa, Artificial Sequence
  CPP
LALALALALALALALAKKLKKLKKLKKLKKLKKLKYAK

SEQ ID NO: 727 - protein, 35 aa, Artificial Sequence
  CPP
LALALALALALALALAKLAKLAKLAKLAKLAKKIK

SEQ ID NO: 728 - protein, 25 aa, Artificial Sequence
  CPP
LAQLLAQLLAQLGGGGRRRRRRRRR

SEQ ID NO: 729 - protein, 9 aa, Artificial Sequence
  CPP
RRRRRRRRR

SEQ ID NO: 730 - protein, 9 aa, Artificial Sequence
  CPP
RRRRRRRRR

SEQ ID NO: 731 - protein, 7 aa, Artificial Sequence
  CPP
RRRRRRR

SEQ ID NO: 732 - protein, 9 aa, Artificial Sequence
  CPP
RRRRRRRRR

SEQ ID NO: 733 - protein, 3 aa, Artificial Sequence
  CPP
LCL

SEQ ID NO: 734 - protein, 4 aa, Artificial Sequence
  CPP
LCLE

SEQ ID NO: 735 - protein, 4 aa, Artificial Sequence
  CPP
LCLH

SEQ ID NO: 736 - protein, 4 aa, Artificial Sequence
  CPP
LCLK

SEQ ID NO: 737 - protein, 4 aa, Artificial Sequence
  CPP
LCLN

SEQ ID NO: 738 - protein, 4 aa, Artificial Sequence
  CPP
LCLQ

SEQ ID NO: 739 - protein, 4 aa, Artificial Sequence
  CPP
LCLR

SEQ ID NO: 740 - protein, 5 aa, Artificial Sequence
  CPP
LCLRP

SEQ ID NO: 741 - protein, 7 aa, Artificial Sequence
  CPP
LCLRPVG

SEQ ID NO: 742 - protein, 12 aa, Artificial Sequence
  CPP
LDITPFLSLTLP

SEQ ID NO: 743 - protein, 27 aa, Artificial Sequence
  CPP
LDTYSPELFCTIRNFYDADRPDRGAAA

SEQ ID NO: 744 - protein, 12 aa, Artificial Sequence
  CPP
LFDIIKKIAESF

SEQ ID NO: 745 - protein, 25 aa, Artificial Sequence
  CPP
LFDIIKKIAESGFLFDIIKKIAESF

SEQ ID NO: 746 - protein, 18 aa, Artificial Sequence
  CPP
LGISYGRKKRRQRRRPPQ

SEQ ID NO: 747 - protein, 18 aa, Artificial Sequence
  CPP
LGLLLRHLRHHSNLLANI

SEQ ID NO: 748 - protein, 24 aa, Artificial Sequence
  CPP
LGTYTQDFNKFHTFPQTAIGVGAP

SEQ ID NO: 749 - protein, 22 aa, Artificial Sequence
  CPP
LHHLLHHLLHLLHHLLHHLHHL

SEQ ID NO: 750 - protein, 12 aa, Artificial Sequence
  CPP
LIIFAIAASHKK

SEQ ID NO: 751 - protein, 12 aa, Artificial Sequence
  CPP
LIIFAILISHKK

SEQ ID NO: 752 - protein, 12 aa, Artificial Sequence
  CPP
LIIFRIAASHKK

SEQ ID NO: 753 - protein, 10 aa, Artificial Sequence
  CPP
LIIFRILISH

SEQ ID NO: 754 - protein, 12 aa, Artificial Sequence
  CPP
LIIFRILISHHH

SEQ ID NO: 755 - protein, 11 aa, Artificial Sequence
  CPP
LIIFRILISHK

SEQ ID NO: 756 - protein, 12 aa, Artificial Sequence
  CPP
LIIFRILISHKK

SEQ ID NO: 757 - protein, 11 aa, Artificial Sequence
  CPP
LIIFRILISHR

SEQ ID NO: 758 - protein, 12 aa, Artificial Sequence
  CPP
LIIFRILISHRR

SEQ ID NO: 759 - protein, 13 aa, Artificial Sequence
  CPP
LIKKLKALKKLNI

SEQ ID NO: 760 - protein, 14 aa, Artificial Sequence
  CPP
LIKKALAALAKLNI

SEQ ID NO: 761 - protein, 15 aa, Artificial Sequence
  CPP
LILIGRRRRRRRRGC

SEQ ID NO: 762 - protein, 38 aa, Artificial Sequence
  CPP
LILILILILILILILIKRKKRKKRKKRKKRAKRAKHSK

SEQ ID NO: 763 - protein, 23 aa, Artificial Sequence
  CPP
LIRLWSHLIHIWFQNRRLKWKKK

SEQ ID NO: 764 - protein, 24 aa, Artificial Sequence
  CPP
LIRLWSHLIHIWFQNRRLKWKKKC

SEQ ID NO: 765 - protein, 26 aa, Artificial Sequence
  CPP
LIRLWSHLIHIWFQNRRLKWKKKGGC

SEQ ID NO: 766 - protein, 15 aa, Artificial Sequence
  CPP
LKKLAELAHKLLKLG

SEQ ID NO: 767 - protein, 16 aa, Artificial Sequence
  CPP
LKKLCKLLKKLCKLAG

SEQ ID NO: 768 - protein, 16 aa, Artificial Sequence
  CPP
LKKLLKLLKKLLKLAG

SEQ ID NO: 769 - protein, 15 aa, Artificial Sequence
  CPP
LKLLKKLLKKLLKLL

SEQ ID NO: 770 - protein, 35 aa, Artificial Sequence
  CPP
LKRWGTIKKSKAINVLRGFRKEIGRMLNILNRRRR

SEQ ID NO: 771 - protein, 18 aa, Artificial Sequence
  CPP
LKTLATALTKLAKTLTTL

SEQ ID NO: 772 - protein, 18 aa, Artificial Sequence
  CPP
LKTLTETLKELTKTLTEL

SEQ ID NO: 773 - protein, 18 aa, Artificial Sequence
  CPP
LLAILRRRIRKQAHAHSK

SEQ ID NO: 774 - protein, 33 aa, Artificial Sequence
  CPP
LLETLLKPFQCRICMRNFSTRQARRNHRRRHRR

SEQ ID NO: 775 - protein, 38 aa, Artificial Sequence
  CPP
LLGDFFRKSKEKIGKEFKRIVQRIKDFLRNLVPRTESC

SEQ ID NO: 776 - protein, 18 aa, Artificial Sequence
  CPP
LLGKINLKALAALAKKIL

SEQ ID NO: 777 - protein, 18 aa, Artificial Sequence
  CPP
LLHILRRSIRKQAHAIRK

SEQ ID NO: 778 - protein, 18 aa, Artificial Sequence
  CPP
LLHILRRSIRRQAHAIRR

SEQ ID NO: 779 - protein, 18 aa, Artificial Sequence
  CPP
LLIALRRRIRKQAHAHSK

SEQ ID NO: 780 - protein, 18 aa, Artificial Sequence
  CPP
LLIIARRRIRKQAHAHSK

SEQ ID NO: 781 - protein, 18 aa, Artificial Sequence
  CPP
LLIILARRIRKQAHAHSK

SEQ ID NO: 782 - protein, 18 aa, Artificial Sequence
  CPP
LLIILRARIRKQAHAHSK

SEQ ID NO: 783 - protein, 18 aa, Artificial Sequence
  CPP
LLIILRRAIRKQAHAHSK

SEQ ID NO: 784 - protein, 18 aa, Artificial Sequence
  CPP
LLIILRRRARKQAHAHSK

SEQ ID NO: 785 - protein, 19 aa, Artificial Sequence
  CPP
LLIILRRRIARKQAHAHSK

SEQ ID NO: 786 - protein, 18 aa, Artificial Sequence
  CPP
LLIILRRRIRAQAHAHSK

SEQ ID NO: 787 - protein, 18 aa, Artificial Sequence
  CPP
LLIILRRRIRKAAHAHSK

SEQ ID NO: 788 - protein, 18 aa, Artificial Sequence
  CPP
LLIILRRRIRKQAAAHSK

SEQ ID NO: 789 - protein, 18 aa, Artificial Sequence
  CPP
LLIILRRRIRKQAHAASK

SEQ ID NO: 790 - protein, 18 aa, Artificial Sequence
  CPP
LLIILRRRIRKQAHAHAK

SEQ ID NO: 791 - protein, 18 aa, Artificial Sequence
  CPP
LLIILRRRIRKQAHAHSA

SEQ ID NO: 792 - protein, 18 aa, Artificial Sequence
  CPP
LLIILRRRIRKQAHAHSK

SEQ ID NO: 793 - protein, 30 aa, Artificial Sequence
  CPP
LLIILRRRIRKQAHAHSKNHQQQNPHQPPM

SEQ ID NO: 794 - protein, 18 aa, Artificial Sequence
  CPP
LLIILRRRIRRRARARSR

SEQ ID NO: 795 - protein, 16 aa, Artificial Sequence
  CPP
LLKKRKVVRLIKFLLK

SEQ ID NO: 796 - protein, 19 aa, Artificial Sequence
  CPP
LLKLLKKLLKLLKKLLKLL

SEQ ID NO: 797 - protein, 18 aa, Artificial Sequence
  CPP
LLKTTALLKTTALLKTTA

SEQ ID NO: 798 - protein, 18 aa, Artificial Sequence
  CPP
LLKTTELLKTTELLKTTE

SEQ ID NO: 799 - protein, 5 aa, Artificial Sequence
  CPP
LLLLR

SEQ ID NO: 800 - protein, 4 aa, Artificial Sequence
  CPP
LLLR

SEQ ID NO: 801 - protein, 5 aa, Artificial Sequence
  CPP
LLLRR

SEQ ID NO: 802 - protein, 14 aa, Artificial Sequence
  CPP
LLKKLAAAALKKLL

SEQ ID NO: 803 - protein, 3 aa, Artificial Sequence
  CPP
LLR

SEQ ID NO: 804 - protein, 15 aa, Artificial Sequence
  CPP
LLRARWRRRRSRRFR

SEQ ID NO: 805 - protein, 18 aa, Artificial Sequence
  CPP
LLRHLRRHIRRARRHIRR

SEQ ID NO: 806 - protein, 18 aa, Artificial Sequence
  CPP
LLRILRRSIRRARRAIRR

SEQ ID NO: 807 - protein, 18 aa, Artificial Sequence
  CPP
LLYWFRRRHRHHRRRHRR

SEQ ID NO: 808 - protein, 20 aa, Artificial Sequence
  CPP
LNSAGYLLGKALAALAKKIL

SEQ ID NO: 809 - protein, 24 aa, Artificial Sequence
  CPP
LNSAGYLLGKINLKALAALAKKIL

SEQ ID NO: 810 - protein, 19 aa, Artificial Sequence
  CPP
LNSAGYLLGKLKALAALAK

SEQ ID NO: 811 - protein, 21 aa, Artificial Sequence
  CPP
LNSAGYLLGKLKALAALAKIL

SEQ ID NO: 812 - protein, 12 aa, Artificial Sequence
  CPP
LNVPPSWFLSQR

SEQ ID NO: 813 - protein, 12 aa, Artificial Sequence
  CPP
LPHPVLHMGPLR

SEQ ID NO: 814 - protein, 29 aa, Artificial Sequence
  CPP
LRHHLRHLLRHLRHLLRHLRHHLRHLLRH

SEQ ID NO: 815 - protein, 15 aa, Artificial Sequence
  CPP
LRHLLRHLLRHLRHL

SEQ ID NO: 816 - protein, 29 aa, Artificial Sequence
  CPP
LRHLLRHLLRHLRHLLRHLRHLLRHLLRH

SEQ ID NO: 817 - protein, 16 aa, Artificial Sequence
  CPP
LRRERQSRLRRERQSR

SEQ ID NO: 818 - protein, 28 aa, Artificial Sequence
  CPP
LSTAADMQGVVTDGMASGLDKDYLKPDD

SEQ ID NO: 819 - protein, 12 aa, Artificial Sequence
  CPP
LTMPSDLQPVLW

SEQ ID NO: 820 - protein, 12 aa, Artificial Sequence
  CPP
LTRNYEAWVPTP

SEQ ID NO: 821 - protein, 18 aa, Artificial Sequence
  CPP
LVVLGKLYGRKKRRQRRR

SEQ ID NO: 822 - protein, 5 aa, Artificial Sequence
  CPP
MAARL

SEQ ID NO: 823 - protein, 8 aa, Artificial Sequence
  CPP
MAARLCCQ

SEQ ID NO: 824 - protein, 15 aa, Artificial Sequence
  CPP
MAARLCCQLDPARDV

SEQ ID NO: 825 - protein, 20 aa, Artificial Sequence
  CPP
MAARLCCQLDPARDVLCLRP

SEQ ID NO: 826 - protein, 9 aa, Artificial Sequence
  CPP
MAIYRDLIS

SEQ ID NO: 827 - protein, 29 aa, Artificial Sequence
  CPP
MAMPGEPRRANVMAHKLEPASLQLRNSCA

SEQ ID NO: 828 - protein, 28 aa, Artificial Sequence
  CPP
MANLGCWMLVLFVATWSDLGLCKKRPKP

SEQ ID NO: 829 - protein, 28 aa, Artificial Sequence
  CPP
MANLGYWLLALFVTMWTDVGLCKKRPKP

SEQ ID NO: 830 - protein, 29 aa, Artificial Sequence
  CPP
MAPQRDTVGGRTTPPSWGPAKAQLRNSCA

SEQ ID NO: 831 - protein, 24 aa, Artificial Sequence
  CPP
MDAQTRRRERRAEKQAQWKAANGC

SEQ ID NO: 832 - protein, 12 aa, Artificial Sequence
  CPP
MDCRWRWKCCKK

SEQ ID NO: 833 - protein, 12 aa, Artificial Sequence
  CPP
  misc_feature (10)..(10): Xaa can be any naturally occurring amino acid
MDCRWRWKCXKK

SEQ ID NO: 834 - protein, 12 aa, Artificial Sequence
  CPP
  misc_feature (9)..(9): Xaa can be any naturally occurring amino acid
MDCRWRWKXCKK

SEQ ID NO: 835 - protein, 12 aa, Artificial Sequence
  CPP
  misc_feature (9)..(10): Xaa can be any naturally occurring amino acid
MDCRWRWKXXKK

SEQ ID NO: 836 - protein, 29 aa, Artificial Sequence
  CPP
RKKRRRESWVHLPPPVHLPPPGGHHHHHH

SEQ ID NO: 837 - protein, 23 aa, Artificial Sequence
  CPP
MGLGLHLLVLAAALQGAKKKRKV

SEQ ID NO: 838 - protein, 27 aa, Artificial Sequence
  CPP
MGLGLHLLVLAAALQGAWSQPKKKRKV

SEQ ID NO: 839 - protein, 32 aa, Artificial Sequence
  CPP
MGVADLIKKFESISKEEGGGGKGGRRRRRRRR

SEQ ID NO: 840 - protein, 32 aa, Artificial Sequence; identical to SEQ ID NO: 839
  CPP

SEQ ID NO: 841 - protein, 32 aa, Artificial Sequence; identical to SEQ ID NO: 839
  CPP

SEQ ID NO: 842 - protein, 12 aa, Artificial Sequence
  CPP
MHKRPTTPSRKM

SEQ ID NO: 843 - protein, 9 aa, Artificial Sequence
  CPP
MIAYRDLIS

SEQ ID NO: 844 - protein, 9 aa, Artificial Sequence
  CPP
MIIARDLIS

SEQ ID NO: 845 - protein, 12 aa, Artificial Sequence
  CPP
MIIFAIAASHKK

SEQ ID NO: 846 - protein, 12 aa, Artificial Sequence
  CPP
MIIFKIAASHKK

SEQ ID NO: 847 - protein, 12 aa, Artificial Sequence
  CPP
MIIFRAAASHKK

SEQ ID NO: 848 - protein, 12 aa, Artificial Sequence
  CPP
MIIFRALISHKK

SEQ ID NO: 849 - protein, 10 aa, Artificial Sequence
  CPP
MIIFRDLISH

SEQ ID NO: 850 - protein, 12 aa, Artificial Sequence
  CPP
MIIFRIAASHKK

SEQ ID NO: 851 - protein, 12 aa, Artificial Sequence
  CPP
MIIFRIAATHKK

SEQ ID NO: 852 - protein, 12 aa, Artificial Sequence
  CPP
MIIFRIAAYHKK

SEQ ID NO: 853 - protein, 12 aa, Artificial Sequence
  CPP
MIIFRILISHKK

SEQ ID NO: 854 - protein, 10 aa, Artificial Sequence
  CPP
MIIRRDLISE

SEQ ID NO: 855 - protein, 10 aa, Artificial Sequence
  CPP
MIISRDLISH

SEQ ID NO: 856 - protein, 9 aa, Artificial Sequence
  CPP
MIIYADLIS

SEQ ID NO: 857 - protein, 10 aa, Artificial Sequence
  CPP
MIIYARRAEE

SEQ ID NO: 858 - protein, 10 aa, Artificial Sequence
  CPP
MIIYRAEISH

SEQ ID NO: 859 - protein, 9 aa, Artificial Sequence
  CPP
MIIYRALIS

SEQ ID NO: 860 - protein, 12 aa, Artificial Sequence
  CPP
MIIYRALISHKK

SEQ ID NO: 861 - protein, 6 aa, Artificial Sequence
  CPP
MIIYRD

SEQ ID NO: 862 - protein, 9 aa, Artificial Sequence
  CPP
MIIYRDAIS

SEQ ID NO: 863 - protein, 10 aa, Artificial Sequence
  CPP
MIIYRDKKSH

SEQ ID NO: 864 - protein, 7 aa, Artificial Sequence
  CPP
MIIYRDL

SEQ ID NO: 865 - protein, 9 aa, Artificial Sequence
  CPP
MIIYRDLAS

SEQ ID NO: 866 - protein, 8 aa, Artificial Sequence
  CPP
MIIYRDLI

SEQ ID NO: 867 - protein, 9 aa, Artificial Sequence
  CPP
MIIYRDLIA

SEQ ID NO: 868 - protein, 9 aa, Artificial Sequence
  CPP
MIIYRDLIS

SEQ ID NO: 869 - protein, 10 aa, Artificial Sequence
  CPP
MIIYRDLISH

SEQ ID NO: 870 - protein, 11 aa, Artificial Sequence
  CPP
MIIYRDLISKK

SEQ ID NO: 871 - protein, 12 aa, Artificial Sequence
  CPP
MIIYRIAASHKK

SEQ ID NO: 872 - protein, 10 aa, Artificial Sequence
  CPP
MLLLTRRRST

SEQ ID NO: 873 - protein, 10 aa, Artificial Sequence
  CPP
HEHEHEHEHE

SEQ ID NO: 874 - protein, 10 aa, Artificial Sequence
  CPP
RGRGRGRGRG

SEQ ID NO: 875 - protein, 30 aa, Artificial Sequence
  CPP
MRRIRPRPPRLPRPRPRPLPFPRPGGCYPG

SEQ ID NO: 876 - protein, 12 aa, Artificial Sequence
  CPP
MTPSSLSTLPWP

SEQ ID NO: 877 - protein, 30 aa, Artificial Sequence
  CPP
MVKSKIGSWILVLFVAMWSDVGLCKKRPKP

SEQ ID NO: 878 - protein, 22 aa, Artificial Sequence
  CPP
MVRRFLVTLRIRRACGPPRVRV

SEQ ID NO: 879 - protein, 37 aa, Artificial Sequence
  CPP
MVRRFLVTLRIRRACGPPRVRVFVVHIPRLTGEWAAP

SEQ ID NO: 880 - protein, 22 aa, Artificial Sequence
  CPP
MVTVLFKRLRIRRACGPPRVKV

SEQ ID NO: 881 - protein, 22 aa, Artificial Sequence
  CPP
MVTVLFRRLRIRRACGPPRVRV

SEQ ID NO: 882 - protein, 11 aa, Artificial Sequence
  CPP
RRRRRRRRRRR

SEQ ID NO: 883 - protein, 19 aa, Artificial Sequence
  CPP
NAKTRRHERRRKLAIERGC

SEQ ID NO: 884 - protein, 8 aa, Artificial Sequence
  CPP
GGGGGGGG

SEQ ID NO: 885 - protein, 12 aa, Artificial Sequence
  CPP
GGGGGGGGGGGG

SEQ ID NO: 886 - protein, 12 aa, Artificial Sequence
  CPP
GGGGGGGGGGGG

SEQ ID NO: 887 - protein, 14 aa, Artificial Sequence
  CPP
NFLGTLVNLAKKIL

SEQ ID NO: 888 - protein, 12 aa, Artificial Sequence
  CPP
NHQQQNPHQPPM

SEQ ID NO: 889 - protein, 30 aa, Artificial Sequence
  CPP
NHQQQNPHQPPMLLIILRRRIRKQAHAHSK

SEQ ID NO: 890 - protein, 12 aa, Artificial Sequence
  CPP
NIENSTLATPLS

SEQ ID NO: 891 - protein, 8 aa, Artificial Sequence
  CPP
NKPILVFY

SEQ ID NO: 892 - protein, 12 aa, Artificial Sequence
  CPP
NKRILIRIMTRP

SEQ ID NO: 893 - protein, 13 aa, Artificial Sequence
  CPP
GGGGGGGGGGGGG

SEQ ID NO: 894 - protein, 12 aa, Artificial Sequence
  CPP
GGGGGGGGGGGG

SEQ ID NO: 895 - protein, 12 aa, Artificial Sequence
  CPP
GGGGGGGGGGGG

SEQ ID NO: 896 - protein, 12 aa, Artificial Sequence
  CPP
GGGGGGGGGGGG

SEQ ID NO: 897 - protein, 9 aa, Artificial Sequence
  CPP
GGGGGGGGG

SEQ ID NO: 898 - protein, 12 aa, Artificial Sequence
  CPP
GGGGGGGGGGGG

SEQ ID NO: 899 - protein, 12 aa, Artificial Sequence
  CPP
GGGGGGGGGGGG

SEQ ID NO: 900 - protein, 13 aa, Artificial Sequence
  CPP
NNNAAGRKRKKRT

SEQ ID NO: 901 - protein, 11 aa, Artificial Sequence
  CPP
NRARRNRRRVR

SEQ ID NO: 902 - protein, 13 aa, Artificial Sequence
  CPP
NRHFRFFFNFTNR

SEQ ID NO: 903 - protein, 8 aa, Artificial Sequence
  CPP
NRRMKWKK

SEQ ID NO: 904 - protein, 12 aa, Artificial Sequence
  CPP
NSGTMQSASRAT

SEQ ID NO: 905 - protein, 9 aa, Artificial Sequence
  CPP
NTCTWLKYH

SEQ ID NO: 906 - protein, 10 aa, Artificial Sequence
  CPP
NTCTWLKYHS

SEQ ID NO: 907 - protein, 10 aa, Artificial Sequence
  CPP
NTGTWLKYHS

SEQ ID NO: 908 - protein, 10 aa, Artificial Sequence
  CPP
NYQRRCKNQN

SEQ ID NO: 909 - protein, 10 aa, Artificial Sequence
  CPP
NYQWRCKNQN

SEQ ID NO: 910 - protein, 10 aa, Artificial Sequence
  CPP
NYRRRCKNQN

SEQ ID NO: 911 - protein, 7 aa, Artificial Sequence
  CPP
NYRWRCK

SEQ ID NO: 912 - protein, 8 aa, Artificial Sequence
  CPP
NYRWRCKN

SEQ ID NO: 913 - protein, 9 aa, Artificial Sequence
  CPP
NYRWRCKNQ

SEQ ID NO: 914 - protein, 10 aa, Artificial Sequence
  CPP
NYRWRCKNQN

SEQ ID NO: 915 - protein, 12 aa, Artificial Sequence
  CPP
NYTTYKSHFQDR

SEQ ID NO: 916 - protein, 11 aa, Artificial Sequence
  CPP
PARAARRAARR

SEQ ID NO: 917 - protein, 6 aa, Artificial Sequence
  CPP
PFVYLI

SEQ ID NO: 918 - protein, 12 aa, Artificial Sequence
  CPP
PIRRRKKLRRLK

SEQ ID NO: 919 - protein, 7 aa, Artificial Sequence
  CPP
PKKKRKV

SEQ ID NO: 920 - protein, 51 aa, Artificial Sequence
  CPP
PKKKRKVAGYLLGKINLKALAALAKKILPQMQQNVFQYPGAGMVPQGEANF

SEQ ID NO: 921 - protein, 37 aa, Artificial Sequence
  CPP
PKKKRKVRRRRRRRPQMQQNVFQYPGAGMVPQGEANF

SEQ ID NO: 922 - protein, 32 aa, Artificial Sequence
  CPP
PKKKRKVRRRRRRRYSQTSHKLVQLLTTAEQQ

SEQ ID NO: 923 - protein, 20 aa, Artificial Sequence
  CPP
PKKKRKVALWKTLLKKVLKA

SEQ ID NO: 924 - protein, 18 aa, Artificial Sequence
  CPP
PKKKRKVWKLLQQFFGLM

SEQ ID NO: 925 - protein, 12 aa, Artificial Sequence
  CPP
PLSSIFSRIGDP

SEQ ID NO: 926 - protein, 5 aa, Artificial Sequence
  CPP
PMLKE

SEQ ID NO: 927 - protein, 11 aa, Artificial Sequence
  CPP
PNTRVRPDVSF

SEQ ID NO: 928 - protein, 12 aa, Artificial Sequence
  CPP
PPHNRIQRRLNM

SEQ ID NO: 929 - protein, 15 aa, Artificial Sequence
  CPP
PPKKSAQCLRYKKPE

SEQ ID NO: 930 - protein, 18 aa, Artificial Sequence
  CPP
PPRLPRPRPRPLPFPRPG

SEQ ID NO: 931 - protein, 12 aa, Artificial Sequence
  CPP
PPRLRKRRQLNM

SEQ ID NO: 932 - protein, 12 aa, Artificial Sequence
  CPP
PQNRLQIRRHSK

SEQ ID NO: 933 - protein, 10 aa, Artificial Sequence
  CPP
PRPLPFPRPG

SEQ ID NO: 934 - protein, 20 aa, Artificial Sequence
  CPP
PRPPRLPRPRPRPLPFPRPG

SEQ ID NO: 935 - protein, 12 aa, Artificial Sequence
  CPP
PRPRPLPFPRPG

SEQ ID NO: 936 - protein, 14 aa, Artificial Sequence
  CPP
PRPRPRPLPFPRPG

SEQ ID NO: 937 - protein, 12 aa, Artificial Sequence
  CPP
PSKRLLHNNLRR

SEQ ID NO: 938 - protein, 12 aa, Artificial Sequence
  CPP
PSSSSSSRIGDP

SEQ ID NO: 939 - protein, 12 aa, Artificial Sequence
  CPP
QAASRVENYMHR

SEQ ID NO: 940 - protein, 10 aa, Artificial Sequence
  CPP
QIISRDLISH

SEQ ID NO: 941 - protein, 15 aa, Artificial Sequence
  CPP
QIKIWFQNRRMKWKK

SEQ ID NO: 942 - protein, 18 aa, Artificial Sequence
  CPP
QLALQLALQALQAALQLA

SEQ ID NO: 943 - protein, 5 aa, Artificial Sequence
  CPP
QLPVM

SEQ ID NO: 944 - protein, 9 aa, Artificial Sequence
  CPP
QNRRMKWKK

SEQ ID NO: 945 - protein, 12 aa, Artificial Sequence
  CPP
QPIIITSPYLPS

SEQ ID NO: 946 - protein, 15 aa, Artificial Sequence
  CPP
QQHLLIAINGYPRYN

SEQ ID NO: 947 - protein, 12 aa, Artificial Sequence
  CPP
QRIRKSKISRTL

SEQ ID NO: 948 - protein, 12 aa, Artificial Sequence
  CPP
QSPTDFTFPNPL

SEQ ID NO: 949 - protein, 15 aa, Artificial Sequence
  CPP
QTRRRERRAEKQAQW

SEQ ID NO: 950 - protein, 10 aa, Artificial Sequence
  CPP
QWQRNMRKVR

SEQ ID NO: 951 - protein, 19 aa, Artificial Sequence
  CPP
QWQRNMRKVRGPPVSCIKR

SEQ ID NO: 952 - protein, 17 aa, Artificial Sequence
  CPP
RAGLQFPVGRVHRLLRK

SEQ ID NO: 953 - protein, 16 aa, Artificial Sequence
  CPP
RAIKIWFQNRRMKWKK

SEQ ID NO: 954 - protein, 9 aa, Artificial Sequence
  CPP
RAKRRQRRR

SEQ ID NO: 955 - protein, 32 aa, Artificial Sequence
  CPP
RARARARARARARARARARARARARARARARA

SEQ ID NO: 956 - protein, 15 aa, Artificial Sequence
  CPP
RAWMRWYSPTTRRYG

SEQ ID NO: 957 - protein, 15 aa, Artificial Sequence
  CPP
RFTFHFRFEFTFHFE

SEQ ID NO: 958 - protein, 25 aa, Artificial Sequence
  CPP
RFTFHFRFEFTFHFEGGGRRRRRRR

SEQ ID NO: 959 - protein, 15 aa, Artificial Sequence
  CPP
RGDADDARRRRRRRR

SEQ ID NO: 960 - protein, 11 aa, Artificial Sequence
  CPP
RGDRRRRRRRR

SEQ ID NO: 961 - protein, 11 aa, Artificial Sequence
  CPP
RGDRRRRRRRR

SEQ ID NO: 962 - protein, 5 aa, Artificial Sequence
  CPP
RGDFK

SEQ ID NO: 963 - protein, 15 aa, Artificial Sequence
  CPP
RGDGPRRRPRKRRGR

SEQ ID NO: 964 - protein, 19 aa, Artificial Sequence
  CPP
RGDRGDRRDLRLDRGDLRC

SEQ ID NO: 965 - protein, 19 aa, Artificial Sequence
  CPP
RGDRLDRRDLRLDRRDLRC

SEQ ID NO: 966 - protein, 19 aa, Artificial Sequence
  CPP
RGERGERRELRLERGELRC

SEQ ID NO: 967 - protein, 19 aa, Artificial Sequence
  CPP
RGERLERRELRLERRELRC

SEQ ID NO: 968 - protein, 17 aa, Artificial Sequence
  CPP
RGGRLAYLRRRWAVLGR

SEQ ID NO: 969 - protein, 18 aa, Artificial Sequence
  CPP
RGGRLSYSRRRFSTSTGR

SEQ ID NO: 970 - protein, 19 aa, Artificial Sequence
  CPP
RGGRLSYSRRRFSTSTGRA

SEQ ID NO: 971 - protein, 15 aa, Artificial Sequence
  CPP
RGPRRQPRRHRRPRR

SEQ ID NO: 972 - protein, 10 aa, Artificial Sequence
  CPP
RGRGRGRGRG

SEQ ID NO: 973 - protein, 36 aa, Artificial Sequence
  CPP
RGSRRAVTRAQRRDGRRRRRSRRESYSVYVYRVLRQ

SEQ ID NO: 974 - protein, 11 aa, Artificial Sequence
  CPP
RHHLRHLRRHL

SEQ ID NO: 975 - protein, 22 aa, Artificial Sequence
  CPP
RHHLRHLRRHLRHLLRHLRHHL

SEQ ID NO: 976 - protein, 33 aa, Artificial Sequence
  CPP
RHHLRHLRRHLRHLLRHLRHHLRHLRRHLRHLL

SEQ ID NO: 977 - protein, 22 aa, Artificial Sequence
  CPP
RHHRRHHRRHRRHHRRHHRHHR

SEQ ID NO: 978 - protein, 16 aa, Artificial Sequence
  CPP
RHIKIWFQNRRMKWKK

SEQ ID NO: 979 - protein, 14 aa, Artificial Sequence
  CPP
RHNFRFFFNFRTNR

SEQ ID NO: 980 - protein, 21 aa, Artificial Sequence
  CPP
RHNHRFNFRFFFNFRFNTRTN

SEQ ID NO: 981 - protein, 15 aa, Artificial Sequence
  CPP
RHRHRRHRHRRHRHR

SEQ ID NO: 982 - protein, 10 aa, Artificial Sequence
  CPP
RHVYHVLLSQ

SEQ ID NO: 983 - protein, 6 aa, Artificial Sequence
  CPP
RIFIGC

SEQ ID NO: 984 - protein, 10 aa, Artificial Sequence
  CPP
RIFIHFRIGC

SEQ ID NO: 985 - protein, 8 aa, Artificial Sequence
  CPP
RIFIRIGC

SEQ ID NO: 986 - protein, 30 aa, Artificial Sequence
  CPP
RIKAERKRMRNRIAASKSRKRKLERIARGC

SEQ ID NO: 987 - protein, 11 aa, Artificial Sequence
  CPP
RILQQLLFIHF

SEQ ID NO: 988 - protein, 15 aa, Artificial Sequence
  CPP
RILQQLLFIHFRIGC

SEQ ID NO: 989 - protein, 17 aa, Artificial Sequence
  CPP
RILQQLLFIHFRIGCRH

SEQ ID NO: 990 - protein, 20 aa, Artificial Sequence
  CPP
RILQQLLFIHFRIGCRHSRI

SEQ ID NO: 991 - protein, 13 aa, Artificial Sequence
  CPP
RIMRILRILKLAR

SEQ ID NO: 992 - protein, 12 aa, Artificial Sequence
  CPP
RIRMIQNLIKKT

SEQ ID NO: 993 - protein, 9 aa, Artificial Sequence
  CPP
RKARRQRRR

SEQ ID NO: 994 - protein, 6 aa, Artificial Sequence
  CPP
RKKAAA

SEQ ID NO: 995 - protein, 9 aa, Artificial Sequence
  CPP
RKKARQRRR

SEQ ID NO: 996 - protein, 10 aa, Artificial Sequence
  CPP
RKKNPNCRRH

SEQ ID NO: 997 - protein, 9 aa, Artificial Sequence
  CPP
RKKRAQRRR

SEQ ID NO: 998 - protein, 19 aa, Artificial Sequence
  CPP
  misc_feature (9)..(9): Xaa can be any naturally occurring amino acid
  misc_feature (12)..(12): Xaa can be any naturally occurring amino acid
  misc_feature (15)..(15): Xaa can be any naturally occurring amino acid
  misc_feature (18)..(18): Xaa can be any naturally occurring amino acid
RKKRKKKRXRHXRHXRHXR

SEQ ID NO: 999 - protein, 9 aa, Artificial Sequence
  CPP
RKKRRARRR

SEQ ID NO: 1000 - protein, 9 aa, Artificial Sequence
  CPP
RKKRRQARR

SEQ ID NO: 1001 - protein, 7 aa, Artificial Sequence
  CPP
RKKRRQR

SEQ ID NO: 1002 - protein, 9 aa, Artificial Sequence
  CPP
RKKRRQRAR

SEQ ID NO: 1003 - protein, 8 aa, Artificial Sequence
  CPP
RKKRRQRR

SEQ ID NO: 1004 - protein, 9 aa, Artificial Sequence
  CPP
RKKRRQRRA

SEQ ID NO: 1005 - protein, 9 aa, Artificial Sequence
  CPP
RKKRRQRRR

SEQ ID NO: 1006 - protein, 11 aa, Artificial Sequence
  CPP
RKKRRQRRRGC

SEQ ID NO: 1007 - protein, 12 aa, Artificial Sequence
  CPP
RKKRRQRRRGGG

SEQ ID NO: 1008 - protein, 27 aa, Artificial Sequence
  CPP
RKKRRQRRRGGGKLLKLLLKLLLKLLK

SEQ ID NO: 1009 - protein, 15 aa, Artificial Sequence
  CPP
RKKRRQRRRHRRKKR

SEQ ID NO: 1010 - protein, 29 aa, Artificial Sequence
  CPP
RKKRRQRRRPPQCAAVALLPAVLLALLAP

SEQ ID NO: 1011 - protein, 18 aa, Artificial Sequence
  CPP
RKKRRQRRRRKKRRQRRR

SEQ ID NO: 1012 - protein, 16 aa, Artificial Sequence
  CPP
RKKRRRESRKKRRRES

SEQ ID NO: 1013 - protein, 17 aa, Artificial Sequence
  CPP
RKKRRRESRKKRRRESC

SEQ ID NO: 1014 - protein, 18 aa, Artificial Sequence
  CPP
RKKRRRESRRARRSPRHL

SEQ ID NO: 1015 - protein, 29 aa, Artificial Sequence
  CPP
RKKRRRESWVHLPPPVHLPPPGGHHHHHH

SEQ ID NO: 1016 - protein, 6 aa, Artificial Sequence
  CPP
RKKWFW

SEQ ID NO: 1017 - protein, 20 aa, Artificial Sequence
  CPP
RKLTTIFPLNWKYRKALSLG

SEQ ID NO: 1018 - protein, 18 aa, Artificial Sequence
  CPP
RLALRLALRALRAALRLA

SEQ ID NO: 1019 - protein, 15 aa, Artificial Sequence
  CPP
RLAMRWYSPTTRRYG

SEQ ID NO: 1020 - protein, 15 aa, Artificial Sequence
  CPP
RLFMRFYSPTTRRYG

SEQ ID NO: 1021 - protein, 15 aa, Artificial Sequence
  CPP
RLHHRLHRRLHRLHR

SEQ ID NO: 1022 - protein, 29 aa, Artificial Sequence
  CPP
RLHHRLHRRLHRLHRRLHRLHHRLHRRLH

SEQ ID NO: 1023 - protein, 18 aa, Artificial Sequence
  CPP
RLHLRLHLRHLRHHLRLH

SEQ ID NO: 1024 - protein, 15 aa, Artificial Sequence
  CPP
RLHRRLHRRLHRLHR

SEQ ID NO: 1025 - protein, 29 aa, Artificial Sequence
  CPP
RLHRRLHRRLHRLHRRLHRLHRRLHRRLH

SEQ ID NO: 1026 - protein, 15 aa, Artificial Sequence
  CPP
RLIMRIYAPTTRRYG

SEQ ID NO: 1027 - protein, 15 aa, Artificial Sequence
  CPP
RLIMRIYSPTTRRYG

SEQ ID NO: 1028 - protein, 15 aa, Artificial Sequence
  CPP
RLLMRLYSPTTRRYG

SEQ ID NO: 1029 - protein, 18 aa, Artificial Sequence
  CPP
RLLRLLLRLWRRLLRLLR

SEQ ID NO: 1030 - protein, 9 aa, Artificial Sequence
  CPP
RLLRLLRLL

SEQ ID NO: 1031 - protein, 9 aa, Artificial Sequence
  CPP
  misc_feature (9)..(9): Xaa can be any naturally occurring amino acid
RLLRLLRLX

SEQ ID NO: 1032 - protein, 19 aa, Artificial Sequence
  CPP
RLLRLLRRLLRLLRRLLRC

SEQ ID NO: 1033 - protein, 9 aa, Artificial Sequence
  CPP
  misc_feature (6)..(6): Xaa can be any naturally occurring amino acid
  misc_feature (9)..(9): Xaa can be any naturally occurring amino acid
RLLRLXRLX

SEQ ID NO: 1034 - protein, 16 aa, Artificial Sequence
  CPP
RLPRPRPRPLPFPRPG

SEQ ID NO: 1035 - protein, 38 aa, Artificial Sequence
  CPP
RLRLRLRLRLRLRLRLKLLKLLKLLKLLKKKKKKKGYK

SEQ ID NO: 1036 - protein, 38 aa, Artificial Sequence
  CPP
RLRLRLRLRLRLRLRLKNNKNNKNNKNNKKKKKKKGYK

SEQ ID NO: 1037 - protein, 38 aa, Artificial Sequence
  CPP
RLRLRLRLRLRLRLRLKRLKRLKRLKRLKKKKKKKGYK

SEQ ID NO: 1038 - protein, 14 aa, Artificial Sequence
  CPP
RLSGMNEVLSFRWL

SEQ ID NO: 1039 - protein, 15 aa, Artificial Sequence
  CPP
RLVMRVYSPTTRRYG

SEQ ID NO: 1040 - protein, 15 aa, Artificial Sequence
  CPP
RLWARWYSPTTRRYG

SEQ ID NO: 1041 - protein, 15 aa, Artificial Sequence
  CPP
RLWMAWYSPTTRRYG

SEQ ID NO: 1042 - protein, 15 aa, Artificial Sequence
  CPP
RLWMRAYSPTTRRYG

SEQ ID NO: 1043 - protein, 15 aa, Artificial Sequence
  CPP
RLWMRWASPTTRRYG

SEQ ID NO: 1044 - protein, 15 aa, Artificial Sequence
  CPP
RLWMRWYAPTTRRYG

SEQ ID NO: 1045 - protein, 15 aa, Artificial Sequence
  CPP
RLWMRWYSPATRRYG

SEQ ID NO: 1046 - protein, 15 aa, Artificial Sequence
  CPP
RLWMRWYSPRTRAYG

SEQ ID NO: 1047 - protein, 15 aa, Artificial Sequence
  CPP
RLWMRWYSPTARRYG

SEQ ID NO: 1048 - protein, 15 aa, Artificial Sequence
  CPP
RLWMRWYSPTTARYG

SEQ ID NO: 1049 - protein, 15 aa, Artificial Sequence
  CPP
RLWMRWYSPTTRAYG

SEQ ID NO: 1050 - protein, 15 aa, Artificial Sequence
  CPP
RLWMRWYSPTTRRAG

SEQ ID NO: 1051 - protein, 15 aa, Artificial Sequence
  CPP
RLWMRWYSPTTRRYA

SEQ ID NO: 1052 - protein, 15 aa, Artificial Sequence
  CPP
RLWMRWYSPTTRRYG

SEQ ID NO: 1053 - protein, 15 aa, Artificial Sequence
  CPP
RLWMRWYSPWTRRWG

SEQ ID NO: 1054 - protein, 15 aa, Artificial Sequence
  CPP
RLWMRWYSPWTRRYG

SEQ ID NO: 1055 - protein, 16 aa, Artificial Sequence
  CPP
RLWRALPRVLRRLLRP

SEQ ID NO: 1056 - protein, 9 aa, Artificial Sequence
  CPP
  misc_feature (3)..(3): Xaa can be any naturally occurring amino acid
  misc_feature (6)..(6): Xaa can be any naturally occurring amino acid
  misc_feature (9)..(9): Xaa can be any naturally occurring amino acid
RLXRLXRLX

SEQ ID NO: 1057 - protein, 9 aa, Artificial Sequence
  CPP
  misc_feature (3)..(3): Xaa can be any naturally occurring amino acid
  misc_feature (6)..(6): Xaa can be any naturally occurring amino acid
  misc_feature (8)..(9): Xaa can be any naturally occurring amino acid
RLXRLXRXX

SEQ ID NO: 1058 - protein, 8 aa, Artificial Sequence
  CPP
  misc_feature (3)..(3): Xaa can be any naturally occurring amino acid
  misc_feature (5)..(5): Xaa can be any naturally occurring amino acid
  misc_feature (7)..(8): Xaa can be any naturally occurring amino acid
RLXRXRXX

SEQ ID NO: 1059 - protein, 15 aa, Artificial Sequence
  CPP
RLYMRYYSPTTRRYG

SEQ ID NO: 1060 - protein, 6 aa, Artificial Sequence
  CPP
RMKWKK

SEQ ID NO: 1061 - protein, 17 aa, Artificial Sequence
  CPP
  misc_feature (13)..(13): Xaa can be any naturally occurring amino acid
  misc_feature (16)..(16): Xaa can be any naturally occurring amino acid
RMKWKKILFQYRXRRXR

SEQ ID NO: 1062 - protein, 8 aa, Artificial Sequence
  CPP
RNRSRHRR

SEQ ID NO: 1063 - protein, 7 aa, Artificial Sequence
  CPP
RPARPAR

SEQ ID NO: 1064 - protein, 16 aa, Artificial Sequence
  CPP
RQAKIWFQNRRMKWKK

SEQ ID NO: 1065 - protein, 22 aa, Artificial Sequence
  CPP
RQARRNRRRALWKTLLKKVLKA

SEQ ID NO: 1066 - protein, 10 aa, Artificial Sequence
  CPP
RQARRNRRRC

SEQ ID NO: 1067 - protein, 27 aa, Artificial Sequence
  CPP
RQGAARVTSWLGRQLRIAGKRLEGRSK

SEQ ID NO: 1068 - protein, 16 aa, Artificial Sequence
  CPP
RQIAIWFQNRRMKWKK

SEQ ID NO: 1069 - protein, 16 aa, Artificial Sequence
  CPP
RQIKAWFQNRRMKWKK

SEQ ID NO: 1070 - protein, 16 aa, Artificial Sequence
  CPP
RQIKIAFQNRRMKWKK

SEQ ID NO: 1071 - protein, 16 aa, Artificial Sequence
  CPP
RQIKIFFQNRRMKFKK

SEQ ID NO: 1072 - protein, 16 aa, Artificial Sequence
  CPP
RQIKIFFQNRRMKWKK

SEQ ID NO: 1073 - protein, 15 aa, Artificial Sequence
  CPP
RQIKIQFQNRRKWKK

SEQ ID NO: 1074 - protein, 6 aa, Artificial Sequence
  CPP
RQIKIW

SEQ ID NO: 1075 - protein, 16 aa, Artificial Sequence
  CPP
RQIKIWAQNRRMKWKK

SEQ ID NO: 1076 - protein, 16 aa, Artificial Sequence
  CPP
RQIKIWFANRRMKWKK

SEQ ID NO: 1077 - protein, 16 aa, Artificial Sequence
  CPP
RQIKIWFPNRRMKWKK

SEQ ID NO: 1078 - protein, 8 aa, Artificial Sequence
  CPP
RQIKIWFQ

SEQ ID NO: 1079 - protein, 16 aa, Artificial Sequence
  CPP
RQIKIWFQARRMKWKK

SEQ ID NO: 1080 - protein, 9 aa, Artificial Sequence
  CPP
RQIKIWFQN

SEQ ID NO: 1081 - protein, 16 aa, Artificial Sequence
  CPP
RQIKIWFQNARMKWKK

SEQ ID NO: 1082 - protein, 16 aa, Artificial Sequence
  CPP
RQIKIWFQNMRRKWKK

SEQ ID NO: 1083 - protein, 10 aa, Artificial Sequence
  CPP
RQIKIWFQNR

SEQ ID NO: 1084 - protein, 16 aa, Artificial Sequence
  CPP
RQIKIWFQNRAMKWKK

SEQ ID NO: 1085 - protein, 11 aa, Artificial Sequence
  CPP
RQIKIWFQNRR

SEQ ID NO: 1086 - protein, 16 aa, Artificial Sequence
  CPP
RQIKIWFQNRRAKWKK

SEQ ID NO: 1087 - protein, 12 aa, Artificial Sequence
  CPP
RQIKIWFQNRRM

SEQ ID NO: 1088 - protein, 16 aa, Artificial Sequence
  CPP
RQIKIWFQNRRMAWKK

SEQ ID NO: 1089 - protein, 13 aa, Artificial Sequence
  CPP
RQIKIWFQNRRMK

SEQ ID NO: 1090 - protein, 16 aa, Artificial Sequence
  CPP
RQIKIWFQNRRMKAKK

SEQ ID NO: 1091 - protein, 14 aa, Artificial Sequence
  CPP
RQIKIWFQNRRMKW

SEQ ID NO: 1092 - protein, 16 aa, Artificial Sequence
  CPP
RQIKIWFQNRRMKWAK

SEQ ID NO: 1093 - protein, 15 aa, Artificial Sequence
  CPP
RQIKIWFQNRRMKWK

SEQ ID NO: 1094 - protein, 16 aa, Artificial Sequence
  CPP
RQIKIWFQNRRMKWKA

SEQ ID NO: 1095 - protein, 16 aa, Artificial Sequence
  CPP
RQIKIWFQNRRMKWKK

SEQ ID NO: 1096 - protein, 17 aa, Artificial Sequence
  CPP
RQIKIWFQNRRMKWKKC

SEQ ID NO: 1097 - protein, 35 aa, Artificial Sequence
  CPP
RQIKIWFQNRRMKWKKDIMGEWGNEIFGAIAGFLG

SEQ ID NO: 1098 - protein, 18 aa, Artificial Sequence
  CPP
RQIKIWFQNRRMKWKKGC

SEQ ID NO: 1099 - protein, 18 aa, Artificial Sequence
  CPP
RQIKIWFQNRRMKWKKGG

SEQ ID NO: 1100 - protein, 17 aa, Artificial Sequence
  CPP
RQIKIWFQNRRMKWKKK

SEQ ID NO: 1101 - protein, 31 aa, Artificial Sequence
  CPP
RQIKIWFQNRRMKWKKRQIKIWFQNRRMKWK

SEQ ID NO: 1102 - protein, 33 aa, Artificial Sequence
  CPP
RQIKIWFQNRRMKWKKTYADFIASGRTGRRNAI

SEQ ID NO: 1103 - protein, 16 aa, Artificial Sequence
  CPP
RQIRIWFQNRRMRWRR

SEQ ID NO: 1104 - protein, 17 aa, Artificial Sequence
  CPP
RQIRIWFQNRRMRWRRC

SEQ ID NO: 1105 - protein, 15 aa, Artificial Sequence
  CPP
RQLRIAGRRLRGRSR

SEQ ID NO: 1106 - protein, 16 aa, Artificial Sequence
  CPP
RQPKIWFPNRRKPWKK

SEQ ID NO: 1107 - protein, 12 aa, Artificial Sequence
  CPP
RQRSRRRPLNIR

SEQ ID NO: 1108 - protein, 15 aa, Artificial Sequence
  CPP
RRARRPRRLRPAPGR

SEQ ID NO: 1109 - protein, 4 aa, Artificial Sequence
  CPP
RRGC

SEQ ID NO: 1110 - protein, 6 aa, Artificial Sequence
  CPP
RRGRRG

SEQ ID NO: 1111 - protein, 13 aa, Artificial Sequence
  CPP
RRHHCRSKAKRSR

SEQ ID NO: 1112 - protein, 22 aa, Artificial Sequence
  CPP
RRHLRRHLRHLRRHLRRHLRHL

SEQ ID NO: 1113 - protein, 10 aa, Artificial Sequence
  CPP
RRIPNRRPRR

SEQ ID NO: 1114 - protein, 7 aa, Artificial Sequence
  CPP
RRIRPRP

SEQ ID NO: 1115 - protein, 15 aa, Artificial Sequence
  CPP
RRIRPRPPRLPRPRP

SEQ ID NO: 1116 - protein, 17 aa, Artificial Sequence
  CPP
RRIRPRPPRLPRPRPRP

SEQ ID NO: 1117 - protein, 24 aa, Artificial Sequence
  CPP
RRIRPRPPRLPRPRPRPLPFPRPG

SEQ ID NO: 1118 - protein, 11 aa, Artificial Sequence
  CPP
RRKLSQQKEKK

SEQ ID NO: 1119 - protein, 9 aa, Artificial Sequence
  CPP
RRLLRRLRR

SEQ ID NO: 1120 - protein, 18 aa, Artificial Sequence
  CPP
RRLRHLRHHYRRRWHRFR

SEQ ID NO: 1121 - protein, 10 aa, Artificial Sequence
  CPP
RRLSYSRRRF

SEQ ID NO: 1122 - protein, 7 aa, Artificial Sequence
  CPP
RRMKWKK

SEQ ID NO: 1123 - protein, 12 aa, Artificial Sequence
  CPP
RRQRRTSKLMKR

SEQ ID NO: 1124 - protein, 3 aa, Artificial Sequence
  CPP
RRR

SEQ ID NO: 1125 - protein, 9 aa, Artificial Sequence
  CPP
RRRERRAEK

SEQ ID NO: 1126 - protein, 9 aa, Artificial Sequence
  CPP
RRRGRKKRR

SEQ ID NO: 1127 - protein, 15 aa, Artificial Sequence
  CPP
RRRQKRIVVRRRLIR

SEQ ID NO: 1128 - protein, 9 aa, Artificial Sequence
  CPP
RRRQRRKKR

SEQ ID NO: 1129 - protein, 26 aa, Artificial Sequence
  CPP
RRRQRRKKRGYCKCKYGRKKRRQRRR

SEQ ID NO: 1130 - protein, 29 aa, Artificial Sequence
  CPP
RRRQRRKRGGDIMGEWGNEIFGAIAGFLG

SEQ ID NO: 1131 - protein, 4 aa, Artificial Sequence
  CPP
RRRR

SEQ ID NO: 1132 - protein, 17 aa, Artificial Sequence
  CPP
RRRRNRTRRNRRRVRGC

SEQ ID NO: 1133 - protein, 5 aa, Artificial Sequence
  CPP
RRRRR

SEQ ID NO: 1134 - protein, 8 aa, Artificial Sequence
  CPP
RRRRRHHH

SEQ ID NO: 1135 - protein, 6 aa, Artificial Sequence
  CPP
RRRRRR

SEQ ID NO: 1136 - protein, 9 aa, Artificial Sequence
  CPP
RRRRRRHHH

SEQ ID NO: 1137 - protein, 7 aa, Artificial Sequence
  CPP
RRRRRRR

SEQ ID NO: 1138 - protein, 23 aa, Artificial Sequence
  CPP
RRRRRRRGGIYLATALAKWALKQ

SEQ ID NO: 1139 - protein, 25 aa, Artificial Sequence
  CPP
RRRRRRRGGIYLATALAKWALKQGF

SEQ ID NO: 1140 - protein, 23 aa, Artificial Sequence
  CPP
RRRRRRRGGKLAKLAKKLAKLAK

SEQ ID NO: 1141 - protein, 10 aa, Artificial Sequence
  CPP
RRRRRRRHHH

SEQ ID NO: 1142 - protein, 25 aa, Artificial Sequence
  CPP
RRRRRRRQIKILFQNRRMKWKKGGC

SEQ ID NO: 1143 - protein, 8 aa, Artificial Sequence
  CPP
RRRRRRRR

SEQ ID NO: 1144 - protein, 13 aa, Artificial Sequence
  CPP
RRRRRRRRRGDFK

SEQ ID NO: 1145 - protein, 11 aa, Artificial Sequence
  CPP
RRRRRRRRRGD

SEQ ID NO: 1146 - protein, 9 aa, Artificial Sequence
  CPP
RRRRRRRRC

SEQ ID NO: 1147 - protein, 10 aa, Artificial Sequence
  CPP
RRRRRRRRGC

SEQ ID NO: 1148 - protein, 11 aa, Artificial Sequence
  CPP
RRRRRRRRHHH

SEQ ID NO: 1149 - protein, 9 aa, Artificial Sequence
  CPP
RRRRRRRRK

SEQ ID NO: 1150 - protein, 9 aa, Artificial Sequence
  CPP
RRRRRRRRR

SEQ ID NO: 1151 - protein, 10 aa, Artificial Sequence
  CPP
RRRRRRRRRC

SEQ ID NO: 1152 - protein, 16 aa, Artificial Sequence
  CPP
RRRRRRRRRCQCRRKN

SEQ ID NO: 1153 - protein, 24 aa, Artificial Sequence
  CPP
RRRRRRRRRGGLAASGWKHHHHHH

SEQ ID NO: 1154 - protein, 24 aa, Artificial Sequence
  CPP
RRRRRRRRRGPGVTWTPQAWFQWV

SEQ ID NO: 1155 - protein, 12 aa, Artificial Sequence
  CPP
RRRRRRRRRHHH

SEQ ID NO: 1156 - protein, 10 aa, Artificial Sequence
  CPP
RRRRRRRRRK

SEQ ID NO: 1157 - protein, 10 aa, Artificial Sequence
  CPP
RRRRRRRRRR

SEQ ID NO: 1158 - protein, 11 aa, Artificial Sequence
  CPP
RRRRRRRRRRR

SEQ ID NO: 1159 - protein, 12 aa, Artificial Sequence
  CPP
RRRRRRRRRRRR

SEQ ID NO: 1160 - protein, 14 aa, Artificial Sequence
  CPP
RRRRRRRRRRRRGC

SEQ ID NO: 1161 - protein, 15 aa, Artificial Sequence
  CPP
RRRRRRRRRRRRRRR

SEQ ID NO: 1162 - protein, 16 aa, Artificial Sequence
  CPP
RRRRRRRRRRRRRRRR

SEQ ID NO: 1163 - protein, 18 aa, Artificial Sequence
  CPP
RRRRRRRRRRRRRRRRGC

SEQ ID NO: 1164 - protein, 28 aa, Artificial Sequence
  CPP
RRRRRRRRRRRTYADFIASGRTGRRNAI

SEQ ID NO: 1165 - protein, 8 aa, Artificial Sequence
  CPP
RRRRRRRW

SEQ ID NO: 1166 - protein, 8 aa, Artificial Sequence
  CPP
RRRRWWWW

SEQ ID NO: 1167 - protein, 12 aa, Artificial Sequence
  CPP
RRRRWWWWRRRR

SEQ ID NO: 1168 - protein, 23 aa, Artificial Sequence
  CPP
RRVTSWLGRQLRIAGKRLEGRSK

SEQ ID NO: 1169 - protein, 15 aa, Artificial Sequence
  CPP
RRVWRRYRRQRWCRR

SEQ ID NO: 1170 - protein, 15 aa, Artificial Sequence
  CPP
RRWRRWNRFNRRRCR

SEQ ID NO: 1171 - protein, 16 aa, Artificial Sequence
  CPP
RRWRRWWRRWWRRWRR

SEQ ID NO: 1172 - protein, 9 aa, Artificial Sequence
  CPP
RRWWRRWRR

SEQ ID NO: 1173 - protein, 16 aa, Artificial Sequence
  CPP
RSRGRLRRGAIRLQRG

SEQ ID NO: 1174 - protein, 23 aa, Artificial Sequence
  CPP
RSVTTEINTLFQTLTSIAEKVDP

SEQ ID NO: 1175 - protein, 15 aa, Artificial Sequence
  CPP
RTLVNEYKNTLKFSK

SEQ ID NO: 1176 - protein, 10 aa, Artificial Sequence
  CPP
RTRRNRRRVR

SEQ ID NO: 1177 - protein, 16 aa, Artificial Sequence
  CPP
RVIRVWFQNKRCKDKK

SEQ ID NO: 1178 - protein, 15 aa, Artificial Sequence
  CPP
RVIRWFQNKRCKDKK

SEQ ID NO: 1179 - protein, 15 aa, Artificial Sequence
  CPP
RVIRWFQNKRSKDKK

SEQ ID NO: 1180 - protein, 15 aa, Artificial Sequence
  CPP
RVREWWYTITLKQES

SEQ ID NO: 1181 - protein, 13 aa, Artificial Sequence
  CPP
RVRILARFLRTRV

SEQ ID NO: 1182 - protein, 22 aa, Artificial Sequence
  CPP
RVRSWLGRQLRIAGKRLEGRSK

SEQ ID NO: 1183 - protein, 13 aa, Artificial Sequence
  CPP
RVRVFVVHIPRLT

SEQ ID NO: 1184 - protein, 22 aa, Artificial Sequence
  CPP
RVTSWLGRQLRIAGKRLEGRSK

SEQ ID NO: 1185 - protein, 8 aa, Artificial Sequence
  CPP
RWRCKNQN

SEQ ID NO: 1186 - protein, 12 aa, Artificial Sequence
  CPP
RWRRWRRWRRWR

SEQ ID NO: 1187 - protein, 9 aa, Artificial Sequence
  CPP
RWRRWWRRW

SEQ ID NO: 1188 - protein, 9 aa, Artificial Sequence
  CPP
RWRWKCCKK

SEQ ID NO: 1189 - protein, 9 aa, Artificial Sequence
  CPP
  misc_feature (6)..(6): Xaa can be any naturally occurring amino acid
RWRWKXCKK

SEQ ID NO: 1190 - protein, 9 aa, Artificial Sequence
  CPP
  misc_feature (6)..(7): Xaa can be any naturally occurring amino acid
RWRWKXXKK

SEQ ID NO: 1191 - protein, 8 aa, Artificial Sequence
  CPP
RWRWRWRW

SEQ ID NO: 1192 - protein, 14 aa, Artificial Sequence
  CPP
  misc_feature (2)..(2): Xaa can be any naturally occurring amino acid
  misc_feature (8)..(8): Xaa can be any naturally occurring amino acid
  misc_feature (13)..(13): Xaa can be any naturally occurring amino acid
RXRRBRRXRRBRXB

SEQ ID NO: 1193 - protein, 22 aa, Artificial Sequence
  CPP
  misc_feature (2)..(2): Xaa can be any naturally occurring amino acid
  misc_feature (8)..(8): Xaa can be any naturally occurring amino acid
  misc_feature (16)..(16): Xaa can be any naturally occurring amino acid
  misc_feature (20)..(20): Xaa can be any naturally occurring amino acid
RXRRBRRXRYQFLIRXRBRXRB

SEQ ID NO: 1194 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
RXRRXRAAAAARXRRXR

SEQ ID NO: 1195 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
RXRRXRFLQIYRXRRXR

SEQ ID NO: 1196 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
RXRRXRIEFQYRXRRXR

SEQ ID NO: 1197 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
RXRRXRIKFQYRXRRXR

SEQ ID NO: 1198 - protein, 17 aa, Artificial Sequence
  CPP
  misc_feature (2)..(2): Xaa can be any naturally occurring amino acid
  misc_feature (5)..(5): Xaa can be any naturally occurring amino acid
RXRRXRILFQYKKWKMR

SEQ ID NO: 1199 - protein, 17 aa, Artificial Sequence
  CPP
  misc_feature (2)..(2): Xaa can be any naturally occurring amino acid
  misc_feature (5)..(5): Xaa can be any naturally occurring amino acid
RXRRXRILFQYRMKWKK

SEQ ID NO: 1200 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
RXRRXRILFQYRXRRXR

SEQ ID NO: 1201 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
RXRRXRIPFQYRXRRXR

SEQ ID NO: 1202 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
RXRRXRIWFQYRXRRXR

SEQ ID NO: 1203 - protein, 12 aa, Artificial Sequence
  CPP
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
  misc_feature (11)..(11): Xaa can be any naturally occurring amino acid
RXRRXRRXRRXR

SEQ ID NO: 1204 - protein, 14 aa, Artificial Sequence
  CPP
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
  misc_feature (11)..(11): Xaa can be any naturally occurring amino acid
  misc_feature (13)..(13): Xaa can be any naturally occurring amino acid
RXRRXRRXRRXRXB

SEQ ID NO: 1205 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
RXRRXRYQFLIRXRRXR

SEQ ID NO: 1206 - protein, 17 aa, Artificial Sequence
  CPP
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
  misc_feature (8)..(8): Xaa can be any naturally occurring amino acid
  misc_feature (10)..(10): Xaa can be any naturally occurring amino acid
  misc_feature (12)..(12): Xaa can be any naturally occurring amino acid
  misc_feature (14)..(14): Xaa can be any naturally occurring amino acid
  misc_feature (16)..(16): Xaa can be any naturally occurring amino acid
RXRXRXRXRXRXRXRXB

SEQ ID NO: 1207 - protein, 8 aa, Artificial Sequence
  CPP
  misc_feature (2)..(3): Xaa can be any naturally occurring amino acid
  misc_feature (5)..(5): Xaa can be any naturally occurring amino acid
  misc_feature (7)..(8): Xaa can be any naturally occurring amino acid
RXXRXRXX

SEQ ID NO: 1208 - protein, 13 aa, Artificial Sequence
  CPP
SAETVESCLAKSH

SEQ ID NO: 1209 - protein, 16 aa, Artificial Sequence
  CPP
SARHHCRSKAKRSRHH

SEQ ID NO: 1210 - protein, 12 aa, Artificial Sequence
  CPP
SATGAPWKMWVR

SEQ ID NO: 1211 - protein, 12 aa, Artificial Sequence
  CPP
SFHQFARATLAS

SEQ ID NO: 1212 - protein, 37 aa, Artificial Sequence
  CPP
SGRGKQGGKARAKAKTRSSRAGLQFPVGRVHRLLRKG

SEQ ID NO: 1213 - protein, 38 aa, Artificial Sequence
  CPP
SGRGKQGGKARAKAKTRSSRAGLQFPVGRVHRLLRKGC

SEQ ID NO: 1214 - protein, 12 aa, Artificial Sequence
  CPP
SHAFTWPTYLQL

SEQ ID NO: 1215 - protein, 12 aa, Artificial Sequence
  CPP
SHNWLPLWPLRP

SEQ ID NO: 1216 - protein, 8 aa, Artificial Sequence
  CPP
SKKKKTKV

SEQ ID NO: 1217 - protein, 60 aa, Artificial Sequence
  CPP
SKRTRQTYTRYQTLELEKEFHFNRYITRRRRIDIANALSLSERQIKIWFQNRRMKSKKDR

SEQ ID NO: 1218 - protein, 12 aa, Artificial Sequence
  CPP
SLGWMLPFSPPF

SEQ ID NO: 1219 - protein, 12 aa, Artificial Sequence
  CPP
SMLKRNHSTSNR

SEQ ID NO: 1220 - protein, 12 aa, Artificial Sequence
  CPP
SNPWDSLLSVST

SEQ ID NO: 1221 - protein, 12 aa, Artificial Sequence
  CPP
SPMQKTMNLPPM

SEQ ID NO: 1222 - protein, 14 aa, Artificial Sequence
  CPP
SQMTRQARRLYBGC

SEQ ID NO: 1223 - protein, 16 aa, Artificial Sequence
  CPP
SRAHHCRSKAKRSRHH

SEQ ID NO: 1224 - protein, 16 aa, Artificial Sequence
  CPP
SRRAHCRSKAKRSRHH

SEQ ID NO: 1225 - protein, 19 aa, Artificial Sequence
  CPP
SRRARRSPRESGKKRKRKR

SEQ ID NO: 1226 - protein, 14 aa, Artificial Sequence
  CPP
SRRARRSPRHLGSG

SEQ ID NO: 1227 - protein, 16 aa, Artificial Sequence
  CPP
SRRHACRSKAKRSRHH

SEQ ID NO: 1228 - protein, 16 aa, Artificial Sequence
  CPP
SRRHHARSKAKRSRHH

SEQ ID NO: 1229 - protein, 16 aa, Artificial Sequence
  CPP
SRRHHCRAKAKRSRHH

SEQ ID NO: 1230 - protein, 16 aa, Artificial Sequence
  CPP
SRRHHCRSAAKRSRHH

SEQ ID NO: 1231 - protein, 16 aa, Artificial Sequence
  CPP
SRRHHCRSKAARSRHH

SEQ ID NO: 1232 - protein, 16 aa, Artificial Sequence
  CPP
SRRHHCRSKAKASRHH

SEQ ID NO: 1233 - protein, 16 aa, Artificial Sequence
  CPP
SRRHHCRSKAKRARHH

SEQ ID NO: 1234 - protein, 16 aa, Artificial Sequence
  CPP
SRRHHCRSKAKRSAHH

SEQ ID NO: 1235 - protein, 12 aa, Artificial Sequence
  CPP
SRRKRQRSNMRI

SEQ ID NO: 1236 - protein, 10 aa, Artificial Sequence
  CPP
SRRRRRRRRR

SEQ ID NO: 1237 - protein, 10 aa, Artificial Sequence
  CPP
SRWRWKCCKK

SEQ ID NO: 1238 - protein, 10 aa, Artificial Sequence
  CPP
SRWRWKCSKK

SEQ ID NO: 1239 - protein, 10 aa, Artificial Sequence
  CPP
SRWRWKSCKK

SEQ ID NO: 1240 - protein, 10 aa, Artificial Sequence
  CPP
SRWRWKSSKK

SEQ ID NO: 1241 - protein, 12 aa, Artificial Sequence
  CPP
SSSIFPPWLSFF

SEQ ID NO: 1242 - protein, 12 aa, Artificial Sequence
  CPP
SWAQHLSLPPVL

SEQ ID NO: 1243 - protein, 12 aa, Artificial Sequence
  CPP
SWLPYPWHVPSS

SEQ ID NO: 1244 - protein, 12 aa, Artificial Sequence
  CPP
SWWTPWHVHSES

SEQ ID NO: 1245 - protein, 17 aa, Artificial Sequence
  CPP
  misc_feature (2)..(2): Xaa can be any naturally occurring amino acid
  misc_feature (5)..(5): Xaa can be any naturally occurring amino acid
  misc_feature (13)..(13): Xaa can be any naturally occurring amino acid
  misc_feature (16)..(16): Xaa can be any naturally occurring amino acid
SXRSXRILFQYSXRSXR

SEQ ID NO: 1246 - protein, 12 aa, Artificial Sequence
  CPP
SYIQRTPSTTLP

SEQ ID NO: 1247 - protein, 20 aa, Artificial Sequence
  CPP
TAKTRYKARRAELIAERRGC

SEQ ID NO: 1248 - protein, 36 aa, Artificial Sequence
  CPP
TAMRAVDKLLLHLKKLFREGQFNRNFESIIICRDRT

SEQ ID NO: 1249 - protein, 22 aa, Artificial Sequence
  CPP
TARRITPKDVIDVRSVTTEINT

SEQ ID NO: 1250 - protein, 8 aa, Artificial Sequence
  CPP
TCTWLKYH

SEQ ID NO: 1251 - protein, 9 aa, Artificial Sequence
  CPP
TCTWLKYHS

SEQ ID NO: 1252 - protein, 12 aa, Artificial Sequence
  CPP
TFPQTAIGVGAP

SEQ ID NO: 1253 - protein, 23 aa, Artificial Sequence
  CPP
TKAARITPKDVIDVRSVTTEINT

SEQ ID NO: 1254 - protein, 22 aa, Artificial Sequence
  CPP
TKRRITPDDVIDVRSVTTEINT

SEQ ID NO: 1255 - protein, 13 aa, Artificial Sequence
  CPP
TKRRITPKDVIDV

SEQ ID NO: 1256 - protein, 22 aa, Artificial Sequence
  CPP
TKRRITPKDVIDVESVTTEINT

SEQ ID NO: 1257 - protein, 22 aa, Artificial Sequence
  CPP
TKRRITPKDVIDVRSVTTEINT

SEQ ID NO: 1258 - protein, 22 aa, Artificial Sequence
  CPP
TKRRITPKDVIDVRSVTTKINT

SEQ ID NO: 1259 - protein, 22 aa, Artificial Sequence
  CPP
TKRRITPKKVIDVRSVTTEINT

SEQ ID NO: 1260 - protein, 12 aa, Artificial Sequence
  CPP
TLPSPLALLTVH

SEQ ID NO: 1261 - protein, 9 aa, Artificial Sequence
  CPP
TPFKLSLHL

SEQ ID NO: 1262 - protein, 12 aa, Artificial Sequence
  CPP
TPKTMTQTYDFS

SEQ ID NO: 1263 - protein, 24 aa, Artificial Sequence
  CPP
TPWWRLWTKWHHKRRDLPRKPEGC

SEQ ID NO: 1264 - protein, 17 aa, Artificial Sequence
  CPP
TRQARRNRRRRWRERQR

SEQ ID NO: 1265 - protein, 19 aa, Artificial Sequence
  CPP
TRQARRNRRRRWRERQRGC

SEQ ID NO: 1266 - protein, 15 aa, Artificial Sequence
  CPP
TRRQRTRRARRNRGC

SEQ ID NO: 1267 - protein, 12 aa, Artificial Sequence
  CPP
TRRSKRRSHRKF

SEQ ID NO: 1268 - protein, 24 aa, Artificial Sequence
  CPP
TRSSRAGLQWPVGRVHRLLRKGGC

SEQ ID NO: 1269 - protein, 12 aa, Artificial Sequence
  CPP
TSHTDAPPARSP

SEQ ID NO: 1270 - protein, 12 aa, Artificial Sequence
  CPP
TSPLNIHNGQKL

SEQ ID NO: 1271 - protein, 19 aa, Artificial Sequence
  CPP
TVDNPASTTNKDKLFAVRK

SEQ ID NO: 1272 - protein, 6 aa, Artificial Sequence
  CPP
TWLKYH

SEQ ID NO: 1273 - protein, 4 aa, Artificial Sequence
  CPP
VCVR

SEQ ID NO: 1274 - protein, 18 aa, Artificial Sequence
  CPP
VELPPPVELPPPVELPPP

SEQ ID NO: 1275 - protein, 27 aa, Artificial Sequence
  CPP
VGALAVVVWLWLWLWAGSGPKKKRKVC

SEQ ID NO: 1276 - protein, 6 aa, Artificial Sequence
  CPP
VHLPPP

SEQ ID NO: 1277 - protein, 12 aa, Artificial Sequence
  CPP
VHLPPPVHLPPP

SEQ ID NO: 1278 - protein, 18 aa, Artificial Sequence
  CPP
VHLPPPVHLPPPVHLPPP

SEQ ID NO: 1279 - protein, 13 aa, Artificial Sequence
  CPP
VIRVHFRLPVRTV

SEQ ID NO: 1280 - protein, 6 aa, Artificial Sequence
  CPP
VKLPPP

SEQ ID NO: 1281 - protein, 12 aa, Artificial Sequence
  CPP
VKLPPPVKLPPP

SEQ ID NO: 1282 - protein, 18 aa, Artificial Sequence
  CPP
VKLPPPVKLPPPVKLPPP

SEQ ID NO: 1283 - protein, 15 aa, Artificial Sequence
  CPP
VKRFKKFFRKLKKKV

SEQ ID NO: 1284 - protein, 15 aa, Artificial Sequence
  CPP
VKRFKKFFRKLKKLV

SEQ ID NO: 1285 - protein, 15 aa, Artificial Sequence
  CPP
VKRFKKFFRKLKKSV

SEQ ID NO: 1286 - protein, 19 aa, Artificial Sequence
  CPP
VKRGLKLRHVRPRVTRMDV

SEQ ID NO: 1287 - protein, 20 aa, Artificial Sequence
  CPP
VKRKKKPALWKTLLKKVLKA

SEQ ID NO: 1288 - protein, 5 aa, Artificial Sequence
  CPP
VLCLR

SEQ ID NO: 1289 - protein, 12 aa, Artificial Sequence
  CPP
VLGQSGYLMPMR

SEQ ID NO: 1290 - protein, 21 aa, Artificial Sequence
  CPP
VNADIKATTVFGGKYVSLTTP

SEQ ID NO: 1291 - protein, 5 aa, Artificial Sequence
  CPP
VPALK

SEQ ID NO: 1292 - protein, 5 aa, Artificial Sequence
  CPP
VPALR

SEQ ID NO: 1293 - protein, 5 aa, Artificial Sequence
  CPP
VPMIK

SEQ ID NO: 1294 - protein, 5 aa, Artificial Sequence
  CPP
VPMLK

SEQ ID NO: 1295 - protein, 5 aa, Artificial Sequence
  CPP
VPTLE

SEQ ID NO: 1296 - protein, 5 aa, Artificial Sequence
  CPP
VPTLK

SEQ ID NO: 1297 - protein, 5 aa, Artificial Sequence
  CPP
VPTLQ

SEQ ID NO: 1298 - protein, 15 aa, Artificial Sequence
  CPP
VQAILRRNWNQYKIQ

SEQ ID NO: 1299 - protein, 8 aa, Artificial Sequence
  CPP
VQLRRRWC

SEQ ID NO: 1300 - protein, 10 aa, Artificial Sequence
  CPP
VQRKRQKLMP

SEQ ID NO: 1301 - protein, 6 aa, Artificial Sequence
  CPP
VRLPPP

SEQ ID NO: 1302 - protein, 12 aa, Artificial Sequence
  CPP
VRLPPPVRLPPP

SEQ ID NO: 1303 - protein, 18 aa, Artificial Sequence
  CPP
VRLPPPVRLPPPVRLPPP

SEQ ID NO: 1304 - protein, 13 aa, Artificial Sequence
  CPP
VRRFLVTLRIRRA

SEQ ID NO: 1305 - protein, 5 aa, Artificial Sequence
  CPP
VSALK

SEQ ID NO: 1306 - protein, 5 aa, Artificial Sequence
  CPP
VSGKK

SEQ ID NO: 1307 - protein, 12 aa, Artificial Sequence
  CPP
VSKQPYYMWNGN

SEQ ID NO: 1308 - protein, 5 aa, Artificial Sequence
  CPP
VSLKK

SEQ ID NO: 1309 - protein, 14 aa, Artificial Sequence
  CPP
VSRRRRRRGGRRRR

SEQ ID NO: 1310 - protein, 15 aa, Artificial Sequence
  CPP
VSRRRRRRGGRRRRK

SEQ ID NO: 1311 - protein, 21 aa, Artificial Sequence
  CPP
VTPHHVLVDEYTGEWVDSQFK

SEQ ID NO: 1312 - protein, 17 aa, Artificial Sequence
  CPP
VVLGKLYGRKKRRQRRR

SEQ ID NO: 1313 - protein, 4 aa, Artificial Sequence
  CPP
VVVR

SEQ ID NO: 1314 - protein, 15 aa, Artificial Sequence
  CPP
VWPLGLVICKALKIC

SEQ ID NO: 1315 - protein, 13 aa, Artificial Sequence
  CPP
WEYGRKKRRQRRR

SEQ ID NO: 1316 - protein, 30 aa, Artificial Sequence
  CPP
WEAALAEALAEALAEHLAEALAEALEALAA

SEQ ID NO: 1317 - protein, 30 aa, Artificial Sequence
  CPP
WEAKLAKALAKALAKHLAKALAKALKACEA

SEQ ID NO: 1318 - protein, 24 aa, Artificial Sequence
  CPP
WEARLARALARALARHLARALARA

SEQ ID NO: 1319 - protein, 30 aa, Artificial Sequence
  CPP
WEARLARALARALARHLARALARALRACEA

SEQ ID NO: 1320 - protein, 17 aa, Artificial Sequence
  CPP
WEAVVAYGRKKRRQRRR

SEQ ID NO: 1321 - protein, 17 aa, Artificial Sequence
  CPP
WEAVVLYGRKKRRQRRR

SEQ ID NO: 1322 - protein, 14 aa, Artificial Sequence
  CPP
WELYGRKKRRQRRR

SEQ ID NO: 1323 - protein, 15 aa, Artificial Sequence
  CPP
WELVYGRKKRRQRRR

SEQ ID NO: 1324 - protein, 16 aa, Artificial Sequence
  CPP
WELVVYGRKKRRQRRR

SEQ ID NO: 1325 - protein, 17 aa, Artificial Sequence
  CPP
WELVVAYGRKKRRQRRR

SEQ ID NO: 1326 - protein, 17 aa, Artificial Sequence
  CPP
WELVVLYGRKKRRQRRR

SEQ ID NO: 1327 - protein, 18 aa, Artificial Sequence
  CPP
WELVVLGYGRKKRRQRRR

SEQ ID NO: 1328 - protein, 19 aa, Artificial Sequence
  CPP
WELVVLGKYGRKKRRQRRR

SEQ ID NO: 1329 - protein, 20 aa, Artificial Sequence
  CPP
WELVVLGKLYGRKKRRQRRR

SEQ ID NO: 1330 - protein, 11 aa, Artificial Sequence
  CPP
WFQNRRMKWKK

SEQ ID NO: 1331 - protein, 12 aa, Artificial Sequence
  CPP
WIIFKIAASHKK

SEQ ID NO: 1332 - protein, 12 aa, Artificial Sequence
  CPP
WIIFRAAASHKK

SEQ ID NO: 1333 - protein, 12 aa, Artificial Sequence
  CPP
WIIFRALISHKK

SEQ ID NO: 1334 - protein, 12 aa, Artificial Sequence
  CPP
WIIFRIAASHKK

SEQ ID NO: 1335 - protein, 12 aa, Artificial Sequence
  CPP
WIIFRIAATHKK

SEQ ID NO: 1336 - protein, 12 aa, Artificial Sequence
  CPP
WIIFRIAAYHKK

SEQ ID NO: 1337 - protein, 15 aa, Artificial Sequence
  CPP
WKARRQCFRVLHHWN

SEQ ID NO: 1338 - protein, 15 aa, Artificial Sequence
  CPP
WKCRRQAFRVLHHWN

SEQ ID NO: 1339 - protein, 15 aa, Artificial Sequence
  CPP
WKCRRQCFRVLHHWN

SEQ ID NO: 1340 - protein, 14 aa, Artificial Sequence
  CPP
WKQSHKKGGKKGSG

SEQ ID NO: 1341 - protein, 19 aa, Artificial Sequence
  CPP
WLKLLKKWLKLWKKLLKLW

SEQ ID NO: 1342 - protein, 12 aa, Artificial Sequence
  CPP
WLKLWKKWLKLW

SEQ ID NO: 1343 - protein, 20 aa, Artificial Sequence
  CPP
WLKYLLKKWLKLWKKLLKLW

SEQ ID NO: 1344 - protein, 19 aa, Artificial Sequence
  CPP
WLKLLKKWLKLWKKLLKLW

SEQ ID NO: 1345 - protein, 19 aa, Artificial Sequence
  CPP
WLKLLRKWLRLWKRLLKLW

SEQ ID NO: 1346 - protein, 19 aa, Artificial Sequence
  CPP
WLRLLKRWLKLWRKLLRLW

SEQ ID NO: 1347 - protein, 23 aa, Artificial Sequence
  CPP
WLRRIKAWLRRIKALNRQLGVAA

SEQ ID NO: 1348 - protein, 20 aa, Artificial Sequence
  CPP
WRFKAAVALLPAVLLALLAP

SEQ ID NO: 1349 - protein, 10 aa, Artificial Sequence
  CPP
WRFKKSKRKV

SEQ ID NO: 1350 - protein, 8 aa, Artificial Sequence
  CPP
WRFKWRFK

SEQ ID NO: 1351 - protein, 12 aa, Artificial Sequence
  CPP
WRFKWRFKWRFK

SEQ ID NO: 1352 - protein, 9 aa, Artificial Sequence
  CPP
WRRRRRRRR

SEQ ID NO: 1353 - protein, 8 aa, Artificial Sequence
  CPP
WRWKKKKA

SEQ ID NO: 1354 - protein, 14 aa, Artificial Sequence
  CPP
WRWRWRWRWRWRWR

SEQ ID NO: 1355 - protein, 10 aa, Artificial Sequence
  CPP
WWRRRRRRRR

SEQ ID NO: 1356 - protein, 11 aa, Artificial Sequence
  CPP
WWWRRRRRRRR

SEQ ID NO: 1357 - protein, 12 aa, Artificial Sequence
  CPP
WWWWRRRRRRRR

SEQ ID NO: 1358 - protein, 20 aa, Artificial Sequence
  CPP
YWLKLLKKWLKLWKKLLKLW

SEQ ID NO: 1359 - protein, 11 aa, Artificial Sequence
  CPP
YARAAARQARA

SEQ ID NO: 1360 - protein, 22 aa, Artificial Sequence
  CPP
YARAAARQARAKALARQLGVAA

SEQ ID NO: 1361 - protein, 11 aa, Artificial Sequence
  CPP
YARAARRAARR

SEQ ID NO: 1362 - protein, 11 aa, Artificial Sequence
  CPP
YAREARRAARR

SEQ ID NO: 1363 - protein, 11 aa, Artificial Sequence
  CPP
YARKARRAARR

SEQ ID NO: 1364 - protein, 11 aa, Artificial Sequence
  CPP
YARVRRRGPRR

SEQ ID NO: 1365 - protein, 11 aa, Artificial Sequence
  CPP
YEREARRAARR

SEQ ID NO: 1366 - protein, 34 aa, Artificial Sequence
  CPP
YGDCLPHLKLCKENKDCCSKKCKRRGTNIEKRCR

SEQ ID NO: 1367 - protein, 11 aa, Artificial Sequence
  CPP
YGRAARRAARR

SEQ ID NO: 1368 - protein, 11 aa, Artificial Sequence
  CPP
YGRGGRRGRRR

SEQ ID NO: 1369 - protein, 12 aa, Artificial Sequence
  CPP
YGRKKKRRQRRR

SEQ ID NO: 1370 - protein, 11 aa, Artificial Sequence
  CPP
YGRKKRPQRRR

SEQ ID NO: 1371 - protein, 11 aa, Artificial Sequence
  CPP
YGRKKRRQRRR

SEQ ID NO: 1372 - protein, 16 aa, Artificial Sequence
  CPP
YGRKKRRQRRRDYQQD

SEQ ID NO: 1373 - protein, 18 aa, Artificial Sequence
  CPP
YGRKKRRQRRRENAEYLR

SEQ ID NO: 1374 - protein, 16 aa, Artificial Sequence
  CPP
YGRKKRRQRRRNYQQN

SEQ ID NO: 1375 - protein, 11 aa, Artificial Sequence
  CPP
YGRKKRRQRRR

SEQ ID NO: 1376 - protein, 18 aa, Artificial Sequence
  CPP
YGRKKRRQRRRQNAQYLR

SEQ ID NO: 1377 - protein, 12 aa, Artificial Sequence
  CPP
YGRKKRRQRRRC

SEQ ID NO: 1378 - protein, 29 aa, Artificial Sequence
  CPP
YGRKKRRQRRRAYFNGCSSPTAPLSPMSP

SEQ ID NO: 1379 - protein, 12 aa, Artificial Sequence
  CPP
YGRKKRRQRRRC

SEQ ID NO: 1380 - protein, 48 aa, Artificial Sequence
  CPP
YGRKKRRQRRRDPYHATSGALSPAKDCGSQKYAYFNGCSSPTLSPMSP

SEQ ID NO: 1381 - protein, 13 aa, Artificial Sequence
  CPP
YGRKKRRQRRRGC

SEQ ID NO: 1382 - protein, 25 aa, Artificial Sequence
  CPP
YGRKKRRQRRRGCYGRKKRRQRRRG

SEQ ID NO: 1383 - protein, 34 aa, Artificial Sequence
  CPP
YGRKKRRQRRRGLFGAIAGFIENGWEGMIDGWYG

SEQ ID NO: 1384 - protein, 23 aa, Artificial Sequence
  CPP
YGRKKRRQRRRGTALDWSWLQTE

SEQ ID NO: 1385 - protein, 15 aa, Artificial Sequence
  CPP
YGRKKRRQRRRPPQG

SEQ ID NO: 1386 - protein, 25 aa, Artificial Sequence
  CPP
YGRKKRRQRRRQRRRPTAPLSPMSP

SEQ ID NO: 1387 - protein, 22 aa, Artificial Sequence
  CPP
YGRKKRRQRRRYGRKKRRQRRR

SEQ ID NO: 1388 - protein, 33 aa, Artificial Sequence
  CPP
YGRKKRRQRRRYGRKKRRQRRRYGRKKRRQRRR

SEQ ID NO: 1389 - protein, 21 aa, Artificial Sequence
  CPP
YGRKKRRQRRTALDASALQTE

SEQ ID NO: 1390 - protein, 21 aa, Artificial Sequence
  CPP
YGRKKRRQRRTALDWSWLQTE

SEQ ID NO: 1391 - protein, 11 aa, Artificial Sequence
  CPP
YGRRARRAARR

SEQ ID NO: 1392 - protein, 11 aa, Artificial Sequence
  CPP
YGRRARRRARR

SEQ ID NO: 1393 - protein, 11 aa, Artificial Sequence
  CPP
YGRRARRRRRR

SEQ ID NO: 1394 - protein, 11 aa, Artificial Sequence
  CPP
YGRRRRRRRRR

SEQ ID NO: 1395 - protein, 16 aa, Artificial Sequence
  CPP
YIVLRRRRKRVNTKRS

SEQ ID NO: 1396 - protein, 12 aa, Artificial Sequence
  CPP
YKALRISRKLAK

SEQ ID NO: 1397 - protein, 14 aa, Artificial Sequence
  CPP
YKQCHKKGGKKGSG

SEQ ID NO: 1398 - protein, 42 aa, Artificial Sequence
  CPP
YKQCHKKGGHCFPKEKICLPPSSDFGKMDCRWRWKCCKKGSG

SEQ ID NO: 1399 - protein, 14 aa, Artificial Sequence
  CPP
YKQCHKKGGKKGSG

SEQ ID NO: 1400 - protein, 15 aa, Artificial Sequence
  CPP
  misc_feature (10)..(10): Xaa can be any naturally occurring amino acid
YKQCHKKGGXKKGSG

SEQ ID NO: 1401 - protein, 14 aa, Artificial Sequence
  CPP
YKQSHKKGGKKGSG

SEQ ID NO: 1402 - protein, 11 aa, Artificial Sequence
  CPP
YKRAARRAARR

SEQ ID NO: 1403 - protein, 11 aa, Artificial Sequence
  CPP
YKRKARRAARR

SEQ ID NO: 1404 - protein, 10 aa, Artificial Sequence
  CPP
YNNFAYSVFL

SEQ ID NO: 1405 - protein, 11 aa, Artificial Sequence
  CPP
YPRAARRAARR

SEQ ID NO: 1406 - protein, 12 aa, Artificial Sequence
  CPP
YPYDANHTRSPT

SEQ ID NO: 1407 - protein, 10 aa, Artificial Sequence
  CPP
YQKQAKIMCS

SEQ ID NO: 1408 - protein, 10 aa, Artificial Sequence
  CPP
YRDRFAFQPH

SEQ ID NO: 1409 - protein, 4 aa, Artificial Sequence
  CPP
YRFK

SEQ ID NO: 1410 - protein, 13 aa, Artificial Sequence
  CPP
YRFKYRFKYRLFK

SEQ ID NO: 1411 - protein, 14 aa, Artificial Sequence
  CPP
YRQSHRRGGRRGSG

SEQ ID NO: 1412 - protein, 11 aa, Artificial Sequence
  CPP
YRRAARRAARA

SEQ ID NO: 1413 - protein, 11 aa, Artificial Sequence
  CPP
YRRRRRRRRRR

SEQ ID NO: 1414 - protein, 8 aa, Artificial Sequence
  CPP
YRWRCKNQ

SEQ ID NO: 1415 - protein, 9 aa, Artificial Sequence
  CPP
YRWRCKNQN

SEQ ID NO: 1416 - protein, 12 aa, Artificial Sequence
  CPP
YSHIATLPFTPT

SEQ ID NO: 1417 - protein, 24 aa, Artificial Sequence
  CPP
YSSYSAPVSSSLSVRRSYSSSSGS

SEQ ID NO: 1418 - protein, 16 aa, Artificial Sequence
  CPP
YTAIAWVKAFIRKLRK

SEQ ID NO: 1419 - protein, 12 aa, Artificial Sequence
  CPP
YTFGLKTSFNVQ

SEQ ID NO: 1420 - protein, 24 aa, Artificial Sequence
  CPP
YTFGLKTSFNVQYTFGLKTSFNVQ

SEQ ID NO: 1421 - protein, 21 aa, Artificial Sequence
  CPP
YTQDFNKFHTFPQTAIGVGAP

SEQ ID NO: 1422 - protein, 13 aa, Artificial Sequence
  CPP
YYYAAGRKRKKRT

SEQ ID NO: 1423 - protein, 7 aa, Artificial Sequence
  SV40 NLS
PKKKRKV

SEQ ID NO: 1424 - DNA, 20 bp, Artificial Sequence
  sgRNA
cgtgtgctcgggtatcccaa

SEQ ID NO: 1425 - DNA, 20 bp, Artificial Sequence
  sgRNA
ccgccatgagaataacacca

SEQ ID NO: 1426 - DNA, 20 bp, Artificial Sequence
  sgRNA
ccttggtgttattctcatgg

SEQ ID NO: 1427 - DNA, 20 bp, Artificial Sequence
  sgRNA
ttgtcaagctaaggcgacag

SEQ ID NO: 1428 - DNA, 20 bp, Artificial Sequence
  sgRNA
tcacaataatcagaaacacc

SEQ ID NO: 1429 - DNA, 20 bp, Artificial Sequence
  crRNA
ttcagtggtcccattgtggt

SEQ ID NO: 1430 - DNA, 20 bp, Artificial Sequence
  crRNA
gtggaatacaatcagtttgg

SEQ ID NO: 1431 - DNA, 20 bp, Artificial Sequence
  crRNA
ttctcggcttccaggccttc

SEQ ID NO: 1432 - DNA, 20 bp, Artificial Sequence
  crRNA
cattctctgctggatgacgt

SEQ ID NO: 1433 - DNA, 20 bp, Artificial Sequence
  crRNA
aattctctctccattcttca

SEQ ID NO: 1434 - protein, 14 aa, Artificial Sequence
  endosomal escape peptide
FLFPLITSFLSKVL

SEQ ID NO: 1435 - protein, 13 aa, Artificial Sequence
  endosomal escape peptide
FISAIASMLGKFL

SEQ ID NO: 1436 - protein, 13 aa, Artificial Sequence
  endosomal escape peptide
GWFDVVKHIASAV

SEQ ID NO: 1437 - protein, 13 aa, Artificial Sequence
  endosomal escape peptide
FFGSVLKLIPKIL

SEQ ID NO: 1438 - protein, 13 aa, Artificial Sequence
  endosomal escape peptide
GLFDIIKKIAESF

SEQ ID NO: 1439 - protein, 13 aa, Artificial Sequence
  endosomal escape peptide
HGVSGHGQHGVHG

SEQ ID NO: 1440 - protein, 13 aa, Artificial Sequence
  endosomal escape peptide
FLPLIGRVLSGIL

SEQ ID NO: 1441 - protein, 13 aa, Artificial Sequence
  endosomal escape peptide
GLFDIIKKIAESI

SEQ ID NO: 1442 - protein, 16 aa, Artificial Sequence
  endosomal escape peptide
GLLDIVKKVVGAFGSL

SEQ ID NO: 1443 - protein, 16 aa, Artificial Sequence
  endosomal escape peptide
GLFDIVKKVVGALGSL

SEQ ID NO: 1444 - protein, 16 aa, Artificial Sequence
  endosomal escape peptide
GLFDIVKKVVGAIGSL

SEQ ID NO: 1445 - protein, 16 aa, Artificial Sequence
  endosomal escape peptide
GLFDIVKKVVGTLAGL

SEQ ID NO: 1446 - protein, 16 aa, Artificial Sequence
  endosomal escape peptide
GLFDIVKKVVGAFGSL

SEQ ID NO: 1447 - protein, 16 aa, Artificial Sequence
  endosomal escape peptide
GLFDIAKKVIGVIGSL

SEQ ID NO: 1448 - protein, 17 aa, Artificial Sequence
  endosomal escape peptide
GLFDIVKKIAGHIAGSI

SEQ ID NO: 1449 - protein, 17 aa, Artificial Sequence
  endosomal escape peptide
GLFDIVKKIAGHIASSI

SEQ ID NO: 1450 - protein, 17 aa, Artificial Sequence
  endosomal escape peptide
GLFDIVKKIAGHIVSSI

SEQ ID NO: 1451 - protein, 13 aa, Artificial Sequence
  endosomal escape peptide
FVQWFSKFLGRIL

SEQ ID NO: 1452 - protein, 16 aa, Artificial Sequence
  endosomal escape peptide
GLFDVIKKVASVIGGL

SEQ ID NO: 1453 - protein, 16 aa, Artificial Sequence
  endosomal escape peptide
GLFDIIKKVASVVGGL

SEQ ID NO: 1454 - protein, 16 aa, Artificial Sequence
  endosomal escape peptide
GLFDIIKKVASVIGGL

SEQ ID NO: 1455 - protein, 15 aa, Artificial Sequence
  endosomal escape peptide
VWPLGLVICKALKIC

SEQ ID NO: 1456 - protein, 14 aa, Artificial Sequence
  endosomal escape peptide
NFLGTLVNLAKKIL

SEQ ID NO: 1457 - protein, 13 aa, Artificial Sequence
  endosomal escape peptide
FLPLIGKILGTIL

SEQ ID NO: 1458 - protein, 13 aa, Artificial Sequence
  endosomal escape peptide
FLPIIAKVLSGLL

SEQ ID NO: 1459 - protein, 13 aa, Artificial Sequence
  endosomal escape peptide
FLPIVGKLLSGLL

SEQ ID NO: 1460 - protein, 13 aa, Artificial Sequence
  endosomal escape peptide
FLSSIGKILGNLL

SEQ ID NO: 1461 - protein, 13 aa, Artificial Sequence
  endosomal escape peptide
FLSGIVGMLGKLF

SEQ ID NO: 1462 - protein, 9 aa, Artificial Sequence
  endosomal escape peptide
TPFKLSLHL

SEQ ID NO: 1463 - protein, 14 aa, Artificial Sequence
  endosomal escape peptide
GILDAIKAIAKAAG

SEQ ID NO: 1464 - protein, 12 aa, Artificial Sequence
  endosomal escape peptide
LFDIIKKIAESF

SEQ ID NO: 1465 - protein, 25 aa, Artificial Sequence
  endosomal escape peptide
LFDIIKKIAESGFLFDIIKKIAESF

SEQ ID NO: 1466 - protein, 24 aa, Artificial Sequence
  endosomal escape peptide
GLLNGLALRLGKRALKKIIKRLCR

SEQ ID NO: 1467 - protein, 14 aa, Artificial Sequence
  endosomal escape peptide
GHHHHHHHHHHHHH

SEQ ID NO: 1468 - protein, 10 aa, Artificial Sequence
  endosomal escape peptide
FKCRRWQWRM

SEQ ID NO: 1469 - protein, 10 aa, Artificial Sequence
  endosomal escape peptide
KTCENLADTY

SEQ ID NO: 1470 - protein, 13 aa, Artificial Sequence
  endosomal escape peptide
ALFDIIKKIAESF

SEQ ID NO: 1471 - protein, 13 aa, Artificial Sequence
  endosomal escape peptide
GAFDIIKKIAESF

SEQ ID NO: 1472 - protein, 13 aa, Artificial Sequence
  endosomal escape peptide
GLADIIKKIAESF

SEQ ID NO: 1473 - protein, 13 aa, Artificial Sequence
  endosomal escape peptide
GLFAIIKKIAESF

SEQ ID NO: 1474 - protein, 13 aa, Artificial Sequence
  endosomal escape peptide
GLFDAIKKIAESF

SEQ ID NO: 1475 - protein, 13 aa, Artificial Sequence
  endosomal escape peptide
GLFDIAKKIAESF

SEQ ID NO: 1476 - protein, 13 aa, Artificial Sequence
  endosomal escape peptide
GLFDIIAKIAESF

SEQ ID NO: 1477 - protein, 13 aa, Artificial Sequence
  endosomal escape peptide
GLFDIIKAIAESF

SEQ ID NO: 1478 - protein, 13 aa, Artificial Sequence
  endosomal escape peptide
GLFDIIKKAAESF

SEQ ID NO: 1479 - protein, 13 aa, Artificial Sequence
  endosomal escape peptide
GLFDIIKKIAASF

SEQ ID NO: 1480 - protein, 13 aa, Artificial Sequence
  endosomal escape peptide
GLFDIIKKIAEAF

SEQ ID NO: 1481 - protein, 13 aa, Artificial Sequence
  endosomal escape peptide
GLFDIIKKIAESA

SEQ ID NO: 1482 - protein, 13 aa, Artificial Sequence
  endosomal escape peptide
GLFDIIHKIAESF

SEQ ID NO: 1483 - protein, 13 aa, Artificial Sequence
  endosomal escape peptide
GLFDIIKHIAESF

SEQ ID NO: 1484 - protein, 13 aa, Artificial Sequence
  endosomal escape peptide
GLFDIIKKIAHSF

SEQ ID NO: 1485 - protein, 13 aa, Artificial Sequence
  endosomal escape peptide
GLFDIIRKIAESF

SEQ ID NO: 1486 - protein, 13 aa, Artificial Sequence
  endosomal escape peptide
GLFDIIKRIAESF

SEQ ID NO: 1487 - protein, 13 aa, Artificial Sequence
  endosomal escape peptide
GLFDIIKKIARSF

SEQ ID NO: 1488 - protein, 13 aa, Artificial Sequence
  endosomal escape peptide
GLFDIIKKIADSF

SEQ ID NO: 1489 - protein, 20 aa, Artificial Sequence
  endosomal escape peptide
GDIMGEWGNEIFGAIAGFLG

SEQ ID NO: 1490 - protein, 20 aa, Artificial Sequence
  endosomal escape peptide
GLFGAIAGFIENGWEGMIDG

SEQ ID NO: 1491 - protein, 23 aa, Artificial Sequence
  endosomal escape peptide
GLFEAIEGFIENGWEGMIDGWYG

SEQ ID NO: 1492 - protein, 25 aa, Artificial Sequence
  endosomal escape peptide
GLFEAIAEFIEGGWEGLIEGCAKKK

SEQ ID NO: 1493 - protein, 20 aa, Artificial Sequence
  endosomal escape peptide
GLFGAIAGFIENGQWGMIDG

SEQ ID NO: 1494 - protein, 48 aa, Artificial Sequence
  endosomal escape peptide
GLFEAIEGFIENGWEGMIDGWYGCGLFEAIEGFIENGWEGMIDGWYGC

SEQ ID NO: 1495 - protein, 11 aa, Artificial Sequence
  endosomal escape peptide
GLLEALAELLE

SEQ ID NO: 1496 - protein, 14 aa, Artificial Sequence
  endosomal escape peptide
LAEALAEALEALAA

SEQ ID NO: 1497 - protein, 26 aa, Artificial Sequence
  endosomal escape peptide
GIGAVLKVLTTGLPALISWIKRKRQQ

SEQ ID NO: 1498 - protein, 26 aa, Artificial Sequence
  endosomal escape peptide
CIGAVLKVLTTGLPALISWIKRKRQQ

SEQ ID NO: 1499 - protein, 16 aa, Artificial Sequence
  endosomal escape peptide
RQIKIWFQNRRMKWKK

SEQ ID NO: 1500 - protein, 30 aa, Artificial Sequence; identical to SEQ ID NO: 877
  endosomal escape peptide

SEQ ID NO: 1501 - protein, 24 aa, Artificial Sequence
  endosomal escape peptide
GALFLGWLGAAGSTMGAPKSKRKV

SEQ ID NO: 1502 - protein, 23 aa, Artificial Sequence
  endosomal escape peptide
LIRLWSHLIHIWFQNRRLKWKKK

SEQ ID NO: 1503 - protein, 23 aa, Artificial Sequence
  endosomal escape peptide
GLFEAIAEFIENGWEGLIEGWYG

SEQ ID NO: 1504 - protein, 34 aa, Artificial Sequence
  endosomal escape peptide
CKYGRRRQRRKKRGGDIMGEWGNEIFGAIAGFLG

SEQ ID NO: 1505 - protein, 27 aa, Artificial Sequence
  endosomal escape peptide
GLFEAIEGFIENGWEGMIWDYGSGSCG

SEQ ID NO: 1506 - protein, 21 aa, Artificial Sequence
  endosomal escape peptide
KETWWETWWTEWSQPKKKRKV

SEQ ID NO: 1507 - protein, 19 aa, Artificial Sequence
  endosomal escape peptide
LLIILRRRRIRKQAHAHSK

SEQ ID NO: 1508 - protein, 26 aa, Artificial Sequence
  endosomal escape peptide
DPKGDPKGVTVTVTVTVTGKGDPKPD

SEQ ID NO: 1509 - protein, 17 aa, Artificial Sequence
  endosomal escape peptide
CSIPPEVKFNKPFVYLI

SEQ ID NO: 1510 - protein, 27 aa, Artificial Sequence
  endosomal escape peptide
GWTLNSAGYLLGKINLKALAALAKKIL

SEQ ID NO: 1511 - protein, 21 aa, Artificial Sequence
  endosomal escape peptide
AGYLLGKINLKALAALAKKIL

SEQ ID NO: 1512 - protein, 17 aa, Artificial Sequence
  endosomal escape peptide
GALFLGFLGAAGSTMGA

SEQ ID NO: 1513 - protein, 20 aa, Artificial Sequence
  endosomal escape peptide
HGLASTLTRWAHYNALIRAF

SEQ ID NO: 1514 - protein, 20 aa, Artificial Sequence
  endosomal escape peptide
GLWRALWRLLRSLWRLLWRA

SEQ ID NO: 1515 - protein, 30 aa, Artificial Sequence; identical to SEQ ID NO: 1316
  endosomal escape peptide

SEQ ID NO: 1516 - protein, 24 aa, Artificial Sequence
  endosomal escape peptide
GLFEAIEGFIENGWEGMIDGWYGC

SEQ ID NO: 1517 - protein, 23 aa, Artificial Sequence
  endosomal escape peptide
GLFGAIAGFIENGWEGMIDGWYG

SEQ ID NO: 1518 - protein, 36 aa, Artificial Sequence
  endosomal escape peptide
GLFGAIAGFIENGWEGMIDGRQIKIWFQNRRMKWKK

SEQ ID NO: 1519 - protein, 26 aa, Artificial Sequence
  endosomal escape peptide
GLFGAIAGFIENGWEGMIDGSSKKKK

SEQ ID NO: 1520 - protein, 24 aa, Artificial Sequence
  endosomal escape peptide
GLFEAIAGFIENGWEGMIDGGGYC

SEQ ID NO: 1521 - protein, 23 aa, Artificial Sequence
  endosomal escape peptide
GLFHAIAHFIHGGWHGLIHGWYG

SEQ ID NO: 1522 - protein, 30 aa, Artificial Sequence
  endosomal escape peptide
GLFEAIEGFIENGWEGLAEALAEALEALAA

SEQ ID NO: 1523 - protein, 29 aa, Artificial Sequence
  endosomal escape peptide
KWKLFKKIGAVLKVLTTGYGRKKRRQRRR